SEQUENCE LISTING (142 sequences)

SEQ ID NO: 1 - DNA, 19 bp, Artificial Sequence
  Primer SEQ ID No. 1
cctccccacccatttcacc

SEQ ID NO: 2 - DNA, 20 bp, Artificial Sequence
  Primer SEQ ID No. 2
ctgtaagcactgtgaggagc

SEQ ID NO: 3 - DNA, 20 bp, Artificial Sequence
  Primer SEQ ID No. 3
cctaccatcctatgagcgag

SEQ ID NO: 4 - DNA, 21 bp, Artificial Sequence
  Primer SEQ ID No. 4
ccctacaagttggcagaagtg

SEQ ID NO: 5 - DNA, 24 bp, Artificial Sequence
  Primer SEQ ID No. 5
ctactgagaagaatgctttgtcta

SEQ ID NO: 6 - DNA, 23 bp, Artificial Sequence
  Primer SEQ ID No. 6
gcctgaacttaatattggagagg

SEQ ID NO: 7 - DNA, 26 bp, Artificial Sequence
  Primer SEQ ID No. 7
ctactgagaagaatgcttttaattcc

SEQ ID NO: 8 - DNA, 23 bp, Artificial Sequence
  Primer SEQ ID No. 8
gcctgaacttaatattggagagg

SEQ ID NO: 9 - DNA, 24 bp, Artificial Sequence
  Primer SEQ ID No. 9
ctactgagaagaatgctttgtcta

SEQ ID NO: 10 - DNA, 20 bp, Artificial Sequence
  Primer SEQ ID No. 10
ctcttggtagtagtcggtgc

SEQ ID NO: 11 - DNA, 20 bp, Artificial Sequence
  Primer SEQ ID No 11
ccagcaccgactactaccaa

SEQ ID NO: 12 - DNA, 20 bp, Artificial Sequence
  Primer SEQ ID No. 12
ctcttggtagtagtcggtgc

SEQ ID NO: 13 - DNA, 20 bp, Artificial Sequence
  Primer SEQ ID No. 13
caccgactactaccaagagc

SEQ ID NO: 14 - DNA, 26 bp, Artificial Sequence
  Primer SEQ ID No. 14
ctactgagaagaatgcttttaattcc

SEQ ID NO: 15 - DNA, 23 bp, Artificial Sequence
  Primer SEQ ID No. 15
gcctgaacttaatattggagagg

SEQ ID NO: 16 - DNA, 26 bp, Artificial Sequence
  Primer SEQ ID No. 16
ctactgagaagaatgcttttaattcc

SEQ ID NO: 17 - DNA, 20 bp, Artificial Sequence
  Primer SEQ ID No. 17
cggcactgacagacagccat

SEQ ID NO: 18 - DNA, 20 bp, Artificial Sequence
  Primer SEQ ID No. 18
ggcactgacagacagccatt

SEQ ID NO: 19 - DNA, 26 bp, Artificial Sequence
  Priemr SEQ ID No. 19
ctactgagaagaatgcttttaattcc

SEQ ID NO: 20 - DNA, 19 bp, Artificial Sequence
  Primer SEQ ID No. 20
caccccagccccagacatt

SEQ ID NO: 21 - DNA, 24 bp, Artificial Sequence
  Primer SEQ ID No. 21
ctactgagaagaatgcttttttgc

SEQ ID NO: 22 - DNA, 22 bp, Artificial Sequence
  Primer SEQ ID No. 22
aggctgcttccgttttatactg

SEQ ID NO: 23 - DNA, 24 bp, Artificial Sequence
  Primer SEQ ID No. 23
cctctccaatattaagttcagtga

SEQ ID NO: 24 - DNA, 21 bp, Artificial Sequence
  Primer SEQ ID No. 24
acagacagccaaggcaatgag

SEQ ID NO: 25 - DNA, 24 bp, Artificial Sequence
  Primer SEQ ID No. 25
cctctccaatattaagttcagtct

SEQ ID NO: 26 - DNA, 23 bp, Artificial Sequence
  Primer SEQ ID No. 26
cctctccaatattaagttcagtc

SEQ ID NO: 27 - DNA, 21 bp, Artificial Sequence
  Primer SEQ ID No. 27
acagacagccaaggcaatgag

SEQ ID NO: 28 - DNA, 21 bp, Artificial Sequence
  Primer SEQ ID No. 28
ccactctgatactcctaccac

SEQ ID NO: 29 - DNA, 22 bp, Artificial Sequence
  Primer SEQ ID No 29
gaaagagaccccagtagacaac

SEQ ID NO: 30 - DNA, 19 bp, Artificial Sequence
  Primer SEQ ID No. 30
cctccccacccatttcacc

SEQ ID NO: 31 - DNA, 20 bp, Artificial Sequence
  Primer SEQ ID No. 31
ctgtaagcactgtgaggagc

SEQ ID NO: 32 - DNA, 21 bp, Artificial Sequence
  Primer SEQ ID No. 32
cacttctgccaacttgtaggg

SEQ ID NO: 33 - DNA, 21 bp, Artificial Sequence
  Primer SEQ ID No. 33
ccctacaagttggcagaagtg

SEQ ID NO: 34 - DNA, 22 bp, Artificial Sequence
  Probe SEQ ID No. 34
tgacaccgggcacccagtctcc

SEQ ID NO: 35 - DNA, 22 bp, Artificial Sequence
  Probe SEQ ID No. 35
ccaccatgacaccgggcaccca

SEQ ID NO: 36 - DNA, 23 bp, Artificial Sequence
  Probe SEQ ID No. 36
tgcaggtaatggtggcagcagcc

SEQ ID NO: 37 - DNA, 26 bp, Artificial Sequence
  Probe SEQ ID No. 37
agcaccgactactaccaagagctgca

SEQ ID NO: 38 - DNA, 33 bp, Artificial Sequence
  Probe SEQ ID No. 38
tttcctgtcttttcacatttcaaacctccagtt

SEQ ID NO: 39 - DNA, 26 bp, Artificial Sequence
  Probe SEQ ID No. 39
cagcaccgactactaccaagagctgc

SEQ ID NO: 40 - DNA, 33 bp, Artificial Sequence; identical to SEQ ID NO: 38
  Probe SEQ ID No. 40

SEQ ID NO: 41 - DNA, 25 bp, Artificial Sequence
  Probe SEQ ID No. 41
atggctgtctgtcagtgccgccgaa

SEQ ID NO: 42 - DNA, 26 bp, Artificial Sequence
  Probe SEQ ID No. 42
agcaccgactactaccaagagctgca

SEQ ID NO: 43 - DNA, 26 bp, Artificial Sequence
  Probe SEQ ID No. 43
cagcaccgactactaccaagagctgc

SEQ ID NO: 44 - DNA, 26 bp, Artificial Sequence
  Probe SEQ ID No. 44
agcaccgactactaccaagagctgca

SEQ ID NO: 45 - DNA, 26 bp, Artificial Sequence
  Probe SEQ ID No. 45
agcaccgactactaccaagagctgca

SEQ ID NO: 46 - DNA, 26 bp, Artificial Sequence
  Probe SEQ ID No. 46
cagcaccgactactaccaagagctgc

SEQ ID NO: 47 - DNA, 26 bp, Artificial Sequence
  Probe SEQ ID No. 47
ttgactctggccttccgagaaggtac

SEQ ID NO: 48 - DNA, 28 bp, Artificial Sequence
  Probe SEQ ID No. 48
cttccgagaaggtaccatcaatgtccac

SEQ ID NO: 49 - DNA, 23 bp, Artificial Sequence
  Probe SEQ ID No. 49
catcgcgctgctggtgctggtct

SEQ ID NO: 50 - DNA, 27 bp, Artificial Sequence
  Probe SEQ ID No. 50
tgtgccatttcctttctctgcccagtc

SEQ ID NO: 51 - DNA, 23 bp, Artificial Sequence
  Probe SEQ ID No. 51
catcgcgctgctggtgctggtct

SEQ ID NO: 52 - DNA, 25 bp, Artificial Sequence
  Probe SEQ ID No. 52
agccatagcaccaagactgatgcca

SEQ ID NO: 53 - DNA, 26 bp, Artificial Sequence
  Probe SEQ ID No. 53
acctcctctcacctcctccaatcaca

SEQ ID NO: 54 - DNA, 22 bp, Artificial Sequence
  Primer SEQ ID No. 54
  misc_feature (9)..(9): i
cgtttgagntccatgcccaatc

SEQ ID NO: 55 - DNA, 21 bp, Artificial Sequence
  Primer SEQ ID no. 55
  misc_feature (11)..(11): i
tcctctgctgntcacctcttg

SEQ ID NO: 56 - DNA, 18 bp, Artificial Sequence
  Primer SEQ ID No. 56
cacccactcccctctgac

SEQ ID NO: 57 - DNA, 20 bp, Artificial Sequence
  Primer SEQ ID No. 57
  misc_feature (7)..(7): i
cagcagntctccgcatcgtg

SEQ ID NO: 58 - DNA, 21 bp, Artificial Sequence
  Primer SEQ ID No. 58
gtgaaacctgacctctcctac

SEQ ID NO: 59 - DNA, 19 bp, Artificial Sequence
  Primer SEQ ID No. 59
cagcagtctccgcatcgtg

SEQ ID NO: 60 - DNA, 29 bp, Artificial Sequence
  Probe SEQ ID No. 60
  misc_feature (8)..(8): i
ctgcctgntccctacaactacctttctac

SEQ ID NO: 61 - DNA, 26 bp, Artificial Sequence
  Probe SEQ ID No. 61
atcctcatcaagcgacggcagcagaa

SEQ ID NO: 62 - DNA, 29 bp, Artificial Sequence
  Probe SEQ ID No. 62
agcagagagccagccctctgacgtccatc

SEQ ID NO: 63 - DNA, 19 bp, Artificial Sequence
  Priemr SEQ ID No. 63
ccactcaacagcgtgtctc

SEQ ID NO: 64 - DNA, 20 bp, Artificial Sequence
  Primer SEQ ID no. 64
gctcgttctccaggtagtag

SEQ ID NO: 65 - DNA, 23 bp, Artificial Sequence
  Probe SEQ ID No. 65
tgtcgcctttcctgcagccccac

SEQ ID NO: 66 - DNA, 25 bp, Artificial Sequence
  Primer SEQ ID No. 66
cttacaaaacttcttgataacttgc

SEQ ID NO: 67 - DNA, 25 bp, Artificial Sequence
  Primer SEQ ID No. 67
ctgtactgcttgaatacatttatcc

SEQ ID NO: 68 - DNA, 20 bp, Artificial Sequence
  Primer SEQ ID No. 68
ggtttcaccatccctgccaa

SEQ ID NO: 69 - DNA, 33 bp, Artificial Sequence
  Probe SEQ ID No. 69
cttcatctgtactgcttgaatacatttatccag

SEQ ID NO: 70 - DNA, 33 bp, Artificial Sequence
  Probe SEQ ID No. 70
atgatgtctgaagttattgctgcacaattaccc

SEQ ID NO: 71 - DNA, 690 bp, Artificial Sequence
  Nucleotide sequence of the obtained “hybrid” -696 -43 DF3 - -39 -1 minimal CMV promoter
ggaccctagggttcatcggagcccaggtttactcccttaagtggaaatttcttcccccactccctccttggctttctccaaggagggaacccaggctgctggaaagtccggctggggcggggactgtgggtttcagggtagaactgcgtgtggaacgggacagggagcggttagaagggtggggctattccgggaagtggtggggggagggagcccaaaactagcacctagtccactcattatccagccctcttatttctcggccccgctctgcttcagtggacccggggagggcggggaagtggagtgggagacctaggggtgggcttcccgaccttgctgtacaggacctcgacctagctggctttgttccccatccccacgttagttgttgccctgaggctaaaactagagcccaggggccccaagttccagactgcccctcccccctcccccggagccagggagtggttggtgaaagggggaggccagctggagaacaaacgggtagtcagggggttgagcgattagagcccttgtaccctacccaggaatggttggggaggaggaggaagaggtaggaggtaggggagggggcggggttttgtcacctgtcacctgctccggctgtgcctagggcgggcgggcggggagtgggaggtctatataagcagagctggtttagtgaac

SEQ ID NO: 72 - DNA, 1853 bp, Homo sapiens
  misc_feature (1)..(1853): MUC1,acc.# gi|324120957|ref|NM_001204286.1| Homo sapiens mucin 1, cell surface associated (MUC1), transcript variant 10, mRNA (long variant)
cgctccacctctcaagcagccagcgcctgcctgaatctgttctgccccctccccacccatttcaccaccaccatgacaccgggcacccagtctcctttcttcctgctgctgctcctcacagtgcttacagctaccacagcccctaaacccgcaacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaaaaaaaaaaaaaaaaa

SEQ ID NO: 73 - DNA, 130 bp, Homo sapiens
  misc_feature (1)..(130): acc.#  gi|3241209571-130 exon 1 Homo sapiens mucin 1, cell surface associated (MUC1), transcript variant 10, mRNA
cgctccacctctcaagcagccagcgcctgcctgaatctgttctgccccctccccacccatttcaccaccaccatgacaccgggcacccagtctcctttcttcctgctgctgctcctcacagtgcttacag

SEQ ID NO: 74 - DNA, 454 bp, Homo sapiens
  misc_feature (1)..(454): MUC1, acc.# gi|324120957534-987 exon 3a Homo sapiens mucin 1, cell surface associated (MUC1), transcript variant 10, mRNA
gccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatg

SEQ ID NO: 75 - DNA, 150 bp, Homo sapiens
  misc_feature (1)..(150): MUC1, acc.# gi|3241209571303-1452 exon 7 Homo sapiens mucin 1, cell surface associated (MUC1), transcript variant 10, mRNA
gctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaag

SEQ ID NO: 76 - DNA, 386 bp, Homo sapiens
  misc_feature (1)..(386): MUC1, acc.# gi|3241209571453-1838 exon 8 Homo sapiens mucin 1, cell surface associated (MUC1), transcript variant 10, mRNA
gtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 77 - DNA, 4624 bp, Homo sapiens
  misc_feature (1)..(4624): ERBB2 isoform A, acc.# gi|54792095|ref| NM_004448.2| Homo sapiens v-erb-b2 erythroblastic leukemia viral oncogene homolog 2, neuro/glioblastoma derived oncogene homolog (avian) (ERBB2), transcript variant 1, mRNA
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 78 - DNA, 142 bp, Homo sapiens
  misc_feature (1)..(142): ERBB2 Total form (wt), acc.# gi| 547920971190-1331 exon 11 Homosapiens v-erb-b2 erythroblastic leukemia viral oncogene homolog 2, neuro/glioblastoma derived oncogene homolog (avian) (ERBB2), transcript variant 2, mRNA
gcctgcctccacttcaaccacagtggcatctgtgagctgcactgcccagccctggtcacctacaacacagacacgtttgagtccatgcccaatcccgagggccggtatacattcggcgccagctgtgtgactgcctgtccct

SEQ ID NO: 79 - DNA, 120 bp, Homo sapiens
  misc_feature (1)..(120): ERBB2 Total form (wt),acc.# gi| 547920971332-1451 exon 12 Homosapiens v-erb-b2 erythroblastic leukemia viral oncogene homolog 2, neuro/glioblastoma derived oncogene homolog (avian) (ERBB2), transcript variant 2, mRNA
acaactacctttctacggacgtgggatcctgcaccctcgtctgccccctgcacaaccaagaggtgacagcagaggatggaacacagcggtgtgagaagtgcagcaagccctgtgcccgag

SEQ ID NO: 80 - DNA, 161 bp, Homo sapiens
  misc_feature (1)..(161): ERBB2 Mutant form, acc.# gi|547920972168-2328 exon 19 Homo sapiens v-erb-b2 erythroblastic leukemia viral oncogene homolog 2, neuro/glioblastoma derived oncogene homolog (avian) (ERBB2), transcript variant 2, mRNA
gaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactc

SEQ ID NO: 81 - DNA, 139 bp, Homo sapiens
  misc_feature (1)..(139): ERBB2 Mutant form acc.# gi|547920972377-2515 exon 21 Homo sapiens v-erb-b2 erythroblastic leukemia viral oncogene homolog 2,neuro/glioblastoma derived oncogene homolog (avian) (ERBB2), transcript variant 2, mRNA
ccctctgacgtccatcatctctgcggtggttggcattctgctggtcgtggtcttgggggtggtctttgggatcctcatcaagcgacggcagcagaagatccggaagtacacgatgcggagactgctgcaggaaacggag

SEQ ID NO: 82 - DNA, 2170 bp, Homo sapiens
  misc_feature (1)..(2170): ESR1, acc.# gi|170295798|ref|NM_000125.3| Homo sapiens estrogen receptor 1 (ESR1), transcript variant 1, mRNA
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgctt

SEQ ID NO: 83 - DNA, 686 bp, Homo sapiens
  misc_feature (1)..(686): ESR1, acc.# gi|1702957981-686 exon 1 Homo sapiens estrogen receptor 1 (ESR1), transcript variant 1, mRNA
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacag

SEQ ID NO: 84 - DNA, 2870 bp, Homo sapiens
  misc_feature (1)..(2870): PGR, acc.# gi|321117149|ref|NM_001202474.1| Homo sapiens progesterone receptor (PGR), transcript variant 1, mRNA
agctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggtatgtctttttgttttggtcaggattatgaggtcttga

SEQ ID NO: 85 - DNA, 158 bp, Homo sapiens
  misc_feature (1)..(158): PGR, acc.#gi|1603587833232-3389 exon 7 Homo sapiens progesterone receptor (PGR), transcript variant 2, mRNA
ttcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgat

SEQ ID NO: 86 - DNA, 350 bp, Homo sapiens
  misc_feature (1)..(350): PGR, acc.#gi|1603587833390-13037 exon 8 Homo sapiens progesterone receptor (PGR), transcript variant 2, mRNA
cttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggtatgtctttttgttttggtcaggattatgaggtcttgagtttttataatgttcttctgaaagccttacatttataacatcatagtgtgtaaatttaaaagaaaaattgtgaggttctaattattttcttttataaagtataattagaatgtttaact

SEQ ID NO: 87 - DNA, 1131 bp, HSV-2
  misc_feature (1)..(1131): Initial sequence of TK2 gene
atggcttctcacgccggccaacagcacgcgcctgcgttcggtcaggctgctcgtgcgagcgggcctaccgacggccgcgcggcgtcccgtcctagccatcgccagggggcctccgaagcccgcggggatccggagctgcccacgctgctgcgggtttatatagacggaccccacggggtgggggagaccaccacctccgcgcagctgatggaggccctggggccgcgcgacgatatcgtctacgtccccgagccgatgacttactggcaggtgctgggggcctccgagaccctgacgaacatctacaacacgcagcaccgtctggaccgcggcgagatatcggccggggaggcggcggtggtaatgaccagcgcccagataacaatgagcacgccttatgcggcgacggacgccgtttttgctcctcatatcgggggggaggctgtgggcccgcaagccccgcccccggccctcacccttgttttcgaccggcaccctatcgcctccctgctgtgctacccggccgcgcggtacctcatgggaagcatgaccccccaggccgtgttggcgttcgtggccctcatgcccccgaccgcgcccggcacgaacctggtcctgggtgtccttccggaggccgaacacgccgaccgcctggccagacgccaacgcccgggcgagcggcttgacctggccatgctgtccgccattcgccgtgtctacgacctactcgccaacacggtgcggtacctgcagcgcggcgggaggtggcgggaggactggggccggctgacgggggtcgccgcggcgaccccgcgccccgaccccgaggacggcgcggggtctctgccccgcatcgaggacacgctgtttgccctgttccgcgttcccgagctgctggcccccaacggggacttgtaccacatttttgcctgggtcttggacgtcttggccgaccgcctccttccgatgcatctatttgtcctggattacgatcagtcgcccgtcgggtgtcgagacgccctgttgcgcctcaccgccgggatgatcccagcccgcgtcacaaccgccgggtccatcgccgagatacgcgacctggcgcgcacgtttgcccgcgagatgggggaagtttag

SEQ ID NO: 88 - DNA, 1131 bp, HSV-2
  misc_feature (1)..(1131): HSV TK2 entire nucleotide sequence without mutation in mut1 site.
atggcttctcacgccggccaacagcacgcgcctgcgttcggtcaggctgctcgtgcgagcgggcctaccgacggccgcgcggcgtcccgtcctagccatcgccagggggcctccgaagcccgcggggatccggagctgcccacgctgctgcgggtttatatagacggaccccacggggtggggaagaccaccacctccgcgcagctgatggaggccctggggccgcgcgacaatatcgtctacgtccccgagccgatgacttactggcaggtgctgggggcctccgagaccctgacgaacatctacaacacgcagcaccgtctggaccgcggcgagatatcggccggggaggcggcggtggtaatgaccagcgcccagataacaatgagcacgccttatgcggcgacggacgccgttttggctcctcatatcgggggggaggctgtgggcccgcaagccccgcccccggccctcacccttgttttcgaccggcaccctatcgcctccctgctgtgctacccggccgcgcggtacctcatgggaagcatgaccccccaggccgtgttggcgttcgtggccctcatgcctccgaccgcgcccggcacgaacctggtcctgggtgtccttccggaggccgaacacgccgaccgcctggccagacgccaacgcccgggcgagcggcttgacctggccatgctgtccgccattcgccgtgtctacgatctactcgccaacacggtgcggtacctgcagcgcggcgggaggtggcgggaggactggggccggctgacgggggtcgctgcggcgaccccgcggccggacccggaggacggcgcggggtctctgccccgcatcgaggacacgctgtttgccctgttccgcgttcccgagctgctggcccccaacggggacttgtaccacatttttgcctgggtcttggacgtcttggccgaccgcctccttccgatgcatctatttgtcctggattacgatcagtcgcccgtcgggtgtcgagacgccctgttgcgcctcaccgccgggatgatcccaacccgcgtcacaaccgccgggtccatcgccgagatacgcgacctggcgcgcacgtttgcccgcgaggtggggggagtttag

SEQ ID NO: 89 - protein, 376 aa, HSV-2
  MISC_FEATURE (1)..(376): Deduced amino acid sequence of HSV TK2 entire nucleotide sequence without mutation in mut1 site
MASHAGQQHAPAFGQAARASGPTDGRAASRPSHRQGASEARGDPELPTLLRVYIDGPHGVGKTTTSAQLMEALGPRDNIVYVPEPMTYWQVLGASETLTNIYNTQHRLDRGEISAGEAAVVMTSAQITMSTPYAATDAVLAPHIGGEAVGPQAPPPALTLVFDRHPIASLLCYPAARYLMGSMTPQAVLAFVALMPPTAPGTNLVLGVLPEAEHADRLARRQRPGERLDLAMLSAIRRVYDLLANTVRYLQRGGRWREDWGRLTGVAAATPRPDPEDGAGSLPRIEDTLFALFRVPELLAPNGDLYHIFAWVLDVLADRLLPMHLFVLDYDQSPVGCRDALLRLTAGMIPTRVTTAGSIAEIRDLARTFAREVGGV

SEQ ID NO: 90 - DNA, 1131 bp, HSV-2
  misc_feature (1)..(1131): HSV TK2 entire nucleotide sequence containing mutation in mut1 site
atggcttctcacgccggccaacagcacgcgcctgcgttcggtcaggctgctcgtgcgagcgggcctaccgacggccgcgcggcgtcccgtcctagccatcgccagggggcctccgaagcccgcggggatccggagctgcccacgctgctgcgggtttatatagacggaccccacggggtggggaagaccaccacctccgcgcagctgatggaggccctggggccgcgcgacgatatcgtctacgtccccgagccgatgacttactggcaggtgctgggggcctccgagaccctgacgaacatctacaacacgcagcaccgtctggaccgcggcgagatatcggccggggaggcggcggtggtaatgaccagcgcccagataacaatgagcacgccttatgcggcgacggacgccgttttggctcctcatatcgggggggaggctgtgggcccgcaagccccgcccccggccctcacccttgttttcgaccggcaccctatcgcctccctgctgtgctacccggccgcgcggtacctcatgggaagcatgaccccccaggccgtgttggcgttcgtggccctcatgcctccgaccgcgcccggcacgaacctggtcctgggtgtccttccggaggccgaacacgccgaccgcctggccagacgccaacgcccgggcgagcggcttgacctggccatgctgtccgccattcgccgtgtctacgatctactcgccaacacggtgcggtacctgcagcgcggcgggaggtggcgggaggactggggccggctgacgggggtcgctgcggcgaccccgcggccggacccggaggacggcgcggggtctctgccccgcatcgaggacacgctgtttgccctgttccgcgttcccgagctgctggcccccaacggggacttgtaccacatttttgcctgggtcttggacgtcttggccgaccgcctccttccgatgcatctatttgtcctggattacgatcagtcgcccgtcgggtgtcgagacgccctgttgcgcctcaccgccgggatgatcccaacccgcgtcacaaccgccgggtccatcgccgagatacgcgacctggcgcgcacgtttgcccgcgaggtggggggagtttag

SEQ ID NO: 91 - protein, 376 aa, HSV-2
  MISC_FEATURE (1)..(376): Deduced amino acid sequence of HSV TK2 entire nucleotide sequence containing mutation in mut1 site
MASHAGQQHAPAFGQAARASGPTDGRAASRPSHRQGASEARGDPELPTLLRVYIDGPHGVGKTTTSAQLMEALGPRDDIVYVPEPMTYWQVLGASETLTNIYNTQHRLDRGEISAGEAAVVMTSAQITMSTPYAATDAVLAPHIGGEAVGPQAPPPALTLVFDRHPIASLLCYPAARYLMGSMTPQAVLAFVALMPPTAPGTNLVLGVLPEAEHADRLARRQRPGERLDLAMLSAIRRVYDLLANTVRYLQRGGRWREDWGRLTGVAAATPRPDPEDGAGSLPRIEDTLFALFRVPELLAPNGDLYHIFAWVLDVLADRLLPMHLFVLDYDQSPVGCRDALLRLTAGMIPTRVTTAGSIAEIRDLARTFAREVGGV

SEQ ID NO: 92 - DNA, 86 bp, Homo sapiens
aaagaattcgagctctctagagtcgactgcagaagcttgatatccctgcctgaatctgttctgccccctccccacccatttcacca

SEQ ID NO: 93 - DNA, 174 bp, Homo sapiens
ccaccatgacaccgggcacccagtctcctttcttcctgctgctgctcctcacagtgcttacagctcaattcccactctgatactcctaccacccttgccagccatagcaccaagactgatgccagtagcactcaccatagcacggtacctcctctcacctcctccaatcacagc

SEQ ID NO: 94 - DNA, 174 bp, Homo sapiens
acttctccccagttgtctactggggtctcttccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccag

SEQ ID NO: 95 - DNA, 87 bp, Homo sapiens
cagtggcagccacttctgccaacttgtaggggcacgtcgcgtttgagtccatgcccaatcccgagggccggtatacattcggcgcca

SEQ ID NO: 96 - DNA, 87 bp, Homo sapiens
gctgtgtgactgcctgtccctacaactacctttctacggacgtgggatcctgcaccctcgtctgccccctgcacaaccaagaggtga

SEQ ID NO: 97 - DNA, 87 bp, Homo sapiens
cagcagaggatggaacctgcacccactcccctctgacgtccatcatctctgcggtggttggcattctgctggtcgtggtcttggggg

SEQ ID NO: 98 - DNA, 87 bp, Homo sapiens
tggtctttgggatcctcatcaagcgacggcagcagaagatccggaagtacacgatgcggagactgctgcaggaaaccttacaaaact

SEQ ID NO: 99 - DNA, 87 bp, Homo sapiens
tcttgataacttgcatgatcttgtcaaacaacttcatctgtactgcttgaatacatttatccagtcccgggcactgagtgttgaatt

SEQ ID NO: 100 - DNA, 87 bp, Homo sapiens
tccagaaatgatgtctgaagttattgctgcacaattacccaagatattggcagggatggtgaaaccccttctctccactcaacagcg

SEQ ID NO: 101 - DNA, 87 bp, Homo sapiens
tgtctccgagcccgctgatgctactgcacccgccgccgcagctgtcgcctttcctgcagccccacggccagcaggtgccctactacc

SEQ ID NO: 102 - DNA, 85 bp, Homo sapiens
tggagaacgagcccagcggcgatatcaagcttgtcgactgcagtctagagagctcgaattcgcggccgcctcgagatttaaataa

SEQ ID NO: 103 - DNA, 28 bp, Artificial Sequence
  MUC1 For St EcoRV primer
atagatatcacctctcaagcagccagcg

SEQ ID NO: 104 - DNA, 31 bp, Artificial Sequence
  Muc1 Rev2 St EcoRV
ttagatatcagaacctgagtggagtggaatg

SEQ ID NO: 105 - DNA, 32 bp, Artificial Sequence
  ER1 for1 st primer
aaacgatatcaccctccacaccaaagcatctg

SEQ ID NO: 106 - DNA, 36 bp, Artificial Sequence
  ER1 rev2 St EcoRV
atagatatcgtaaaatgcagcagggattatctgaac

SEQ ID NO: 107 - DNA, 30 bp, Artificial Sequence
  PR for2 st primer
ttttgatatcagctcttggtgcctgtttgg

SEQ ID NO: 108 - DNA, 36 bp, Artificial Sequence
  PR rev2 St EcoRV primer
tatgatatccactttttatgaaagagaaggggtttc

SEQ ID NO: 109 - DNA, 29 bp, Artificial Sequence
  Her2Neu st for1 primer
ataagatatcgctccgccacctctaccag

SEQ ID NO: 110 - DNA, 29 bp, Artificial Sequence
  HER2Neu st rev1 primer
tttagatatcagcccacaccagccatcac

SEQ ID NO: 111 - DNA, 22 bp, Artificial Sequence
  Probe sequence Ex1 for muc1 gene (exon1)
tgacaccgggcacccagtctcc

SEQ ID NO: 112 - DNA, 20 bp, Artificial Sequence
  Rev EX1- 1 primer
ctgtaagcactgtgaggagc

SEQ ID NO: 113 - DNA, 29 bp, Artificial Sequence
  Probe sequence for beta-2 microglobulin gene (B2M)
ctgcttacatgtctcgatcccacttaact

SEQ ID NO: 114 - DNA, 22 bp, Artificial Sequence
  Primer for B2M For Mod
gtgtgaaccatgtgactttgtc

SEQ ID NO: 115 - DNA, 22 bp, Artificial Sequence
  Primer for B2M Rev Mod
tccaaatgcggcatcttcaaac

SEQ ID NO: 116 - DNA, 24 bp, Artificial Sequence
  Primer for nearly entire B2M amplification For
aatataagtggaggcgtcgcgctg

SEQ ID NO: 117 - DNA, 27 bp, Artificial Sequence
  Primer for nearly entire B2M amplification rev
accagattaaccacaaccatgccttac

SEQ ID NO: 118 - DNA, 20 bp, Artificial Sequence
  Primer for nearly entire muc1 amplification (transcription variant 1) For (in exon 1)
acctctcaagcagccagcgc

SEQ ID NO: 119 - DNA, 21 bp, Artificial Sequence
  Primers for nearly entire muc1 amplification (transcription variant 1) Rev (in last exon)
ttggcgcagtgggagaccacg

SEQ ID NO: 120 - DNA, 31 bp, Artificial Sequence
  TKI for BamHI primer
ataggatccatggcctcgtaccccggccatc

SEQ ID NO: 121 - DNA, 35 bp, Artificial Sequence
  TKI rev EcoRI primer
atagaattcttatcacatctcacgggcaaacgtgc

SEQ ID NO: 122 - DNA, 31 bp, Artificial Sequence
  TKII for EcoRI primer
atagaattcatggcttctcacgccggccaac

SEQ ID NO: 123 - DNA, 33 bp, Artificial Sequence
  TKII rev XhoI primer
atactcgagtcactaaactccccccacctcgcg

SEQ ID NO: 124 - DNA, 21 bp, Artificial Sequence
  TK2 for mut1 D-N primer
ccgcgcgacaatatcgtctac

SEQ ID NO: 125 - DNA, 21 bp, Artificial Sequence
  TK2 rev mut1 D-N primer
gtagacgatattgtcgcgcgg

SEQ ID NO: 126 - DNA, 19 bp, Artificial Sequence
  TK2 for mut2 F-L primer
gacgccgttttggctcctc

SEQ ID NO: 127 - DNA, 19 bp, Artificial Sequence
  TK2 rev mut2 F-L primer
gaggagccaaaacggcgtc

SEQ ID NO: 128 - DNA, 21 bp, Artificial Sequence
  TK2 for mut3 A-T primer
gatgatcccaacccgcgtcac

SEQ ID NO: 129 - DNA, 21 bp, Artificial Sequence
  TK2 rev mut3 A-T primer
gtgacgcgggttgggatcatc

SEQ ID NO: 130 - DNA, 21 bp, Artificial Sequence
  TK2 for mut4 D-N primer
ggtggggaagaccaccacctc

SEQ ID NO: 131 - DNA, 21 bp, Artificial Sequence
  TK2 rev mut4 D-N primer
gaggtggtggtcttccccacc

SEQ ID NO: 132 - DNA, 28 bp, Artificial Sequence
  TK2 for mut5 primer
ggccggacccggaggacggcgcggggtc

SEQ ID NO: 133 - DNA, 36 bp, Artificial Sequence
  TK2 rev mut5 primer
gtcctccgggtccggccgcggggtcgccgcggcgac

SEQ ID NO: 134 - DNA, 31 bp, Artificial Sequence; identical to SEQ ID NO: 122
  TK 2-10 For EcoRI primer

SEQ ID NO: 135 - DNA, 33 bp, Artificial Sequence; identical to SEQ ID NO: 123
  TK 2-10 Rev XhoI-2Tr primer

SEQ ID NO: 136 - DNA, 31 bp, Artificial Sequence
  TK2-10 For BglII primer
ataagatctatggcttctcacgccggccaac

SEQ ID NO: 137 - DNA, 34 bp, Artificial Sequence
  TK 2-10 Rev HindIII-2Tr primer
aataaagctttcactaaactccccccacctcgcg

SEQ ID NO: 138 - DNA, 32 bp, Artificial Sequence
  TK2-10 For BglII N primer
ataagatctcatggcttctcacgccggccaac

SEQ ID NO: 139 - DNA, 34 bp, Artificial Sequence; identical to SEQ ID NO: 137
  TK 2-10 Rev HindIII-2Tr primer

SEQ ID NO: 140 - DNA, 30 bp, Artificial Sequence
  For-696 DF3 Ase I primer
aaattaatggaccctagggttcatcggagc

SEQ ID NO: 141 - DNA, 79 bp, Artificial Sequence
  Rev-43 DF3 min CMV NheI primer
ttatgctagcggatctgacggttcactaaaccagctctgcttatatagacctcccactccccgcccgcccgccctaggc

SEQ ID NO: 142 - DNA, 79 bp, Artificial Sequence
  Rev-43 DF3 minCMV NheI reverse complement sequence
gcctagggcgggcgggcggggagtgggaggtctatataagcagagctggtttagtgaaccgtcagatccgctagcataa

(19) United States
(12) Patent Application Publication
(10) Pub. No.: US 2017/0342497 A1
(43) Pub. Date: Nov. 30, 2017

(54) Kits and Methods for Monitoring Therapy and/or for Adapting Therapy of an Epithelial Cancer Patient

(71) Applicant: Technologie Integrale Ltd., Ramsey, Isle of Mann (GB)

(72) Inventor: Elena Yu Filinova, Moscow (RU)

(73) Assignee: Technologie Integrale Ltd., Ramsey, Isle of Mann (GB)

(21) Appl. No.: 15/326,631

(22) PCT Filed: Jul. 16, 2015
(86) PCT No.: PCT/EP2015/066234

§ 371 Date: Jan. 16, 2017

(30) Foreign Application Priority Data
Jul. 16, 2014 (DE) 10 2014 010 587.3

(51) Int. Cl. C12Q 1/68

(52) U.S. Cl. CPC C12Q 1/6886; C12Q 2600/106; C12Q 2600/158

(57) ABSTRACT

The present invention relates to kits and methods for monitoring therapy and/or for adapting therapy of an epithelial cancer patient, and for determining malignancy grade or progression of a tumor of a patient suffering from an epithelial tumor.

The present invention relates to kits and methods for monitoring therapy and/or for adapting therapy of an epithelial cancer patient, and for determining malignancy grade or progression of a tumor of a patient suffering from an epithelial tumor.

### INTRODUCTION

According to World Health Organization population statistics on death rate in the world in 2010, cancer diseases were the first reason for dying worldwide and even went ahead heart & vessels failures which moved to the second place from their traditional first one. Now every 8^{th} woman in the world will obtain breast cancer during her life (12.5 percent of female population worldwide), and every 6^{th} man will obtain prostate cancer during his life (16.6 percent of male population), together with indication for these indices growth for population of countries with developed and stable economy.

The search for targeted tumor-specific markers is the crucial task for the development of selective cancer therapy approaches and targeted cancer therapy of the future. There are two main groups of candidates for selective anti-tumor therapy:

1) receptors on cancer cell membrane and their corresponding genes;

2) enzymes/ferments hyper-activated during malignant transformation and their metabolic substrates.

The first “receptors” group is more related to a diagnostic application. However, it is necessary to identify those membrane proteins which are cancer-specific. This is not an easy task because in general, the expression of tissue-specific proteins is at least several times decreased in tumor cells as compared to normally differentiating healthy cells. The second group consists of the developing class of modern antitumor therapeutic agents that is intended to provide targeted and personalized medical treatment. MUC1 (carcinoma associated mucine-like membrane glycoprotein) belongs to the first group. It is highly expressed in some cancer tissues [32], especially in epithelium-originated types of tumors [3] (carcinomas, adenocarcinomas): breast cancer [31], ovarian cancer [35], lung cancer [21], prostate, colon, bladder, gastric, pancreas [40] cancers, etc. MUC1 antigen expression measurement is a position in the panel of five standard women cancer-specific diagnostic markers (i.e. HER2-neu or erb2 (cancer-associated transformed growth factor receptor), ER (estrogen receptor), PR (progesterone receptor), AFP (alpha fetoprotein)) in routine breast cancer immunodiagnostic [1] and monitoring in Western Europe countries, USA and Canada.

Clinic evidence for hyperexpression of MUC1 glycoprotein in 95-98% of breast cancer cases, especially in 30% of ER-negative and 65% of HER2-neo-negative primary tumors, made this antigen one of the most important diagnostic markers in genotyping and proteomics assays [15, 24, 31]. In the last years MUC1 antigen was included into prognostic markers phenotyping tests in classification of breast and ovarian tumors [9]. Hyperexpression was found in 95% of metastatic breast cancer patients who developed a disease recurrence after surgery and who are often resistant to tamoxifen therapy and demonstrate low response to chemotherapy treatment [9]. It was also shown that MUC1 gene and its promoter have a crucial influence for cancer transformation transduction events through estrogen receptor transcription regulation pathway [15, 28]. MUC1 is an active participant in proliferation and growth of malignant cells in tyrosine kinase phosphorylation alterations in p53-dependant signaling [48], beta-catenin signaling [39, 41], Bcl-x [37], MAPK and ERK1/2 kinases [37, 48], c-Src [38], Ras, c-Myc, EGFR expression [15, 20, 25, 42] and also in caspase-8 kinase expression-phosphorylation [22].

Clinical data received by measurements of MUC1, HER2-neu, ER and PR in surgery samples of 98 breast cancer patients with different stages of disease within 2008-2010 with the kits and methods of the present invention suggest that 15-18% of all patients are triple-negative (i.e. ER-, PR-, HER2-). Of note, 45-50% of such triple-negative breast cancer patients demonstrate hyperexpression of MUC1 antigen. In case of advanced disease, this MUC1 is the prominent target for therapeutic treatment for these women, who are admitted to be reluctant to existing aromatase inhibitors and chemotherapy regimes and their modern combinations. The second advantage of the presented kits and methods of the present invention is that they allow for quantitative measurement of hormone receptors and HER2-neu expression during treatment in dynamics, in order to catch when suppression with hormone and/or antibody therapy causes the loss of therapy response and, eventually, disease progression. It is even possible to take samples for these via biopsy from normal breast tissue from patients objected for the surgery but having signs of metastatic advanced breast cancer disease.

A number of methods are currently used as standard hormone receptors diagnostics for breast cancer patients. These include reactions with fluorescent-labeled or pure ER, PR, HER2-neo and MUC1-specific monoclonal antibodies (mAbs) and polyclonal antibodies, such as for use in immune histochemistry, ELISA, flow cytometry and their modifications, and, also for research laboratory purpose, Western blot and fluorescent microscopy. These routine methods have several drawbacks such as the high cost of MUC1-specific monoclonal antibodies, time-consuming laborious tests performance and poor quantitative resolution. However, the main problem for antibody-based diagnostics for cancer cell receptors is that a wide range of isoforms (FIG. 1) exist and that especially malignant less-glycosylated variants exist in the human organism [47]. Whereas “normal” MUC1 antigen can have up to 39 repeats of a 23 amino acid sequence in the outer cellular domain [33], cancer-specific forms can have not only a lower degree of glycosylation, and a lower number of repeat [19] but exons may also be absent or shorter or some longer exons may be present instead of another one. Therefore MUC1-specific monoclonal antibodies (mAbs) in general do not match all isoforms of the protein in the human body, which are called “total MUC1 transcripts”. Moreover, anti-MUC1 mAbs cannot distinguish the communities of malignant and “normal” transcripts.

In one preferred embodiment, the method of the invention described below in more detail is a Real-Time RT-PCR method. The RT-PCR method is designed for quantitative determination of human MUC1, HER2-neu (erb2), ER, PR gene expression level in breast cancer samples, MUC1 expression level in the other epithelium-originated malignant tissues, such as ovarian, prostate, lung, bladder, colon and pancreatic cancers, by reverse transcription and real-time PCR. The methods and kits of the invention allow to determine the total number of copies of “normal” full-length MUC1 mRNA variant in the tissue sample and also the majority of MUC1 mRNA forms generated during alternative splicing of MUC1 pre-mRNA, including splice variants MUC1/A and MUC1/D and short forms MUC1/X, MUC1/Y, MUC1/Z known to be associated with the presence of malignancy [4, 35].

According to World Health Organization population statistics for death rate in the world for 2010 cancer diseases were the first reason for dying worldwide and even went ahead heart & vessels failures which moved to the second place from their traditional the first one. Now every 8^{th} woman in the world will obtain breast cancer during her life (12.5 percent of female population worldwide), and every 6^{th} man will obtain prostate cancer during his life (16.6 percent of male population), together with indication for these indices growth for population of countries with developed and stable economy.

The search for targeted tumor-specific markers is the crucial task for development of selective cancer therapy approaches—targeted cancer therapy of the future. There are two main groups of candidates for selective antitumor therapy:

1) receptors on cancer cell's membrane and their genes;

2) hyper-activated during malignant transformation enzymes/ferments and their metabolic substrates.

The first “receptors” group is more related to diagnostic application. However, it is necessary to find those membrane proteins which are cancer-specific. This is not an easy task because in general tumors cells expression of tissue-specific proteins is at least several times decreased compared to normally differentiating healthy cells. The second group consists of the developing class of modern antitumor therapeutic agents that is intended to provide targeted and personal medical treatment. MUC1 -carcinoma associated mucine-like membrane glycoprotein—belongs to the first group, it is highly expressed in some cancer tissues [32], especially in epithelium-originated types of tumors [3] (carcinomas, adenocarcinomas): breast cancer [31], ovarian cancer [35], lung cancer [21], prostate, colon, bladder, gastric, pancreas [40] cancers, etc. MUC1 antigen expression measurement is a position in the panel of five standard women cancer-specific diagnostic markers (HER2-neu or erb2 (cancer-associated transformed growth factor receptor), ER (estrogen receptor), PR (progesterone receptor), AFP (alphafetoprotein)) in routine breast cancer immunodiagnostic [1] and monitoring in Western Europe countries, USA and Canada.

Clinic evidences of MUC1 glycoprotein hyperexpression in 95-98% of breast cancer cases, especially in 30% of ER-negative and 65% of HER2-neo-negative primary tumors, made this antigen one of the most important diagnostic markers in genotyping and proteomics assays [15, 24, 31]. Last years MUC1 antigen was included into prognostic markers phenotyping tests in classification of breast and ovarian tumors [9]. Its hyperexpression is being found in 95% of metastatic breast cancer patients who developed disease recurrence after the surgery and often are resistant to tamoxifen therapy and demonstrate low response to chemotherapy treatment [9]. It was also shown that MUC1 gene and its promoter have a crucial influence for cancer transformation transduction events through estrogen receptor transcription regulation pathway [15, 28], MUC1 is an active participant in proliferation and growth of malignant cells in tyrosine kinases phosphorylation alterations in p53-dependant signaling [48] beta-catenin signaling [39, 41], Bcl-x [37], MAPK and ERK1/2 kinases [37, 48], c-Src [38], Ras, c-Myc, EGFR expression [15, 20, 25, 42] and also in caspase-8 kinase expression-phosphorylation [22].

Clinical data received from measurements of MUC1, HER2-neu, ER and PR in surgery samples of 98 breast cancer patients with different stages of disease within 2008-2010 with the presented novel test system suggest that 15-18 percent of total number of patients are triple-negative (ER-, PR-, HER2-). The most significant is that 45-50 percent of triple-negative breast cancer patients demonstrate hyperexpression of MUC1 antigen which in case of advanced disease is the prominent target for therapeutic treatment in these women who are admitted to be reluctant to existing aromatase inhibitors and chemotherapy regimes and their modern combinations. The second advantage of the presented novel test system is its quantitative measurement of hormone receptors and HER2-neu expression which is possible to make under the treatment in dynamics to catch when suppression with hormone or antibodies therapy causes the loose of therapy responses and disease progression. Samples for these measurements is even possible to take with biopsy method from normal breast tissue of patients objected for the surgery but having signs of metastatic advanced breast cancer disease.

The number of methods being used for standard hormone receptors diagnostic for breast cancer patients includes reactions with fluorescent-labeled or pure ER, PR, HER2-neo and MUC1-specific monoclonal antibodies (mAbs) or polyclonal antibodies such as immune histochemistry, ELISA, flow cytometry and their modifications, also for research laboratory purpose—Western blot and fluorescent microscopy. These routine methods have several backwards such as the high cost of MUC1-specific monoclonal antibodies, time-consuming laborious tests performance and poor quantitative resolution. But the main problem for antibodies-based cancer cell's receptors diagnostics is a wide range of isoforms (FIG. 1) and especially malignant less-glycosylated variants existing in human organism [47]. But when “normal” MUC1 antigen can have up to 39 repeats of 23 amino acid sequence in outer cellular domain [33], cancer-specific forms can have not only lower glycosylation, shorter repeats number [19] but also absent or shorter exons or some longer exons instead of another ones. Therefore MUC1-specific monoclonal antibodies (mAbs) in general do not match all isoforms of the protein which we named as “total MUC1 transcripts”. Moreover MUC1 mAbs cannot distinguish the communities of malignant and “normal” transcripts.

The presenting method is a Real-Time RT-PCR test system which is designed for quantitative determination of human MUC1, HER2-neu (erb2), ER, PR gene expression level in breast cancer samples, MUC1 expression level in the other epithelium-originated malignant tissues (ovarian, prostate, lung, bladder, colon and pancreatic cancers) by reverse transcription—real-time PCR method. This test system allows to determine the total number of copies of “normal” full-length MUC1 mRNA variant in the tissue sample and also the majority of MUC1 mRNA forms generated during alternative splicing of MUC1 pre-mRNA, including splice variants MUC1/A and MUC1/D and short forms MUC1/X, MUC1/Y, MUC1/Z known to be associated with the presence of malignancy [4, 35].

### BACKGROUND

I. MUC1—the Gospel and Evil in Cancer Diagnostic and Therapy

Mucin 1 (MUC1), cell surface associated epithelial heavily glycosylated phosphoprotein, is encoded by the MUC1 gene in humans. It is overexpressed in the apical surface of epithelial cells in the lungs, breast, stomach, intestines, urinary tract, eyes and other organs. Normal MUC1 is a transmembrane protein with a core mass of 120-225 kDa with extensive O-linked glycosylation of its extracellular domain which increases its molecular weight to 250-500. It extends 200-500 nm beyond the surface of the cell [32].

In normal epithelium, mucin 1 protects the body from many infections, preventing the pathogen from reaching the cell surface and has a multiple influence in a cell signaling pathways. Overexpression and changes in glycosylation of MUC1 protein are often associated with breast, colon, ovarian, lung, bladder and pancreatic carcinomas and adenocarcinomas [4, 21, 24, 31, 35]. 18 types of mucin-like glycoproteins with gene modifications are known, each of them consists of many isoforms. Not all of mucin-like antigens are cancer-associated. Rather, normal epithelium tissues usually contain several types of hyperexpressed MUC proteins. Regarding the MUC1 protein, its name “cancer-associated antigen” originated mostly from the source of its tissue discovery (namely a breast cancer patient surgery sample) than from its tumor-specific expression. Human MUC1 is highly expressed in lung bronchoepithelia, intestinum, gastric, cervical, bladder and other types of normal epithelium, as well as in normal women breast tissue [19, 33]. The difference in tumor specificity of MUC1 expression is mostly based on the level of glycosylation of the maturated isoform of the protein which is built into the cell membrane: in quickly dividing cancer cells, MUC1 glycosylation especially in the extracellular domain, is suppressed and is much lower than it is in non-malignant epithelium cells [10]. Also, the number of tandem repeats is decreased, some isoforms can be almost without extracellular domain, some can happen to be spliced without transmembrane domain and float in the outer cellular space. The molecular weight of malignant MUC1 isoforms can shrink to 80-200 kDa, instead of 250-500 kDa in normal epithelium [19, 47].

MUC1 performs multiple functions in cell biochemical metabolism and the regulation of organisms. Regarding the cell signal membrane receptor function, the “general” protein is anchored to the apical surface of almost all types of human epithelia by a transmembrane domain. In normal epithelia, the extracellular “apical” domain includes a 20 amino acid variable number tandem repeat (VNTR) domain, with the number of repeats varying from 20 to 120 in different individuals. These repeats are rich in serine, threonine and proline residues, which permits heavy 0-glycosylation [5, 47], and this outer domain epitopes serve as targets for MUC1-specific MAbs. Beyond the transmembrane domain is a SEA domain that contains a cleavage site for release of the large extracellular domain. The release of mucins performed by sheddases [5] causes so called mucosal immune response which was tried to be exploited for stimulation of anti-tumor immunization with tumor lysates, extracts, recombinant MUC antigen's fragments, etc. The mechanism of cleavage and its role in anti-tumor mucosal immune response formation are not clearly investigated.

Thereby, MUC1 is cleaved in the endoplasmic reticulum into two pieces. The cytoplasmic tail including the transmembrane domain MUC1 is 72 amino acids long and contains several phosphorylation sites [13]. This tail should have been involved in the challenging of intracellular growth factors signal from the cell differentiation way to malignant-associated endless proliferation [16, 44]. The MUC1 cytoplasmic tail was shown to interact with Beta-catenin [27]. In cancer cells, increased expression of MUC1 promotes cancer cell invasion through beta-catenin, resulting in the initiation of epithelial-mesenchymal transition which promotes the formation of metastases.

MUC1 overexpression, aberrant intracellular localization, and alterations in glycosylation have been associated with carcinomas. In breast adenocarcinoma and a variety of epithelial tumors, its transcription is dramatically upregulated, steroid hormones also stimulate the expression of the MUC1 gene. Insulin stimulates the expression of the MUC1 in in vitro breast cancer cell cultures [9]. The MUC1 gene directs expression of decades of protein isoforms (20 are known, FIG. 1), and many of these isoforms are tissue- (epithelia type-) specific.

The ability of chemotherapeutic drugs to access the cancer cells is inhibited by the heavy glycosylation in the extracellular domain of MUC1. The glycosylation creates a highly hydrophilic region which prevents hydrophobic chemotherapeutic drugs from passing through [36]. This prevents the drugs from reaching their targets within tumor cells. It is known that MUC1 glycosylation has been shown to bind to growth factors, and hyperexpression of MUC1 concentrates growth factors near receptors, increasing receptor activity and the growth of cancer cells. MUC1 also prevents the interaction of immune cells with receptors to inhibit an anti-tumor immune response [4, 52].

MUC1 cytoplasmic tail has been shown to associate to p53. This interaction is increased by genotoxic stress. MUC1 and p53 were found to be associated with the p53 response element of the p21 gene promoter [48]. This results in activation of p21 which results in cell cycle arrest. Overexpression of MUC1 in cancer results in inhibition of p53-mediated apoptosis and promotion of p53-mediated cell cycle arrest [51]. The MUC1 cytoplasmic tail is shuttled to the mitochondria through interaction with heat shock protein 90. This interaction is induced through phosphorylation of the MUC1 cytoplasmic tail by Src protein which is activated by the EGF receptor family ligand Neuregulin. The cytoplasmic tail is then inserted into the mitochondrial outer membrane [15, 52]. Localization of MUC1 to the mitochondria prevents the activation of apoptotic mechanisms also through caspase 8, 9-mediated signal transduction pathway [22].

All strategies using MUC1 hyperexpression for antitumor therapy comprise the formation of immune response against MUC1-hyperexpressing tumors [11] and can be classified into several groups:

MUC1-targeted monoclonal antibodies [36]. The drawback is the low targeting due to the limited number (or even only a single isoform) of MUC1 types that can be bound with mAbs

recombinant peptides and their mixtures of MUC1 domains and regions [4] (earlier tumor lysates were used instead of recombinant peptides), to be administrated in vivo either with purpose to:

1) boost mucose-carbohydrate enhanced anti-tumor response [4, 21, 23] highly but non-specifically, or

2) raise cytokine's profile immunity stimulation [21, 26]

incubation of dendritic cells with MUC1-derivates or peptides and their further transfer back to a patient [46]. This method provides very good results in tumor remission, disease stabilization and life expectancy for more than 50 percent of patients in advanced stages, even it is possible to get positive effect for triple-negative breast cancer cases. Its drawback is the very high cost of personal in vitro and patient administration like for transplantation operations. Up to now these cost normally cannot be covered by medical insurance, and therefore the technique is poorly available for majority of patients.

Immune therapy against any type of cancer has its natural restriction for a wide application. First of all, immune therapy of cancer is the immune response against the own human organism cells, which is either toxic to many other types of cells and tissues except tumor cells (MUC1 peptides, [4]). This approach will always cause immune toxicity against normal epithelium cells of all organs similar to bystander effects of a new class of antitumor medicines such as tyrosine kinase inhibitors. In the other case a therapy with anti-MUC1 antibodies is highly specific to one type of malignant cells receptors and leaves the other malignant cells along because their altered receptors are different from a current monoclonal antibody [35]. We suppose the future of MUC1-targeted cancer therapy is hidden under inability of existing immune compositions to distinguish the difference between malignant [18, 24, 35, 51] and normal [5, 19, 47] isoforms of MUC1 protein (FIG. 2) which more likely can be found not only in its outer cellular domain but also in alterations in cytoplasmic domain phosphorylation signals pathways. However some opinions defend the idea that MUC1 metabolic complex (intracellular domain activity) is the same for normal and tumor cells and tissues [14]. We suppose this statement as rather not true and sure there are many not yet studied differences in MUC1 pathway leading to apoptosis or proliferation of cells. Prior to success of therapeutic applications diagnostic problems should be solved.

II. Gene HER-2/neu (ERBB2, v-erb-b2 Erythroblastic Leukemia Viral Oncogene Homolog 2, also known as NEU; NGL; HER2; TKR1; CD340; MLN 19).

ERBB2 gene encodes a member of the epidermal growth factor (EGF) receptor family of receptor tyrosine kinases. This protein has no ligand binding domain of its own and, therefore, cannot bind growth factors. However, it does bind tightly to other ligand-bound EGF receptor family members to form a heterodimer, stabilizing ligand binding and enhancing kinase-mediated activation of downstream signaling pathways, such as those involving mitogen-activated protein kinase and phosphatidylinositol-3 kinase, and activated ErbB2-neu forms can induce mammary tumors formation in transgenic mice [43]. Allelic variations at amino acid positions 654 and 655 of isoform a (positions 624 and 625 of isoform b) have been reported, with the most common allele, lle654/lle655. There are only two RNA transcripts forms of ErbB2 (FIG. 3, [33]), but transcripts consist of 27 exons (with different possible numeration of exons 14-17 or exons 19-22 for the same regions) and have the most complicated product's structure capable to make hundreds of protein isoforms, some of which are cancer-specific and some belong to “wild type” or “furin” family (FIG. 4). Amplification and/or overexpression of this gene has been reported in numerous cancers, including breast and ovarian tumors [6, 43]. Alternative splicing brings up several additional transcript variants, results in some encoding different isoforms and others that have not been fully characterized [30, 33]. The large diversity of HER2 protein isoforms can be concluded from the clinical trials information about advantage of combination of Pertuzumab and Transtuzumab neoadjuvant therapy in patients with advanced inflammatory HER2-positive breast cancer compared with the same regimes of treatment with only one of the mentioned ErbB2-specific therapeutic antibodies [30].

Erbb-2 hyperexpression in routine immunohistochemistry assays is found in approximately 25 percent of women diagnosed with breast cancer [6]. However the therapeutic efficacy and disease regression provided by the treatment with HER2-specific humanized therapeutic antibodies (trastuzumab (Herceptin®)) are proven for 12.5 percent of treated HER2-positive and negative breast cancer patients [1].

MUC1 has been shown to interact with HER2-neu [32]. Together with elongation of the average lifespan in developed countries, a wide spread of breast cancer in women for last decades lead to the development of several methods of immuno- and molecular breast cancer diagnostics. Besides routine immunohistochemistry and microscopy analysis, multiple assays were presented to estimate HER2-neu, ESR1 and PRG1 expression in breast cancer specimen samples. The problem is that tests include not only these three membrane proteins significant for cancer development and choosing methods of treatment of patients, but many other proteins too.

The first drawback of recent test systems for breast cancer diagnostics is their complexity and intention to measure all possible cancer markers such as 56 markers by Prediction Sciences [24], 48 markers in Oncotype DX by Cigna Medical [7], 21 markers in Mammaprint or Multiplex by Celera [12] and attempts of their “fingerprints” data interpretation as a prognostic value thereof.

Second, quantification is a poor feature in these test systems. In case of antibody-antigen, antigen-ligand, peptide-receptor-based [24] onco-markers kit, the absence of calibration curves for so many (56) testing parameters is obvious, and comparison with “control sample” from another so called “positive” patient or tumor tissue [24] is not a quantitative method. In case the diagnostic system is a genetic markers analysis using the extraction of breast tumor RNA from paraffin-embedded frozen slides of surgery samples with following RT-PCR and DNA hybridization with 48 [7] or 21 [12] oncogenes fluorescent bands, the current method of RNA isolation simply cannot provide entire mRNA for quantitative reverse transcription PCR. RNA is being destroyed during water-alcohol-based stages of paraffin embedding and following thawing of these blocks in fists step of extraction, and in case many small pieces of oncogene are being amplified into cDNA primer pairs will give false quantitative information in RT. Also the fish method of DNA oncomarkers bands hybridization does not have calibration calculation. Quantitative analysis of so many parameters as 21 or 48 is rather too complicated and expensive, but some of these markers like estrogen and progesterone receptors expression indeed need to be measured for patient's treatment sake, as determined by us.

The third drawback is that, breast cancer molecular markers test systems are good for retrospective studies only and can have prognostic value for “cancer molecular subtypes” classification [7, 12], but in reality do not have any practical connection to patient's treatment regimes and adjustments in their current therapy in advanced disease.

III. Estrogen Receptor Gene ESR1 (ER-α)

Estrogen receptor is the main acceptor for women sex hormone in breast tissues and reproductive system responsible for hormonal p450-dependent regulation of physiologic processes in human organism and female development and reproduction. Estrogen receptor is the most important target for aromatase inhibitors (hormone therapy) for hormone-positive breast, endometrium and ovarian cancer patients [1]. ESR1 gene has two isotypes: ESR1-alpha (ER-α) and ESR-beta (ER-β), ER-α different spliced variants are confirmed to associate with cancer-transformed cells and tissues [12, 44]. Estrogen receptor 1 has a very long intron zones in genomic structure [33] but not many RNA transcripts (four only, FIG. 5). The differences between a lot of malignant-associated isoforms of ER-alpha starts after translation and complicated schemes of protein splicing [17]. FIG. 6 from the review [17] represents some of these schemes. Domains of ER-α, the mRNA sequence of ER-α alternative promoters are shown to the left of +1. The shaded box shows the ER-α coding region. Exons are numbered in the corresponding blocked region with the nucleotide number above. ATG start codon and the TAG stop codon are shown on FIG. 6. Protein domains are labeled A-F, nucleotide numbers corresponding to the start of each domain are above, with amino acid numbers are below. Relative positions of some of the known functional domains are represented by solid bars below. There is a predicted 96% homology in the DNA binding domain (BD), and a 53% homology between the E/F domains, but the A, B, and hinge (D) domains are not well conserved between ER-α and ER-β. This information analysis is very important for primers choice for quantitative measurement of RNA expression of total ER-α variants in women with hormone-dependent cancers. It is estimated that only 7-10% of the epithelial cells in the normal human breast express ER-α, and it has been shown that this expression fluctuates with the menstrual cycle. Although only a small percentage of the cells in the normal breast express ER-α, these are not the same cells as those that are proliferating. In contrast, ER-β expression is relatively high in the normal breast, with 80-85% of the cells expressing ER-β, which is again inversely correlated with cellular proliferation. In contrast, ER-β expression does not appear to change during the menstrual cycle [33]. The level of ER1, presumably ER-α expression and its dynamic alterations during aromatase inhibitors-chemotherapy treatment of breast cancer patients is the crucial key for correct and in-time adjustments or so called “personalized medicine” adjustments in these patients therapy and the rate of advanced breast cancer cases remission or stabilization. However earlier attempts of ER1 RNA levels quantitative assays [13] were not systematic and used fixed in paraffin blocks biomaterial. The source of ESR1 RNA extraction is limiting the quantitative measurement value dramatically due to destroying of RNA with fixation.

ESR1 splice variants have been detected in a number of different normal tissues, including the breast, endometrium, and pituitary tissues, as well as smooth muscle cells and peripheral blood mononuclear cells [17]. Additionally, ESR1 mRNA splice variants have been detected in various tumor types including breast cancer [3], endometrial carcinoma [44], prolactinoma, systemic lupus erythematosus, and meningiomas (FIG. 7 from [17] Table 1). In the vast majority of cases, wild-type ESR1 is co-expressed along with variant ESR1 mRNAs. Although many of these variants have been predominately detected in diseased tissues, a number of studies have been unable to demonstrate differences in the expression levels, or individual patterns of mRNA splice variants when comparing normal controls from unaffected patients to diseased tissues, suggesting that these variants may also play a role in normal physiological processes. Zhang et al. [52] examined the mRNA ratios of wild-type ESR1 to a number of exon deletion variants in 109 breast cancer specimens and found that the expression of wild-type ER-α was greater than the expression of any of the deletion variants in the majority of cases (data are presented in FIG. 7 table taken from [52]). In all samples, ESR1Δ2 expression was less than wild-type ESR1; ESR1Δ3 and wild type ER1 were expressed at similar levels in 7% of the cases; and higher levels of Δ3 were found in 14% of the cases. Wild-type ESR1 was expressed at similar levels as Δ4 and Δ5 in 16 and 6% of the cases, respectively, and 12% of the cases had increased Δ4 or Δ5. ER-α Δ7 was expressed at higher levels in only 9% of the cases, but the expression of Δ7 equaled that of wild-type ER-α in about 20% of the breast cancers examined. These data demonstrate that although a large number of tumor specimens may express variant ER-α, wild-type ER-α is the predominant isoform in most tumors. That is why in spite of not so many RNA transcripts estrogen receptor's gene was the only one in our diagnostic system for which the choice of primers and fluorescent band was not possible to settle in different exons but only in the first exon. Different exons primers position can exclude the random genomic DNA amplification in RT-PCR cycles, so, primers deviation in two neighbor or not neighbor exons can give possibility to use ethanol fixation of biomaterial before RNA extraction.

MUC1 has been shown to stabilize and to activate ER-α [48], and, contrariwise, ER-α takes part in regulation of MUC1 gene expression [51]. We have strong indications that in case of MUC1 hyperexpression in breast cancer patients with advanced disease who were subjected to aromatase inhibitors 2^{nd} generation therapy for 12-36 months and whose ER-α and PR1 expression was either negative from the beginning or decreased dramatically during hormone deprivation MUC1 malignant isoforms start to replace ER-α [49] and EGFR (epithelial growth factor) [4] receptors in membrane-initiated phosphorylation signaling regulation of nuclear-initiated cell proliferation and apoptosis avoiding which is normally triggered/regulated with steroid/growth factor signaling pathways.

IV. Progesterone Receptor Gene (PR or PGR)

Progesterone receptor expression level is the second important value in breast cancer routine diagnostic together with ER1. The second place is determined by its approximately 10 times or more lower presentation/expression on breast normal and tumor tissues cell surface then ER1 protein presentation/expression level (shown in our data with the same units of Universal Standard for both ER-α (ER1) and PR1 gene's RNA quantification) and, therefore, next in line involvement/influence in aromatase inhibitors therapy effect [1].

The human PR gene consists of eight coding exons separated by seven non-coding introns (FIG. 8). The two main nuclear isoforms, PR-A and PR-B, are independently regulated from defined promoter regions within the PR gene [8]. PR-A is a truncated form of PR-B, lacking the amino terminal 164 amino acids that form the third transactivation domain (AF-3). Other than this, the two forms are structurally identical. PR-C lacks a complete DBD and the first two transactivation domains (AF-3 and AF-1, see FIG. 9).

The balance of PR isoform expression is also important in breast cancer management [8]. Overexpression of PR-A protein compared to PR-B is common in breast cancer, changing progestin responsiveness of cells. Predominant PR-A protein expression signifies a poorer outcome of hormonal therapies, and predominance of PR-B poorer outcome of chemotherapy. Predominance of one isoform is also seen in women at high risk of breast cancer, for example, women with a BRCA1 or BRCA2 mutation commonly exhibit a lack of PR-B. As well as PR-A, PR-B and PR-C, several other smaller isoforms encoded by the PR gene have also been described [8]. PR exon 6 deleted mRNA transcripts are different in breast cancer and normal breast tissue cells [3, 29].

ER1, PR and HER2-neu are the most important breast cancer indicators directly connected to hormone-positive and HER2-positive patient's therapy. Therefore these three markers are always included into all routine surgery/biopsy tests for breast cancer patients [1, 50] and novel molecular subtypes kit systems [7, 12, 24]. Several reports presenting data of quantitative measurement of ER and PR expression levels are available. Thus, in attempt to distinguish metastatic cancer cells in blood of advanced cancer patients Real Time Reverse Transcription PCR was used [2]. Some investigators run RT-PCR for ERBB2 and ERBB3 expression evaluation in transgenic mice [43]. Unfortunately, first, Reverse Transcription and/or RealTime PCR without exact quantitative calibration (diluted standards expression measurements and calculation) do not provide the quantitative ER, PR expression level data. Second, we also tried to fish out the difference in blood with breast cancer cells gradual dilutions in healthy people blood, this pure blood and breast cancer patients blood with our MUC1, ER-α, PR and ERBB2 four markers TaqMan RealTime test system. Baker with co-authors [2] were not able to distinguish certain alterations in expression level with barrier density gradient centrifugation for enrichment altogether. We also made the data-confirmed conclusion that sensitivity of the method is good enough but the difference in expression levels of markers RNA transcripts is too low to admit RT-RealTime measurements good for metastatic cells diagnostic in bloodstream (see Detailed Description Results).

There are published data of a single-tube quantitative assay for mRNA levels for ER1, PR and HER2-neu in breast cancer specimens [13]. This work demonstrates the urgency of such method development and the correct approach for hormone and growth factors receptors expression evaluation. However, as we show further, primers choice for adequate total isoforms expression analysis is dramatically important. Regarding the quantification of receptor's expression we tried the single tube with reference gene mRNA expression measurement presented in [13] and concluded it to be insufficient with data accuracy.

In the examples, the newly developed Universal Standard dilutions Real Time PCR measurements were used for quantitative analysis of the expression level of MUC1, ER-α, PR and ERBB2 transcripts.

### DESCRIPTION OF THE INVENTION

In one embodiment, the present invention relates to an in vitro method for monitoring therapy and/or for adapting therapy of an epithelial cancer patient, who is subject to a cancer treatment, comprising:
(a) obtaining a tissue sample comprising cancer cells from said patient at a first time point,
(b) determining the expression level of
   (i) total membrane-bound Muc1 mRNA, or
   (ii) total membrane bound Muc1 protein in said tissue sample,
(c) determining the expression level of
   (i) the long forms of Muc1 mRNA, or
   (ii) the long forms of Muc1 protein
   in said tissue sample,
(d) determining the ratio between the expression levels of (b) and (c),
(e) repeating steps (a) to (d) at a second time point, which is at least 1 day later than the first time point, preferably at least 1 week later than the first time point, more preferably at least 1 month later than the first time point, even more preferably at least 3, 6, 9 or 12 months later than the first time point,
(f) comparing the ratio of expression levels determined at the first time point and the second time point,
   wherein
   an increase in ratio between the expression level of (b) and (c) at the second time point compared to the first time point indicates that
   (i) the patient is less responsive to said cancer treatment, and
   (ii) is responsive to Muc1 based therapy.

It was surprisingly found that the time course of the ratio r between the expression level of:

the total membrane-bound Muc1 mRNA or protein and

the long forms of Muc1 mRNA or protein

in a cancer patient, who suffers from an epithelial cancer and who is subject to a cancer treatment, is indicative for the responsiveness of the patient to the cancer treatment. In particular, it was surprisingly found an increase in ratio between the expression level of (b) and (c) at the second time point compared to the first time point, i.e. at a later time point, indicates that the patient is less responsive to said cancer treatment.

Therefore, the ratio r is calculated as follows:

$$r = \frac{expression level of total membrane \text{-} bound Muc 1 mRNA}{expression level of the long forms of Muc 1 mRNA}$$

$$or$$

$$r = \frac{expression level of total membrane \text{-} bound Muc 1 protein}{expression level of the long forms of Muc 1 protein}$$

In a preferred embodiment of the present invention, the long forms of Muc1 RNA are all Muc1 mRNA molecules encoding at least exons III to VII of Muc1. The sequence of the exons are known to a skilled person and exon sequences are disclosed herein.

In a further preferred embodiment of the present invention, the long forms of Muc1 RNA are all Muc1 mRNA molecules encoding a Muc1 protein comprising up to 39 repeats in the variable number tandem repeat (VNTR) domain. The VNTR are highly conserved repeats of 20 amino acids.

In an even more preferred embodiment, the long forms of Muc1 RNA are all Muc1 mRNA molecules encoding at least exons III to VII of Muc1, and which are encoding a Muc1 protein comprising up to 39 repeats in the variable number tandem repeat (VNTR) domain.

In a more preferred embodiment of the present invention, the long forms of Muc1 protein comprise at least a part of the variable number tandem repeat (VNTR) domain.

As described in the examples, suitable primers may be used for amplifying long forms of Muc1 mRNA after reverse transcription, for example by taking into account the sequences of exons III and VII of the Muc1 mRNA. Preferred primers suitable in this context are shown in the examples.

According to the present invention, total membrane-bound Muc1 mRNA is understood as all Muc1 mRNAs encoding Muc1 proteins which contain a transmembrane domain. As described in the examples, suitable primers may be used for amplifying total membrane-bound Muc1 mRNA after reverse transcription, for example by taking into account the sequence of the Muc1 mRNA encoding the transmembrane domain. Preferred primers suitable in this context are shown in the examples.

An epithelial cancer patient is a patient who suffers from an epithelial cancer. An epithelial cancer is a cancer derived from epithelial cells. Preferred epithelial cancers are epithelial cancers developing in the breast, prostate, lung, pancreas, and colon, i.e. breast cancer prostate cancer, lung cancer, pancreatic cancer and colon cancer.

The present method applies to patients who are subject to a cancer treatment. This allows monitoring and/or adapting therapy if necessary.

In step (a), the method involves obtaining a tissue sample comprising cancer cells from said patient. In one preferred embodiment, a biopsy may be taken from the patient in order to retrieve cancer cells. For example, a breast tissue biopsy may be taken in case of breast cancer patients. In another preferred embodiment, a blood sample may be obtained In case such sample contains cancer cells or is suspected to contain cancer cells.

Such samples may be stored under appropriate conditions e.g. by freezing and/or the addition of RNAse inhibitors and may be used at a later point for determining the expression level of mRNAs or proteins in question, or they may be used directly after obtaining the sample for determining the expression level of mRNAs or proteins in question.

Typically, mRNA is isolated from the tissue sample prior to determining the expression level of mRNA. Methods for isolating mRNA from tissues are well-known to a skilled person. The methods which can be employed depend on the tissue type of the sample.

Methods for determining the expression level of protein are also known to a skilled person. For example, antibody-based assays like ELISA can be used to determine the expression level of such proteins. Antibody-based assays typically make use of antibodies or fragments thereof, which bind specifically to the protein in question, i.e. the long forms of Muc1 protein or total membrane-bound Muc1 protein.

In a preferred embodiment, the expression level of total membrane-bound Muc1 mRNA is measured in step (b), and the expression level of the long forms of Muc1 mRNA are measured in step (c). Such measurements are for example described in the Examples. The measurement of the expression level of mRNAs is particularly preferred.

Various methods are known to the skilled person for quantifying mRNA in a sample. In a preferred embodiment, the quantification may be performed by a reverse transcription step and a PCR step, more preferably a Real-time PCR step.

In another preferred embodiment, the expression level of total membrane-bound Muc1 protein is measured in step (b), and the expression level of the long forms of Muc1 protein are measured in step (c).

The expression level may be the amount or concentration. In case the amount of the mRNA or protein in a sample is determined, the sample if preferably of equal size and/or weight and/or of the same location in the body of the patient.

In case the concentration of the mRNA or protein is determined, the size or weight of the sample may differ. In this embodiment, it is preferred that the sample is of the same location in the body of the patient.

For example, all tissue samples taken at different time points are a biopsy from the breast epithelium of a breast cancer patient, or are a blood sample of an epithelial cancer patient, in particular a breast cancer patient.

It is often found during treatment of epithelial cancer patients, in particular of breast cancer patients, that patients are well responsive to a treatment in the beginning, but start to be less responsive and may even become unresponsive at some time during treatment. The present method surprisingly allows monitoring a cancer therapy closely and enables to determine very early, preferentially before clinical signs of disease recurrence occur, that the patient has become less responsive to a treatment and/or that a relapse occurs.

In clinical practice, an existing treatment for cancer is applied for a long, pre-determined time, without determining whether the patient has a benefit therefrom or continues to have a benefit therefrom. The present method of the invention allows determining a reduction in responsiveness to a cancer treatment very early by measuring the expression level of total membrane-bound Muc1 mRNA or protein in step (b) of the method of the invention, and the expression level of the long forms of Muc1 mRNA or protein in step (c) in dynamics, i.e. as a time course. According to one method of the invention, the ratio is determined at 2 different time points, an earlier, first time point and a later, second time point.

According to the present method, the ratio between the expression levels of (b) and (c) is measured at two different time points during a treatment. Thus, samples are obtained at two time points during an existing cancer treatment of such epithelial cancer patients. The second time point is at least 1 day later than the first time point, in order to determine a change in the ratio. Preferably, a longer interval may be used in order to determine a change in ratio, and thereby a change in responsiveness to the existing treatment may be determined. Therefore, the second sample is preferably obtained at least 1 week later than the first time point, more preferably at least 1 month later than the first time point, even more preferably at least 3, 6, 9 or 12 months later than the first time point.

The ratio between the expression level of (b) and (c) is understood as the value of: expression level of (b)/expression level of (c) of the method of the invention described above.

According to the method of the invention, it is determined whether an increase in ratio between the expression level of (b) and (c) has occurred. An “increase in ratio” is understood as an increase in ratio of expression levels by at least 10%, more preferably by at least 20%, even more preferably by at least 30%, most preferably by at least 50% or 100% at the second time point compared to the first time point.

The present method of the invention allows determining that the patient is less responsive to said cancer treatment, and is responsive to Muc1 based therapy. In such event, the present method allows adapting therapy of the patient in time. For example, the existing treatment may be stopped, and/or a Muc-1 based therapy may be initiated. Alternatively, the dosage of an existing therapy may be increased or the intervals of administration may be shortened in order to compensate for the reduction in responsiveness.

In a preferred embodiment, no further tumor markers are determined, in particular by determining their expression and/or activity.

In another embodiment, the expression levels of (i) Her-2 mRNA, (ii) Estrogen Receptor 1 (ER1) isotype 1 mRNA, and (iii) Progesterone Receptor (PR) mRNA are in addition determined, in order to obtain more detailed information on the cancer.

Therefore, in another preferred embodiment, the method of the invention further comprises following steps:
(a1) determining the expression level of
   (i) Her-2 mRNA,
   (ii) Estrogen Receptor 1 (ER1) isotype 1 mRNA, and
   (iii) Progesterone Receptor (PR) mRNA

in said tissue sample of said first time point (a1),
(b1) repeating steps (al) at said second time point (b1), which is at least 1 day later than said first time point, preferably at least 1 week later than said first time point, more preferably at least 1 month later than said first time point, even more preferably at least 3, 6, 9 or 12 months later than said first time point,
(c1) comparing the ratio of expression levels determined at said first time point (al) and said second time point (b1),

wherein
(i) an increase in ratio between the expression level in said tissue sample of (b) total membrane-bound Muc1 mRNA, or total membrane bound Muc1 protein and (c) the long forms of Muc1 mRNA, or the long forms of Muc1 protein at the second time point compared to the first time point, and
(ii) a decrease in expression level of Estrogen Receptor 1 (ER1) isotype alpha mRNA, and Progesterone receptor (PR) mRNA and optionally Her-2 mRNA at said second time point compared to said first time point,

indicates that the patient is less responsive to said cancer treatment, and is responsive to a Muc1 based therapy.

It was surprisingly found that an even better determination of responsiveness of an epithelial cancer patient can be obtained, when in addition to the time course or dynamics of the ratio of expression levels above, the expression level of the following mRNAs is determined: (i) Her-2 mRNA, (ii) Estrogen Receptor 1 (ER1) isotype 1 mRNA, and (iii) Progesterone Receptor (PR) mRNA. The expression level of these mRNAs is determined at the same first and second time points as the ratio of the method of the invention above. Thereby, the dynamics of a small panel of markers of an epithelial cancer patient under cancer treatment is determined.

Therefore, excellent prediction of responsiveness to a treatment is obtained by determining a small number of expression parameters.

Thus, in a preferred embodiment, no further markers, in particular tumor markers are determined, in particular by determining their expression and/or activity. Thus, in such preferred embodiment, no further tumor markers are determined in addition to a) the ratio of expression levels above and b) (i) Her-2 mRNA, (ii) Estrogen Receptor 1 (ER1) isotype 1 mRNA, and (iii) Progesterone Receptor (PR) mRNA expression levels.

It was surprisingly found that an increase in ratio between the expression level of (b) and (c) at the second time point compared to the first time point of the method as described above, and a decrease in expression level of Estrogen Receptor 1 (ER1) isotype alpha mRNA, and Progesterone receptor (PR) mRNA and optionally Her-2 mRNA at said second time point compared to said first time point, indicates that the patient is less responsive to said cancer treatment, and is responsive to a Muc1 based therapy.

A decrease in expression level of Estrogen Receptor 1 (ER1) isotype alpha mRNA, and Progesterone receptor (PR) mRNA further show a loss of these receptors on cancer cells.

Such loss of receptors is particular found in case of resistance to a therapy targeting Estrogen Receptor 1 (ER1) isotype alpha, and/or Progesterone receptor (PR). Therefore, in a preferred embodiment, the cancer therapy is a therapy targeting Estrogen Receptor 1 (ER1) isotype alpha, and/or Progesterone receptor (PR). In case an increase in ratio between the expression level of (b) and (c) at the second time point compared to the first time point of the method as described above is found, and a decrease in expression level of Estrogen Receptor 1 (ER1) isotype alpha mRNA, and Progesterone receptor (PR) mRNA, the existing treatment with an anti-ER1 and/or anti-PR treatment may be stopped, and/or a Muc-1 based therapy may be initiated, as described above. Alternatively, the dosage of an existing therapy may be increased or the intervals of administration may be shortened in order to compensate for the reduction in responsiveness.

In case no reduction in Her-2 mRNA expression is found at the second time point, a therapy targeting Her-2 may be initiated, e.g. by administration of an anti-Her2 antibody, such as trastuzumab.

The preferred embodiment of the present invention allows efficient and reliable monitoring therapy and/or for adapting therapy of an epithelial cancer patient, who is subject to a cancer treatment without determining an extensive panel of biomarkers.

In a more preferred embodiment, the epithelial cancer is breast cancer.

The method of the invention is in particular useful for breast cancer patients, as shown in the examples. Breast cancer has a high likelihood of recurrence and/or metastasis and monitoring therapy is therefore crucial.

In a preferred embodiment, the breast cancer patient is female or male, preferably female.

In a further preferred embodiment, the patient is already undergone surgery, in particular mastectomy or lumpectomy.

The tissue sample may be any suitable tissue which contains or is suspected to contain cancer cells. It is preferred that the tissue sample is obtained from the same location in the body for the different time points. For example, the tissue sample is always obtained from the tumor, e.g. by biopsy, or is always a blood sample.

Therefore, in case of breast cancer, the tissue sample is preferably a blood sample or a breast epithelium sample.

The cancer treatment to which the cancer patient is subject to may be any treatment aiming treating, ameliorating or slowing down the disease. A typical treatment regime for epithelial cancer is e.g. chemotherapy and/or irradiation.

In a yet further preferred embodiment, the cancer treatment is chemotherapy, treatment with aromatase inhibitor(s), a hormone therapy, a treatment with at least one agent directed against HER-2, or a combination thereof. Such treatment is in particular useful for treating breast cancer.

Aromatase inhibitors (AIs) are well known to a skilled person and are inhibitors of the enzyme aromatase. Aromatase is the enzyme that synthesizes estrogen. As breast and ovarian cancers require estrogen to grow, Als are taken to either block the production of estrogen or block the action of estrogen on receptors. There are 2 types of aromatase inhibitors (AIs) which are currently approved to treat breast cancer: Irreversible steroidal inhibitors, such as exemestane, forms a permanent and deactivating bond with the aromatase enzyme, and non-steroidal inhibitors, such as anastrozole and letrozole, which inhibit the synthesis of estrogen via reversible competition for the aromatase enzyme. Preferred selective aromatase inhibitors include anastrozole, letrozole, exemestane, vorozole, formestane, and fadrozole. Preferred non-selective aromatase inhibitors include aminoglutethimide and testolactone.

Chemotherapy is a category of cancer treatment that uses one or more anti-cancer drugs (so-called chemotherapeutic agents) that are given as part of a standardized chemotherapy regimen. Traditional chemotherapeutic agents act by killing cells that divide rapidly, one of the main properties of most cancer cells. Some newer anticancer drugs, for example, various monoclonal antibodies directed to specific cancer targets, are not indiscriminately cytotoxic, but rather target proteins that are abnormally expressed in cancer cells and that are essential for their growth. Such treatments are often referred to as targeted therapy as distinct from classic chemotherapy and are often used alongside traditional chemotherapeutic agents in antineoplastic treatment regimens. Chemotherapy may use one drug at a time (single-agent chemotherapy) or several drugs at once (combination chemotherapy or polychemotherapy). The combination of chemotherapy and radiotherapy is chemoradiotherapy. Preferred chemotherapeutic agents are alkylating agents, in particular selected from nitrogen mustards, nitrosoureas, tetrazines, aziridines, cisplatins and derivatives, and non-classical alkylating agents, more preferably selected from mechlorethamine, cyclophosphamide, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin and oxaliplatin. Further preferred chemotherapeutic agents are anti-metabolites, in particular selected from anti-metabolites are selected from anti-folates, fluoropyrimidines, deoxynucleoside analogues and thiopurine, more preferably selected from methotrexate, pemetrexed, fluorouracil, capecitabine, cytarabine, gemcitabine, decitabine, Vidaza, fludarabine, nelarabine, cladribine, clofarabine, pentostatin, thioguanine and mercaptopurine. Further preferred chemotherapeutic agents are anti-microtubule agents, in particular selected from taxanes, in particular paclitaxel and docetaxel, vincristine, vinblastine, vinorelbine, vindesine, vinflunine, etoposide and teniposide. Further preferred chemotherapeutic agents are topoisomerase inhibitors, such as irinotecan, topotecan, etoposide, doxorubicin, mitoxantrone, teniposide, novobiocin, merbarone, and aclarubicin. Preferred chemotherapeutic agents are also cytotoxic antibiotics, such as anthracyclines, including actinomycin, bleomycin, plicamycin, mitomycin, doxorubicin and daunorubicin.

In another embodiment, the breast cancer patient is treated with an anti-estrogen agent, in particular tamoxifen.

Suitable anti-cancer agents approved for the therapy of breast cancer are:
Methotrexate
Paclitaxel Albumin-stabilized Nanoparticle Formulation
Ado-Trastuzumab Emtansine
Doxorubicin Hydrochloride
Fluorouracil
Everolimus
Anastrozole
Pamidronate Disodium
Exemestane
Cyclophosphamide
Docetaxel
Epirubicin Hydrochloride
Toremifene
Fulvestrant
Letrozole
Methotrexate
Gemcitabine Hydrochloride
Trastuzumab
Ixabepilone
Lapatinib Ditosylate
Letrozole
Megestrol Acetate
Cyclophosphamide
Tamoxifen Citrate
Pertuzumab
Paclitaxel
Docetaxel
Trastuzumab
Capecitabine, and
Goserelin Acetate.

Drug combinations used in Breast Cancer are for example AC, AC-T, CAF, CMF, FEC and TAC. For example, CMF is known to be a combination therapy of cyclophosphamide, methotrexate and 5-fluorouracil. AC is known to be a combination therapy of cyclophosphamide and doxorubicin.

In a more preferred embodiment, the combination is a combination therapy of chemotherapy and treatment with aromatase inhibitor(s).

In another more preferred embodiment, a treatment with aromatase inhibitor(s) is an adjuvant therapy.

Adjuvant therapy, also called adjuvant care, is a treatment that is given in addition to the primary, main or initial treatment.

In one embodiment of the methods of the invention, the patient is a breast cancer patient. In a more preferred embodiment of the present invention, said patient has undergone breast cancer surgery.

Breast cancer surgery represents a standard initial treatment for removing cancerous cells.

In a further preferred embodiment of the present invention, the cells are obtained after breast surgery, in particular after 2, 3, 6 or more months after breast surgery.

For example, such method is useful for monitoring therapy and/or adapting therapy of an epithelial cancer patient, preferably a breast cancer patient, who was confirmed to be HER-2-positive, Estrogen Receptor 1 (ESR1) isotype a-positive and/or progesterone receptor (PR)-positive and/or responsive to an agent directed against Her-2 or hormone therapy before or at the time of surgery. Such person was therefore considered eligible for treatment with an agent directed against Her-2 and/or for hormone therapy. Thus, such person has in a preferred embodiment been subject to administration of an agent directed against Her-2 and/or to hormone therapy. When the method of the invention is performed, in particular by determining both the (i) the ratio (b)/(c) wherein (b) is the expression level of total membrane-bound Muc-1 mRNA or protein and (c) is the expression level of the long forms of Muc1 mRNA or protein, and (ii) the expression levels of Her-2, ESR1 and PR mRNAs, and in case it is found that the ratio increases and expression levels of the ESR1 and PR mRNAs and optionally the Her2 mRNA decreases, this indicates that the patient is less responsive to such treatment. The hormone therapy may be either stopped, or the strength or dosage of the hormone therapy may be increased. Alternatively or in addition, a Muc1-based therapy may be started, e.g. by administering an anti-Muc1 antibody. In case the expression level of Her2 was not decreased, the administration of an agent directed against Her-2 may be initiated or continued, respectively.

Therefore, in a further preferred embodiment of the present invention, the patient was confirmed to be HER-2-positive, Estrogen Receptor 1 (ESR1) isotype a-positive and/or progesterone receptor (PR)-positive and/or responsive to an agent directed against Her-2 or hormone therapy before or at the time of surgery.

In a yet further preferred embodiment of the present invention, the patient was confirmed to be HER-2-negative, Estrogen Receptor 1 (ESR1) isotype a-negative and/or progesterone receptor (PR)-negative and/or non-responsive to an agent directed against Her-2 or hormone therapy before or at the time of surgery.

In such event, the patient was therefore considered ineligible for treatment with an agent directed against Her-2 and/or for hormone therapy. Thus, such person has in a preferred embodiment not been subject to administration of an agent directed against Her-2 and/or to hormone therapy and has received chemotherapy and/or radiotherapy. Performing the method of the invention may give further insight on any amendments in the expression status of the tumor of such patient. In case such patient does not show an increase in the ratio between the expression level of total membrane-bound Muc-1 mRNA or protein and the expression level of the long forms of Muc1, the existing therapy may be continued. In case of an increase in the ratio is determined, a Muc1 -based therapy may be initiated.

In another embodiment, the present invention relates to a method for determining malignancy grade or progression of a tumor of a patient suffering from an epithelial tumor, comprising:
(a) obtaining a tissue sample comprising tumor cells from said patient,
(b) determining the expression level of
   (i) total membrane-bound Muc1 mRNA, or
   (ii) total membrane bound Muc1 protein
   in said tissue sample,
(c) determining the expression level of
   (i) the long forms of Muc1 RNA, or
   (ii) the long forms of Muc1 protein

in said tissue sample,

wherein

an expression level of (b) higher than the expression level of (c) indicates
(α) that said tissue sample is malignant, and/or
(β) that the tumor has increased its malignancy grade, and/or
(γ) that the patient is progressing and/or is less responsive to the currently applied tumor therapy.

It was surprisingly found that an expression level of total membrane-bound Muc1 mRNA or protein which is higher than expression level of the long forms of Muc1 mRNA in a tissue sample from an epithelial cancer patient indicates that that said tissue sample is malignant. In a preferred embodiment, an expression level of total membrane-bound Muc1 mRNA or protein which is at least 10%, 20%, 30%, 40%, 50% or 100% higher than the expression level of the long forms of Muc1 mRNA or protein in a tissue sample from an epithelial cancer patient indicates that the tissue sample is malignant.

The tissue sample comprising tumor cells may be any suitable tissue sample, like the tumor tissue or blood.

Therefore, the method of the invention is in particular useful for determining whether a tissue sample contains malignant cells. Such assessment is crucial for prognosis of the disease and for determining treatment options. Such method can be applied to samples from patients, which do not have undergone a cancer treatment, e.g. shortly after diagnosis, or it may applied to samples from patients, which are currently subject to a treatment, or to samples from patients which have completed a treatment or therapy.

It was further surprisingly found that an expression level of total membrane-bound Muc1 mRNA or protein which is higher than expression level of the long forms of Muc1 mRNA in a tissue sample from an epithelial cancer patient indicates that the tumor has increased its malignancy grade. In a preferred embodiment, an expression level of total membrane-bound Muc1 mRNA or protein which is at least 10%, 20%, 30%, 40%, 50% or 100% higher than the expression level of the long forms of Muc1 mRNA or protein in a tissue sample from an epithelial cancer patient indicates that the tumor has increased its malignancy grade.

In this embodiment of the invention, it may be determined whether a tumor which is known to exhibit a certain degree of malignancy, has further increased its malignancy grade.

It was further surprisingly found that an expression level of total membrane-bound Muc1 mRNA or protein which is higher than expression level of the long forms of Muc1 mRNA or protein in a tissue sample from an epithelial cancer patient indicates that the patient is progressing and/or is less responsive to the currently applied tumor therapy.

In one preferred embodiment, the method is applied to samples from a patient to whom a tumor therapy is applied. By determining that the expression level of total membrane-bound Muc1 mRNA or protein is higher than expression level of the long forms of Muc1 mRNA or protein in a tissue sample from such epithelial cancer patient, it is determined that the patient is progressing and/or is less responsive to the currently applied tumor therapy.

A malignant tumor contrasts with a non-cancerous benign tumor in that a malignant tumor is not self-limited in its growth, is capable of invading into adjacent tissues, and may be capable of spreading to distant tissues. A benign tumor has none of those properties. Malignancy in cancer is characterized by anaplasia, invasiveness, and metastasis.

In a preferred embodiment of the present invention, the long forms of Muc1 RNA are all Muc1 mRNA molecules encoding at least exons III to VII of Muc1.

In a further preferred embodiment of the present invention, the long forms of Muc1 RNA are all Muc1 mRNA molecules encoding a Muc1 protein comprising up to 39 repeats in the variable number tandem repeat (VNTR) domain.

In an even more preferred embodiment, the long forms of Muc1 RNA are all Muc1 mRNA molecules encoding at least exons III to VII of Muc1, and which are encoding a Muc1 protein comprising up to 39 repeats in the variable number tandem repeat (VNTR) domain.

In a more preferred embodiment of the present invention, the long forms of Muc1 protein comprise at least a part of the variable number tandem repeat (VNTR) domain.

As described in the examples, suitable primers may be used for amplifying long forms of Muc1 mRNA after reverse transcription, for example by taking into account the sequences of exons III and VII of the Muc1 mRNA. Preferred primers suitable in this context are shown in the examples.

According to the present invention, total membrane-bound Muc1 mRNA is understood as all Muc1 mRNAs encoding Muc1 proteins which contain a transmembrane domain. As described in the examples, suitable primers may be used for amplifying total membrane-bound Muc1 mRNA after reverse transcription, for example by taking into account the sequence of the Muc1 mRNA encoding the transmembrane domain. Preferred primers suitable in this context are shown in the examples.

In a preferred embodiment of the present invention, the epithelial cancer is selected from breast cancer, colon cancer, esophageal cancer, gastric cancer, lung cancer, melanoma, bladder cancer, ovarian cancer, prostate cancer and pancreatic cancer.

In one embodiment of the methods of the invention, the patient is a breast cancer patient. In a more preferred embodiment of the present invention, said patient has undergone breast cancer surgery.

Breast cancer surgery represents a standard initial treatment for removing cancerous cells.

In a further preferred embodiment of the present invention, the cells are obtained after breast surgery, in particular after 2, 3, 6 or more months after breast surgery.

In a preferred embodiment of the present invention, hormone therapy is a treatment with at least one agent directed against Estrogen Receptor 1 (ESR1) isotype a and/or progesterone receptor (PR). In particular, hormone therapy may be treatment with tamoxifen.

In a preferred embodiment of the present invention, the expression level of a Muc1 mRNA or Muc1 protein is the amount or concentration of the Muc1 mRNA or Muc1 protein, which is preferably normalized.

In order to determine the expression level of a Muc1 mRNA or Muc1 protein, the amount or concentration of the Muc1 mRNA or Muc1 protein is preferably determined. In a more preferred embodiment, the determined amount or concentration of the Muc1 RNA or Muc1 protein is preferably normalized. This can be performed by methods known to a skilled person.

It was surprisingly found that RT-PCR (Real-time PCR) methods are in particular useful for determining the amount or concentration of Muc1 mRNAs, as shown in the examples. Therefore, in a preferred embodiment of the present invention, the expression levels of total membrane-bound Muc1 mRNA and the long forms of Muc1 mRNA are determined.

It was found that additional information on the tumor status can be obtained by determining the expression levels of one or more of the biomarkers ESR1, PR and Her2. Therefore, in a more preferred embodiment of the method of the present invention for determining malignancy grade or progression of a tumor of a patient suffering from an epithelial tumor, the expression levels of 1, 2, or 3, preferably 3, of the following mRNAs is determined in addition: (i) HER-2, (ii) Estrogen Receptor 1 (ESR1) isotype α, (iii) progesterone receptor (PR) mRNA.

As described above, the method of the invention for monitoring therapy and/or for adapting therapy of an epithelial cancer patient, who is subject to a cancer treatment, involves determining a ratio of expression at two different time points. In a further preferred embodiment, the ratio of expression is determined at further time points, like 1, 2, 3, 4, 5, 6, 7, 8, 9, 10, 11, 12, 13, 14, 15 or more time points. Thereby, changes of the ratio of the expression level of total membrane-bound Muc-1 mRNA or protein to the expression level of the long forms of Muc1 mRNA or protein, in particular mRNA, can be determined over time. Thereby, the success of the therapy can be monitored. Steps (a) to (f) of the method of the invention may be repeated several times. A sample is obtained at a time point at least 1 day later than the previous sample. Preferably, a sample is obtained at a time point at least 1 week or 1 month later than the previous sample. The time intervals for obtaining a sample may be the same or may be different for each repetition. For example, it is possible to obtain a sample s2 1 week after a previous sample s1, and then again to obtain a further sample s3 2 months after the respective previous sample s2.

Thereby, a time course of the ratio of expression levels can be determined. In case no increase or an increase in the ratio of expression levels of less than 10% is determined, the patient is responsive to the ongoing cancer treatment. In this case, the cancer treatment may be continued. As soon as an increase in the ratio of expression levels by at least 10%, more preferably by at least 20%, even more preferably by at least 30%, most preferably by at least 50% or 100% at the latest time point compared to the previous time point is determined, this indicates that (i) the patient is less responsive to said treatment, and (ii) is responsive to Muc1 based therapy.

In this case, the current, existing treatment may be stopped, and/or a Muc-1 based therapy may be initiated, as described above. Alternatively, the dosage of an existing therapy may be increased or the intervals of administration may be shortened in order to compensate for the reduction in responsiveness.

Therefore, in a preferred embodiment of the present invention, the method of the invention for monitoring therapy and/or for adapting therapy of an epithelial cancer patient, who is subject to a cancer treatment, further comprises following steps:
(g) repeating steps (a) to (f) of the method of the invention further 1, 2, 3 or more times at a time point at least 1 day later than the respective previous repetition, preferably at least 1 week later than the respective previous repetition, more preferably at least 1 month later than the respective previous repetition, even more preferably at least 3, 6, 9 or 12 months later than the respective previous repetition,
(h) comparing the ratio of expression levels determined at the different time points, wherein

an increase in ratio between the expression level of (b) and (c) at a later time point compared to an earlier time point indicates that
(i) the patient is less responsive to said treatment, and
(ii) is responsive to Muc1 based therapy.

As described above, it was surprisingly found that an even better determination of responsiveness of an epithelial cancer patient can be obtained, when in addition to the time course or dynamics of the ratio of expression levels above, the expression level of the following mRNAs is determined: (i) Her-2 mRNA, (ii) Estrogen Receptor 1 (ER1) isotype 1 mRNA, and (iii) Progesterone Receptor (PR) mRNA. Therefore, it is preferred that the expression levels of (i) Her-2 mRNA, (ii) Estrogen Receptor 1 (ER1) isotype 1 mRNA, and (iii) Progesterone Receptor (PR) mRNA are also determined at further time points, like 1, 2, 3, 4, 5, 6, 7, 8, 9, 10, 11, 12, 13, 14, 15 or more time points. The expression level of these mRNAs is preferably determined at the same time points as the ratio of expression levels above, in the same samples. Thereby, the time course or dynamics of a small panel of markers of an epithelial cancer patient under cancer treatment can be determined.

Therefore, excellent prediction of responsiveness and changes in the responsiveness over therapy time is obtained by determining a small number of expression parameters.

In a preferred embodiment, no further markers, in particular tumor markers are determined. In particular, no further tumor markers are determined by determining their expression level and/or activity. Therefore, in such preferred embodiment, no further tumor markers are determined in addition to a) the ratio of expression levels above and b) (i) Her-2 mRNA, (ii) Estrogen Receptor 1 (ER1) isotype 1 mRNA, and (iii) Progesterone Receptor (PR) mRNA.

Therefore, in a further preferred embodiment, the method of the invention for monitoring therapy and/or for adapting therapy of an epithelial cancer patient, who is subject to a cancer treatment, further comprises following steps:
(i) repeating step (al) of the method of the invention further 1, 2, 3 or more times at a time point at least 1 day later than the respective previous repetition, preferably at least 1 week later than the respective previous repetition, more preferably at least 1 month later than the respective previous repetition, even more preferably at least 3, 6, 9 or 12 months later than the respective previous repetition,
(j) comparing the expression levels determined at the different time points, wherein
   (i) an increase in ratio between
      (b) the expression level of total membrane-bound Muc1 mRNA, or total membrane bound Muc1 protein and
      (c) the long forms of Muc1 mRNA, or the long forms of Muc1 protein at a later time point compared to an earlier time point, and
   (ii) a decrease in expression level of Estrogen Receptor 1 (ER1) isotype 1 mRNA, and Progesterone receptor (PR) mRNA, and optionally Her-2 mRNA at a later time point compared to an earlier time point

in said tissue sample indicates that the patient is less responsive to said treatment, and is responsive to a Muc1 based therapy.

A decrease in expression level of Estrogen Receptor 1 (ER1) isotype alpha mRNA, and Progesterone receptor (PR) mRNA at a later time point compared to the respective earlier time point shows a loss of these receptors on cancer cells.

Such loss of receptors is particular found in case of resistance to a therapy targeting Estrogen Receptor 1 (ER1) isotype alpha, and/or Progesterone receptor (PR). Therefore, in a preferred embodiment, the cancer therapy is a therapy targeting Estrogen Receptor 1 (ER1) isotype alpha, and/or Progesterone receptor (PR). In case an increase in ratio between the expression level of (b) and (c) at the at a later time point compared to the respective earlier time point of the method as described above is found, and a decrease in expression level of Estrogen Receptor 1 (ER1) isotype alpha mRNA, and Progesterone receptor (PR) mRNA, the existing treatment with an anti-ER1 and/or anti-PR treatment stopped, and/or a Muc-1 based therapy may be initiated, as described above. Alternatively, the dosage of an existing therapy may be increased or the intervals of administration may be shortened in order to compensate for the reduction in responsiveness.

In case no reduction in Her-2 mRNA expression is found at the later time point, a therapy targeting Her-2 may be initiated, e.g. by administration of an anti-Her2 antibody.

In case the patient is determined to be less responsive to the existing, ongoing treatment, the patient will typically encounter progressive disease, as the current treatment will be less effective or not effective anymore. Therefore, chemotherapy treatment with high dosage and/or short intervals of chemotherapeutic agent(s) to be administered should be started in one preferred embodiment for such patient.

In case the patient is determined to be less responsive to the existing, ongoing treatment, and is determined to be responsive to a Muc1 based therapy, the patient is preferably determined to suffer from progressive disease. Further, in case the patient is determined to be less responsive to the existing, ongoing treatment, and is determined to be responsive to a Muc1 based therapy, the patient is preferably determined to be responsive to a chemotherapy treatment with high dosage and/or short intervals of chemotherapeutic agent(s) to be administered.

Suitable chemotherapeutic agent(s) and chemotherapeutic regimens are described above.

As described above, the present invention relates in one embodiment to a method for determining malignancy grade or progression of a tumor of a patient suffering from an epithelial tumor by determining the expression levels of total membrane-bound Muc1 mRNA or protein and the long forms of Muc1 mRNA or protein in a tissue sample comprising tumor cells. An expression level of total membrane-bound Muc1 mRNA or protein which is higher than expression level of the long forms of Muc1 mRNA in a tissue sample from an epithelial cancer patient indicates that that said tissue sample is malignant. By determining the expression levels of total membrane-bound Muc1 mRNA or protein and the long forms of Muc1 mRNA or protein in a tissue sample comprising tumor cells obtained at two different time points from said patient, a change of the expression levels and a change in the difference between expression levels of total membrane-bound Muc1 mRNA or protein and the long forms of Muc1 mRNA or protein can be obtained. As long as the difference (a)-(b) between (a) the expression level of total membrane-bound Muc1 mRNA, or total membrane bound Muc1 protein and (b) the expression level of the long forms of Muc1 RNA, or the long forms of Muc1 protein at a later time point compared to the respective earlier time point does not increase, or increases by less than 10%, the tissue sample is not become more malignant, the tumor has not further increased its malignancy grade, the patient is not further progressing and is responsive to the currently applied tumor therapy. As soon as it is determined that the proportion of total membrane-bound Muc1 mRNA, or total membrane bound Muc1 protein increases as compared to long forms of Muc1 mRNA or protein, as thereby the difference (a)-(b) between (a) the expression level of total membrane-bound Muc1 mRNA, or total membrane bound Muc1 protein and (b) the expression level of the long forms of Muc1 RNA, or the long forms of Muc1 protein is higher at a later time point compared to the respective earlier time point, preferably, wherein the difference is at least 10%, 20%, 30%, 40%, 50% or 100% higher at a later time point compared to the respective earlier time point, the tissue sample has become more malignant, the tumor has further increased its malignancy grade, the patient is further progressing and is less responsive to the currently applied tumor therapy.

Therefore, in a further preferred embodiment, the method of the invention for determining malignancy grade or progression of a tumor of a patient suffering from an epithelial tumor further comprises following steps:
(d) repeating steps (a) to (c) at a time point at least 1 day later, preferably at least 1 week later, more preferably at least 1 month later, even more preferably at least 3, 6, 9 or 12 months later,

wherein an increase in the difference (a)-(b) between the expression level of
(a) total membrane-bound Muc1 mRNA, or total membrane bound Muc1 protein and
(b) the expression level of the long forms of Muc1 RNA, or the long forms of Muc1 protein at the later time point compared to the earlier time point indicates that:
(α) that said tissue sample has become more malignant, and/or
(β) that the tumor has further increased its malignancy grade, and/or
(γ) that the patient is further progressing and/or is less responsive to the currently applied tumor therapy.

In a further preferred embodiment, the difference in expression level between the expression level of
(a) total membrane-bound Muc1 mRNA, or total membrane bound Muc1 protein and
(b) the expression level of the long forms of Muc1 RNA, or the long forms of Muc1 protein

is determined at further time points, like 1, 2, 3, 4, 5, 6, 7, 8, 9, 10, 11, 12, 13, 14, 15 or further time points.

Thus, samples are obtained at different time points and the expression levels are determined. Thereby, changes in the difference between the expression level of total membrane-bound Muc-1 mRNA or protein and the expression level of the long forms of Muc1 mRNA or protein, in particular mRNA, over time can be determined. Thereby, the success of the therapy can be monitored. Steps (a) to (c) of the method of the invention may be repeated several times. A sample is obtained at a time point at least 1 day later than the previous sample. Preferably, a sample is obtained at a time point at least 1 week or 1 month later than the previous sample. The time intervals for obtaining a sample may be the same or may be different for each repetition. For example, it is possible to obtain a sample s2 1 week after a previous sample s1, and then again to obtain a further sample s3 2 months after the respective previous sample s2.

Thereby, a time course of the difference in expression level levels can be determined. In case no increase or an increase in the difference in expression levels of less than 10% is determined, the tissue sample has not become more malignant, and/or the tumor has not further increased its malignancy grade, and/or the patient is not further progressing and/or is responsive to the currently applied tumor therapy. In this case, the current cancer treatment may be continued. As soon as an increase in the difference in expression level levels by at least 10%, more preferably by at least 20%, even more preferably by at least 30%, most preferably by at least 50% or 100% at the latest time point compared to the previous time point is determined, this indicates that the tissue sample has become more malignant and/or that the tumor has further increased its malignancy grade and/or that the patient is further progressing and/or is less responsive to the currently applied tumor therapy.

Therefore, in a further more preferred embodiment, the method of the invention for determining malignancy grade or progression of a tumor of a patient suffering from an epithelial tumor further comprises following steps:
(e) further repeating steps (a) to (c) further 1, 2, 3 or more times at a time point at least 1 day later than the respective previous repetition, preferably at least 1 week later than the respective previous repetition, more preferably at least 1 month later than the respective previous repetition, even more preferably at least 3, 6, 9 or 12 months later than the respective previous repetition,

wherein an increase in the difference (a)-(b) between the expression level of
(a) total membrane-bound Muc1 mRNA, or total membrane bound Muc1 protein and
(b) the expression level of the long forms of Muc1 RNA, or the long forms of Muc1 protein at a later time point compared to an earlier time point indicates that:
(α) that said tissue sample has become more malignant, and/or
(β) that the tumor has further increased its malignancy grade, and/or
(γ) that the patient is further progressing and/or is less responsive to the currently applied tumor therapy.

In a more preferred embodiment of the present invention, the tissue is blood. A blood sample may be obtained easily from a patient, also repetitively. Moreover, RNA may be obtained from a blood sample by methods known in the art.

In a preferred embodiment, mRNA expression levels are determined in the methods of the present invention, in particular by RT-PCR.

“RT-PCR” is understood as “Real time PCR” according to the present invention. Real time PCR is also called qPCR. Its key feature is that the amplified DNA is detected as the reaction progresses, so-called in “real time”. Known methods for the detection of products in Real time PCR are: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence to quantify messenger RNA (mRNA) and non-coding RNA in cells or tissues. Both methods may be used according to the invention, preferably the use of sequence-specific DNA probes is preferred. The general principle of Real time PCR with a sequence-specific DNA probe is shown in FIG. 16. In this embodiment, fluorescent reporter probes detect only the DNA containing the probe sequence; therefore, use of the reporter probe significantly increases specificity, and enables quantification even in the presence of non-specific DNA amplification. The method relies on a DNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the Taq polymerase breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter. Fluorescence is detected and measured in a real-time PCR machine, and its geometric increase corresponding to exponential increase of the product is used to determine the quantification cycle (Cq) in each reaction.

Therefore, the methods of the invention preferably comprise the step of reverse transcription of mRNA and subsequent Real-time PCR.

As shown in the examples, the time of courses of expression levels could be established reliably using RT-PCR. Also, ratio of expression levels and differences of expressions levels could be determined reliably starting from expression levels established by RT-PCR. Therefore, in a yet more preferred embodiment of the present invention, the expression level(s) of each mRNA in methods of the inventions is determined by Real-time PCR.

In a preferred embodiment, the method of the invention is a Real-Time RT-PCR method.

The Real-Time RT-PCR method is designed for quantitative determination of human MUC1, HER2-neu (erb2), ER, PR gene expression level in breast cancer samples, MUC1 expression level in the other epithelium-originated malignant tissues, such as ovarian, prostate, lung, bladder, colon and pancreatic cancers, by reverse transcription and real-time PCR. The methods and kits of the invention allow to determine the total number of copies of “normal” full-length MUC1 mRNA variant in the tissue sample and also the majority of MUC1 mRNA forms generated during alternative splicing of MUC1 pre-mRNA, including splice variants MUC1/A and MUC1/D and short forms MUC1/X, MUC1/Y, MUC1/Z known to be associated with the presence of malignancy [4, 35].

In order to determine the expression level, in particular amount or concentration of an mRNA in a sample by RT-PCR, normalization of the determined values is preferably performed. As shown in the examples, it was surprisingly found that normalization by determining the total amount of RNA by spectrometry or fluorometry leads to clearly superior results as compared to normalization to the expression of a reference gene, such as the for beta-2 microglobulin (B2M) gene.

Therefore, in a further preferred embodiment, normalization is performed in the context of Real-time PCR,

and wherein normalization
(a) is not performed by normalization to the expression of a reference gene, and/or
(b) is performed by determining the total amount of RNA by spectrometry or fluorometry.

Moreover, it was surprisingly found that clearly superior results and reliable results are obtained in case the RT-PCR by using a single primer pair per Real-time PCR reaction in contrast to multiplex Real-time PCR.

Therefore, in a further preferred embodiment, Real-time PCR
a) is not performed as multiplex Real-time PCR, and/or
b) is performed by using a single primer pair per Real-time PCR reaction.

As shown in the examples, the expression level of total membrane-bound Muc1 mRNA could be determined successfully by RT-PCR starting from tissue samples of cancer patients using specifically designed primers.

Thus, in a more preferred embodiment, the method of the invention comprises steps for determining total membrane-bound Muc1 mRNA:
(a) isolating total RNA from the tissue sample,
(b) reverse transcribing the RNA into cDNA,
(c) performing Real-time PCR using one or more of the following primer pairs (i) to (xii) for determining total membrane-bound Muc1 mRNA:

(i)

(SEQ ID No. 1)

CCTCCCCACCCATTTCACC
an

(SEQ ID No. 2)

CTGTAAGCACTGTGAGGAGC
(ii)

(SEQ ID no. 3)

CCTACCATCCTATGAGCGAG
and

(SEQ ID No. 4)

CCCTACAAGTTGGCAGAAGTG
(iii)

(SEQ ID No. 5)

CTACTGAGAAGAATGCTTTGTCTA
and

(SEQ ID No. 6)

GCCTGAACTTAATATTGGAGAGG
(iv)

(SEQ ID No. 7)

CTACTGAGAAGAATGCTTTTAATTCC
and

(SEQ ID No. 8)

GCCTGAACTTAATATTGGAGAGG
(v)

(SEQ ID No. 9)

CTACTGAGAAGAATGCTTTGTCTA
and

(SEQ ID No. 10)

CTCTTGGTAGTAGTCGGTGC
(vi)

(SEQ ID No. 11)

(CCAGCACCGACTACTACCAA
or

(SEQ ID No. 13))

CACCGACTACTACCAAGAGC
and

(SEQ ID No. 12)

CTCTTGGTAGTAGTCGGTGC
(vii)

(SEQ ID No. 14)

CTACTGAGAAGAATGCTTTTAATTCC
and

(SEQ ID No. 15)

GCCTGAACTTAATATTGGAGAGG
(viii)

(SEQ ID No. 16)

CTACTGAGAAGAATGCTTTTAATTCC
and

(SEQ ID No. 17)

(CGGCACTGACAGACAGCCAT
or

(SEQ ID No. 18))

GGCACTGACAGACAGCCATT
(ix)

(SEQ ID No. 19)

CTACTGAGAAGAATGCTTTTAATTCC
and

(SEQ ID No. 20)

CACCCCAGCCCCAGACATT
(x)

(SEQ ID No. 21)

CTACTGAGAAGAATGCTTTTTTGC
and

(SEQ ID No. 22)

AGGCTGCTTCCGTTTTATACTG
(xi)

(SEQ ID No. 23)

CCTCTCCAATATTAAGTTCAGTGA
and

(SEQ ID No. 24)

ACAGACAGCCAAGGCAATGAG
(xii)

(SEQ ID No. 25)

(CCTCTCCAATATTAAGTTCAGTCT
or

(SEQ ID No. 26))

CCTCTCCAATATTAAGTTCAGTC
and

(SEQ ID No. 27)

ACAGACAGCCAAGGCAATGAG,

and
(d) determining the expression level of total Muc1 mRNA.

In a preferred embodiment, one or more of the primers according to SEQ ID No. 2, 4, 6, 8, 10, 12, 15, 17, 18, 20, 22, 24 and 27 are used in step (b) for reverse transcribing the RNA into cDNA.

As further shown in the examples, the expression level of the long forms of Muc1 mRNA could be determined successfully by RT-PCR starting from tissue samples of cancer patients using specifically designed primers.

Therefore, in a more preferred embodiment, the method of the invention comprises steps for determining long forms of Muc1 mRNA:
(a) isolating total RNA from the tissue sample,
(b) reverse transcribing the RNA into cDNA,
(c) performing Real-time PCR using one or more of the following primer pairs (1)-(3) for determining the long forms of Muc1 mRNA:

(1)

(SEQ ID No. 28)

CCACTCTGATACTCCTACCAC
and

(SEQ ID No. 29)

GAAAGAGACCCCAGTAGACAAC,
(2)

(SEQ ID No. 30)

CCTCCCCACCCATTTCACC
and

(SEQ ID No. 31)

CTGTAAGCACTGTGAGGAGC,
(3)

(SEQ ID No. 32)

CACTTCTGCCAACTTGTAGGG,
and

(SEQ ID No. 33)

CCCTACAAGTTGGCAGAAGTG,

and
(d) determining the expression level of long forms of Muc1 mRNA.

RT-PCR employs typically further employs probe molecules which are labelled, in particular with a fluorescent label and a quencher moiety (see FIG. 16). Specifically designed probes were developed successfully for RT-PCR as shown in the examples. In the probes of the examples, the fluorescent label is ROX or FAM and the quencher moiety is BHQ2.

In a more preferred embodiment of the method of the invention, following probes are used:

(a)

(SEQ ID No. 34)

TGACACCGGGCACCCAGTCTCC
and/or

(SEQ ID No. 35)

CCACCATGACACCGGGCACCCA
for primer pair (i)
(b)

(SEQ ID No. 36)

TGCAGGTAATGGTGGCAGCAGCC
for primer pair (ii)
(c)

(SEQ ID No. 37)

AGCACCGACTACTACCAAGAGCTGCA
and/or

(SEQ ID No. 38)

TTTCCTGTCTTTTCACATTTCAAACCTCCAGTT
for primer pair (iii)
(d)

(SEQ ID No. 39)

CAGCACCGACTACTACCAAGAGCTGC
for primer pair (iv)
(e)

(SEQ ID No. 40)

TTTCCTGTCTTTTCACATTTCAAACCTCCAGTT
for primer pair (v)
(f)

(SEQ ID No. 41)

ATGGCTGTCTGTCAGTGCCGCCGAA
for primer pair (vi)
(g)

(SEQ ID No. 42)

AGCACCGACTACTACCAAGAGCTGCA
and/or

(SEQ ID No. 43)

CAGCACCGACTACTACCAAGAGCTGC
for primer pair (vii)
(h)

(SEQ ID No. 44)

AGCACCGACTACTACCAAGAGCTGCA
for primer pair (viii)
(i)

(SEQ ID No. 45)

AGCACCGACTACTACCAAGAGCTGCA
and/or

(SEQ ID No. 46)

CAGCACCGACTACTACCAAGAGCTGC
for primer pair (ix)
(j)

(SEQ ID No. 47)

TTGACTCTGGCCTTCCGAGAAGGTAC
and/or

(SEQ ID No. 48)

CTTCCGAGAAGGTACCATCAATGTCCAC
for primer pair (x)
(k)

(SEQ ID No. 49)

CATCGCGCTGCTGGTGCTGGTCT
and/or

(SEQ ID No. 50)

TGTGCCATTTCCTTTCTCTGCCCAGTC
for primer pair (xi), and
(l)

(SEQ ID No. 51)

CATCGCGCTGCTGGTGCTGGTCT
for primer pair (xii),

wherein the probes are labeled,

preferably labeled with a fluorescent label and a quencher moiety,

more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe.

In a more preferred embodiment of the method of the invention, following probes are used:

(m)

(SEQ ID No. 52)

AGCCATAGCACCAAGACTGATGCCA
and/or

(SEQ ID No. 53)

ACCTCCTCTCACCTCCTCCAATCACA
for primer pairs (1) to (3),

wherein the probes are labeled, preferably labeled with a fluorescent label and a quencher moiety,

more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe,

even more preferably wherein the fluorescent label is ROX or FAM and the quencher moiety is BHQ2.

As described above for various methods of the invention, it is preferred that the expression levels of
(i) total membrane-bound Muc1 mRNA,
(ii) the long forms of Muc1 RNA,
(iii) Her-2 mRNA,
(iv) Estrogen Receptor 1 (ER1) isotype 1 mRNA, and
(v) Progesterone Receptor (PR) mRNA are determined.

In an even more preferred embodiment, no further markers, are determined. In particular no further tumor markers are determined, in particular by determining their expression level and/or activity.

In the examples, RT-PCR methods could be established successfully for determining the expression levels of all mRNAs (i) to (v) by specifically identifying suitable primer pairs.

Thus, in a more preferred embodiment of the method of the invention, the expression level of
(i) total membrane-bound Muc1 mRNA,
(ii) the long forms of Muc1 RNA,
(iii) Her-2 mRNA,
(iv) Estrogen Receptor 1 (ER1) isotype 1 mRNA, and
(v) Progesterone Receptor (PR) mRNA

is determined,

and wherein
(a) determining the expression level of human HER-2 with Real-time PCR is performed using one or more of the following primer pairs:

(1)

(SEQ ID No. 54)

CGTTTGAGITCCATGCCCAATC
and

(SEQ ID No. 55)

TCCTCTGCTGITCACCTCTTG,
(2)

(SEQ ID No. 56)

CACCCACTCCCCTCTGAC
and

(SEQ ID No. 57)

CAGCAGITCTCCGCATCGTG
(3)

(SEQ ID No. 58)

GTGAAACCTGACCTCTCCTAC
and

(SEQ ID No. 59)

CAGCAGTCTCCGCATCGTG,

preferably wherein followina probes are used:

(SEQ ID No. 60)

CTGCCTGITCCCTACAACTACCTTTCTAC,
for primer pair (1)

(SEQ ID No. 61)

ATCCTCATCAAGCGACGGCAGCAGAA,
for primer pair (2)
and/or

(SEQ ID No. 62)

AGCAGAGAGCCAGCCCTCTGACGTCCATC
for primer pair (3)

and

wherein the probes are labeled, preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2,
and/or
(b) determining the expression level of human Estrogen Receptor 1 (ESR1) isotype a with Real-time PCR is performed using the following primer pair:

(1)

(SEQ ID No. 63)

CCACTCAACAGCGTGTCTC
and

(SEQ ID No. 64)

GCTCGTTCTCCAGGTAGTAG,

preferably wherein following probe is used:

(SEQ ID No. 65)

TGTCGCCTTTCCTGCAGCCCCAC

and
wherein the probe is labeled, preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2,
and/or
(c) determining the expression level of human progesterone receptor (PR) with Real-time PCR is performed using one or more of the following primer pairs:

(1)

(SEQ ID No. 66)

CTTACAAAACTTCTTGATAACTTGC
and

(SEQ ID No. 68)

GGTTTCACCATCCCTGCCAA
(2)

(SEQ ID No. 67)

CTGTACTGCTTGAATACATTTATCC
and

(SEQ ID No. 68)

GGTTTCACCATCCCTGCCAA,

preferably wherein following probes are used:

(SEQ ID No. 69)

CTTCATCTGTACTGCTTGAATACATTTATCCAG,
for primer pair (1)
and/or

(SEQ ID No. 70)

ATGATGTCTGAAGTTATTGCTGCACAATTACCC
for primer pair (2)

and,
wherein the probes are labeled, preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2.

In a more preferred embodiment, one primer pair of each (a) and (b) and (c), respectively, is used.

In a further embodiment, the present method relates to a method of treating an epithelial cancer patient. In such embodiment, a therapeutically effective amount of at least one agent for treating cancer is administered. Such agent may be a chemotherapeutic agent, or a combination of 2, 3, 4, or more chemotherapeutic agents, one or more aromatase inhibitors, one or more agents directed against HER-2, or one or more agents for hormone therapy, or combinations thereof.

Preferably, the one or more agents are administered two or more times to the patient in a therapeutically effective amount.

An exemplary treatment regime for the treatment of breast cancer is paclitaxel, at a dose of 175 mg/m² intravenously over 3 hours every 3 weeks for 4 courses administered sequentially to doxorubicin-containing combination chemotherapy. For example 4 courses of doxorubicin and cyclophosphamide may be used.

An exemplary treatment regime for the treatment of breast cancer after failure of initial chemotherapy for metastatic disease or relapse within 6 months of adjuvant chemotherapy, is the administration of paclitaxel at a dose of 175 mg/m² administered intravenously over 3 hours every 3 weeks.

The skilled person is aware of therapeutically effective amounts and administration regimes for agent for treating cancer as well as of suitable combination treatment regimes.

Trastuzumab is a suitable agent directed against Her-2. For example, trastuzumab may be administered to a breast cancer patient alone or in combination with paclitaxel. Initial dose: 4 mg/kg IV infusion over 90 minutes. Subsequent therapy: 2 mg/kg IV infusion over 30 minutes once weekly until disease progression. As adjuvant therapy, following treatment dosage and regime may be used: 1) Initiate trastuzumab during and following paclitaxel, docetaxel, or docetaxel/carboplatin: Initial dose: 4 mg/kg IV infusion over 90 minutes then 2 mg/kg IV infusion over 30 minutes weekly during chemotherapy for the first 12 weeks (paclitaxel or docetaxel) or 18 weeks (docetaxel/carboplatin). Subsequent therapy: one week after the last weekly dose of trastuzumab, give trastuzumab as 6 mg/kg IV infusion over 30 to 90 minutes every 3 weeks for a total of 52 weeks of therapy, or: Initiate trastuzumab as a single agent within 3 weeks following completion of all chemotherapy. Initial dose: 8 mg/kg IV infusion over 90 minutes. Subsequent therapy: 6 mg/kg IV infusion over 30 to 90 minutes every 3 weeks for a total of 17 doses (52 weeks of therapy).

A suitable agent for hormone therapy of breast cancer is tamoxifen. Following regimes may be used: For the treatment of metastatic breast cancer in women and men: 20 to 40 mg orally dosages greater than 20 mg are given in divided doses (morning and evening). For the treatment of women with ductal carcinoma in situ, following breast surgery and radiation: 20 mg orally daily for 5 years. To reduce the incidence of breast cancer in women at high risk for breast cancer: 20 mg orally daily for 5 years.

As an adjuvant therapy of tamoxifen, following regime may be used: For the treatment of node-positive breast cancer in postmenopausal women following total mastectomy or segmental mastectomy, axillary dissection, and breast irradiation: 10 mg orally 2 to 3 times a day for 5 years.

As aromatase inhibitors, anastrozole, exemestane or letrozole may preferably be used for treating breast cancer. For example, for the first-line treatment of postmenopausal women with hormone receptor positive or hormone receptor unknown locally advanced or metastatic breast cancer: 1 mg tablet of anastrozole (Arimidex®) should be administered once a day. For example, exemestane may be administered as follows for treating breast cancer: Recommended dose: 25 mg orally once daily after a meal. In postmenopausal women with early breast cancer who have been treated with two to three years of tamoxifen, treatment with exemestane should continue in the absence of recurrence or contralateral breast cancer until completion of five years of adjuvant endocrine therapy. For patients with advanced breast cancer, treatment with exemestane should continue until tumor progression is evident. For example letrozole may be used and administered as follows for treating breast cancer: For use as first-line treatment of postmenopausal women with hormone receptor positive or hormone receptor unknown locally advanced or metastatic breast cancer. Letrozole is also indicated for the treatment of advanced breast cancer in postmenopausal women with disease progression following anti-estrogen therapy: 2.5 mg tablet orally administered once a day without regard to meals. In patients with advanced disease, letrozole therapy should continue until tumor progression is evident. As adjuvant therapy in breast cancer, following applies preferably for letrozole: For use as extended adjuvant treatment of early breast cancer in postmenopausal women who have received 5 years of adjuvant tamoxifen therapy: 2.5 mg tablet orally administered once a day without regard to meals. Treatment should be discontinued if there is a tumor relapse.

From such patients under cancer treatment, tissue samples are obtained at two different time points, a first and second time point, and the expression levels both of total membrane-bound Muc1 mRNA or protein and the long forms of Muc1 mRNA or protein are determined, as described above for the method for monitoring therapy and/or for adapting therapy of an epithelial cancer patient, who is subject to a cancer treatment of the present invention.

As described above, the second time point is at least 1 day later than the first time point, preferably at least 1 week later than the first time point, more preferably at least 1 month later than the first time point, even more preferably at least 3, 6, 9 or 12 months later than the first time point.

The ratios of expression levels determined at the first time point and the second time point are compared. In case an increase in ratio between the expression level of total membrane-bound Muc1 mRNA or protein and the long forms of Muc1 mRNA or protein at the second time point compared to the first time point is determined, the current treatment by administration of a therapeutically effective amount of at least one agent for treating cancer is stopped, and/or a therapeutically effective amount of at least one agent directed against Muc1 is administered, and/or a chemotherapeutic regime with high dosage and/or short intervals of chemotherapeutic agent(s) is administered.

Therefore, in another embodiment, the present invention relates to a method of treating an epithelial cancer patient, comprising
(i) administering a therapeutically effective amount of at least one agent for treating cancer,
(ii) performing the method of the invention for monitoring therapy and/or for adapting therapy of an epithelial cancer patient, who is subject to a cancer treatment, wherein in case an increase in ratio between the expression level of
   total membrane-bound Muc1 mRNA or protein and
   the long forms of Muc1 mRNA or protein
   at the second time point compared to the first time point is determined,
      the administration a therapeutically effective amount of at least one agent for treating cancer is stopped, and/or
      a therapeutically effective amount of at least one agent directed against Muc1 is administered, and/or
      a chemotherapeutic regime with high dosage and/or short intervals of chemotherapeutic agent(s) is administered.

For example, in case the initial therapy was already a treatment with a chemotherapeutic agent, the dosage may then be increased by 10%, 50%, or 100%, and/or the interval of administration may be shortened by e.g. 50%.

Suitable chemotherapeutic agents are described above.

Further, the expression levels of (i) Her-2 mRNA, (ii) Estrogen Receptor 1 (ER1) isotype 1 mRNA, and (iii) Progesterone Receptor (PR) mRNA are in addition determined in a preferred embodiment.

Therefore, in another embodiment, the present invention relates to a method of treating an epithelial cancer patient, comprising:
(i) administering a therapeutically effective amount of at least one agent for treating cancer,
(ii) performing the method of the invention for monitoring therapy and/or for adapting therapy of an epithelial cancer patient, who is subject to a cancer treatment as described above, wherein in case
   (α) an increase in ratio between the expression levels of
      total membrane-bound Muc1 mRNA or protein and
      the long forms of Muc1 mRNA or protein
      at the second time point compared to the first time point is determined, and
   (β) a decrease in expression level of Estrogen Receptor 1 (ER1) isotype 1 mRNA, and Progesterone receptor (PR) mRNA and optionally Her-2 mRNA at said second time point compared to said first time point is determined,
      the administration a therapeutically effective amount of at least one agent for treating cancer is stopped, and/or
      a therapeutically effective amount of at least one agent directed against Muc1 is administered, and/or
      a chemotherapeutic regime with high dosage and/or short intervals of chemotherapeutic agent(s) is administered.

For example, if the initial therapy was already a treatment with a chemotherapeutic agent, the dosage may be increased by 10%, 50%, or 100%, and/or the interval of administration may be shortened by e.g. 50%, in case an increase is determined in (α) and a decrease is determined in (β).

In case the initial therapy was a treatment with tamoxifen, such treatment may be stopped, in case an increase is determined in (α) and a decrease is determined in (β), and
a therapeutically effective amount of at least one agent directed against Muc1 is administered, and/or
a chemotherapeutic regime with high dosage and/or short intervals of chemotherapeutic agent(s) is administered.

Suitable chemotherapeutic agents are described above.

In a further preferred embodiment, the at least agent for treating cancer is selected from a chemotherapeutic agent, an aromatase inhibitor, an hormone therapeutic agent, and an agent directed against HER-2, as described above in detail. The administration routes for therapeutic agents depend on the formulation and is known to a skilled person. For example, the at least one agent may be administered intravenously e.g. in case of an infusion, or may be administered orally, in case of tablets.

In a further preferred embodiment, the at least one agent directed against HER-2 is Herceptin® (trastuzumab) or a functionally active derivative thereof. Trastuzumab is a monoclonal antibody that interferes with the HER2/neu receptor.

In a further preferred embodiment, the aromatase inhibitor is an agent for hormone therapy, preferably at least one agent directed against Estrogen Receptor 1 (ESR1) isotype a or progesterone receptor (PR), even more preferably selected from tamoxifen, and a GnRH analogue.

A “Muc-1 based therapy” is understood as a therapy which is targeting Muc-1 RNA or protein. A therapy which targeting Muc-1 RNA or protein is a therapy which influences Muc-1 protein expression and/or activity and/or accessibility in the body. For example, a Muc-1 based therapy refers to the administration of an agent directed against Muc1 protein. In a preferred embodiment, an agent directed against Muc1 protein is an antibody or derivative thereof directed against Muc1. Antibodies directed against Muc1 are known to a skilled person. In a preferred embodiment, Pankomab may be used. Pankomab is PankoMab is a humanized monoclonal antibody recognizing the tumor-specific epitope of mucin-1 (TA-MUC1). It differentiates between tumor MUC1 and non-tumor MUC1 epitopes and may be used for treating epithelial cancer, in particular breast cancer. In another preferred embodiment, Pemtumomab (Theragyn®) may be used. Pemtumomab is a mouse monoclonal antibody. Further, AS1402 anti-MUC1 antibody is known. Alternatively, a Muc1 based therapy or an agent directed against Muc1 may be vaccine directed against Muc1. For example, Stimuvax® (also known as L-BLP25 or BLP25 Liposome Vaccine) is known. This is an investigational therapeutic cancer vaccine designed to induce an immune response to cancer cells that express Muc1.

Further, the present invention also relates to a method of treating a tumor patient suffering from an epithelial tumor in case malignancy or increase in malignancy is found.

In one alternative embodiment, a tissue sample comprising tumor cells is obtained from said patient, and following expression levels are determined in said sample, as described above:
(b) the expression level of
   (i) total membrane-bound Muc1 mRNA, or
   (ii) total membrane bound Muc1 protein
   in said tissue sample, and
(c) the expression level of
   (i) the long forms of Muc1 RNA, or
   (ii) the long forms of Muc1 protein

In case the expression level of (b) is determined to be higher than the expression level of (c), a tumor therapy is initiated, or the amount or strength of an ongoing therapy is increased.

In another alternative embodiment, a tissue sample comprising tumor cells is obtained from said patient at two or more time points, as described above. The expression levels are determined in said sample for each time point, as described above:
(a) the expression level of
   (i) total membrane-bound Muc1 mRNA, or
   (ii) total membrane bound Muc1 protein
   in said tissue sample, and
(b) the expression level of
   (i) the long forms of Muc1 RNA, or
   (ii) the long forms of Muc1 protein,

and the difference (b)-(b) is determined.

In case an increase in the difference (a)-(b) between (a) the expression level of total membrane-bound Muc1 mRNA, or total membrane bound Muc1 protein and (b) the expression level of the long forms of Muc1 RNA, or the long forms of Muc1 protein at the later time point compared to the respective earlier time point is determined, a tumor therapy is initiated, or the amount or strength of an ongoing therapy is increased. In case the difference increases at a later time point, the tumor has become malignant or has increased malignancy grade.

Therefore, in another embodiment, the present invention relates to a method of treating a tumor patient, comprising:

performing a method of the invention for determining malignancy grade or progression of a tumor of a patient suffering from an epithelial tumor described above,

wherein in case
(x) an expression level of (b) higher than the expression level of (c) is determined by performing the method of the invention, wherein
   (b) is total membrane-bound Muc1 mRNA and
   (c) is the long forms of Muc1 RNA , or
(xx) an increase in the difference (a)-(b) between (a) the expression level of total membrane-bound Muc1 mRNA, or total membrane bound Muc1 protein and (b) the expression level of the long forms of Muc1 RNA, or the long forms of Muc1 protein at the later time point compared to the earlier time point is determined by performing the method of the invention further comprising repetition steps,

a tumor therapy is initiated, or the amount or strength of an ongoing therapy is increased.

In a yet further embodiment, the present invention relates to at least one agent directed against HER-2, at least one aromatase inhibitor, at least one chemotherapeutic agent, or irradiation, for use in the treatment of a tumor patient, wherein the tumor of said patient was determined to be malignant, and/or the tumor was determined to have increased its malignancy grade, and/or the tumor disease is determined to be progressing and/or the tumor is determined to be less responsive to the currently applied tumor therapy by performing a method of the invention for determining malignancy grade or progression of a tumor of a patient suffering from an epithelial tumor described above.

In a yet further embodiment, the present invention relates to at least one agent directed against Muc1 and/or at least one chemotherapeutic agent, for use in the treatment of an epithelial cancer patient who is subject to a cancer treatment, wherein said patient was determined to be the patient is less responsive to said cancer treatment, and was determined to be responsive to Muc1 based therapy by performing a method of the invention described above for monitoring therapy and/or for adapting therapy of an epithelial cancer patient, who is subject to a cancer treatment.

The preferred embodiments for methods of the invention, as described above in detail, also apply for the agents for use of the invention.

The primer pairs disclosed herein are surprisingly useful for determining the expression level of total membrane-bound Muc1 mRNA or long forms of Muc1 mRNA.

Therefore, in a further embodiment, the present invention relates to at least one pair of primers selected from (i) to (xv):

(i)

(SEQ ID No. 1)

CCTCCCCACCCATTTCACC
and

(SEQ ID No. 2)

CTGTAAGCACTGTGAGGAGC
(ii)

(SEQ ID no. 3)

CCTACCATCCTATGAGCGAG
and

(SEQ ID No. 4)

CCCTACAAGTTGGCAGAAGTG
(iii)

(SEQ ID No. 5)

CTACTGAGAAGAATGCTTTGTCTA
and

(SEQ ID No. 6)

GCCTGAACTTAATATTGGAGAGG
(iv)

(SEQ ID No. 7)

CTACTGAGAAGAATGCTTTTAATTCC
and

(SEQ ID No. 8)

GCCTGAACTTAATATTGGAGAGG
(v)

(SEQ ID No. 9)

CTACTGAGAAGAATGCTTTGTCTA
and

(SEQ ID No. 10)

CTCTTGGTAGTAGTCGGTGC
(vi)

(SEQ ID No. 11)

(CCAGCACCGACTACTACCAA
or

(SEQ ID No. 13)

CACCGACTACTACCAAGAGC
and

(SEQ ID No. 12)

CTCTTGGTAGTAGTCGGTGC
(vii)

(SEQ ID No. 14)

CTACTGAGAAGAATGCTTTTAATTCC
and

(SEQ ID No. 15)

GCCTGAACTTAATATTGGAGAGG
(viii)

(SEQ ID No. 16)

CTACTGAGAAGAATGCTTTTAATTCC
and

(SEQ ID No. 17)

(CGGCACTGACAGACAGCCAT
or

(SEQ ID No. 18))

GGCACTGACAGACAGCCATT
(ix)

(SEQ ID No. 19)

CTACTGAGAAGAATGCTTTTAATTCC
and

(SEQ ID No. 20)

CACCCCAGCCCCAGACATT
(x)

(SEQ ID No. 21)

CTACTGAGAAGAATGCTTTTTTGC
and

(SEQ ID No. 22)

AGGCTGCTTCCGTTTTATACTG
(xi)

(SEQ ID No. 23)

CCTCTCCAATATTAAGTTCAGTGA
and

(SEQ ID No. 24)

ACAGACAGCCAAGGCAATGAG
(xii)

(SEQ ID No. 25)

(CCTCTCCAATATTAAGTTCAGTCT
or

(SEQ ID No. 26)

CCTCTCCAATATTAAGTTCAGTC
and

(SEQ ID No. 27)

ACAGACAGCCAAGGCAATGAG
(xiii)

(SEQ ID No. 28)

CCACTCTGATACTCCTACCAC
and

(SEQ ID No. 29)

GAAAGAGACCCCAGTAGACAAC,
(xiv)

(SEQ ID No. 30)

CCTCCCCACCCATTTCACC
and

(SEQ ID No. 31)

CTGTAAGCACTGTGAGGAGC,
(xv)

(SEQ ID No. 32)

CACTTCTGCCAACTTGTAGGG,
and

(SEQ ID No. 33)

CCCTACAAGTTGGCAGAAGTG.

Further, probes were successfully developed, which are useful together with their respective primer pairs for RT-PCR methods of the invention.

Therefore, in a further embodiment, the present invention relates to kit comprising at least one pair of primers of the invention, and at least one probe, wherein the at least one probe is selected from:

(a)

(SEQ ID No. 34)

TGACACCGGGCACCCAGTCTCC
and/or

(SEQ ID No. 35)

CCACCATGACACCGGGCACCCA
for primer pair (i)
(b)

(SEQ ID No. 36)

TGCAGGTAATGGTGGCAGCAGCC
for primer pair (ii)
(c)

(SEQ ID No. 37)

AGCACCGACTACTACCAAGAGCTGCA
and/or

(SEQ ID No. 38)

TTTCCTGTCTTTTCACATTTCAAACCTCCAGTT
for primer pair (iii)
(d)

(SEQ ID No. 39)

CAGCACCGACTACTACCAAGAGCTGC
for primer pair (iv)
(e)

(SEQ ID No. 40)

TTTCCTGTCTTTTCACATTTCAAACCTCCAGTT
for primer pair (v)
(f)

(SEQ ID No. 41)

ATGGCTGTCTGTCAGTGCCGCCGAA
for primer pair (vi)
(g)

(SEQ ID No. 42)

AGCACCGACTACTACCAAGAGCTGCA
and/or

(SEQ ID No. 43)

CAGCACCGACTACTACCAAGAGCTGC
for primer pair (vii)
(h)

(SEQ ID No. 44)

AGCACCGACTACTACCAAGAGCTGCA
for primer pair (viii)
(i)

(SEQ ID No. 45)

AGCACCGACTACTACCAAGAGCTGCA
and/or

(SEQ ID No. 46)

CAGCACCGACTACTACCAAGAGCTGC
for primer pair (ix)
(j)

(SEQ ID No. 47)

TTGACTCTGGCCTTCCGAGAAGGTAC
and/or

(SEQ ID No. 48)

CTTCCGAGAAGGTACCATCAATGTCCAC
for primer pair (x)
(k)

(SEQ ID No. 49)

CATCGCGCTGCTGGTGCTGGTCT
and/or

(SEQ ID No. 50)

TGTGCCATTTCCTTTCTCTGCCCAGTC
for primer pair (xi)
(l)

(SEQ ID No. 51)

CATCGCGCTGCTGGTGCTGGTCT
for primer pair (xii)
(m)

(SEQ ID No. 52)

AGCCATAGCACCAAGACTGATGCCA
and/or

(SEQ ID No. 53)

ACCTCCTCTCACCTCCTCCAATCACA
for primer pair (xiii), (xiv) and (xv),

and wherein the probes are labeled,

preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe,

even more preferably wherein the fluorescent label is ROX or FAM and the quencher moiety is BHQ2.

Further, primer pairs and probes were successfully designed for determining the expression levels of ESR1, PR or Her-2 mRNA by RT-PCR.

Therefore, in a preferred embodiment, the kit of the invention further comprises one or more of the following components (a) to (c):
(a) at least one pair of primers selected from (1) to (3):

(1)

(SEQ ID No. 54)

CGTTTGAGITCCATGCCCAATC
and

(SEQ ID No. 55)

TCCTCTGCTGITCACCTCTTG,
(2)

(SEQ ID No. 56)

CACCCACTCCCCTCTGAC
and

(SEQ ID No. 57)

CAGCAGITCTCCGCATCGTG
(3)

(SEQ ID No. 58)

GTGAAACCTGACCTCTCCTAC
and

(SEQ ID No. 59)

CAGCAGTCTCCGCATCGTG,

and optionally at least one probe selected from:

(SEQ ID No. 60)

CTGCCTGITCCCTACAACTACCTTTCTAC,
for primer pair (1)

(SEQ ID No. 61)

ATCCTCATCAAGCGACGGCAGCAGAA,
for primer pair (2)
and

(SEQ ID No. 62)

AGCAGAGAGCCAGCCCTCTGACGTCCATC
for primer pair (3)

(b) the following primer pair:

(1)

(SEQ ID No. 63)

CCACTCAACAGCGTGTCTC
and

(SEQ ID No. 64)

GCTCGTTCTCCAGGTAGTAG,

and optionally following probe:

(SEQ ID No. 65)

TGTCGCCTTTCCTGCAGCCCCAC,

(c) at least one pair of primers selected from (1) and (2):

(1)

(SEQ ID No. 66)

CTTACAAAACTTCTTGATAACTTGC
and

(SEQ ID No. 68)

GGTTTCACCATCCCTGCCAA
(2)

(SEQ ID No. 67)

CTGTACTGCTTGAATACATTTATCC
and

(SEQ ID No. 68)

GGTTTCACCATCCCTGCCAA,

and optionally at least one probe selected from:

(SEQ ID No. 69)

CTTCATCTGTACTGCTTGAATACATTTATCCAG,
for primer pair (1)
and/or

(SEQ ID No. 70)

ATGATGTCTGAAGTTATTGCTGCACAATTACCC,
for primer pair (2)

wherein the optionally present probes are labeled, preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2.

As described in the examples, methods were developed which allow reliable and efficient quantification of expression levels. To that end, efficient kits and methods s for sample storage, reverse transcription and RT-PCR were developed.

In a more preferred embodiment of the present invention, the kit therefore further comprises one, two, three or four of the following components (a) to (d):
(a) means for storing a tissue probe, in particular comprising a solution of 95% ethanol in water,
(b) means for isolating RNA from a tissue probe, in particular comprising a buffer for lysing tissue, a buffer for lysing cells, DNAse I and buffers for eluting RNA from a column and/or washing of RNA, preferably wherein the kit does not comprise paraffin,
(c) means for reverse transcribing RNA, in particular comprising a reverse transcriptase, a mixture of dNTPs, primers, and a reaction buffer, in particular wherein the primers are random sequence primers, Oligo(dT) primers or primers specific for the target sequence(s),
(d) means for performing Real-Time PCR, in particular comprising a DNA polymerase, a mixture of dNTPs, primers, and a reaction buffer, in particular wherein the primers are primers specific for the target sequence(s),

preferably wherein the kit comprises components (c), (c) and (d), or (b), (c) and (d).

In another embodiment, the present invention relates to the use of a kit of the invention as described above, or of at least one pair of primers of the invention as described above, for monitoring therapy and/or for adapting therapy of an epithelial cancer patient, who is subject to a cancer treatment.

In another embodiment, the present invention relates to the use of a kit of the invention as described above, or of at least one pair of primers of the invention as described above, for determining malignancy grade or progression of a tumor of a patient suffering from an epithelial tumor.

The preferred embodiments for methods of the invention, as described above in detail, also apply to the uses of the invention.

In another embodiment, the present invention relates to a promoter consisting of the sequence of SEQ ID No. 71, or a functional variant thereof consisting of a sequence exhibiting at least 90%, preferably at least 95%, more preferably at least 98%, even more preferably at least 99% sequence identity to the sequence of SEQ ID No. 71, with the proviso that a stretch 39 nucleotides of said functional variant exhibits 100% sequence identity to the 3′ terminal 39 nucleotides of the sequence of SEQ ID No. 71.

In another embodiment, the present invention relates to a nucleic acid, in particular a vector, comprising the promoter or functional variant thereof of the invention.

In another embodiment, the present invention relates to an expression construct comprising the promoter or functional variant thereof of the invention and at least one open reading frame.

In a preferred embodiment of the expression construct of the invention, the open reading frame is coding for thymidine kinase (TK) from HSV-1 or HSV-2, preferably thymidine kinase (TK) from HSV-2.

In another embodiment, the present invention relates to a vector comprising the expression construct of the invention.

In a more preferred embodiment, the vector is a vector suitable for transfecting or propagating in human cells.

In another embodiment, the present invention relates to an immunogenic composition, in particular vaccine, comprising the vector of the invention and optionally adjuvants and/or pharmaceutically acceptable excipients.

In another embodiment, the present invention relates to a vector of the invention for use as a medicament, in particular as adjuvant therapy agent.

In another embodiment, the present invention relates to a vector of the invention for use in the treatment of cancer.

In another embodiment, the present invention relates to a vector for use of the invention, in combination with a TK-activated purine or pyrimidine analogue, preferably ganciclovir.

In another embodiment, the present invention relates to the use of the nucleic acid of the invention for expressing a gene or open reading frame, in particular for tissue-specific expression of a gene or open reading frame.

### FIGURE LEGEND

FIG. 1: relates to Mucin 1 (MUC1) genomic structure and RNA transcripts

FIG. 2: relates to human Muc1 RNA exon2 long forms

FIG. 3: relates to HER2-neu (Erbb2) genomic structure and RNA transcripts

FIG. 4: HER2-neu (Erbb2) splicing structure

FIG. 5: Estrogen receptor (ER1) genomic structure and RNA transcripts

FIG. 6: Estrogen1-alpha genomic structure

FIG. 7: Estrogen1-alpha splice variants

FIG. 8: Progesterone receptor PR (PRG) genomic structure and RNA transcripts

FIG. 9: Progesterone receptor PR splicing variants

FIG. 10: MUC1 exon 3a (grey boxes) in different m RNA splice variants. Primers for MUC1 long forms are shown by arrows

FIG. 11: MUC1 primer's pairs: A-“M1”-specific, B-“M1-2”-non-specific, C-“M2”-specific, D-“ML1”-specific, E-“MM2-4.1”, “MM2-4.2”-specific, “MM 2-4.3”-non specific, “MM 2-3-7-specific, low expression, “MM 2-3-6”-specific; cDNA from breast tumor material ##41-59 patients, MT2-lymphocytes cell culture

FIG. 12: HER2-neu (ERBB2)primer's pairs: A-“H1” and “H2” furin/wt -specific; B-“HΔ2-1”-not specific, “HΔ2”-specific; C-“H3”-specific, ##41-60 breast cancer patients

FIG. 13: Estrogen1 receptor primers specificity: A-probes 1, 2, 3, 7, 30, 41, 42, 44, 45, B-probes 2, 5, 8, 28, 33, 41, 44, 60

FIG. 14: A—PR1 primers pair, ##1-45-patient's samples amplification data; B-PR2 primers pair, ##2-53-patient's samples amplification data

FIG. 15: Primers choice HER2neu pairs:
A—temperature gradient 56° C. annealing Pelletier instrument, 52° C. annealing Pelletier instrument, 42, 45, 49 -positive probes; 41, 44, 59-negative probes
B—lines 2-6 -H1 primer pair, lines 7-10 -HA2 primer pair, lines 11-14-H3 primer pair;

FIG. 16: TaqMan RealTime PCR principles

FIG. 17: Titrated Muc1 gene as standard in real-time PCR
A—with calibration curve (adjacent curves differ tenfold in Muc1 gene concentration)
B—standard curves of total and long Muc1 isoforms expression measurements
C—standard curves of reference gene B2M expression measurements

FIG. 18: Scheme of reference gene expression use for estimation of target gene expression levels;

FIG. 19: Results of muc1 and beta-2 microglobulin genes PCR amplification:
A—amplification of Muc1 110 bp part from exon1 and for beta-2 microglobulin (B2M) gene part 95 bp
B—amplification of entire B2M and Muc1 genes of about 1 kb length;

FIG. 20: pTZ57RT vector map

FIG. 21: Multiple amino acid sequence alignment for obtained TKII with reference GenBank data

FIG. 22: Restriction analysis of p2FP-RNAi-TK1 and p2FP-RNAi-TK2 plasmid constructions: 4-p2FP-pRNAi-TK2 No. 4, No. 21, 25, 27, 33, 40-p2FP-pRNAi-TK1 clones No. 21, 25, 27, 33, 40; pl-p2FP-pRNAi Upper row—Pvull digestion, lower row—Bgl II/Eco RI digestion

FIG. 23: Scheme of p2FP-pRNAi-TK1 and p2FP-pRNAi-TK2 plasmid constructions

FIG. 24: PCR amplification of TKI and TKII genes
A—scheme for cloning and further amplification of TKI and TKII genes
B—cloned PCR products analysis

FIG. 25: Scheme of cloning TKI and TKII genes in mammalian expression vector pcDNA4/HisMax C;

FIG. 26: A) Scheme of TKII gene cloning inpDsRed2-C1 vector
B) Scheme of TKII gene cloning in mammalian expression vector pcDNA4/HisMax C

FIG. 27: Confirmation of Thymidine Kinase-I (TKI) nucleus cellular localization
A, B—CHO-K1-DsRed2-TKI, 10 days-old transfectants, fluorescent vs transparent images
C—Cos7-DsRed2 control vector 10days-old transfectants

FIG. 28: Confirmation of Thymidine Kinase-II (TKII) cytoplasmic cellular localization
A, B—CHO-K1DsRed2-TKII, 10 days-old transfectants
C—Cos7-DsRed2-TKII, 21 days-old transfectants, fluorescent vs transparent images

FIG. 29: General scheme of PCR based site-specific mutagenesis

FIG. 30: Computer analysis of DF3 promoter structure (from Zaretsky et al., 2006)

FIG. 31: CMV promoter sequence

FIG. 32: Scheme of the CMV promoter TATA box (between positions −39 and −1) and the proximal CMV enhancer (between positions −39 and −300). Putative binding sites for the various cis-acting elements of the proximal enhancer are shown (Figure taken from Isomura et al., 2004).

FIG. 33: Scheme of DF3 promoter enhancement

FIG. 34: Breast Cancer Markers Profiles, Muc1 Total Quantitative Hyperexpression, Malignancy Grade and ER-PR Histochemistry Data, 38 Patients 2008-2009

FIG. 35: Total Muc1 Expression Levels in Breast Cancer 2008-2009 Patients Compared to Cell Cultures vs PBMC (non-epithelial, healthy donor)

FIG. 36: Breast Cancer Markers Profiles, Total Muc1 Expression and Estrogen Receptors Histochemistry Data in Patients 2008-2009

FIG. 37: Breast Cancer Markers Profiles, Muc1 Total Quantitative Expression and Histochemistry Data, Patients 1-30, 2010

FIG. 38: Breast Cancer Markers Profiles, Total Muc1 Quantitative Expression Histochemistry Data, Patients 31-59, 2010

FIG. 39: Estrogen Alpha Receptor Expression in Immune Histochemistry Data Categories for 38 Breast Cancer Patients 2008-2009

FIG. 40: Estrogen Alpha Receptor Expression for Immune Histochemistry Data Categories for Breast Cancer Patients 1-30, 2010

FIG. 41: Estrogen Alpha Receptor Expression for Immune Histochemistry Data Categories for Breast Cancer Patients 31-59, 2010

FIG. 42: Estrogen Receptor Expression RealTime RT-PCR and Immune Histochemistry 38 Breast Cancer Patients (2008-2009) Data

FIG. 43: Estrogen Receptor Expression RealTime RT-PCR and Immune Histochemistry Breast Cancer Patients 1-30, 2010 Data

FIG. 44: Estrogen Receptor Expression RealTime RT-PCR and Immune Histochemistry Breast Cancer Patients 31-59, 2010 Data

FIG. 45: Estrogen1-Alpha Receptor Expression for immune histochemistry Data Categories for Breast Cancer Patients 2010

FIG. 46: Progesterone Receptor Expression for Immune histochemistry Data Categories for 38 Breast Cancer Patients 2008-2009

FIG. 47: Progesterone Receptor Expression RealTime RT-PCR and Immune Histochemistry, Breast Cancer 38 Patients (2008-2009) Data

FIG. 48: Progesterone Receptor Expression for Immune Histochemistry Data Categories, Breast Cancer Patients 1-30, 2010

FIG. 49: Progesterone Receptor Expression for Immune Histochemistry Data Categories, Breast Cancer Patients 31-59, 2010

FIG. 50: Progesterone Receptor Expression RealTime RT-PCR and Immune Histochemistry, Breast Cancer Patients 1-30, 2010 Data

FIG. 51: Progesterone Receptor Expression RealTime RT-PCR and Immune Histochemistry, Breast Cancer Patients 31-59, 2010 Data

FIG. 52: Progesterone Receptor Expression for Immune Histochemistry Data Categories, Breast Cancer Patients 2010

FIG. 53: HER2-neu Receptor Expression for Immune Histochemistry Data Categories, Breast Cancer Patients 1-59, 2010

FIG. 54: HER2-neu WT (Furin) Expression RealTime RT-PCR and Immune Histochemistry Breast Cancer Patients 1-30, 2010 Data

FIG. 55: HER2-neu WT (Furin) Expression RealTime RT-PCR and Immune Histochemistry Breast Cancer Patients 31-59, 2010 Data

FIG. 56: HER2-neu Receptor Expression for Immune Histochemistry Data Categories Distribution, Breast Cancer Patients 2010

FIG. 57: Breast Cancer Markers Profiles, Muc1 Total Quantitative Expression-MTL-HEP, Breast Cancer Patients 1-30, 2010

FIG. 58: Breast Cancer Markers Profiles, Muc1 Total Quantitative Expression-MTL-HEP, Breast Cancer Patients 31-59, 2010

FIG. 59: Muc1 Total and Muc1 Long Forms Expression Ratio in Breast Tumors, Patients 1-30, 2010

FIG. 60: Muc1 Total and Muc1 Long Forms Expression Ratio in Breast Tumors, Patients 31-59, 2010

FIG. 61: Correspondence (ratio) of Muc1 Long and Muc1 Total Forms Expression in Breast Tumors, Patients 1-30, 2010

FIG. 62: Correspondence (ratio) of Muc1 Long and Muc1 Total Forms Expression in Breast Tumors, Patients 31-59, 2010

FIG. 63: Transfection efficiency for MCF-7 cell line (scheme of 24 well plate)

FIG. 64: Transfection efficiency for T47D and MT-2 cell lines (scheme of 24 well plate)

FIG. 65: Comparison of the −696−43 DF3-−39−1 minimal CMV “hybrid” promoter with −696−1 DF3 promoter and entire CMV promoter

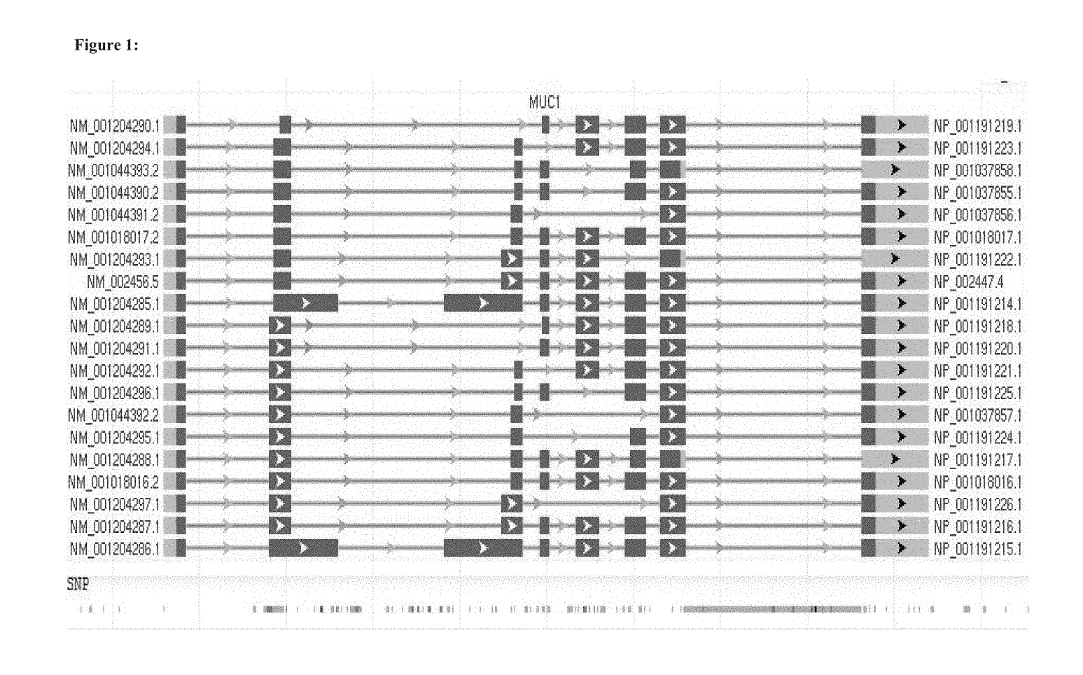

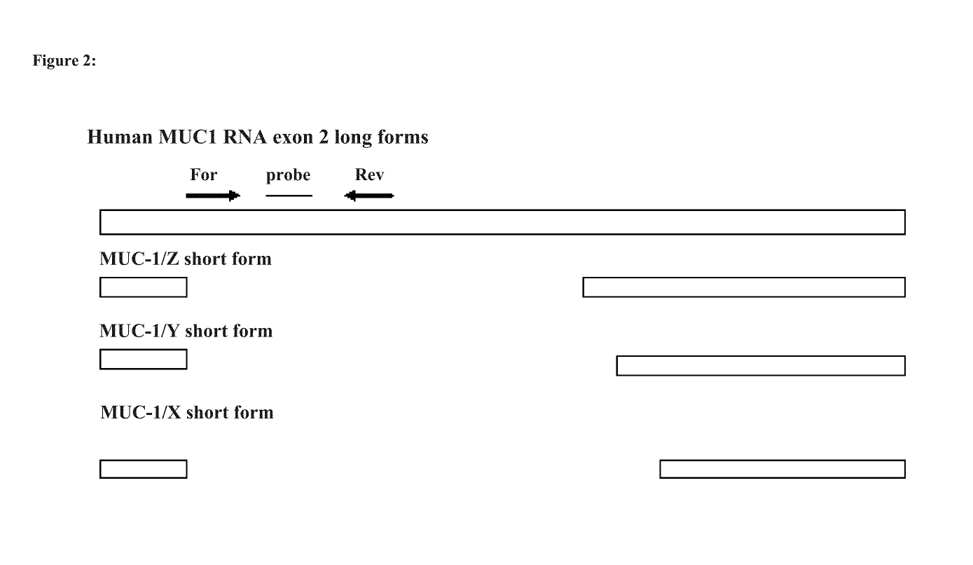

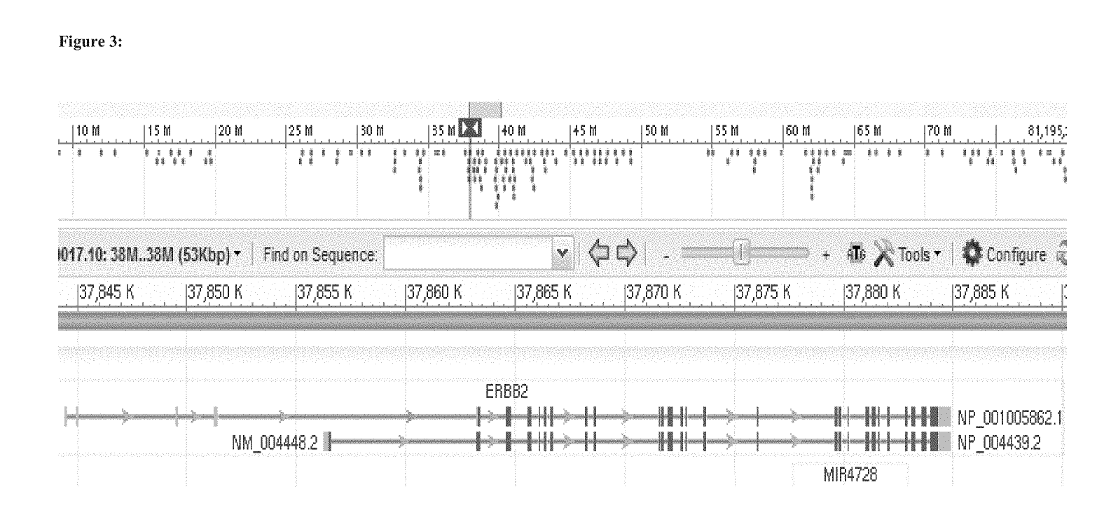

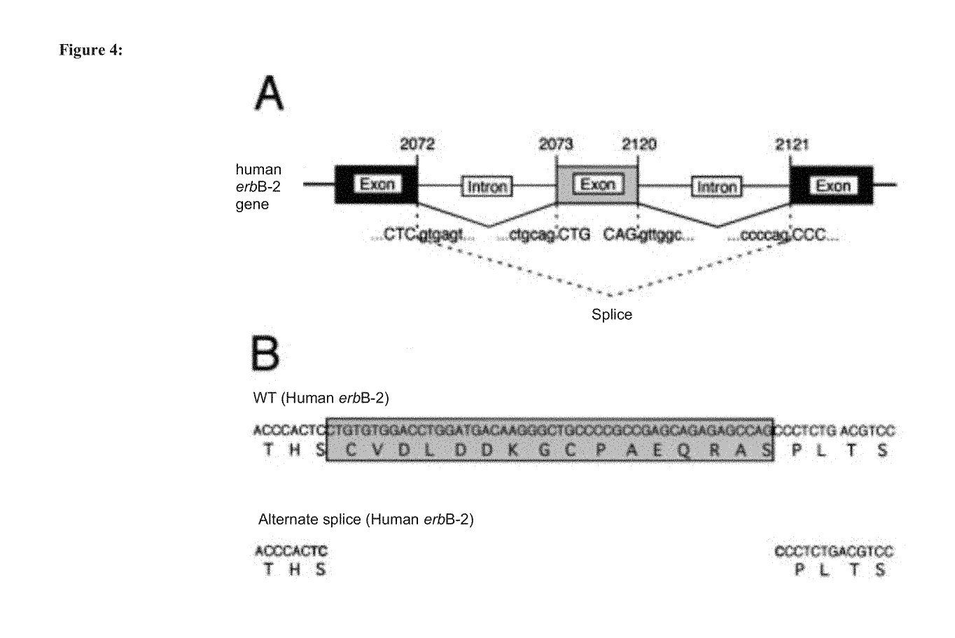

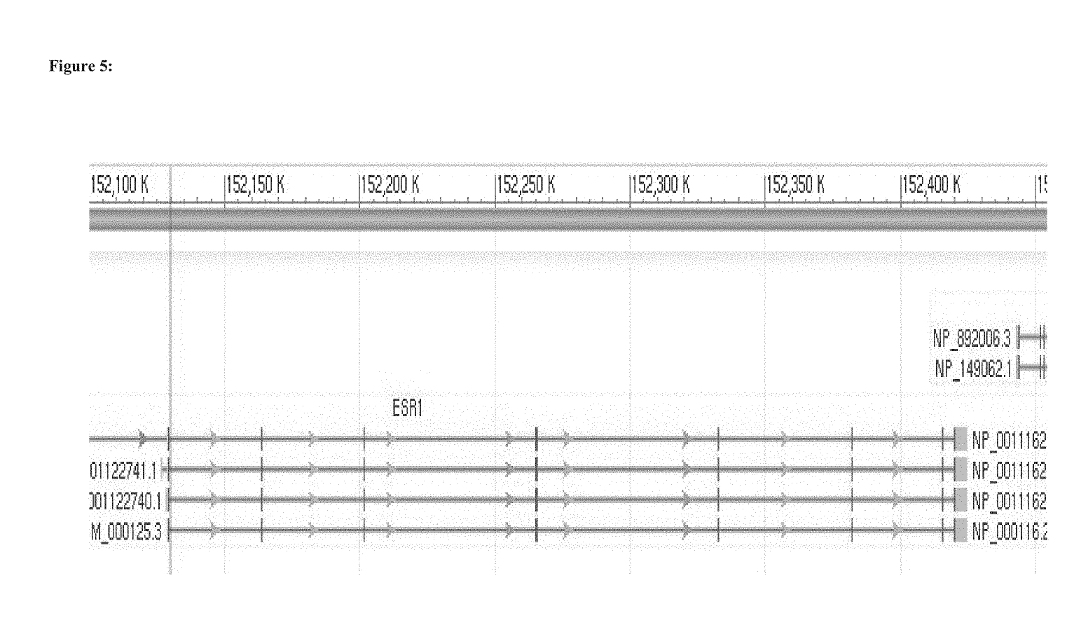

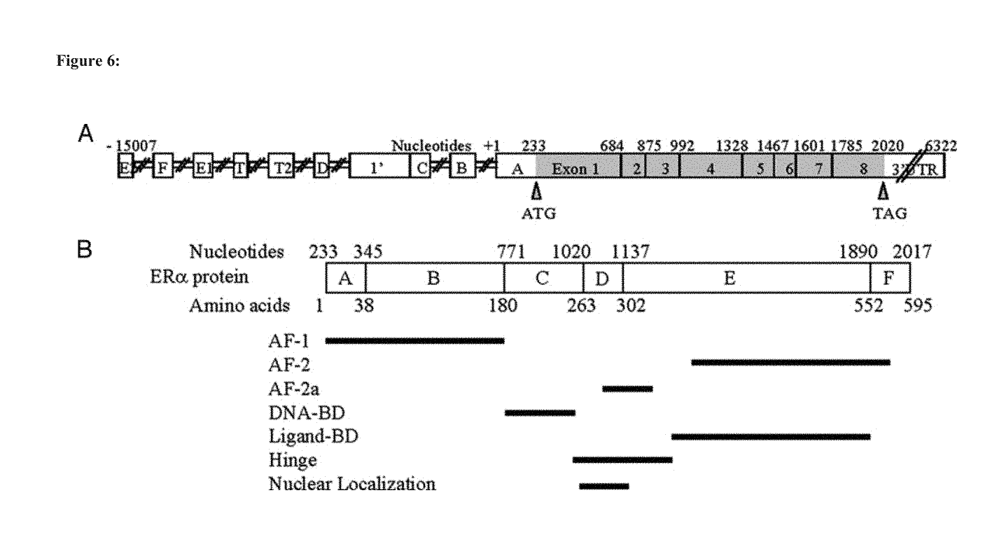

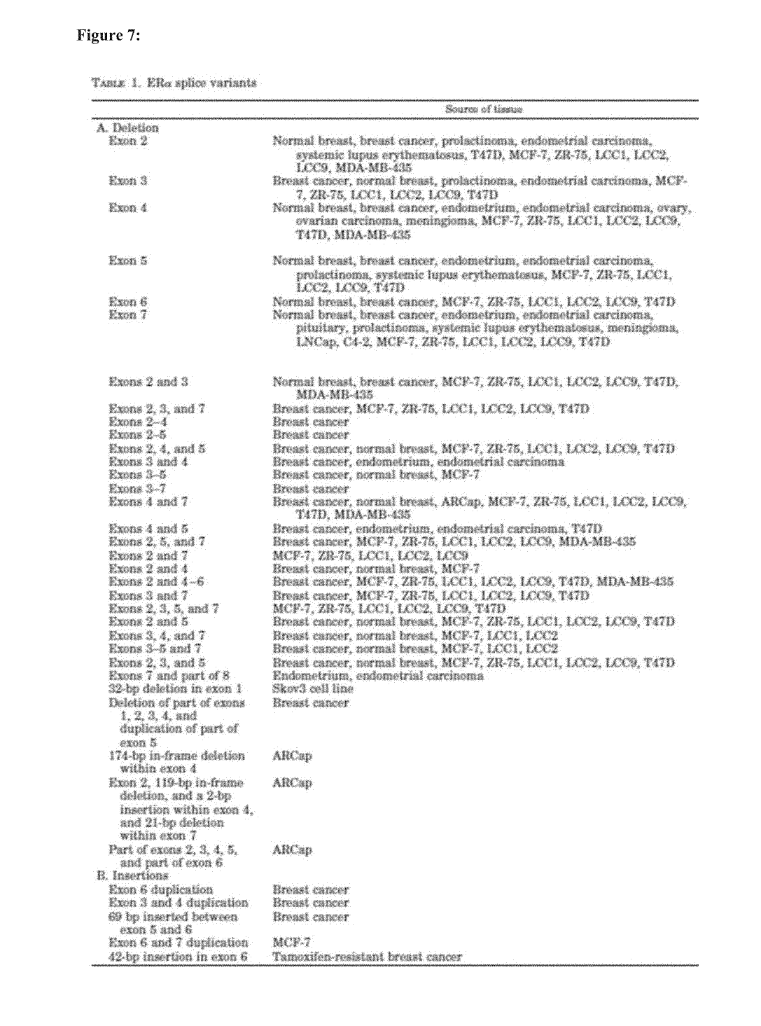

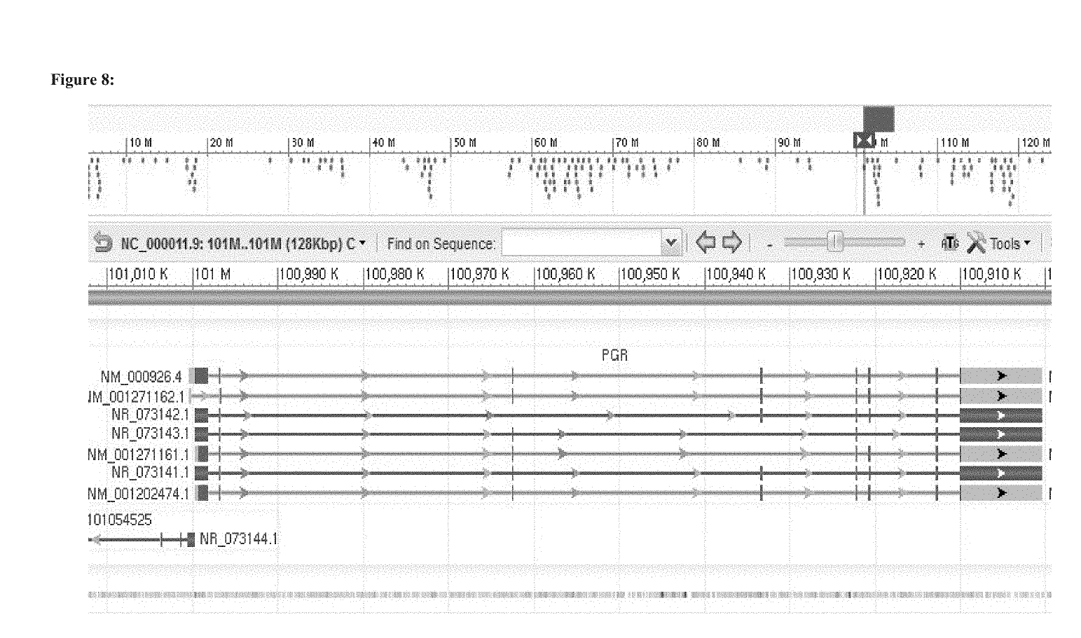

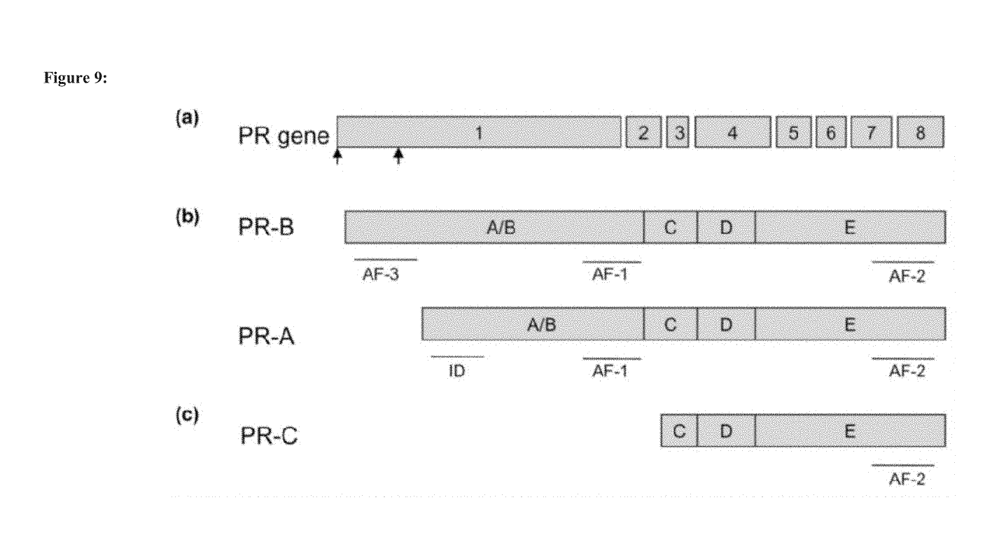

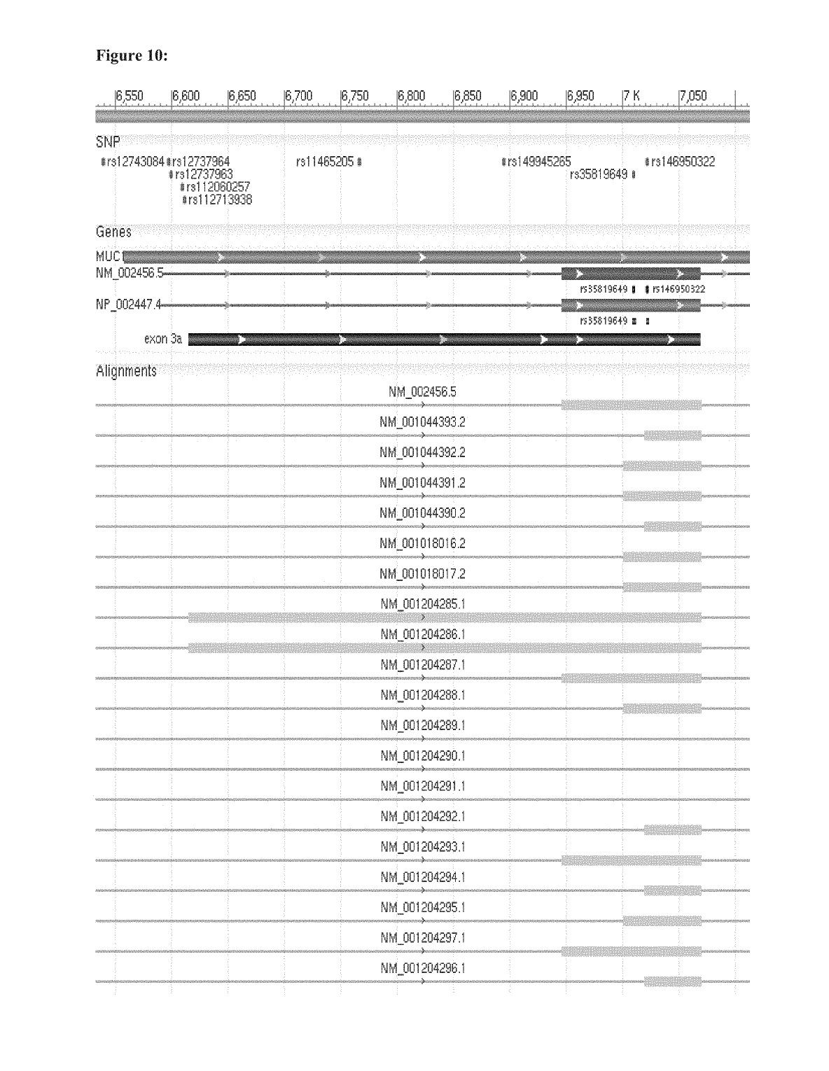

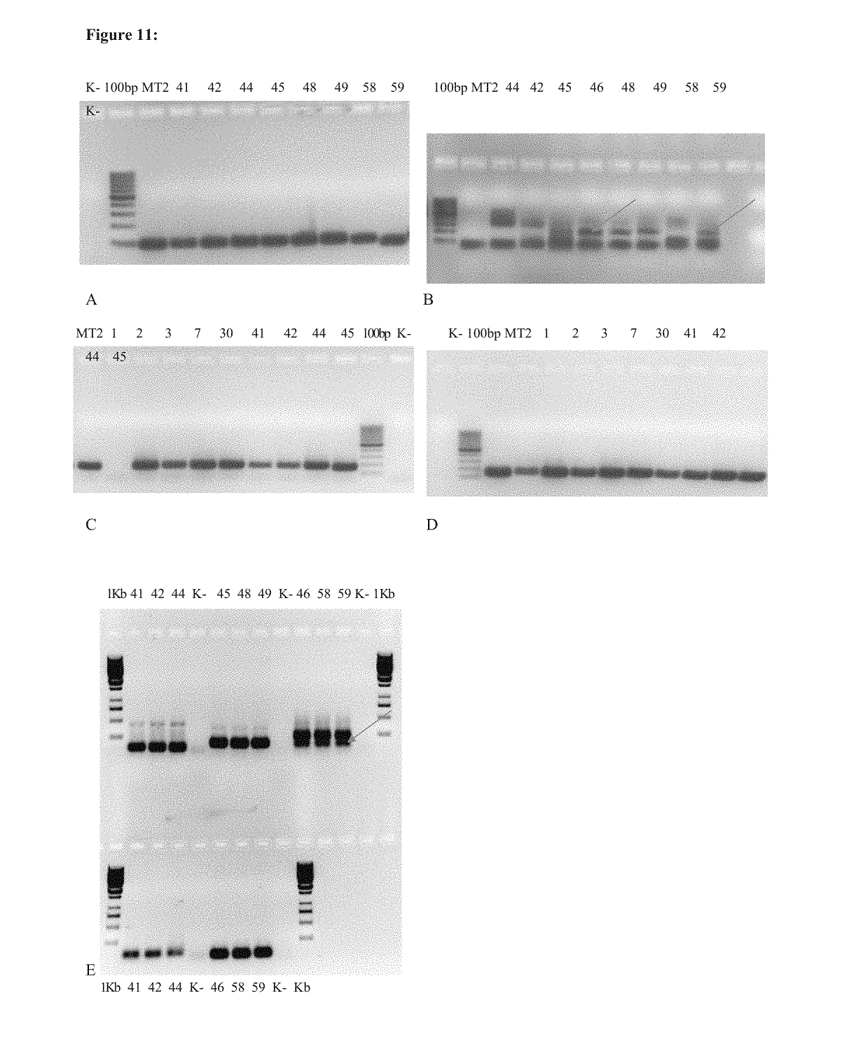

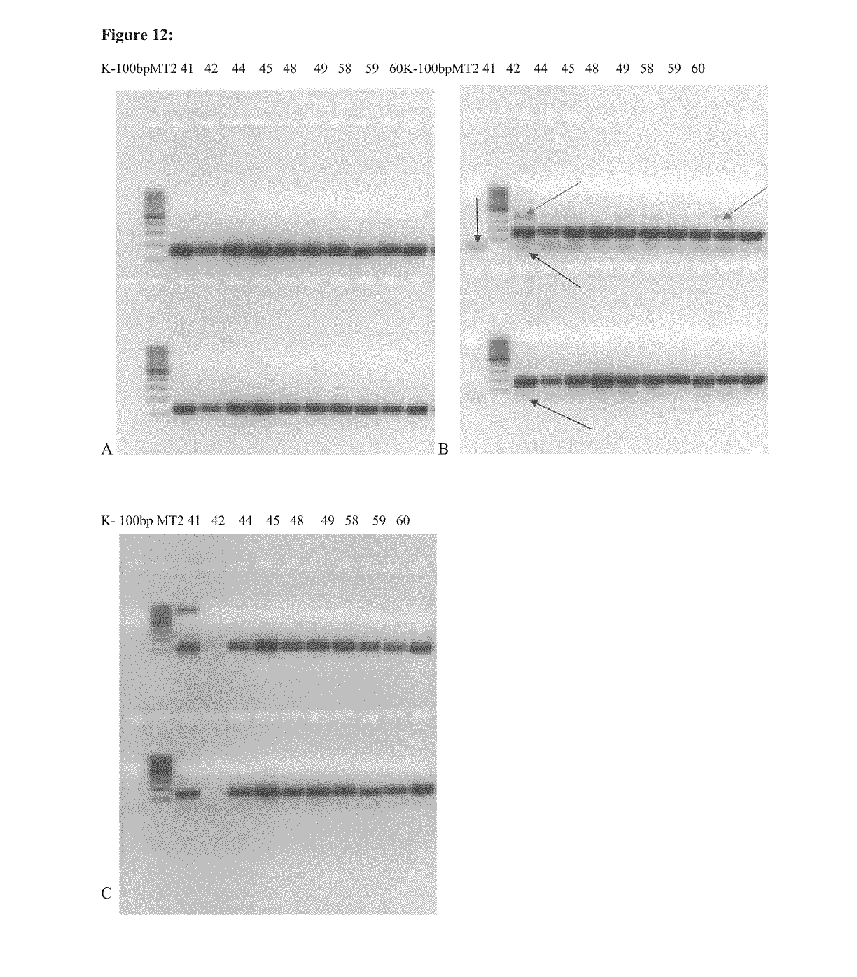

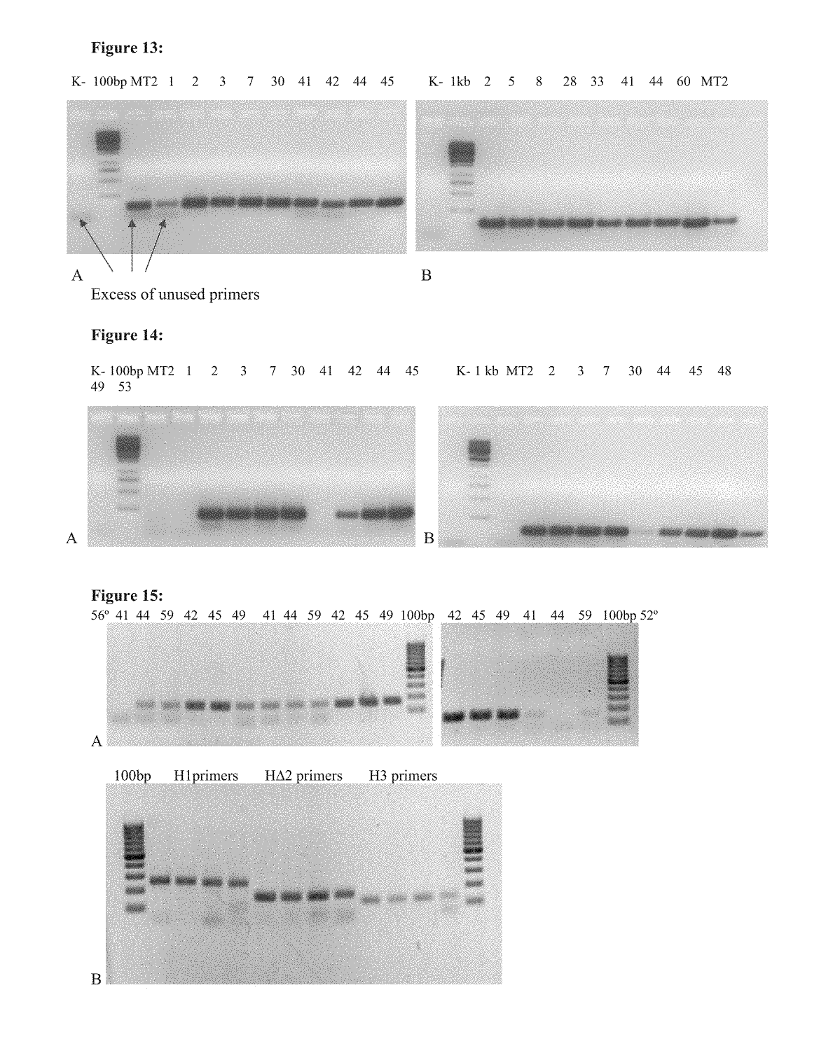

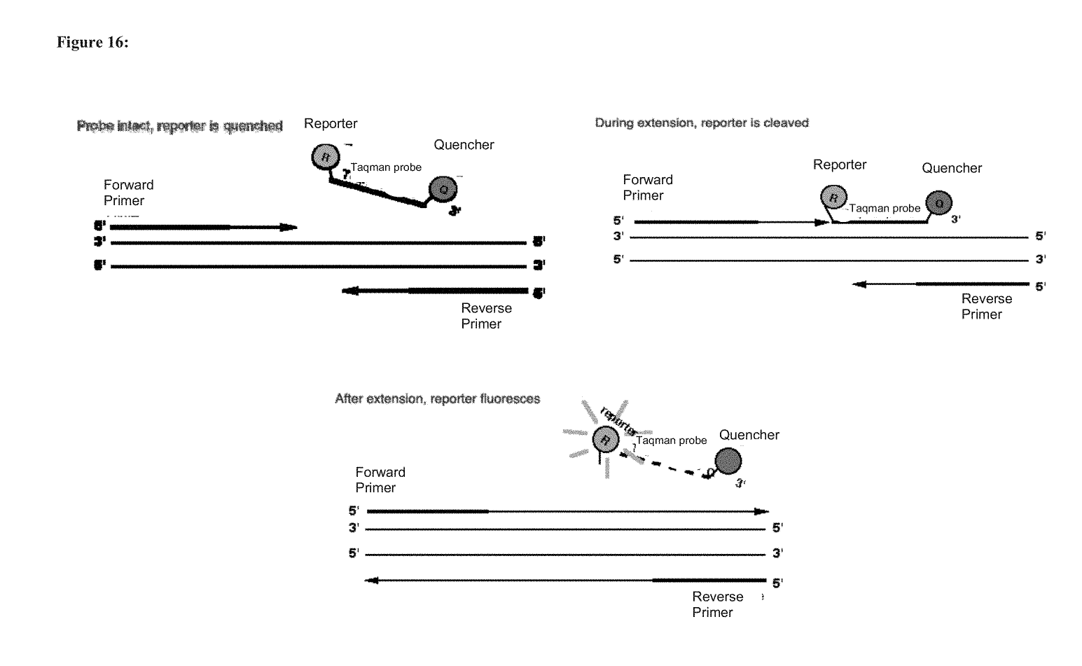

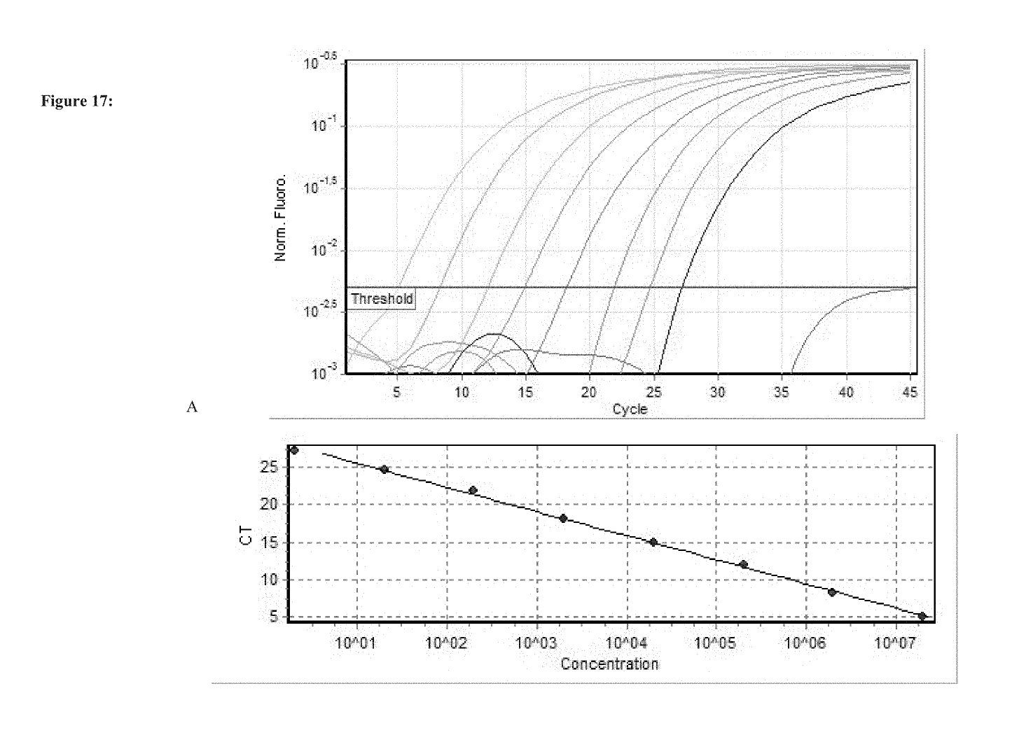

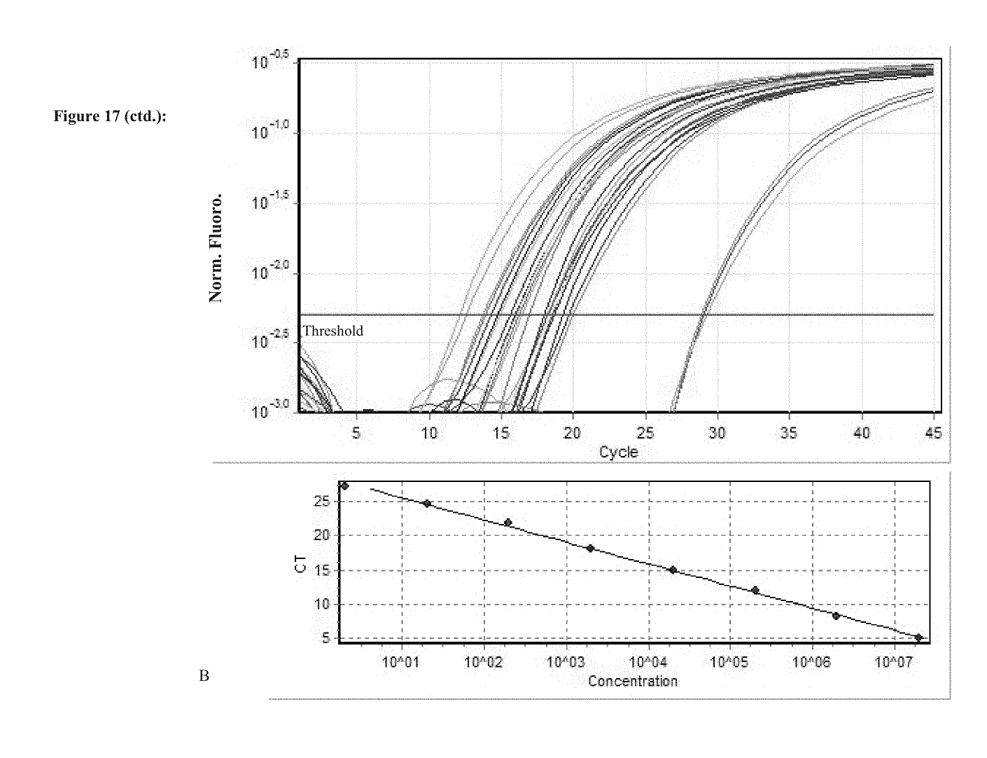

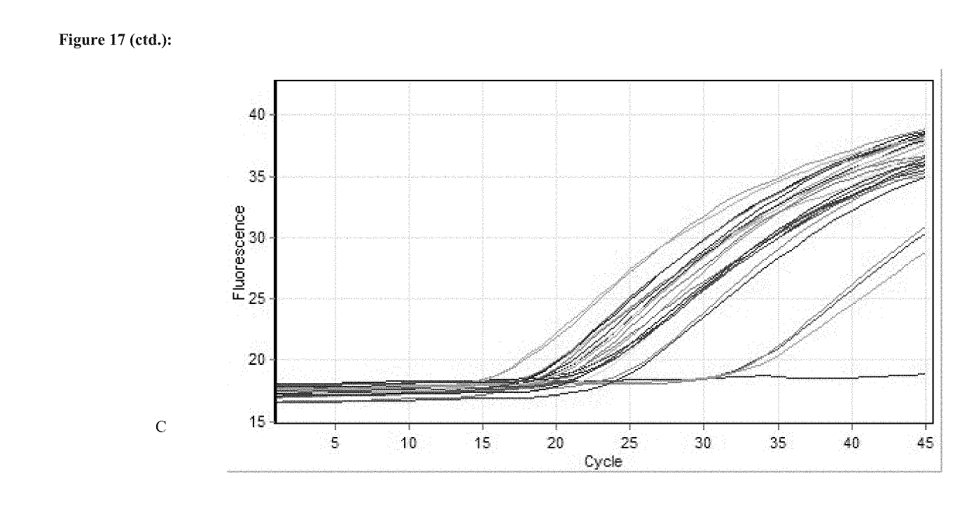

### SELECTED SEQUENCES

SEQ ID No. 71: Nucleotide sequence of the obtained “hybrid” −696−43 DF3-−39−1 minimal CMV promoter. Minimal CMV part is shown in bold.

| −696 | GGACCCTAGG GTTCATCGGA GCCCAGGTTT ACTCCCTTAA GTGGAAATTT | −647 |
|---|---|---|
| −646 | CTTCCCCCAC TCCCTCCTTG GCTTTCTCCA AGGAGGGAAC CCAGGCTGCT | −597 |
| −596 | GGAAAGTCCG GCTGGGGCGG GGACTGTGGG TTTCAGGGTA GAACTGCGTG | −547 |
| −546 | TGGAACGGGA CAGGGAGCGG TTAGAAGGGT GGGGCTATTC CGGGAAGTGG | −497 |
| −496 | TGGGGGGAGG GAGCCCAAAA CTAGCACCTA GTCCACTCAT TATCCAGCCC | −447 |
| −446 | TCTTATTTCT CGGCCCCGCT CTGCTTCAGT GGACCCGGGG AGGGCGGGGA | −397 |
| −396 | AGTGGAGTGG GAGACCTAGG GGTGGGCTTC CCGACCTTGC TGTACAGGAC | −347 |
| −346 | CTCGACCTAG CTGGCTTTGT TCCCCATCCC CACGTTAGTT GTTGCCCTGA | −297 |
| −296 | GGCTAAAACT AGAGCCCAGG GGCCCCAAGT TCCAGACTGC CCCTCCCCCC | −247 |
| −246 | TCCCCCGGAG CCAGGGAGTG GTTGGTGAAA GGGGGAGGCC AGCTGGAGAA | −197 |
| −196 | CAAACGGGTA GTCAGGGGGT TGAGCGATTA GAGCCCTTGT ACCCTACCCA | −147 |
| −146 | GGAATGGTTG GGGAGGAGGA GGAAGAGGTA GGAGGTAGGG GAGGGGGCGG | −97 |
| −96 | GGTTTTGTCA CCTGTCACCT GCTCCGGCTG TGCCTAGGGC GGGCGGGCGG | −47 |
| | minimal CMV part | |
| −46-39 | GGAGTGGGAG GTCTATATAA GCAGAGCTGG TTTAGTGAAC CG | −1 |

SEQ ID No. 72: MUC1,acc.#gi|324120957|ref|NM_001204286.1| Homo sapiens mucin 1, cell surface associated (MUC1), transcript variant 10, mRNA (long variant)

CGCTCCACCTCTCAAGCAGCCAGCGCCTGCCTGAATCTGTTCTGCCCCCTCCCCACCCATTTCACCACCA
CCATGACACCGGGCACCCAGTCTCCTTTCTTCCTGCTGCTGCTCCTCACAGTGCTTACAGCTACCACAGC
CCCTAAACCCGCAACAGTTGTTACGGGTTCTGGTCATGCAAGCTCTACCCCAGGTGGAGAAAAGGAGACT
TCGGCTACCCAGAGAAGTTCAGTGCCCAGCTCTACTGAGAAGAATGCTGTGAGTATGACCAGCAGCGTAC
TCTCCAGCCACAGCCCCGGTTCAGGCTCCTCCACCACTCAGGGACAGGATGTCACTCTGGCCCCGGCCAC
GGAACCAGCTTCAGGTTCAGCTGCCACCTGGGGACAGGATGTCACCTCGGTCCCAGTCACCAGGCCAGCC
CTGGGCTCCACCACCCCGCCAGCCCACGATGTCACCTCAGCCCCGGACAACAAGCCAGCCCCGGGCTCCA
CCGCCCCCCCAGCCCACGGTGTCACCTCGGCCCCGGACACCAGGCCGGCCCCGGGCTCCACCGCCCCCCC
AGCCCATGGTGTCACCTCGGCCCCGGACAACAGGCCCGCCTTGGGCTCCACCGCCCCTCCAGTCCACAAT
GTCACCTCGGCCTCAGGCTCTGCATCAGGCTCAGCTTCTACTCTGGTGCACAACGGCACCTCTGCCAGGG
CTACCACAACCCCAGCCAGCAAGAGCACTCCATTCTCAATTCCCAGCCACCACTCTGATACTCCTACCAC
CCTTGCCAGCCATAGCACCAAGACTGATGCCAGTAGCACTCACCATAGCACGGTACCTCCTCTCACCTCC
TCCAATCACAGCACTTCTCCCCAGTTGTCTACTGGGGTCTCTTTCTTTTTCCTGTCTTTTCACATTTCAA
ACCTCCAGTTTAATTCCTCTCTGGAAGATCCCAGCACCGACTACTACCAAGAGCTGCAGAGAGACATTTC
TGAAATGTTTTTGCAGATTTATAAACAAGGGGGTTTTCTGGGCCTCTCCAATATTAAGTTCAGGCCAGGA
TCTGTGGTGGTACAATTGACTCTGGCCTTCCGAGAAGGTACCATCAATGTCCACGACGTGGAGACACAGT
TCAATCAGTATAAAACGGAAGCAGCCTCTCGATATAACCTGACGATCTCAGACGTCAGCGTGAGTGATGT
GCCATTTCCTTTCTCTGCCCAGTCTGGGGCTGGGGTGCCAGGCTGGGGCATCGCGCTGCTGGTGCTGGTC
TGTGTTCTGGTTGCGCTGGCCATTGTCTATCTCATTGCCTTGGCTGTCTGTCAGTGCCGCCGAAAGAACT
ACGGGCAGCTGGACATCTTTCCAGCCCGGGATACCTACCATCCTATGAGCGAGTACCCCACCTACCACAC
CCATGGGCGCTATGTGCCCCCTAGCAGTACCGATCGTAGCCCCTATGAGAAGGTTTCTGCAGGTAATGGT
GGCAGCAGCCTCTCTTACACAAACCCAGCAGTGGCAGCCACTTCTGCCAACTTGTAGGGGCACGTCGCCC
GCTGAGCTGAGTGGCCAGCCAGTGCCATTCCACTCCACTCAGGTTCTTCAGGGCCAGAGCCCCTGCACCC
TGTTTGGGCTGGTGAGCTGGGAGTTCAGGTGGGCTGCTCACAGCCTCCTTCAGAGGCCCCACCAATTTCT
CGGACACTTCTCAGTGTGTGGAAGCTCATGTGGGCCCCTGAGGGCTCATGCCTGGGAAGTGTTGTGGTGG
GGGCTCCCAGGAGGACTGGCCCAGAGAGCCCTGAGATAGCGGGGATCCTGAACTGGACTGAATAAAACGT
GGTCTCCCACTGCGCCAAAAAAAAAAAAAAAAA

SEQ ID No. 73: MUC1, acc.#gi|324120957:1-130 exon 1 Homo sapiens mucin 1, cell surface associated (MUC1), transcript variant 10, mRNA

CGCTCCACCTCTCAAGCAGCCAGCGCCTGCCTGAATCTGTTCTGCCCCC
TCCCCACCCATTTCACCACCACCATGACACCGGGCACCCAGTCTCCTTT
CTTCCTGCTGCTGCTCCTCACAGTGCTTACAG

SEQ ID No. 74: MUC1, acc.#gi|324120957:534-987 exon 3a Homo sapiens mucin 1, cell surface associated (MUC1), transcript variant 10, mRNA

GCCGGCCCCGGGCTCCACCGCCCCCCCAGCCCATGGTGTCACCTCGGCCC
CGGACAACAGGCCCGCCTTGGGCTCCACCGCCCCTCCAGTCCACAATGTC
ACCTCGGCCTCAGGCTCTGCATCAGGCTCAGCTTCTACTCTGGTGCACAA
CGGCACCTCTGCCAGGGCTACCACAACCCCAGCCAGCAAGAGCACTCCAT
TCTCAATTCCCAGCCACCACTCTGATACTCCTACCACCCTTGCCAGCCAT
AGCACCAAGACTGATGCCAGTAGCACTCACCATAGCACGGTACCTCCTCT
CACCTCCTCCAATCACAGCACTTCTCCCCAGTTGTCTACTGGGGTCTCTT
TCTTTTTCCTGTCTTTTCACATTTCAAACCTCCAGTTTAATTCCTCTCTG
GAAGATCCCAGCACCGACTACTACCAAGAGCTGCAGAGAGACATTTCTGA
AATG

SEQ ID No. 75: MUC1, acc.#gi|324120957:1303-1452 exon 7 Homo sapiens mucin 1, cell surface associated (MUC1), transcript variant 10, mRNA

GCTGTCTGTCAGTGCCGCCGAAAGAACTACGGGCAGCTGGACATCTTTCC
AGCCCGGGATACCTACCATCCTATGAGCGAGTACCCCACCTACCACACCC
ATGGGCGCTATGTGCCCCCTAGCAGTACCGATCGTAGCCCCTATGAGAAG

SEQ ID No. 76: MUC1, acc.#gi|324120957:1453-1838 exon 8 Homo sapiens mucin 1, cell surface associated (MUC1), transcript variant 10, mRNA

GTTTCTGCAGGTAATGGTGGCAGCAGCCTCTCTTACACAAACCCAGCAGT
GGCAGCCACTTCTGCCAACTTGTAGGGGCACGTCGCCCGCTGAGCTGAGT
GGCCAGCCAGTGCCATTCCACTCCACTCAGGTTCTTCAGGGCCAGAGCCC
CTGCACCCTGTTTGGGCTGGTGAGCTGGGAGTTCAGGTGGGCTGCTCACA
GCCTCCTTCAGAGGCCCCACCAATTTCTCGGACACTTCTCAGTGTGTGGA
AGCTCATGTGGGCCCCTGAGGGCTCATGCCTGGGAAGTGTTGTGGTGGGG
GCTCCCAGGAGGACTGGCCCAGAGAGCCCTGAGATAGCGGGGATCCTGAA
CTGGACTGAATAAAACGTGGTCTCCCACTGCGCCAA

SEQ ID No. 77: ERBB2 isoform A, acc.#gi|54792095|ref|NM_004448.21 Homo sapiens v-erb-b2 erythroblastic leukemia viral oncogene homolog 2, neuro/glioblastoma derived oncogene homolog (avian) (ERBB2), transcript variant 1, mRNA

GGAGGAGGTGGAGGAGGAGGGCTGCTTGAGGAAGTATAAGAATGAAGTTGTGAAGCTGAGATTC
CCCTCC
ATTGGGACCGGAGAAACCAGGGGAGCCCCCCGGGCAGCCGCGCGCCCCTTCCCACGGGGCCC
TTTACTGC
GCCGCGCGCCCGGCCCCCACCCCTCGCAGCACCCCGCGCCCCGCGCCCTCCCAGCCGGGTCC
AGCCGGAG
CCATGGGGCCGGAGCCGCAGTGAGCACCATGGAGCTGGCGGCCTTGTGCCGCTGGGGGCTCC
TCCTCGCC
CTCTTGCCCCCCGGAGCCGCGAGCACCCAAGTGTGCACCGGCACAGACATGAAGCTGCGGCTC
CCTGCCA
GTCCCGAGACCCACCTGGACATGCTCCGCCACCTCTACCAGGGCTGCCAGGTGGTGCAGGGAA
ACCTGGA
ACTCACCTACCTGCCCACCAATGCCAGCCTGTCCTTCCTGCAGGATATCCAGGAGGTGCAGGGC
TACGTG
CTCATCGCTCACAACCAAGTGAGGCAGGTCCCACTGCAGAGGCTGCGGATTGTGCGAGGCACC
CAGCTCT
TTGAGGACAACTATGCCCTGGCCGTGCTAGACAATGGAGACCCGCTGAACAATACCACCCCTGT
CACAGG
GGCCTCCCCAGGAGGCCTGCGGGAGCTGCAGCTTCGAAGCCTCACAGAGATCTTGAAAGGAGG
GGTCTTG
ATCCAGCGGAACCCCCAGCTCTGCTACCAGGACACGATTTTGTGGAAGGACATCTTCCACAAGAA
CAACC
AGCTGGCTCTCACACTGATAGACACCAACCGCTCTCGGGCCTGCCACCCCTGTTCTCCGATGTG
TAAGGG
CTCCCGCTGCTGGGGAGAGAGTTCTGAGGATTGTCAGAGCCTGACGCGCACTGTCTGTGCCGGT
GGCTGT
GCCCGCTGCAAGGGGCCACTGCCCACTGACTGCTGCCATGAGCAGTGTGCTGCCGGCTGCACG
GGCCCCA
AGCACTCTGACTGCCTGGCCTGCCTCCACTTCAACCACAGTGGCATCTGTGAGCTGCACTGCCC
AGCCCT
GGTCACCTACAACACAGACACGTTTGAGTCCATGCCCAATCCCGAGGGCCGGTATACATTCGGC
GCCAGC
TGTGTGACTGCCTGTCCCTACAACTACCTTTCTACGGACGTGGGATCCTGCACCCTCGTCTGCCC
CCTGC
ACAACCAAGAGGTGACAGCAGAGGATGGAACACAGCGGTGTGAGAAGTGCAGCAAGCCCTGTG
CCCGAGT
GTGCTATGGTCTGGGCATGGAGCACTTGCGAGAGGTGAGGGCAGTTACCAGTGCCAATATCCAG
GAGTTT
GCTGGCTGCAAGAAGATCTTTGGGAGCCTGGCATTTCTGCCGGAGAGCTTTGATGGGGACCCAG
CCTCCA
ACACTGCCCCGCTCCAGCCAGAGCAGCTCCAAGTGTTTGAGACTCTGGAAGAGATCACAGGTTA
CCTATA
CATCTCAGCATGGCCGGACAGCCTGCCTGACCTCAGCGTCTTCCAGAACCTGCAAGTAATCCGG
GGACGA
ATTCTGCACAATGGCGCCTACTCGCTGACCCTGCAAGGGCTGGGCATCAGCTGGCTGGGGCTG
CGCTCAC
TGAGGGAACTGGGCAGTGGACTGGCCCTCATCCACCATAACACCCACCTCTGCTTCGTGCACAC
GGTGCC
CTGGGACCAGCTCTTTCGGAACCCGCACCAAGCTCTGCTCCACACTGCCAACCGGCCAGAGGAC
GAGTGT
GTGGGCGAGGGCCTGGCCTGCCACCAGCTGTGCGCCCGAGGGCACTGCTGGGGTCCAGGGCC
CACCCAGT
GTGTCAACTGCAGCCAGTTCCTTCGGGGCCAGGAGTGCGTGGAGGAATGCCGAGTACTGCAGG
GGCTCCC
CAGGGAGTATGTGAATGCCAGGCACTGTTTGCCGTGCCACCCTGAGTGTCAGCCCCAGAATGGC
TCAGTG
ACCTGTTTTGGACCGGAGGCTGACCAGTGTGTGGCCTGTGCCCACTATAAGGACCCTCCCTTCT
GCGTGG
CCCGCTGCCCCAGCGGTGTGAAACCTGACCTCTCCTACATGCCCATCTGGAAGTTTCCAGATGA
GGAGGG
CGCATGCCAGCCTTGCCCCATCAACTGCACCCACTCCTGTGTGGACCTGGATGACAAGGGCTGC
CCCGCC
GAGCAGAGAGCCAGCCCTCTGACGTCCATCATCTCTGCGGTGGTTGGCATTCTGCTGGTCGTGG
TCTTGG
GGGTGGTCTTTGGGATCCTCATCAAGCGACGGCAGCAGAAGATCCGGAAGTACACGATGCGGA
GACTGCT
GCAGGAAACGGAGCTGGTGGAGCCGCTGACACCTAGCGGAGCGATGCCCAACCAGGCGCAGAT
GCGGATC
CTGAAAGAGACGGAGCTGAGGAAGGTGAAGGTGCTTGGATCTGGCGCTTTTGGCACAGTCTACA
AGGGCA
TCTGGATCCCTGATGGGGAGAATGTGAAAATTCCAGTGGCCATCAAAGTGTTGAGGGAAAACACA
TCCCC
CAAAGCCAACAAAGAAATCTTAGACGAAGCATACGTGATGGCTGGTGTGGGCTCCCCATATGTCT
CCCGC
CTTCTGGGCATCTGCCTGACATCCACGGTGCAGCTGGTGACACAGCTTATGCCCTATGGCTGCC
TCTTAG
ACCATGTCCGGGAAAACCGCGGACGCCTGGGCTCCCAGGACCTGCTGAACTGGTGTATGCAGAT
TGCCAA
GGGGATGAGCTACCTGGAGGATGTGCGGCTCGTACACAGGGACTTGGCCGCTCGGAACGTGCT
GGTCAAG
AGTCCCAACCATGTCAAAATTACAGACTTCGGGCTGGCTCGGCTGCTGGACATTGACGAGACAG
AGTACC
ATGCAGATGGGGGCAAGGTGCCCATCAAGTGGATGGCGCTGGAGTCCATTCTCCGCCGGCGGT
TCACCCA
CCAGAGTGATGTGTGGAGTTATGGTGTGACTGTGTGGGAGCTGATGACTTTTGGGGCCAAACCT
TACGAT
GGGATCCCAGCCCGGGAGATCCCTGACCTGCTGGAAAAGGGGGAGCGGCTGCCCCAGCCCCC
CATCTGCA
CCATTGATGTCTACATGATCATGGTCAAATGTTGGATGATTGACTCTGAATGTCGGCCAAGATTCC
GGGA
GTTGGTGTCTGAATTCTCCCGCATGGCCAGGGACCCCCAGCGCTTTGTGGTCATCCAGAATGAG
GACTTG
GGCCCAGCCAGTCCCTTGGACAGCACCTTCTACCGCTCACTGCTGGAGGACGATGACATGGGG
GACCTGG
TGGATGCTGAGGAGTATCTGGTACCCCAGCAGGGCTTCTTCTGTCCAGACCCTGCCCCGGGCGC
TGGGGG
CATGGTCCACCACAGGCACCGCAGCTCATCTACCAGGAGTGGCGGTGGGGACCTGACACTAGG
GCTGGAG
CCCTCTGAAGAGGAGGCCCCCAGGTCTCCACTGGCACCCTCCGAAGGGGCTGGCTCCGATGTA
TTTGATG
GTGACCTGGGAATGGGGGCAGCCAAGGGGCTGCAAAGCCTCCCCACACATGACCCCAGCCCTC
TACAGCG
GTACAGTGAGGACCCCACAGTACCCCTGCCCTCTGAGACTGATGGCTACGTTGCCCCCCTGACC
TGCAGC
CCCCAGCCTGAATATGTGAACCAGCCAGATGTTCGGCCCCAGCCCCCTTCGCCCCGAGAGGGC
CCTCTGC
CTGCTGCCCGACCTGCTGGTGCCACTCTGGAAAGGCCCAAGACTCTCTCCCCAGGGAAGAATGG
GGTCGT
CAAAGACGTTTTTGCCTTTGGGGGTGCCGTGGAGAACCCCGAGTACTTGACACCCCAGGGAGGA
GCTGCC
CCTCAGCCCCACCCTCCTCCTGCCTTCAGCCCAGCCTTCGACAACCTCTATTACTGGGACCAGG
ACCCAC
CAGAGCGGGGGGCTCCACCCAGCACCTTCAAAGGGACACCTACGGCAGAGAACCCAGAGTACC
TGGGTCT
GGACGTGCCAGTGTGAACCAGAAGGCCAAGTCCGCAGAAGCCCTGATGTGTCCTCAGGGAGCA
GGGAAGG
CCTGACTTCTGCTGGCATCAAGAGGTGGGAGGGCCCTCCGACCACTTCCAGGGGAACCTGCCAT
GCCAGG
AACCTGTCCTAAGGAACCTTCCTTCCTGCTTGAGTTCCCAGATGGCTGGAAGGGGTCCAGCCTC
GTTGGA
AGAGGAACAGCACTGGGGAGTCTTTGTGGATTCTGAGGCCCTGCCCAATGAGACTCTAGGGTCC
AGTGGA
TGCCACAGCCCAGCTTGGCCCTTTCCTTCCAGATCCTGGGTACTGAAAGCCTTAGGGAAGCTGG
CCTGAG
AGGGGAAGCGGCCCTAAGGGAGTGTCTAAGAACAAAAGCGACCCATTCAGAGACTGTCCCTGAA
ACCTAG
TACTGCCCCCCATGAGGAAGGAACAGCAATGGTGTCAGTATCCAGGCTTTGTACAGAGTGCTTTT
CTGTT
TAGTTTTTACTTTTTTTGTTTTGTTTTTTTAAAGATGAAATAAAGACCCAGGGGGAGAATGGGTGTT
GTA
TGGGGAGGCAAGTGTGGGGGGTCCTTCTCCACACCCACTTTGTCCATTTGCAAATATATTTTGGA
AAACA
GCTA

SEQ ID No. 78: ERBB2 Total form (wt), acc.#gi|54792097:1190-1331 exon 11 Homo sapiens v-erb-b2 erythroblastic leukemia viral oncogene homolog 2, neuro/glioblastoma derived oncogene homolog (avian) (ERBB2), transcript variant 2, mRNA

GCCTGCCTCCACTTCAACCACAGTGGCATCTGTGAGCTGCACTGCCCAGC
CCTGGTCACCTACAACACAGACACGTTTGAGTCCATGCCCAATCCCGAGG
GCCGGTATACATTCGGCGCCAGCTGTGTGACTGCCTGTCCCT

SEQ ID No. 79: ERBB2 Total form (wt),acc.#gi|54792097:1332-1451 exon 12 Homo sapiens v-erb-b2 erythroblastic leukemia viral oncogene homolog 2, neuro/glioblastoma derived oncogene homolog (avian) (ERBB2), transcript variant 2, mRNA

ACAACTACCTTTCTACGGACGTGGGATCCTGCACCCTCGTCTGCCCCCTG
CACAACCAAGAGGTGACAGCAGAGGATGGAACACAGCGGTGTGAGAAGTG
CAGCAAGCCCTGTGCCCGAG

SEQ ID No. 80: ERBB2 Mutant form, acc.#gi|54792097:2168-2328 exon 19 Homo sapiens v-erb-b2 erythroblastic leukemia viral oncogene homolog 2, neuro/glioblastoma derived oncogene homolog (avian) (ERBB2), transcript variant 2, mRNA

GAGGCTGACCAGTGTGTGGCCTGTGCCCACTATAAGGACCCTCCCTTCTG
CGTGGCCCGCTGCCCCAGCGGTGTGAAACCTGACCTCTCCTACATGCCCA
TCTGGAAGTTTCCAGATGAGGAGGGCGCATGCCAGCCTTGCCCCATCAAC
TGCACCCACTC-one for5′

SEQ ID No. 81:ERBB2 Mutant form acc.#gi|54792097:2377-2515 exon 21 Homo sapiens v-erb-b2 erythroblastic leukemia viral oncogene homolog 2,neuro/glioblastoma derived oncogene homolog (avian) (ERBB2), transcript variant 2, mRNA

one for5′-
CCCTCTGACGTCCATCATCTCTGCGGTGGTTGGCATTCTGCTGGTCGTGG
TCTTGGGGGTGGTCTTTGGGATCCTCATCAAGCGACGGCAGCAGAAGATC
CGGAAGTACACGATGCGGAGACTGCTGCAGGAAACGGAG

SEQ ID No. 82: ESR1, acc.# gi|170295798|ref|NM_000125.3| Homo sapiens estrogen receptor 1 (ESR1), transcript variant 1, mRNA

AGGAGCTGGCGGAGGGCGTTCGTCCTGGGACTGCACTTGCTCCCGTCGGGTCGCCCGGCTTCA
CCGGACC
CGCAGGCTCCCGGGGCAGGGCCGGGGCCAGAGCTCGCGTGTCGGCGGGACATGCGCTGCGTC
GCCTCTAA
CCTCGGGCTGTGCTCTTTTTCCAGGTGGCCCGCCGGTTTCTGAGCCTTCTGCCCTGCGGGGACA
CGGTCT
GCACCCTGCCCGCGGCCACGGACCATGACCATGACCCTCCACACCAAAGCATCTGGGATGGCC
CTACTGC
ATCAGATCCAAGGGAACGAGCTGGAGCCCCTGAACCGTCCGCAGCTCAAGATCCCCCTGGAGC
GGCCCCT
GGGCGAGGTGTACCTGGACAGCAGCAAGCCCGCCGTGTACAACTACCCCGAGGGCGCCGCCTA
CGAGTTC
AACGCCGCGGCCGCCGCCAACGCGCAGGTCTACGGTCAGACCGGCCTCCCCTACGGCCCCGG
GTCTGAGG
CTGCGGCGTTCGGCTCCAACGGCCTGGGGGGTTTCCCCCCACTCAACAGCGTGTCTCCGAGCC
CGCTGAT
GCTACTGCACCCGCCGCCGCAGCTGTCGCCTTTCCTGCAGCCCCACGGCCAGCAGGTGCCCTA
CTACCTG
GAGAACGAGCCCAGCGGCTACACGGTGCGCGAGGCCGGCCCGCCGGCATTCTACAGGCCAAA
TTCAGATA
ATCGACGCCAGGGTGGCAGAGAAAGATTGGCCAGTACCAATGACAAGGGAAGTATGGCTATGGA
ATCTGC
CAAGGAGACTCGCTACTGTGCAGTGTGCAATGACTATGCTTCAGGCTACCATTATGGAGTCTGGT
CCTGT
GAGGGCTGCAAGGCCTTCTTCAAGAGAAGTATTCAAGGACATAACGACTATATGTGTCCAGCCAC
CAACC
AGTGCACCATTGATAAAAACAGGAGGAAGAGCTGCCAGGCCTGCCGGCTCCGCAAATGCTACGA
AGTGGG
AATGATGAAAGGTGGGATACGAAAAGACCGAAGAGGAGGGAGAATGTTGAAACACAAGCGCCAG
AGAGAT
GATGGGGAGGGCAGGGGTGAAGTGGGGTCTGCTGGAGACATGAGAGCTGCCAACCTTTGGCCA
AGCCCGC
TCATGATCAAACGCTCTAAGAAGAACAGCCTGGCCTTGTCCCTGACGGCCGACCAGATGGTCAG
TGCCTT
GTTGGATGCTGAGCCCCCCATACTCTATTCCGAGTATGATCCTACCAGACCCTTCAGTGAAGCTT
CGATG
ATGGGCTTACTGACCAACCTGGCAGACAGGGAGCTGGTTCACATGATCAACTGGGCGAAGAGGG
TGCCAG
GCTTTGTGGATTTGACCCTCCATGATCAGGTCCACCTTCTAGAATGTGCCTGGCTAGAGATCCTG
ATGAT
TGGTCTCGTCTGGCGCTCCATGGAGCACCCAGGGAAGCTACTGTTTGCTCCTAACTTGCTCTTGG
ACAGG
AACCAGGGAAAATGTGTAGAGGGCATGGTGGAGATCTTCGACATGCTGCTGGCTACATCATCTC
GGTTCC
GCATGATGAATCTG CAGGGAGAGGAGTTTGTGTGCCTCAAATCTATTATTTTGCTTAATTCTGGAG
TGTA
CACATTTCTGTCCAGCACCCTGAAGTCTCTGGAAGAGAAGGACCATATCCACCGAGTCCTGGACA
AGATC
ACAGACACTTTGATCCACCTGATGGCCAAGGCAGGCCTGACCCTGCAGCAGCAGCACCAGCGG
CTGGCCC
AGCTCCTCCTCATCCTCTCCCACATCAGGCACATGAGTAACAAAGGCATGGAGCATCTGTACAGC
ATGAA
GTGCAAGAACGTGGTGCCCCTCTATGACCTGCTGCTGGAGATGCTGGACGCCCACCGCCTACAT
GCGCCC
ACTAGCCGTGGAGGGGCATCCGTGGAGGAGACGGACCAAAGCCACTTGGCCACTGCGGGCTCT
ACTTCAT
CGCATTCCTTGCAAAAGTATTACATCACGGGGGAGGCAGAGGGTTTCCCTGCCACGGTCTGAGA
GCTCCC
TGGCTCCCACACGGTTCAGATAATCCCTGCTGCATTTTACCCTCATCATGCACCACTTTAGCCAA
ATTCT
GTCTCCTGCATACACTCCGGCATGCATCCAACACCAATGGCTTTCTAGATGAGTGGCCATTCATT
TGCTT
......

SEQ ID No. 83: ESR1, acc.#gi|170295798:1-686 exon 1 Homo sapiens estrogen receptor 1 (ESR1), transcript variant 1, mRNA

AGGAGCTGGCGGAGGGCGTTCGTCCTGGGACTGCACTTGCTCCCGTCGGGTCGCCCGGCTTCA
CCGGACC
CGCAGGCTCCCGGGGCAGGGCCGGGGCCAGAGCTCGCGTGTCGGCGGGACATGCGCTGCGTC
GCCTCTAA
CCTCGGGCTGTGCTCTTTTTCCAGGTGGCCCGCCGGTTTCTGAGCCTTCTGCCCTGCGGGGACA
CGGTCT
GCACCCTGCCCGCGGCCACGGACCATGACCATGACCCTCCACACCAAAGCATCTGGGATGGCC
CTACTGC
ATCAGATCCAAGGGAACGAGCTGGAGCCCCTGAACCGTCCGCAGCTCAAGATCCCCCTGGAGC
GGCCCCT
GGGCGAGGTGTACCTGGACAGCAGCAAGCCCGCCGTGTACAACTACCCCGAGGGCGCCGCCTA
CGAGTTC
AACGCCGCGGCCGCCGCCAACGCGCAGGTCTACGGTCAGACCGGCCTCCCCTACGGCCCCGG
GTCTGAGG
CTGCGGCGTTCGGCTCCAACGGCCTGGGGGGTTTCCCCCCACTCAACAGCGTGTCTCCGAGCC
CGCTGAT
GCTACTGCACCCGCCGCCGCAGCTGTCGCCTTTCCTGCAGCCCCACGGCCAGCAGGTGCCCTA
CTACCTG
GAGAACGAGCCCAGCGGCTACACGGTGCGCGAGGCCGGCCCGCCGGCATTCTACAG

SEQ ID No. 84: PGR, acc.#gi|321117149|ref|NM_001202474.1| Homo sapiens progesterone receptor (PGR), transcript variant 1, mRNA

AGCTGAAGGCAAAGGGTCCCCGGGCTCCCCACGTGGCGGGCGGCCCGCCCTCCCCCGAGGTC
GGATCCCC
ACTGCTGTGTCGCCCAGCCGCAGGTCCGTTCCCGGGGAGCCAGACCTCGGACACCTTGCCTGA
AGTTTCG
GCCATACCTATCTCCCTGGACGGGCTACTCTTCCCTCGGCCCTGCCAGGGACAGGACCCCTCCG
ACGAAA
AGACGCAGGACCAGCAGTCGCTGTCGGACGTGGAGGGCGCATATTCCAGAGCTGAAGCTACAA
GGGGTGC
TGGAGGCAGCAGTTCTAGTCCCCCAGAAAAGGACAGCGGACTGCTGGACAGTGTCTTGGACACT
CTGTTG
GCGCCCTCAGGTCCCGGGCAGAGCCAACCCAGCCCTCCCGCCTGCGAGGTCACCAGCTCTTGG
TGCCTGT
TTGGCCCCGAACTTCCCGAAGATCCACCGGCTGCCCCCGCCACCCAGCGGGTGTTGTCCCCGC
TCATGAG
CCGGTCCGGGTGCAAGGTTGGAGACAGCTCCGGGACGGCAGCTGCCCATAAAGTGCTGCCCCG
GGGCCTG
TCACCAGCCCGGCAGCTGCTGCTCCCGGCCTCTGAGAGCCCTCACTGGTCCGGGGCCCCAGTG
AAGCCGT
CTCCGCAGGCCGCTGCGGTGGAGGTTGAGGAGGAGGATGGCTCTGAGTCCGAGGAGTCTGCG
GGTCCGCT
TCTGAAGGGCAAACCTCGGGCTCTGGGTGGCGCGGCGGCTGGAGGAGGAGCCGCGGCTGTCC
CGCCGGGG
GCGGCAGCAGGAGGCGTCGCCCTGGTCCCCAAGGAAGATTCCCGCTTCTCAGCGCCCAGGGTC
GCCCTGG
TGGAGCAGGACGCGCCGATGGCGCCCGGGCGCTCCCCGCTGGCCACCACGGTGATGGATTTCA
TCCACGT
GCCTATCCTGCCTCTCAATCACGCCTTATTGGCAGCCCGCACTCGGCAGCTGCTGGAAGACGAA
AGTTAC
GACGGCGGGGCCGGGGCTGCCAGCGCCTTTGCCCCGCCGCGGAGTTCACCCTGTGCCTCGTC
CACCCCGG
TCGCTGTAGGCGACTTCCCCGACTGCGCGTACCCGCCCGACGCCGAGCCCAAGGACGACGCGT
ACCCTCT
CTATAGCGACTTCCAGCCGCCCGCTCTAAAGATAAAGGAGGAGGAGGAAGGCGCGGAGGCCTC
CGCGCGC
TCCCCGCGTTCCTACCTTGTGGCCGGTGCCAACCCCGCAGCCTTCCCGGATTTCCCGTTGGGGC
CACCGC
CCCCGCTGCCGCCGCGAGCGACCCCATCCAGACCCGGGGAAGCGGCGGTGACGGCCGCACCC
GCCAGTGC
CTCAGTCTCGTCTGCGTCCTCCTCGGGGTCGACCCTGGAGTGCATCCTGTACAAAGCGGAGGGC
GCGCCG
CCCCAGCAGGGCCCGTTCGCGCCGCCGCCCTGCAAGGCGCCGGGCGCGAGCGGCTGCCTGCT
CCCGCGGG
ACGGCCTGCCCTCCACCTCCGCCTCTGCCGCCGCCGCCGGGGCGGCCCCCGCGCTCTACCCT
GCACTCGG
CCTCAACGGGCTCCCGCAGCTCGGCTACCAGGCCGCCGTGCTCAAGGAGGGCCTGCCGCAGGT
CTACCCG
CCCTATCTCAACTACCTGAGGCCGGATTCAGAAGCCAGCCAGAGCCCACAATACAGCTTCGAGT
CATTAC
CTCAGAAGATTTGTTTAATCTGTGGGGATGAAGCATCAGGCTGTCATTATGGTGTCCTTACCTGTG
GGAG
CTGTAAGGTCTTCTTTAAGAGGGCAATGGAAGGGCAGCACAACTACTTATGTGCTGGAAGAAATG
ACTGC
ATCGTTGATAAAATCCGCAGAAAAAACTGCCCAGCATGTCGCCTTAGAAAGTGCTGTCAGGCTGG
CATGG
TCCTTGGAGGTCGAAAATTTAAAAAGTTCAATAAAGTCAGAGTTGTGAGAGCACTGGATGCTGTT
GCTCT
CCCACAGCCAGTGGGCGTTCCAAATGAAAGCCAAGCCCTAAGCCAGAGATTCACTTTTTCACCAG
GTCAA
GACATACAGTTGATTCCACCACTGATCAACCTGTTAATGAGCATTGAACCAGATGTGATCTATGCA
GGAC
ATGACAACACAAAACCTGACACCTCCAGTTCTTTGCTGACAAGTCTTAATCAACTAGGCGAGAGG
CAACT
TCTTTCAGTAGTCAAGTGGTCTAAATCATTGCCAGGTTTTCGAAACTTACATATTGATGACCAGAT
AACT
CTCATTCAGTATTCTTGGATGAGCTTAATGGTGTTTGGTCTAGGATGGAGATCCTACAAACACGTC
AGTG
GGCAGATGCTGTATTTTGCACCTGATCTAATACTAAATGAACAGCGGATGAAAGAATCATCATTCT
ATTC
ATTATGCCTTACCATGTGGCAGATCCCACAGGAGTTTGTCAAGCTTCAAGTTAGCCAAGAAGAGT
TCCTC
TGTATGAAAGTATTGTTACTTCTTAATACAATTCCTTTGGAAGGGCTACGAAGTCAAACCCAGTTT
GAGG
AGATGAGG TCAAGCTACATTAGAGAGCTCATCAAGGCAATTGGTTTGAGGCAAAAAGGAGTTGTG
TCGAG
CTCACAGCGTTTCTATCAACTTACAAAACTTCTTGATAACTTGCATGATCTTGTCAAACAACTTCA
TCTG
TACTGCTTGAATACATTTATCCAGTCCCGGGCACTGAGTGTTGAATTTCCAGAAATGATGTCTGA
AGTTA
TTGCTGCACAATTACCCAAGATATTGGCAGGGATGGTGAAACCCCTTCTCTTTCATAAAAAGTGA
ATGTC
ATCTTTTTCTTTTAAAGAATTAAATTTTGTGGTATGTCTTTTTGTTTTGGTCAGGATTATGAGGTCTT
GA
.........

SEQ ID No. 85: PGR, acc.#gi|160358783:3232-3389 exon 7 Homo sapiens progesterone receptor (PGR), transcript variant 2, mRNA

TTCCTTTGGAAGGGCTACGAAGTCAAACCCAGTTTGAGGAGATGAGGTCA
AGCTACATTAGAGAGCTCATCAAGGCAATTGGTTTGAGGCAAAAAGGAGT
TGTGTCGAGCTCACAGCGTTTCTATCAACTTACAAAACTTCTTGATAACT
TGCATGAT......

SEQ ID No. 86: PGR, acc.#gi|160358783:3390-13037 exon 8 Homo sapiens progesterone receptor (PGR), transcript variant 2, mRNA

CTTGTCAAACAACTTCATCTGTACTGCTTGAATACATTTATCCAGTCCCG
GGCACTGAGTGTTGAATTTCCAGAAATGATGTCTGAAGTTATTGCTGCAC
AATTACCCAAGATATTGGCAGGGATGGTGAAACCCCTTCTCTTTCATAAA
AAGTGAATGTCATCTTTTTCTTTTAAAGAATTAAATTTTGTGGTATGTCT
TTTTGTTTTGGTCAGGATTATGAGGTCTTGAGTTTTTATAATGTTCTTCT
GAAAGCCTTACATTTATAACATCATAGTGTGTAAATTTAAAAGAAAAATT
GTGAGGTTCTAATTATTTTCTTTTATAAAGTATAATTAGAATGTTTAACT
.........

SEQ ID No. 87: Initial sequence of TK2 gene. Nucleotides different from the majority of TK2 sequences in GenBank are shown underlined bold (except “problem region”).

Problem region is indicated. Nucleotides modified inside the problem region are underlined bold in this region.

| | |
|---|---|
| 1 | ATGGCTTCTC ACGCCGGCCA ACAGCACGCG CCTGCGTTCG GTCAGGCTGC |
| | TCGTGCGAGC GGGCCTACCG ACGGCCGCGC GGCGTCCCGT CCTAGCCATC |
| | BamHI |
| 101 | GCCAGGGGGC CTCCGAAGCC CGCGGGGATC CGGAGCTGCC CACGCTGCTG |
| | A (mut4) |
| | CGGGTTTATA TAGACGGACC CCACGGGGTG GGGGAGACCA CCACCTCCGC |
| | PvuII |
| | A (mut1) |
| 201 | GCAGCTGATG GAGGCCCTGG GGCCGCGCGA CGATATCGTC TACGTCCCCG |
| | AGCCGATGAC TTACTGGCAG GTGCTGGGGG CCTCCGAGAC CCTGACGAAC |
| 301 | ATCTACAACA CGCAGCACCG TCTGGACCGC GGCGAGATAT CGGCCGGGGA |
| | GGCGGCGGTG GTAATGACCA GCGCCCAGAT AACAATGAGC ACGCCTTATG |
| | ApaI |
| | G (mut2) |
| 401 | CGGCGACGGA CGCCGTTTTT GCTCCTCATA TCGGGGGGGA GGCTGTGGGC |
| | CCGCAAGCCC CGCCCCCGGC CCTCACCCTT GTTTTCGACC GGCACCCTAT |
| 501 | CGCCTCCCTG CTGTGCTACC CGGCCGCGCG GTACCTCATG GGAAGCATGA |
| | CCCCCCAGGC CGTGTTGGCG TTCGTGGCCC TCATGCCCCC GACCGCGCCC |
| | SmaI |
| 601 | GGCACGAACC TGGTCCTGGG TGTCCTTCCG GAGGCCGAAC ACGCCGACCG |
| | CCTGGCCAGA CGCCAACGCC CGGGCGAGCG GCTTGACCTG GCCATGCTGT |
| | PstI |
| 701 | CCGCCATTCG CCGTGTCTAC GACCTACTCG CCAACACGGT GCGGTACCTG |
| | CAGCGCGGCG GGAGGTGGCG GGAGGACTGG GGCCGGCTGA CGGGGGTCGC |
| | Problem region (mut 5) |
| | G G G |
| 801 | CGCGGCGACC CCGCGCCCCG ACCCCGA GGA CGGCGCGGGG TCTCTGCCCC |
| | GCATCGAGGA CACGCTGTTT GCCCTGTTCC GCGTTCCCGA GCTGCTGGCC |
| 901 | CCCAACGGGG ACTTGTACCA CATTTTTGCC TGGGTCTTGG ACGTCTTGGC |
| | CGACCGCCTC CTTCCGATGC ATCTATTTGT CCTGGATTAC GATCAGTCGC |
| | A (mut3) |
| 1001 | CCGTCGGGTG TCGAGACGCC CTGTTGCGCC TCACCGCCGG GATGATCCCA |
| | GCCCGCGTCA CAACCGCCGG GTCCATCGCC GAGATACGCG ACCTGGCGCG |
| | G(mut6)G(mut7) |
| 1101 | CACGTTTGCC CGCGAGATGG GGGAAGTTTA G |

Corrected HSV TK2 nucleotide sequences and corresponding deduced protein sequences.

SEQ ID No. 88: HSV TK2 entire nucleotide sequence without mutation in mut1 site.

ATGGCTTCTCACGCCGGCCAACAGCACGCGCCTGCGTTCGGTCAGGCTGC
TCGTGCGAGCGGGCCTACCGACGGCCGCGCGGCGTCCCGTCCTAGCCATC
GCCAGGGGGCCTCCGAAGCCCGCGGGGATCCGGAGCTGCCCACGCTGCTG
CGGGTTTATATAGACGGACCCCACGGGGTGGGGAAGACCACCACCTCCGC
GCAGCTGATGGAGGCCCTGGGGCCGCGCGACAATATCGTCTACGTCCCCG
AGCCGATGACTTACTGGCAGGTGCTGGGGGCCTCCGAGACCCTGACGAAC
ATCTACAACACGCAGCACCGTCTGGACCGCGGCGAGATATCGGCCGGGGA
GGCGGCGGTGGTAATGACCAGCGCCCAGATAACAATGAGCACGCCTTATG
CGGCGACGGACGCCGTTTTGGCTCCTCATATCGGGGGGGAGGCTGTGGGC
CCGCAAGCCCCGCCCCCGGCCCTCACCCTTGTTTTCGACCGGCACCCTAT
CGCCTCCCTGCTGTGCTACCCGGCCGCGCGGTACCTCATGGGAAGCATGA
CCCCCCAGGCCGTGTTGGCGTTCGTGGCCCTCATGCCTCCGACCGCGCCC
GGCACGAACCTGGTCCTGGGTGTCCTTCCGGAGGCCGAACACGCCGACCG
CCTGGCCAGACGCCAACGCCCGGGCGAGCGGCTTGACCTGGCCATGCTGT
CCGCCATTCGCCGTGTCTACGATCTACTCGCCAACACGGTGCGGTACCTG
CAGCGCGGCGGGAGGTGGCGGGAGGACTGGGGCCGGCTGACGGGGGTCGC
TGCGGCGACCCCGCGGCCGGACCCGGAGGACGGCGCGGGGTCTCTGCCCC
GCATCGAGGACACGCTGTTTGCCCTGTTCCGCGTTCCCGAGCTGCTGGCC
CCCAACGGGGACTTGTACCACATTTTTGCCTGGGTCTTGGACGTCTTGGC
CGACCGCCTCCTTCCGATGCATCTATTTGTCCTGGATTACGATCAGTCGC
CCGTCGGGTGTCGAGACGCCCTGTTGCGCCTCACCGCCGGGATGATCCCA
ACCCGCGTCACAACCGCCGGGTCCATCGCCGAGATACGCGACCTGGCGCG
CACGTTTGCCCGCGAGGTGGGGGGAGTTTAG

SEQ ID No. 89: Deduced amino acid sequence of HSV TK2 entire nucleotide sequence without mutation in mut1 site (shown in bold, underlined)

MASHAGQQHAPAFGQAARASGPTDGRAASRPSHRQGASEARGDPELPTLL
RVYIDGPHGVGKTTTSAQLMEALGPRDNIVYVPEPMTYWQVLGASETLTN
IYNTQHRLDRGEISAGEAAVVMTSAQITMSTPYAATDAVLAPHIGGEAVG
PQAPPPALTLVFDRHPIASLLCYPAARYLMGSMTPQAVLAFVALMPPTAP
GTNLVLGVLPEAEHADRLARRQRPGERLDLAMLSAIRRVYDLLANTVRYL
QRGGRWREDWGRLTGVAAATPRPDPEDGAGSLPRIEDTLFALFRVPELLA
PNGDLYHIFAWVLDVLADRLLPMHLFVLDYDQSPVGCRDALLRLTAGMIP
TRVTTAGSIAEIRDLARTFAREVGGV

SEQ ID No. 90: HSV TK2 entire nucleotide sequence containing mutation in mut1 site (shown in bold, underlined)

ATGGCTTCTCACGCCGGCCAACAGCACGCGCCTGCGTTCGGTCAGGCTGC
TCGTGCGAGCGGGCCTACCGACGGCCGCGCGGCGTCCCGTCCTAGCCATC
GCCAGGGGGCCTCCGAAGCCCGCGGGGATCCGGAGCTGCCCACGCTGCTG
CGGGTTTATATAGACGGACCCCACGGGGTGGGGAAGACCACCACCTCCGC
GCAGCTGATGGAGGCCCTGGGGCCGCGCGACGATATCGTCTACGTCCCCG
AGCCGATGACTTACTGGCAGGTGCTGGGGGCCTCCGAGACCCTGACGAAC
ATCTACAACACGCAGCACCGTCTGGACCGCGGCGAGATATCGGCCGGGGA
GGCGGCGGTGGTAATGACCAGCGCCCAGATAACAATGAGCACGCCTTATG
CGGCGACGGACGCCGTTTTGGCTCCTCATATCGGGGGGGAGGCTGTGGGC
CCGCAAGCCCCGCCCCCGGCCCTCACCCTTGTTTTCGACCGGCACCCTAT
CGCCTCCCTGCTGTGCTACCCGGCCGCGCGGTACCTCATGGGAAGCATGA
CCCCCCAGGCCGTGTTGGCGTTCGTGGCCCTCATGCCTCCGACCGCGCCC
GGCACGAACCTGGTCCTGGGTGTCCTTCCGGAGGCCGAACACGCCGACCG
CCTGGCCAGACGCCAACGCCCGGGCGAGCGGCTTGACCTGGCCATGCTGT
CCGCCATTCGCCGTGTCTACGATCTACTCGCCAACACGGTGCGGTACCTG
CAGCGCGGCGGGAGGTGGCGGGAGGACTGGGGCCGGCTGACGGGGGTCGC
TGCGGCGACCCCGCGGCCGGACCCGGAGGACGGCGCGGGGTCTCTGCCCC
GCATCGAGGACACGCTGTTTGCCCTGTTCCGCGTTCCCGAGCTGCTGGCC
CCCAACGGGGACTTGTACCACATTTTTGCCTGGGTCTTGGACGTCTTGGC
CGACCGCCTCCTTCCGATGCATCTATTTGTCCTGGATTACGATCAGTCGC
CCGTCGGGTGTCGAGACGCCCTGTTGCGCCTCACCGCCGGGATGATCCCA
ACCCGCGTCACAACCGCCGGGTCCATCGCCGAGATACGCGACCTGGCGCG
CACGTTTGCCCGCGAGGTGGGGGGAGTTTAG

SEQ ID No. 91: Deduced amino acid sequence of HSV TK2 entire nucleotide sequence containing mutation in mut1 site

MASHAGQQHAPAFGQAARASGPTDGRAASRPSHRQGASEARGDPELPTLL
RVYIDGPHGVGKTTTSAQLMEALGPRDDIVYVPEPMTYWQVLGASETLTN
IYNTQHRLDRGEISAGEAAVVMTSAQITMSTPYAATDAVLAPHIGGEAVG
PQAPPPALTLVFDRHPIASLLCYPAARYLMGSMTPQAVLAFVALMPPTAP
GTNLVLGVLPEAEHADRLARRQRPGERLDLAMLSAIRRVYDLLANTVRYL
QRGGRWREDWGRLTGVAAATPRPDPEDGAGSLPRIEDTLFALFRVPELLA
PNGDLYHIFAWVLDVLADRLLPMHLFVLDYDQSPVGCRDALLRLTAGMIP
TRVTTAGSIAEIRDLARTFAREVGGV

### EXAMPLES

1. Quantitative MUC1, HER-2/neu, ESR1, PGR expression level analysis using RT-PCR for applied to breast cancer (MTL-HEP) and other cancer types (lung, esophageal, gastric, pancreas, bladder, colon—MTL, prostate—MTL-AT, ovarian—MTL-AEP) useful for dynamics-adjusted therapy.

The examples relates to a MUC1-based test on blood samples from advanced and non-advanced cancer patients for determining metastatic activity.

TaqMan Real-Time-Reverse Transcription-Polymerase Chain Reaction Method

A preferred method of the present invention is a Real-Time PCR method which is designed for quantitative determination of human MUC1 gene expression level in normal and malignant tissues by reverse transcription and real-time PCR. The kit and method allows to determine the total number of copies of the “normal” full-length MUC1 mRNA variant in the tissue sample and also of the majority of MUC1 mRNA forms generated during alternative splicing of MUC1 pre-mRNA, including splice variants MUC1/A and MUC1/D and short forms MUC1/X, MUC1/Y, MUC1/Z known to be associated with the presence of malignancy. The Real-Time PCR kit for use in methods of the invention for complete quantitative expression level analysis preferably consists either of three or two modules.

Three Modules:
1) Total sample RNA isolation module;
2) Reverse transcription module;
3) Real-Time PCR composition module;

Two Modules:
1) Total RNA Tumor Tissues Isolation Module (analogous to three module variant)
2) Reverse transcription-real-time PCR module.

Three Modules Variant:

Total RNA Tumor Tissues Isolation Module:

This module is designed to obtain total RNA preparation from human tumor tissues (e.g. breast carcinoma, lung carcinoma, ovarian, prostate, colon, bladder, esophageal, gastric cancers, etc.).

Total RNA Tumor Tissues Isolation Module's buffers:

RLB-RNA lysis buffer—LB#1

4M GITC (guanidine isothiocyanate) blocking RNAses activity buffer,

10 mM Tris (pH 7.5), 1% β-mercaptoethanol 0.97% purity,

Elution buffer—EB#2,

EB-10 mM Tris-HCl (pH 7.5 at 25° C.)

Washing buffer—WB#3

60 mM potassium acetate, 10 mM Tris-HCl (pH 7.5 at 25° C.) and 60% ethanol),

DNase buffer (DNase I Amresco)-DB#4 without MnCl2:

22.5 mM Tris-HCl (pH 8.3), 1.125 M NaCl, 1 mM CaCl2—not absolutely necessary, 10 mM MgCl2

+DNase I enzyme (Amresco, 50 000 units) in storage buffer: 2.5u/μl in 10 mM Tris-HCl pH=7.5 or 10 mM HEPES pH=7.5 and 50% (v/v) glycerol, 10 mM CaCl₂, (10 mM MgCl₂ for Mg-containing DB)

+90 mM MnCl₂ solution,

OR the same DNase buffer #4 with MgCl₂: 40 mM Tris pH 7.0, 10 mM MgCl_{2,} 3 mM CaCl₂
1) stop solution—SB#5

2M GITC, 4 mM Tris-HCl (pH 7.5), 57% ethanol,
2) nuclease-free water, silica nuclease-free Mini Spin columns (for 1.5-2.0 ml Eppendorf tubes), Eppendorf 2 ml and 1.5 ml collection tubes, nuclease-free 1.5 ml microcentrifuge elution tubes.

Fixation of a Tissue Sample for Storage at +4 C for a Short Period of Several Days before RNA Extraction from the Samples:
1. Biopsy of patient's tissues or surgery material of solid tumors collected for diagnostic purpose can be taken as fresh tissue pieces and placed into 14 ml plastic conic tubes with 4-5 ml pure 95% ethanol. These tubes should be put into +4 C fridge and can be stored there for several days (up to 2 weeks) until necessary for the whole kit extraction 20 samples are gathered.
2. Our data show that this fixation preserves nucleic acids intact for further extraction and, moreover, genomic DNA contamination of the samples is not higher than with using GITC lysis buffer LB#1 for the fresh tissues. Our data also prove that in case of necessity of sample's storage before RNA extraction this fixation method is much better for further isolated RNA integrity and quantification than freezing of fresh samples in −85 C or liquid nitrogen with following unavoidable thawing before homogenization and RNA extraction.
3. 4-5 ml pure 95% ethanol is being removed completely before the next step of tissue homogenization.

Disruption and Homogenization of Tumor Materials Procedure using TissueLyser LT:
1. The sample (a piece of tumor tissue) is placed into the tube with 1 ml of ice-cold LB buffer #1 as quickly as possible, and tube with buffer 1 and tumor tissue is weighed in order to calculate the sample weight. In general, the ratio of tissue mass to buffer 1 should be approximately 171 mg/ml. If necessary, ice-cold LB buffer #1 is added to the tissue to achieve this ratio. But up to 30 mg fresh or frozen tissue to one 2 ml microcentrifuge tubes is recommended, so if samples are bigger it is reasonable to cut them smaller keeping on dry ice before next step.
2. Transfer 30 mg samples into 2 ml microcentrifuge tubes containing 1 stainless steel bead (5-7 mm diameter) at room temperature (15-25° C.). If handling tissue samples stabilized with RLB supplied with β-mercaptoethanol (RNA Stabilization Reagent), cooling on dry ice is not necessary. If lysate is too viscous to pipet easily, it should be diluted by adding buffer 1 to make the lysate easy to pipet. The maximum volume of lysate that can be processed in each Spin Column is 175 μl.
3. Place the tubes into the insert of the TissueLyser LT Adapter, and incubate at room temperature for 2 min to avoid freezing of lysis buffer in step 4. Do not incubate for longer than 2 min, otherwise the tissue will thaw, resulting in potential RNA degradation.
4. Place the insert with sample tubes into the base of the TissueLyser LT Adapter, which is attached to the TissueLyser LT. Place the lid of the TissueLyser LT Adapter over the insert, and screw the knob until the lid is securely fastened.
5. Operate the homogenizer for 2-5 min at 50 Hz. The duration depends on the tissue being processed and can be extended until no tissue debris is visible. Debris can reduce isolated RNA yields amounts dramatically. However, a little of debris have no effect on subsequent RNA purification because they are usually digested with proteinase K.
6. Proceed with RNA purification. Do not reuse the stainless steel balls.

Total RNA Isolation is Carried out as Following:

1 ml of ice-cold lysis buffer 1 is transferred to the tube, and the tube with buffer 1 is weighed. The sample (piece of tumor tissue) is placed into the tube with buffer 1 as quickly as possible, and tube with buffer 1 and tumor tissue is weighed one more time in order to calculate sample weight. In general, the ratio of tissue mass to buffer 1 should be approximately 171 mg/ml. If necessary, ice-cold RNA Lysis Buffer is added to the tissue to achieve this ratio. Then the sample is homogenized at high speed using a small homogenizer (Tekmar Tissuemizer or other) or placed to a mortar and grinded under buffer 1. GTC and β-mercaptoethanol presenting in buffer 1 inactivate the ribonucleases in cell extracts. If lysate is too viscous to pipet easily, it should be diluted by adding buffer 1 to make the lysate easy to pipet. The maximum volume of lysate that can be processed in each Spin Column is 175 μl. 175 μl of the tissue lysate is transferred to a 1.5 ml nuclease-free microcentrifuge tube. 350 μl of solution 2 is added and mixed with lysate by inverting 3-4 times. Microcentrifuge tube is then placed in a heating block at 70° C. for 3 minutes (not longer). On this step selective precipitation of cellular proteins occurs, while the RNA remains in solution.

Microcentrifuge tube is centrifuged for 10 minutes at 12,000-14,000×g. The obtained lysate is cleared of precipitated proteins and cellular debris. The cleared lysate solution is transferred to a fresh microcentrifuge tube by pipetting. Disturbance of the pelleted debris must be avoided. The supernatant volume should be approximately 500 μl. 200 μl 95% ethanol is added to the cleared lysate and mixed with it by pipetting 3-4 times. The RNA is selectively precipitated with ethanol. The spin column is placed to the collection tube, and the obtained mixture is transferred to the spin column. Spin column is centrifuged at 12,000-14,000×g for one minute. The RNA is bound to the silica surface of the glass fibers in the spin columns by centrifugation (or, otherwise, by vacuum filtration method). In order to avoid clogging of the membrane in the spin column no more then 30mg of tissue (the maximum volume of lysate is 175 μl) can be processed per purification with one spin column. The liquid in the collection tube is discarded, and spin column is put back into the collection tube. 600μl of solution 3 (washing buffer) is added to the spin column, and column is centrifuged at 12,000-14,000×g for 1 minute. The collection tube is emptied as before and placed back to the collection tube. Fresh DNase incubation mix (do not mix the components prior to this step!) is prepared by combining 40 μl buffer 4, 5 μl 0.09 M MnCl₂ and 5 μl (5 u) of DNase I enzyme per sample in a sterile nuclease-free tube (in this order) and mixing by gentle pipetting (do not vortex). 50 μl of this freshly prepared DNase incubation mix is applied directly to the membrane inside the spin column. The solution must cover the membrane thoroughly. Thus DNase I is applied directly to the silica membrane to digest contaminating genomic DNA. The spin column is incubated for 20 minutes at 20-25° C. and then centrifuged at 12,000-14,000×g for 10 sec. The next fresh portion of DNase incubation mix is prepared, and 50 μl of freshly prepared DNase incubation mix is applied to the membrane inside the spin column. The spin column is incubated for 20 minutes at 20-25° C. 200 μl of stop solution 5 is added to the spin column for DNAse inactivation, and spin column is centrifuged at 12,000-14,000×g for 1 minute. 600 μl of solution 3 (washing buffer) is added to the spin column, and it is centrifuged at 12,000-14,000×g for 1 minute. The collection tube is emptied, and spin column is put back into the collection tube. 250 μl of solution 3 is added to the spin column, and it is centrifuged at 12,000-14,000×g for 2 minutes. By these washing steps the bound total RNA is purified from contaminating salts, proteins and cellular impurities. The collection tube with the flow through is discarded, and the spin column is placed to the 1.5 ml nuclease-free elution tube. 100 μl nuclease-free water is added to the spin column's membrane. Water must cover the membrane thoroughly. The spin column is centrifuged at 12,000-14,000×g for 1 minute. Thus the total RNA is eluted from the silica membrane. The obtained purified RNA solution is used directly for reverse transcription or stored at −70° C.

The yield of total RNA obtained is determined spectrophotometrically by measuring the absorbance at 260 nm. 1 absorbance unit (A260) corresponds to 40 μg of single-stranded RNA/ml. The purity may also be estimated spectrophotometrically from the relative absorbances at 230, 260 and 280 nm (A260/A280 and A260/A230). The expected range of A260/A280 ratios for RNA will be 1.7-2.1 and A260/A230 ratios of 1.8-2.2.

*Lysis buffer 1 with β-Mercaptoethanol must be stored at 4° C.

**It is necessary to use RNase-free pipettes, sterile disposable RNase-free plastic ware and wear gloves when handling RNA and all reagents to reduce risk of RNase contamination.

***If purified RNA samples contain traces of genomic DNA contamination in subsequent control PCR (in rare cases when the initial tissue sample contained too much genomic DNA) it is necessary to perform a post-RNA isolation DNase treatment using RNase-Free DNase I followed by phenol:chloroform extraction.
2) Reverse Transcription Module:

This module is designed to obtain cDNA from RNA preparation isolated from human tumor tissues. Module consists of primer solution (random hexamer primers mixture (0.2 μg/μl), or otherwise Oligo(dT) primers (100 pmol/μl), or otherwise MUC1-specific primer (20 pmol/μl)), 5× reaction buffer for reverse transcriptase (250 mM Tris-HCl (pH 8.3 at 25° C.), 250 mM KCl, 20 mM MgCl₂, 50 mM DTT), 10× dNTP Mix (10 mM each), M-MuLV reverse transcriptase (20 u/μl in 50 mM Tris-HCl (pH=7.5), 0,1 M NaCl, 1 mM EDTA, 5 mM DTT, 0,1% (v/v) Triton X-100 and 50% (v/v) glycerol storage buffer) and nuclease-free water.

Reverse Transcription is Carried out as Following:

100 ng-5 μg of total RNA is mixed on ice in sterile nuclease-free tube with 0.2 μg (100 pmol) of random hexamer primers mixture (0.2 μg/μl), or 100 pmol Oligo(dT) primers, or 20 pmol of reverse gene-specific primer. Nuclease-free water is added to total volume of 12 μl if necessary. Mixture of RNA template with primers is incubated at 70° C. for 5 min to destroy secondary structure of RNA template, chilled on ice for 10 minutes and briefly centrifuged, and then tube is placed on ice.

After that the reaction mixture is prepared by adding in the given order of 5× reaction buffer for reverse transcriptase (4 μl), dNTP Mix (2 μl) and M-MuLV reverse transcriptase (2 μl) to the mixture of RNA template with primers. The final reaction volume is 20 μl. All components are mixed gently, and mixture is briefly centrifuged. The obtained reaction mixture is incubated 10 min at 25° C. (only if random hexamer primers are used).

Reverse Transcription PCR programming:

Initial denaturation:65° C. 5 min

Cooling: 10° C. 5 min

Amplification: 37° C. 60 min

Termination: 70° C. 10 min

The obtained cDNA preparation is diluted with 180 μl of nuclease-free water (tenfold dilution). The obtained cDNA can be directly used as matrix in real-time PCR or stored at −20° C. for 1 week or at −70° C. for 1 year.
3) Real-Time PCR Module:

This module is designed to measure genes copies number in cDNA preparation obtained from human tumor tissue RNA. Module consists of: 10× colorless PCR buffer (200 mM Tris-HCl (pH 8.3), 200 mM KCl, 50mM (NH₄)₂SO₄, 10x dNTP mixture 2 mM each), nuclease-free water; 1-2 mM MgCl₂ and 25 U Taq DNA-polymerase (for example, Maxima™ Hot Start) for each reaction (5 u/μl in 10 mM Tris-HCl (pH 8,3), 1 mM EDTA, 1 mM DTT, 100 mM KCl, 0.5% Tween-20, 50% glycerol storage buffer) are being added freshly into 1× reaction mixture;

| Primers | |
|---|---|
| M | 1 μM for + 1 μM rev |
| ML | 1 μM for + 1 μM rev |
| ER | 1 μM for + 1 μM rev |
| PR | 1.3 μM for + 1.3 μM rev |
| H(19-20-21) | 1.7 μM for + 1.7 μM rev |

0.3 μM (for all genes) of the sample cDNA, for negative control use water instead.

50 μl is a volume of reaction mixture in each PCR tube.

To perform a calibration curve mix −5, −7 and −9 dilutions of standards with the reaction mixture in separate tubes.

Real-Time PCR is Carried out as Following:

The reaction mixture for real-time PCR is prepared as following: 10 μl of 5× colorless PCR buffer, 5μl of 10× dNTP mixture, 5 μl of 10× primers mixture, 5 μl of 10× probe solution, 4.5 μl of nuclease-free H₂O and 5 μl of hot start Taq DNA Polymerase are placed in sterile 1.5 ml microcentrifuge tube per 1 PCR reaction and mixed carefully by pipetting. 30 μl of the reaction mixture is placed to 0.2 μl microcentrifuge tube. 20 μl of the previously obtained cDNA preparation is added to the reaction mixture and mixed carefully by pipetting.

For each experiment it is necessary to perform reaction with 6 standards-human MUC1, ESR, PRG and ERBB2 genes DNA calibrators. For this purpose from three to six DNA calibrators K1-K6 are added to six tubes (20 μl of calibrator per tube). The obtained standard probes contain 5000000 Universal Standard DNA copies/reaction (K1), 500000 copies/reaction (K2), 50000 copies/reaction (K3), 5000 copies/reaction (K4), 500 copies/reaction (K5) and 50 copies/reaction (K6), correspondingly. Negative PCR control is prepared by adding 20 μl of nuclease-free water to the separate tube.

The tubes are placed into the real time thermal cycler, and the instrument is programmed. The following instruction is given for Rotor-Gene 3000/6000 thermal cyclers (Corbett Research). 36-well rotor is used for Rotor-Gene instrument. All experimental samples must be designated as “Unknown” in “Type” column in “Edit Samples” menu. All PCR calibrators must be designated as “Standard” in “Type” column in “Edit Samples” menu and their concentrations must be indicated in the corresponding cells of “Given conc” column. The following amplification program in “Profile Editor” menu should be chosen:

| Hold: | | | |
|---|---|---|---|
| Initial denaturation: 95° C. 4 min | | | |
| Cycling 1: | | | |
| Denaturation: 95° C. | 20 sec | | |
| Annealing: 56° C. | 15 sec | {close oversize bracket} | cycle repeats-5 times |
| Elongation: 72° C. | 15 sec | | |
| Cycling 2: | | | |
| Denaturation: 95° C. | 20 sec | | |
| Annealing: 56° C. | 15 sec., | {close oversize bracket} | acquiring Probe cycle |
| Elongation: 72° C. | 15 sec | | repeats-35 times |

In the “Channel Settings” menu (appearing after “Gain Optimization” button pressing) Green channel must be chosen. “Tube position” 1, “Min Reading” 5, “Max Reading” 10 and “Perform Optimization Before 1^{st} Acquisition” should be chosen.

The results are analyzed using instrument software. In “Quantitation analysis” menu for each channel “Dynamic tube” and “Slope Correct” buttons must be pressed. In “CT Calculation” menu Threshold=0.005 should be chosen for green channel (mud). In “Outlier Removal” menu NTC Threshold=5% for Green channel should be chosen. Standard curve is plotted automatically by the software on the basis of the obtained Ct meanings for DNA calibrators and their standard concentrations. Correlation coefficient (R²) for calibration curves must be >0.98. Otherwise experiment is considered invalid and must be repeated. Calculation of MUC1 copy number for each unknown sample is performed automatically by instrument software using the obtained Ct values and plotted calibration curve. Appearance of Ct meaning for negative PCR control may indicate contamination of reagents. In this case all results of the experiment are considered invalid, contamination source must be found and experiment must be repeated. Linear measurement range is 500-50 000 000 gene copies/reaction. If the obtained result is more than 50 000 000 copies/ml reaction mixture, the corresponding sample must be diluted tenfold with nuclease-free water and the test must be repeated. If the obtained result is less than 500 copies/ml reaction mixture the measurement is rather non thrust-worthy. The obtained genes copy number/reaction data should be normalized to obtained total RNA concentration and finally expressed as MUC1 copy number/pg of total RNA.

Primers-Probes “M . . . ” for measuring MUC1 mRNA

1. M1 for ex1 primer
(SEQ ID No: 1)
5′-CCTCCCCACCCATTTCACC-3′
Tm = 61.6° C.
M1 rev ex1 primer
(SEQ ID No: 2)
5′-CTGTAAGCACTGTGAGGAGC-3′
Tm = 60.5° C.
Probe M1.1
(SEQ ID No: 34)
5′-(FAM)TGACACCGGGCACCCAGTCTCC(BHQ2)-3′;
*Fluorescence is measured at 5° C. (in the
end of annealing step) in Cycling 2.
Green channel-470 nm source/510 nm detection.
Probe M1.2
(SEQ ID No: 35)
5′-(ROX)-CCACCATGACACCGGGCACCCA-(BHQ2)-3′
Tm = 69.6° C. Orange channel 560 nm detection
2. M2 for ex7
(SEQ ID No: 3)
5′-CCTACCATCCTATGAGCGAG-3′
Tm = 60.5° C.
M2 rev ex8
(SEQ ID No: 4)
5′-CCCTACAAGTTGGCAGAAGTG-3′
Tm = 61.3° C.
Probe M2
(SEQ ID No: 36)
5′-(ROX)-TGCAGGTAATGGTGGCAGCAGCC-(BHQ2)-3′
Tm = 68.2° C. Orange channel
3. MM 2-4.1 for ex2-3b
(SEQ ID No: 5)
5′-CTACTGAGAAGAATGCTTTGTCTA
Tm = 60.1° C.
MM 2-4.1 rev ex 4
(SEQ ID No: 6)
5′-GCCTGAACTTAATATTGGAGAGG
Tm = 61.1° C.
Probe MM 2-4.1
(SEQ ID No: 37)
5′(ROX)-AGCACCGACTACTACCAAGAGCTGCA-(BHQ2)-3′
Tm = 69.4° C.
Probe MM2-4.3
(SEQ ID No: 38)

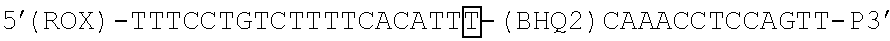

4. MM 2-4.2 for ex 2-3
(SEQ ID No: 7)
5′-CTACTGAGAAGAATGCTTTTAATTCC-3′
Tm = 61.6° C.
MM 2-4.2 rev ex 4
(SEQ ID No: 8)
5′-GCCTGAACTTAATATTGGAGAGG-3′
Tm = 61.1° C.
Probe MM 2-4.2
(SEQ ID No: 39)
5′(ROX)-CAGCACCGACTACTACCAAGAGCTGC-(BHQ2)-3′
Tm = 71.0° C.
5. MM 2-3 for ex2-3b
(SEQ ID No: 9)
5′-CTACTGAGAAGAATGCTTTGTCTA-3′
Tm = 60.1° C.
MM 2-3 rev ex3
(SEQ ID No: 10)
5′-CTCTTGGTAGTAGTCGGTGC-3′
Tm = 60.5° C.
Probe MM2-4.3
(SEQ ID No: 40)

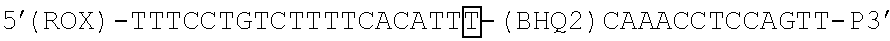

6. MM 3.1 for ex3
(SEQ ID No: 11)
5′-CCAGCACCGACTACTACCAA-3′
Tm = 60.5° C.
MM 3.2 for ex3
(SEQ ID No: 13)
5′-CACCGACTACTACCAAGAGC-3′
Tm = 60.5° C.
MM 3.1 rev ex3
(SEQ ID No: 12)
5′-CTCTTGGTAGTAGTCGGTGC-3′
Tm = 60.5° C.
Probe MM 3.1
(SEQ ID No: 41)
5′(ROX)-ATGGCTGTCTGTCAGTGCCGCCGAA-(BHQ2)-3′
7. MM 2-4.4 for ex2-3c
(SEQ ID No: 14)
5′-CTACTGAGAAGAATGCTTTTAATTCC-3′
Tm = 61.6° C.
MM 2-4.4 rev ex4
(SEQ ID No: 15)
5′-GCCTGAACTTAATATTGGAGAGG-3′
Tm = 61.1° C.
Probe MM 2-4.1
(SEQ ID No: 42)
5′(ROX)-AGCACCGACTACTACCAAGAGCTGCA-(BHQ2)-3′
Tm = 69.4° C.
Probe MM 2-4.2
(SEQ ID No: 43)
5′(ROX)-CAGCACCGACTACTACCAAGAGCTGC-(BHQ2)-3′
Tm = 71.0° C.
8. MM 2-3-7 for ex2-3c
(SEQ ID No: 16)
5′-CTACTGAGAAGAATGCTTTTAATTCC-3′
Tm = 61.6° C.
MM 2-3-7.1 rev ex7-3c
(SEQ ID No: 17)
5′-CGGCACTGACAGACAGCCAT-3′
Tm = 62.5° C.
or
MM 2-3-7.2 rev ex7-3
(SEQ ID No: 18)
5′-GGCACTGACAGACAGCCATT-3′
Tm = 60.5° C.
Probe MM 2-4.1
(SEQ ID No: 44)
5′(ROX)- AGCACCGACTACTACCAAGAGCTGCA-(BHQ2)-3′
Tm = 69.4° C.
9. MM 2-3.1-6 for ex2-3c
(SEQ ID No: 19)
5′-CTACTGAGAAGAATGCTTTTAATTCC-3′
Tm = 61.6° C.
MM 2-3.1-6 rev ex6b-3
(SEQ ID No: 20)
5′-CACCCCAGCCCCAGACATT-3′
Tm = 61.6° C.
Probe MM 2-4.1
(SEQ ID No: 45)
5′(ROX)-AGCACCGACTACTACCAAGAGCTGCA-(BHQ2)-3′
Tm = 69.4° C.
Probe MM 2-4.2
(SEQ ID No: 46)
5′(ROX)-CAGCACCGACTACTACCAAGAGCTGC-(BHQ2)-3′
Tm = 71.0° C.
10. MM 2-4.1-5 for ex2-4a
(SEQ ID No: 21)
5′-CTACTGAGAAGAATGCTTTTTTGC-3′
Tm = 60.1° C.
MM 2-4.1-5 rev ex5
(SEQ ID No: 22)
5′-AGGCTGCTTCCGTTTTATACTG-3′
Tm = 60.3° C.
Probe MM 2-4.1-5.1
(SEQ ID No: 47)
5′(ROX)-TTGACTCTGGCCTTCCGAGAAGGTAC-(BHQ2)-3′
Tm = 69.4° C.
Probe MM 2-4.1-5.2
(SEQ ID No: 48)
5′(ROX)-CTTCCGAGAAGGTACCATCAATGTCCAC-(BHQ2)-3′
Tm = 70.1° C.
11. MM 4-6.1-7-6.1 for ex4-6a
(SEQ ID No: 23)
5′-CCTCTCCAATATTAAGTTCAGTGA-3′
Tm = 60.1° C.
MM 4-6.1-7-6.1 rev ex7-6
(SEQ ID No: 24)
5′-ACAGACAGCCAAGGCAATGAG-3′
Tm = 61.3° C.
Probe MM 4-6.1-7-6.1
(SEQ ID No: 49)
5′(ROX)-CATCGCGCGCTGCTGGTGCTGGTCT-(BHQ2)-3′
Tm = 70.0° C.
Probe MM 4-6.1-7-6.2
(SEQ ID No: 50)
5′(ROX)-TGTGCCATTTCCTTTCTCTGCCCAGTC-(BHQ2)-3′
Tm = 69.8° C.
12. MM 4-6.2-7-6 for ex4-6b
(SEQ ID No: 25)
5′-CCTCTCCAATATTAAGTTCAGTCT-3′
Tm = 60.1° C.
MM 4-6.3-7-6 for ex4-6b
(SEQ ID No: 26)
5′-CCTCTCCAATATTAAGTTCAGTC-3′
Tm = 59.3° C.
MM 4-6.2-7-6 rev ex7-6
(SEQ ID No: 27)
5′-ACAGACAGCCAAGGCAATGAG-3′
Tm = 61.3° C.
Probe MM 4-6.2-7-6
(SEQ ID No: 51)
5′(ROX)-CATCGCGCTGCTGGTGCTGGTCT-(BHQ2)-3′
Tm = 70.0° C.
13. ML1 for
(SEQ ID No: 28)
5′-CCACTCTGATACTCCTACCAC-3′
Tm = 61.3° C.
ML1 rev
(SEQ ID No: 29)
5′-GAAAGAGACCCCAGTAGACAAC-3′
Tm = 62.0° C.
Probe ML1
(SEQ ID No: 52)
5′-(ROX)-AGCCATAGCACCAAGACTGATGCCA-(BHQ2)-3′
Tm = 67.4° C.
Probe ML2
(SEQ ID No: 53)
5′-(ROX)-ACCTCCTCTCACCTCCTCCAATCACA-(BHQ2)-3′
Tm = 69.4° C.

Primers-Probes for measuring HER-2/neu (ERBB2) mRNA

1. H1 for ex11 (HER2-furin-wt) for
(SEQ ID No: 54)
5′-CGTTTGAGITCCATGCCCAATC-3′
Tm = 61.2° C.

H1 rev ex12 (HER2-furin-wt) rev

(SEQ ID No: 55)

5′-TCCTCTGCTGITCACCTCTTG-3′
Tm = 60.5° C.
PCR product size = 145 bp
Probe H1

(SEQ ID No: 60)

5′-(ROX)-CTGCCTGITCCCTACAACTACCTTTCTAC-(BHQ2)-3′
Tm = 70.1° C.
Probe hybridizes with the end of exon 11 and with the start of exon 12.
2. HΔ2 for detection of ΔHER2 transcript variant
without exon 16(20). ΔHER2 mRNA-detection,
wild-type HER2 transcripts-no detection,
alternatively spliced HER-2 RNA form AF177761.2
(herstatin)-no detection.
HΔ2 for ex19-21 delta

(SEQ ID No: 56)

5′-CACCCACTCCCCTCTGAC-3′
Tm = 60.7° C.
HΔ2 rev ex21 delta

(SEQ ID No: 57)

5′-CAGCAGITCTCCGCATCGTG-3′
Tm = 61.6° C.
Probe HΔ2 ex. 19-21

(SEQ ID No: 63)

5′(ROX)-ATCCTCATCAAGCGACGGCAGCAGAA-(BHQ2)-3′
3. H3 for ex 19

(SEQ ID No: 58)

5′-GTGAAACCTGACCTCTCCTAC-3′
H3 rev ex 21

(SEQ ID No: 59)

5′-CAGCAGTCTCCGCATCGTG-3′
Tm = 61.6° C.
Probe H3 ex 20

(SEQ ID No: 62)

5′-(ROX)-AGCAGAGAGCCAGCCCTCT-(BHQ2)-GACGTCCATC-3′

Primers-Probe for measuring Estrogen Receptor total mRNA ER1 (ESR1)

1. ER1 for ex1

(SEQ ID No: 63)

5′-CCACTCAACAGCGTGTCTC-3′
Tm = 59.5° C.
ER1 rev ex1

(SEQ ID No: 64)

5′-GCTCGTTCTCCAGGTAGTAG-3′
Tm = 60.5° C.
Probe ER1

(SEQ ID No: 65)

5′-(ROX)-TGTCGCCTTTCCTGCAGCCCCAC-(BHQ2)-3′
Tm = 70° C.

Primers-Probes for measuring Progesterone Receptor total mRNA PR (RGR)

1. PR1 for ex7

(SEQ ID No: 66)

5′-CTTACAAAACTTCTTGATAACTTGC-3′
Tm = 59.2° C.
PR1 rev ex8

(SEQ ID No: 68)

5′-GGTTTCACCATCCCTGCCAA-3′
Tm = 60.5° C.
PCR product size = 164 bp
Probe PR1 ex8

(SEQ ID No: 69)

5′-(ROX)-CTTCATCTGTACTGCTTGAAT (BHQ2) ACATTTATCCA
G-3′

This primer pair allows the detection of all PGR mRNA forms containing exons 7 and 8. These primers don't “see” PRΔ7 and PRΔ6/7 forms, but these forms were found in human endometrium and may be not very important (if express) in breast cancer cells.

2. PR2 for ex8

(SEQ ID No: 67)

5′-CTGTACTGCTTGAATACATTTATCC-3′
Tm = 60.9° C.
PR2 rev ex8

(SEQ ID No: 68)

5′-GGTTTCACCATCCCTGCCAA-3′
Tm = 60.5° C.
PCR product size = 116 bp
Probe PR2 ex8

(SEQ ID No: 70)

5′-(ROX)-ATGATGTCTGAAGTTATTGCT (BHQ2) GCACAATTACC
C-P3′
Tm = 70.2° C.

(BHQ2) on blue T in the middle and phosphate on the last C on the 3′ end. This primer pair allows the detection of all PGR mRNA forms containing exon8.

Universal standard for human MUC1 total forms, MUC1 long forms, PGR, ESR1, HER2/neu total forms and HER2/neu delta HER2 form

EcoRI SacI XbaI SaII PstI HindIII EcoRV
MUC1 exon1 for
(SEQ ID No: 92)

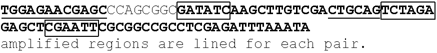

MUC1 exon 1 rev^{*} MUC1
long forms for
CCACCATGACACCGGGCACCCAGTCTCCTTTCTTCCTGCTGCTGCTCCTCA
CAGTGCTTACAGCTCAATTCCCACTCTGATACTCCT
(SEQ ID No: 93)
ACCACCCTTGCCAGCCATAGCACCAAGACTGATGCCAGTAGCACTCACCAT
AGCACGGTACCTCCTCTCACCTCCTCCAATCACAGC
MUC1 long forms rev^{*} MUC1 exon 7 for
ACTTCTCCCCAGTTGTCTACTGGGGTCTCTTCCGGGATACCTACCATCCTA
TGAGCGAGTACCCCACCTACCACACCCATGGGCGCT
(SEQ ID No: 94)
ATGTGCCCCCTAGCAGTACCGATCGTAGCCCCTATGAGAAGGTTTCTGCAG
GTAATGGTGGCAGCAGCCTCTCTTACACAAACCCAG
MUC1 exon 8 rev^{*} HER2/neu for
(SEQ ID No: 95)
CAGTGGCAGCCACTTCTGCCAACTTGTAGGGGCACGTCGCGTTTGAGTCCA
TGCCCAATCCCGAGGGCCGGTATACATTCGGCGCCA
HER2/neu rev^{*}
(SEQ ID No: 96)
GCTGTGTGACTGCCTGTCCCTACAACTACCTTTCTACGGACGTGGGATCCT
GCACCCTCGTCTGCCCCCTGCACAACCAAGAGGTGA
HER2/neu rev^{*} ΔHER2 for
(SEQ ID No: 97)
CAGCAGAGGATGGAACCTGCACCCACTCCCCTCTGACGTCCATCATCTCTG
CGGTGGTTGGCATTCTGCTGGTCGTGGTCTTGGGGG
ΔHER2 rev^{*}
PGR exon 7 for
(SEQ ID No: 98)
TGGTCTTTGGGATCCTCATCAAGCGACGGCAGCAGAAGATCCGGAAGTACA
CGATGCGGAGACTGCTGCAGGAAACCTTACAAAACT
PGR exon 7 for PGR exon 8 for
(SEQ ID No: 99)
TCTTGATAACTTGCATGATCTTGTCAAACAACTTCATCTGTACTGCTTGAA
TACATTTATCCAGTCCCGGGCACTGAGTGTTGAATT
PGR exon 8 rev^{*}
ESR1 exon 1 for
(SEQ ID No: 100)
TCCAGAAATGATGTCTGAAGTTATTGCTGCACAATTACCCAAGATATTGGC
AGGGATGGTGAAACCCCTTCTCTCCACTCAACAGCG
ESR1 exon 1 for
ESR1 exon 1 rev^{*}
(SEQ ID No: 101)
TGTCTCCGAGCCCGCTGATGCTACTGCACCCGCCGCCGCAGCTGTCGCCTT
TCCTGCAGCCCCACGGCCAGCAGGTGCCCTACTACC
ESR1 exon 1 rev^{*} EcoRV HindIII SalI PstI XbaI SacI
EcoRI NotI XhoI SmiI
(SEQ ID No: 102)

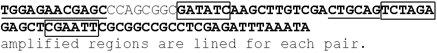

MUC1 For St EcoRV
(SEQ ID No: 103)

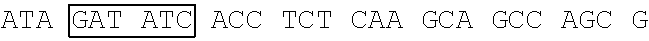

Muc1 Rev2 St EcoRV
(SEQ ID No: 104)

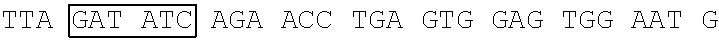

ER1 for1 st
(SEQ ID No: 105)

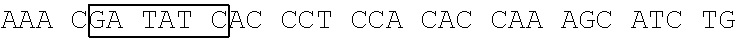

ER1 rev2 St EcoRV
(SEQ ID No: 106)

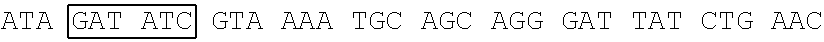

PR for2 st
(SEQ ID No: 107)

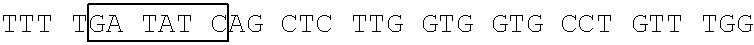

PR rev2 St EcoRV
(SEQ ID No: 108)

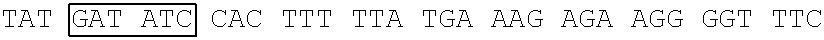

Her2Neu st for1
(SEQ ID No: 109)

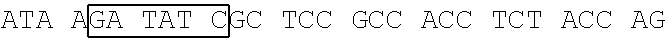

HER2Neu st rev1
(SEQ ID No: 110)

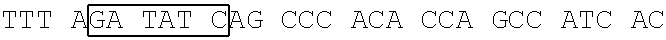

Two Modules Variant
1) Total RNA Tumor Tissues Isolation Module (analogous to three modules variant):

This module is designed to obtain cDNA from RNA isolated from human tumor tissues and to measure MUC1 copy number in this cDNA preparation. Module consists of 5× colorless RT-PCR buffer (250 mM Tris-HCl (pH 8.3 at 25° C.), 250 mM KCl, 20 mM MgCl2, 50 mM DTT), M-MuLV reverse transcriptase (20 u/μl in 50 mM Tris-HCl (pH=7.5), 0.1 M NaCl, 1 mM EDTA, 5 mM DTT, 0.1% (v/v) Triton X-100 and 50% (v/v) glycerol storage buffer), 10× MUC1-specific primers mixture* (400 pmol/μl each), 10× probe solution** (100 pmol/μl), hot start DNA Polymerase TaqF (Amplisense) (5 u/μl in 10 mM Tris-HCl (pH 8.3), 1 mM EDTA, 1mM DTT, 100 mM KCl, 0.5% Tween-20, 50% glycerol storage buffer), 10× dNTP Mix (10 mM each), 6 MUC1 DNA calibrators K1-K6 containing known number of MUC1 gene copies and nuclease-free water.
2) Reverse transcription-real-time PCR module:

The reaction mixture for RT-real-time PCR is prepared as following: 10 μl of 5× colorless RT-PCR buffer, 5 μl of 10× dNTP mixture, 5 μl of 10× primers mixture, 5 pl of 10× probe solution, 2 μl of M-MuLV reverse transcriptase, 0.5 μl of hot start DNA Polymerase TaqF (Amplisence) and 2.5 μl of nuclease-free H2O are placed in sterile 1.5 ml microcentrifuge tube per 1 RT-PCR reaction and mixed carefully by pipetting. 30 μl of the reaction mixture is placed to 0.2 μl microcentrifuge tube. 20 μl of the previously obtained RNA preparation diluted tenfold with nuclease-free water is added to the reaction mixture and mixed carefully by pipetting.

For each experiment it is necessary to perform reaction with 6 standards-human MUC1 gene DNA calibrators. For this purpose six DNA calibrators K1-K6 are added to six tubes (20 μl of calibrator per tube). The obtained standard probes contain 5000000 MUC1 DNA copies/reaction (K1), 500000 copies/reaction (K2), 50000 copies/reaction (K3), 5000 copies/reaction (K4), 500 copies/reaction (K5) and 50 copies/reaction (K6), correspondingly. Negative PCR control is prepared by adding 20 μl of nuclease-free water to the separate tube. The tubes are placed into the real time thermal cycler, and the instrument is programmed. The following instruction is given for Rotor-Gene 3000/6000 thermal cyclers (Corbett Research). 36-well rotor is used for Rotor-Gene instrument. All experimental samples must be designated as “Unknown” in “Type” column in “Edit Samples” menu. All PCR calibrators must be designated as “Standard” in “Type” column in “Edit Samples” menu and their concentrations must be indicated in the corresponding cells of “Given conc” column. The following amplification program in “Profile Editor” menu should be chosen:

Hold1:

37° C. 30 min

Hold2:

95° C. 15 min

Cycling 1:

95° C. 20 sec

58° C. 30 sec, not acquiring

72° C. 25 sec

cycle repeats-5 times

Cycling 2:

95° C. 20 sec

58° C. 30 sec, acquiring to Cycling A (Green channel)***

72° C. 25 sec

cycle repeats-40 times

In the “Channel Settings” menu (appearing after “Gain Optimization” button pressing), Green channel must be chosen. “Tube position” 1, “Min Reading” 5, “Max Reading” 10 and “Perform Optimisation Before 1st Acquisition” should be chosen.

Results Analysis

The results are analyzed using RealTime PCR instrument software. In “Quantitation analysis” menu for each channel “Dynamic tube” and “Slope Correct” buttons must be pressed. In “CT Calculation” menu Threshold=0.005 should be chosen for green channel (mud). In “Outlier Removal” menu NTC Threshold=5% for Green channel should be chosen. Standard curve is plotted automatically by the software on the basis of the obtained Ct meanings for DNA calibrators and their given Universal Standard concentrations. Correlation coefficient (R2) for calibration curves must be □0.98. Otherwise experiment is considered invalid and must be repeated. Calculation of MUC1 copy number for each unknown sample is performed automatically by instrument software using the obtained Ct values and plotted calibration curve. Appearance of Ct meaning for negative PCR control may indicate contamination of reagents. In this case all results of the experiment are considered invalid, contamination source must be found and experiment must be repeated. Linear measurement range is 50-5000000 Universal Standard copies/reaction. If the obtained result is more than 5000000 copies/reaction, the corresponding sample must be diluted tenfold with nuclease-free water and the test must be repeated. The obtained gene's copy number/reaction data should be normalized to obtained total RNA concentration and finally expressed as gene's copy number per μg of total RNA.

Quantitative Analysis of Obtained Data.

The described above method was designed for quantitative determination of the total RNA copies number for ER, PR, HER2 and “normal” full-length MUC1 mRNA variant and the majority of MUC1 mRNA forms generated during alternative splicing of MUC1 pre-mRNA including short splice variants MUC1/X, MUC1/Y, MUC1/Z known to be associated with the presence of malignancy. For ER, PR and HER2neu receptor's RNA the only requirement is Real-Time-RT-PCR numbers correlate with immune histochemistry data being used for routine clinical diagnostic to be able to replace this methods in the nearest future. But it is also important to measure the specific contribution of tumor specific MUC1 RNA forms to this total copy number. As differently spliced MUC1 variants lack different parts of coding material, it is problematic to measure them by real-time PCR altogether. Therefore, we decided to calculate their contribution indirectly by measuring normal or malignant MUC1 RNA forms with it subsequent subtraction from the total copy number of all RNA forms.

Commonly used reference genes are: glyceraldehyde-3-phosphate dehydrogenase mRNA, beta actin mRNA, beta-2 microglobulin (light chain of class I major histocompatibility complex (MHC-I), cyclophilin mRNA, mRNAs for certain ribosomal proteins e.g. RPLP0 (ribosomal protein, large, P0), 28S or 18S rRNAs (ribosomal RNAs) and others. For our experiments we chose beta-2 microglobulin as reference gene. For both target and reference genes plotting of standard calibration curves (showing dependence of real-time PCR threshold cycle (Ct) from gene copy number) is needed. Probes and primers for muc1 and beta-2 microglobulin genes and optimized real-time PCR conditions are shown below. Final muc1 and B2M PCR products are shown on FIG. 19.

Real-time PCR conditions for muc1 and beta-2 microglobulin genes amplification:

| Cycle | Cycle Point |
|---|---|
| Hold 95° C., 15 min 0 secs | |
| Cycling (5 repeats) | Step 1 95° C., hold 20 secs |
| | Step 2 60° C., hold 30 secs |
| | Step 3 72° C., hold 20 secs |
| Cycling 2 (45 repeats) | Step 1 95° C., hold 20 secs |
| | Step 2 60° C., hold 30 secs, acquiring |
| | to Cycling A (Green, Orange channels) |
| | Step 3 72° C., hold 20 secs |

muc 1 and B2M probes 7 pmol/reaction (50 μl); muc1 primers 37.5 pmol/reaction (50 μl); B2M primers 50 pmol/reaction (50 μl).

Probe Ex1 for mud 1 gene (exon1):

(SEQ ID No: 111)

5′-(FAM) tg-aca-ccg-ggc-acc-cag-tct-cc (BHQ1)-3′
Tm = 70° C.
Primers for muc1 exon1:
For EX1-2
Tm = 60° C.
Rev EX1-1

(SEQ ID No: 112)

5′-CTG TAA GCA CTG TGA GGA GC-3′
Tm = 60° C.
Probe for beta-2 microglobulin gene (B2M):

(SEQ ID No: 113)

5′-(ROX) ct-gct-tac-atg-tct-cga-tcc-cac-tta-act
(BHQ2)-3′
Tm = 69° C.
Primers for B2M:
For Mod

(SEQ ID No: 114)

5′-GTG TGA ACC ATG TGA CTT TGT C-3′
Tm = 60° C.
Rev Mod

(SEQ ID No: 115)

5′-TCC AAA TGC GGC ATC TTC AAA C-3′
Tm = 60° C.

Then, difference in target gene expression can be estimated according to the formula:

$$\begin{matrix}ratio target gene expression \\ ( experimental / control cells )\end{matrix} = \frac{fold change in target gene expression ( expt / control )}{fold change in reference gene expression ( expt / cont )}$$

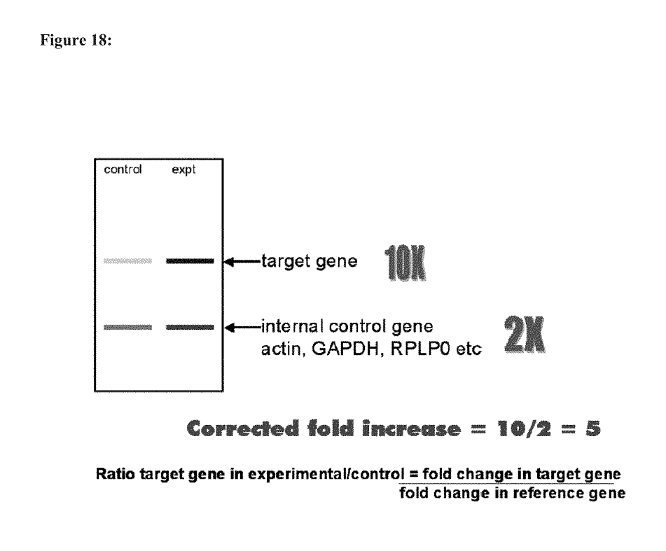

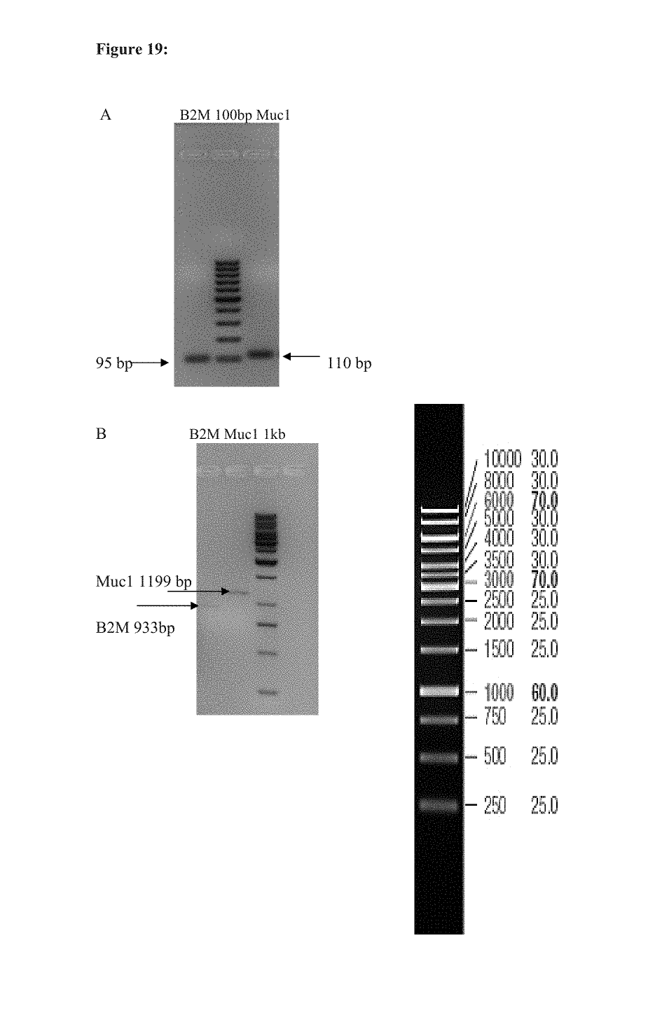

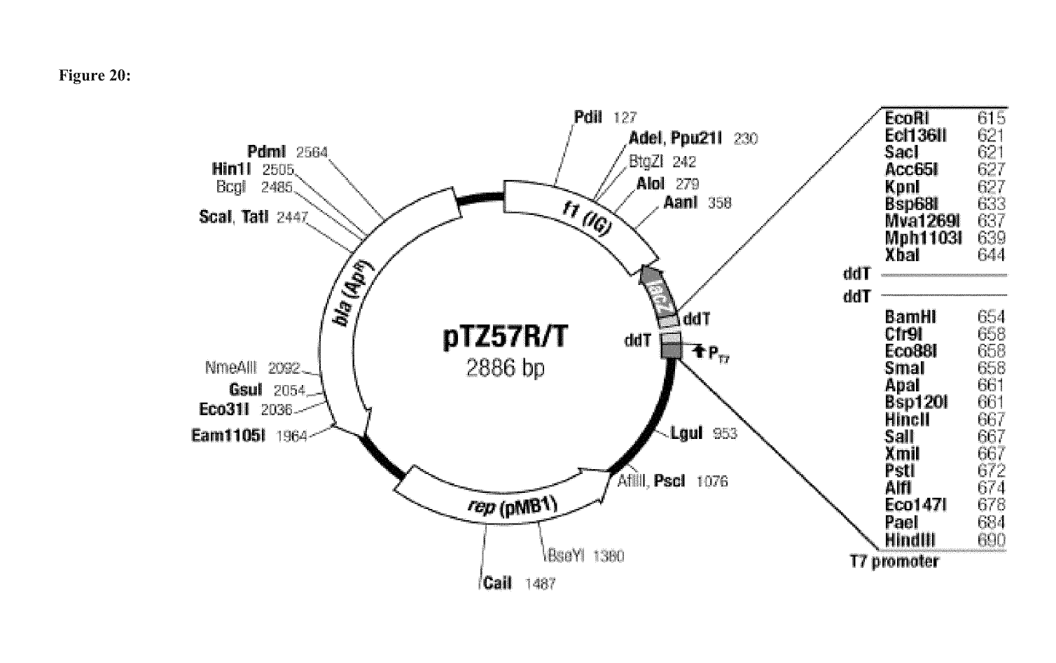

For example, if fold change in target gene expression is 10× and fold change in reference gene expression is 2×, then corrected ratio of target gene expression experimental and control cells is 5× (FIG. 18).

Primers for nearly entire B2M amplification:

For

(SEQ ID No: 116)

5′-AAT ATA AGT GGA GGC GTC GCG CTG-3′
Tm = 67° C.
Rev

(SEQ ID No: 117)

5′-ACC AGA TTA ACC ACA ACC ATG CCT TAC-3′
Tm = 67° C.

Primers for nearly entire muc1 amplification (transcription variant 1):

For (in exon 1)

(SEQ ID No: 118)

5′-ACC TCT CAA GCA GCC AGC GC-3′
Tm = 65° C.
Rev (in last exon)

(SEQ ID No: 119)

5′-TTG GCG CAG TGG GAG ACC ACG-3′
Tm = 67° C.
1 kb

The results are shown in FIG. 19.

Experimental samples of breast cancer tissues were obtained from 39 patients. Total RNA was isolated from these tissue samples using SV Total RNA isolation system (protocol including DNAse treatment). The obtained RNA was used as matrix for cDNA preparation (reverse transcription was performed using Reverta kit (Amplisense) with random hexanucleotide primers mixture). 10 μl of total RNA solution was transformed to 41 μl of cDNA solution in each reverse transcription reaction. Analogous, cDNA samples were prepared from several cultivated tumor (435, T47D, MCF7) and non-tumor (MT2, MT4) cell lines. For each sample 20 μl of obtained cDNA solution was used as matrix for subsequent real-time PCR reaction (total reaction volume 50 μl). Typical results of real-time PCR for muc1 gene are shown on FIG. 17. Analysis of B2M expression levels in different cultivated cell lines normalized to initial total RNA solution concentrations (table 3) showed that B2M seems to be rather bad reference gene for target gene expression analysis in breast cancer/non tumor cells. Literature data for several commonly used reference genes (such as glyceraldehyde-3-phosphate dehydrogenase, β-actin and others) also show excessively high level of difference in reference gene expression levels in normal and tumor cells. Therefore, we decided to reject reference gene approach and calculate mud gene expression levels in breast cancer/non tumor cells by normalizing mud copy number data obtained in real-time PCR to RNA concentration (table 4). The obtained data were then normalized to muc1 gene expression level in MT2 cells (table 4). The majority of breast cancer tissue samples had high level of muc1 gene expression (derepression) compared to non tumor MT2, MT4 cell lines and 435 tumor cell line (table 4).

| | | | | Given Conc | | | |
|---|---|---|---|---|---|---|---|
| | | | | (copies/ | Calc Conc | | |
| No | Name | Type | CT | reaction) | (c/reaction) | % Var | Ct |
| 1 | H2O | unknown | | | | | |
| 2 | MUC-5 20 mkl | standard | 14.88 | 3420520 | 3597115 | 5.2% | 14.88 |
| | Ex1 B2M | | | | | | |
| | probes 7 pmol | | | | | | |
| | B2M primers | | | | | | |
| | 50 pmol Ex1 | | | | | | |
| | primers 37.5 | | | | | | |
| | pmol | | | | | | |
| 4 | MUC-6 20 mkl | standard | 18.61 | 342052 | 319847 | 6.5% | 18.61 |
| | Ex1 B2M | | | | | | |
| | probes 7 pmol | | | | | | |
| | B2M primers | | | | | | |
| | 50 pmol Ex1 | | | | | | |
| | primers 37.5 | | | | | | |
| | pmol | | | | | | |
| 7 | MUC-9 20 mkl | standard | 29.04 | 342 | 368 | 7.5% | 29.14 |
| | Ex1 B2M | | | | | | |
| | probes 7 pmol | | | | | | |
| | B2M primers | | | | | | |
| | 50 pmol Ex1 | | | | | | |
| | primers 37.5 | | | | | | |
| | pmol | | | | | | |
| 8 | MUC-9 20 mkl | standard | 29.24 | 342 | 324 | 5.4% | |
| | Ex1 B2M | | | | | | |
| | probes 7 pmol | | | | | | |
| | B2M primers | | | | | | |
| | 50 pmol Ex1 | | | | | | |
| | primers 37.5 | | | | | | |
| | pmol | | | | | | |
| 16 | 11 cDNA 20 | unknown | 16.38 | | 1355877 | | 16.38 |
| | mkl | | | | | | |
| 17 | 12 cDNA 20 | unknown | 28.87 | | 409 | | 28.87 |
| | mkl | | | | | | |
| 18 | 13 cDNA 20 | unknown | 18.16 | | 427120 | | 18.16 |
| | mkl | | | | | | |
| 19 | 14 cDNA 20 | unknown | 12.54 | | 16377792 | | 12.54 |
| | mkl | | | | | | |
| 20 | 15 cDNA 20 | unknown | 15.93 | | 1816940 | | 15.93 |
| | mkl | | | | | | |
| 21 | 16 cDNA 20 | unknown | 16.87 | | 987431 | | 16.87 |
| | mkl | | | | | | |
| 22 | 17 cDNA 20 | unknown | 19.98 | | 130850 | | 19.98 |
| | mkl | | | | | | |
| 23 | 18 cDNA 20 | unknown | 13.90 | | 6770563 | | 13.90 |
| | mkl | | | | | | |
| 24 | 21 cDNA 20 | unknown | 18.48 | | 347746 | | 18.48 |
| | mkl | | | | | | |
| 25 | 22 cDNA 20 | unknown | 14.75 | | 3904071 | | 14.75 |
| | mkl | | | | | | |
| 26 | 23 cDNA 20 | unknown | 19.68 | | 159045 | | 19.68 |
| | mkl | | | | | | |
| 27 | 24 cDNA 20 | unknown | 15.59 | | 2266926 | | 15.59 |
| | mkl | | | | | | |
| 28 | 25 cDNA 20 | unknown | 18.36 | | 375453 | | 18.36 |
| | mkl | | | | | | |
| 29 | 26 cDNA 20 | unknown | 16.03 | | 1703507 | | 16.03 |
| | mkl | | | | | | |
| 30 | 27 cDNA 20 | unknown | 13.73 | | 7556050 | | 13.73 |
| | mkl | | | | | | |
| 31 | MT2 cDNA | 20 unknown | 16.48 | | 873631 | | 16.18 |
| | mkl | | | | | | |
| 32 | MT4 cDNA | 20 unknown | 18.17 | | 423415 | | 18.17 |
| | mkl | | | | | | |
| 33 | MCF7 cDNA | unknown | 14.30 | | 3768499 | | 14.30 |
| | 20 mkl | | | | | | |
| 34 | T47D cDNA | unknown | 12.05 | | 22528153 | | 12.05 |
| | 20 mkl | | | | | | |
| 35 | 435 cDNA 20 | unknown | 17.91 | | 502250 | | 17.91 |
| | mkl | | | | | | |
| 36 | MT2 new | unknown | 18.71 | | 251428 | | 19.21 |
| | cDNA 20 mkl | | | | | | |

Working over the project of development the targeted therapy for triple-negative breast cancer cases we completed the quantitative diagnostic system for this type of women cancer disease. This kit is based on Real Time reverse transcription polymerase chain reaction (Real Time RT-PCR) and measures expression levels for Muc1 antigen as well as for the other standard breast cancer markers Her2Neu, estrogen and progesterone (ER and PR) receptors in the same test. Originally we intended to choose those breast cancer patients who are eligible for Muc1 therapeutic treatment existing in clinical trials (Muc1 monoclonal antibodies, mucine-immunity boosting therapies) and for our Muc1 -targeted applications which are under laboratory development. The diagnostic system was tested in samples of 98 breast cancer patients undergoing surgery in most cases following with chemotherapy during 2008-2010 years. It occurred that 75 percent of all breast cancer patients have Muc1 antigen tumor hyperexpression (it is higher than 3.4 times compared with lymphocyte cell cultures MT2 and MT4 or 17-34 times higher than measured in healthy humans whole blood RNA extract) Muc1 expression background level, and not 95 percent as it was believed breast cancer tumors show). 30 percent of all 98 breast cancers were ER-PR-negative, it means that these patients had little response for aromatase inhibitor's and chemotherapy and poor survival prognosis from the 3^{rd} stage with beginning of metastatic disease development when this hormone-chemotherapeutic treatment is necessary. The most interesting results were the findings that half of these hormone-negative patients, 85 percent of them also Her2-negative or so called “triple-negative breast cancer”, have hyperexpression of Muc1 antigen and being hopeless for the other hormone-chemotherapeutic treatment at metastatic stage are eligible for Muc1-targeted treatment. This amount is 13.5 percent of total breast cancer incidences and almost 45 percent of triple-negative breast cancers which are admitted to be rather hopeless in their metastatic stages.

It also occurred we were able to distinguish malignant-specific and normal types of Muc1 expression, and as kit system is sensitive enough it is possible to see the marker from patient's blood samples. This might be possible to use the kit as blood diagnostic screening as well as non-invasive indication of metastatic progression in patients after surgery and chemo-radiation therapy. The test system is quantitative, faster, easier and relatively not more expensive than existing routine immunodiagnostic of breast cancer.

It is also occurred that Muc1 malignant forms hyperexpression can be observed (and probably used for diagnostics) in the other cancer types such as ovarian cancer, prostate cancer, lung cancer, colon and bladder cancers. For this application the special commercially reasonable test system development like we made for breast cancer screening would be recommended for each cancer type.

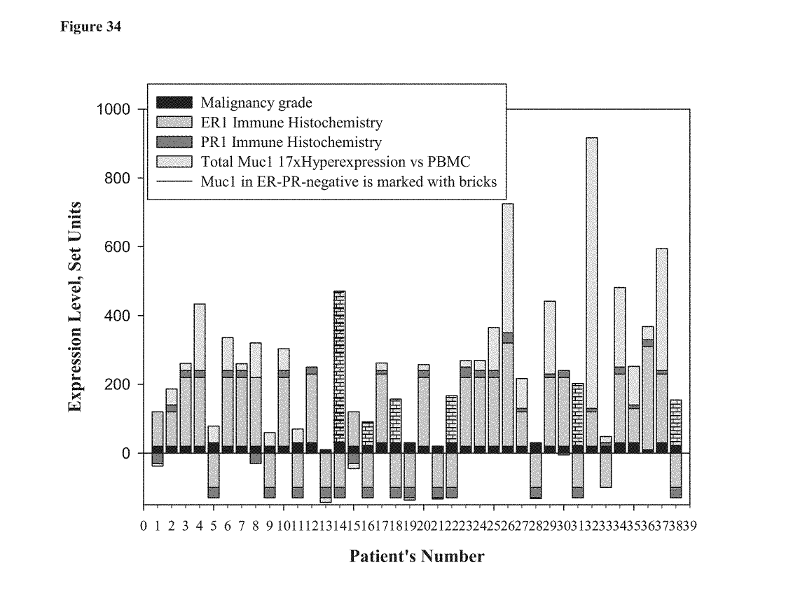

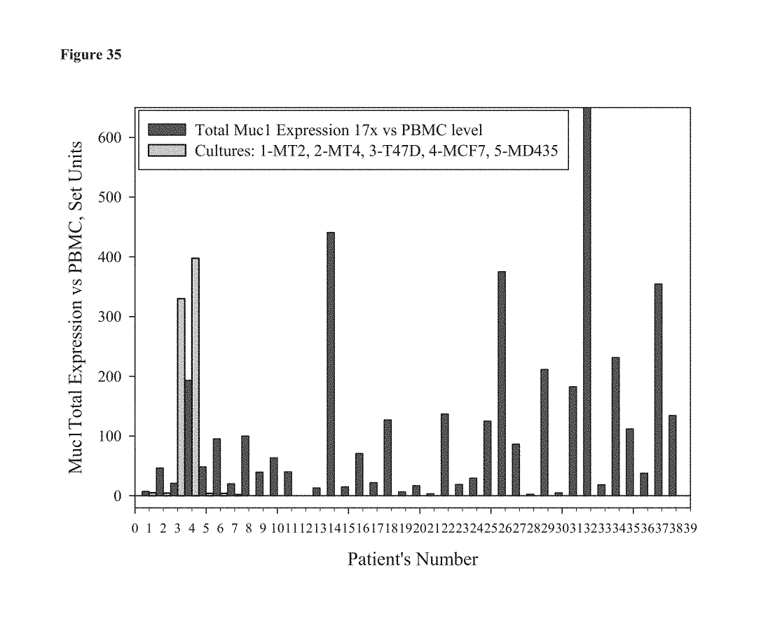

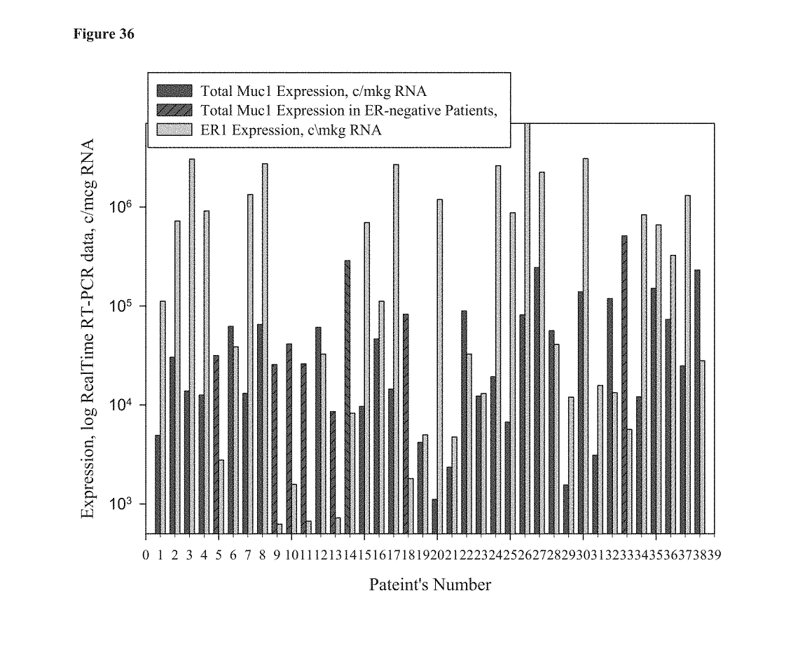

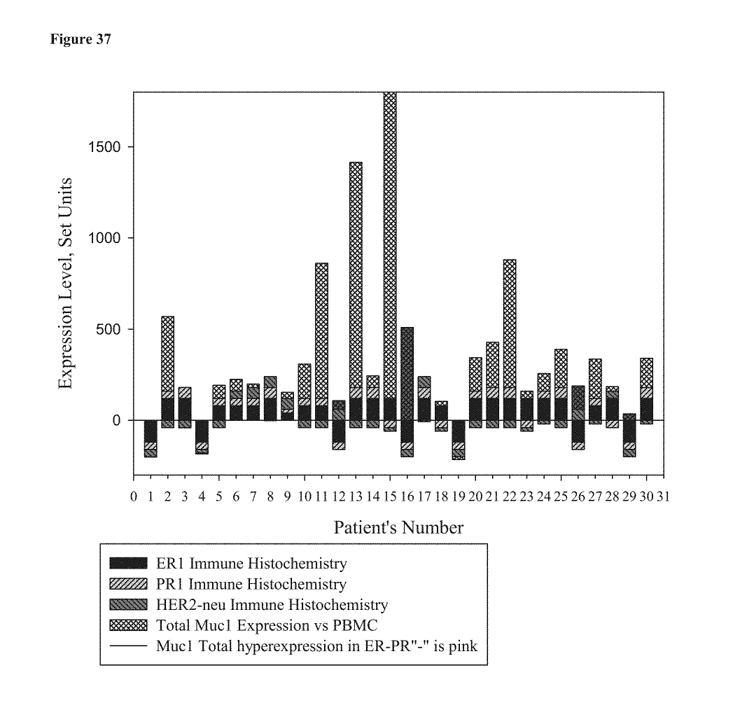

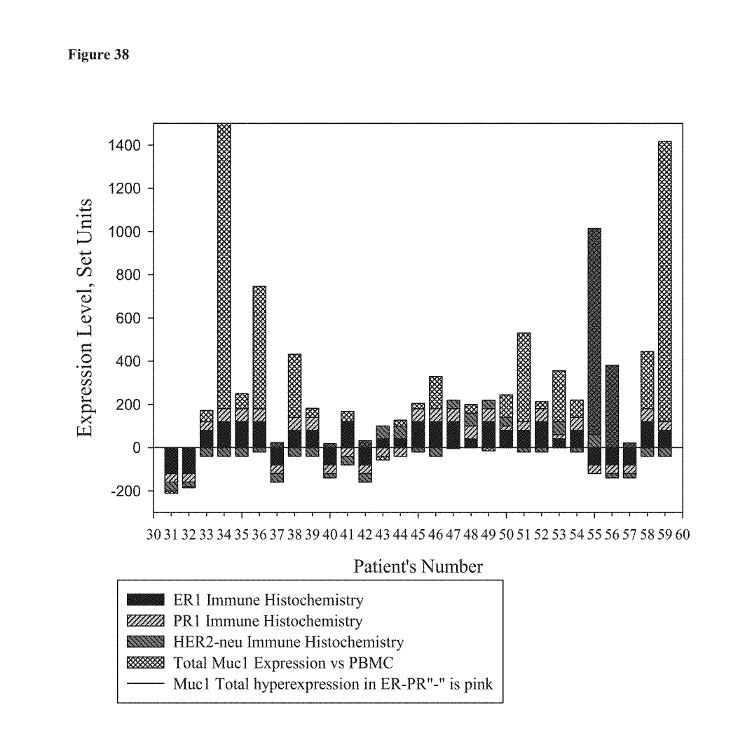

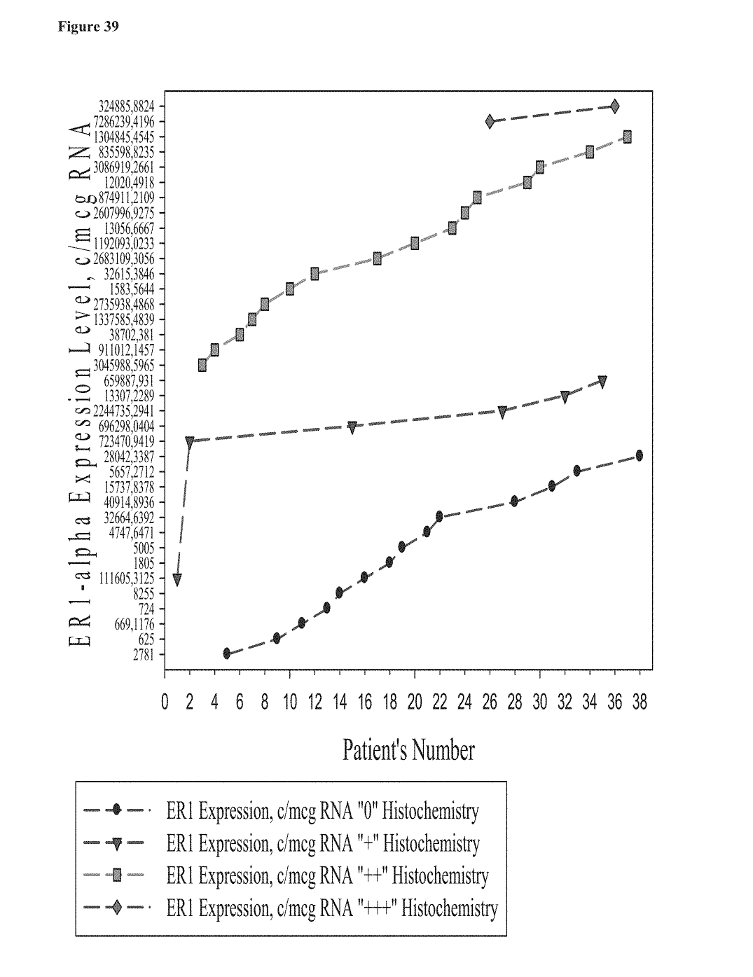

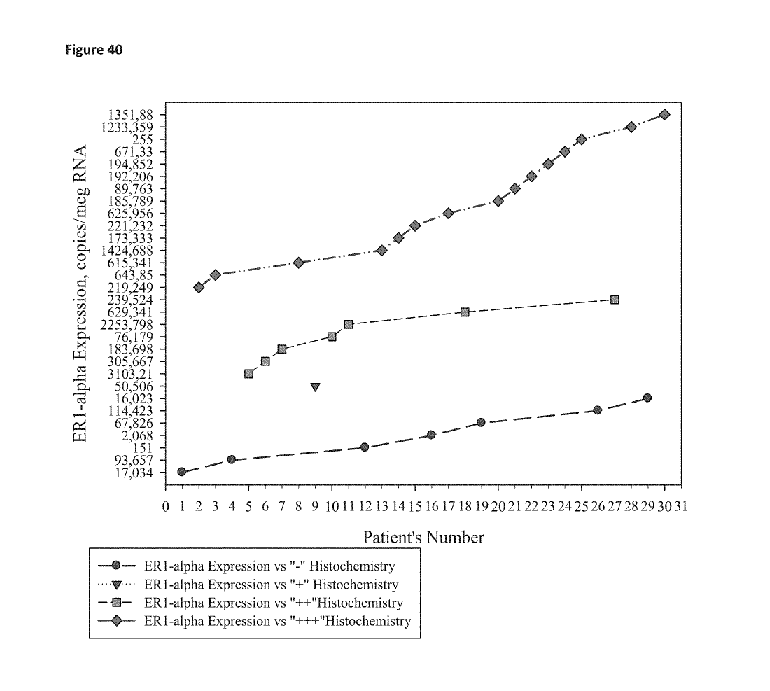

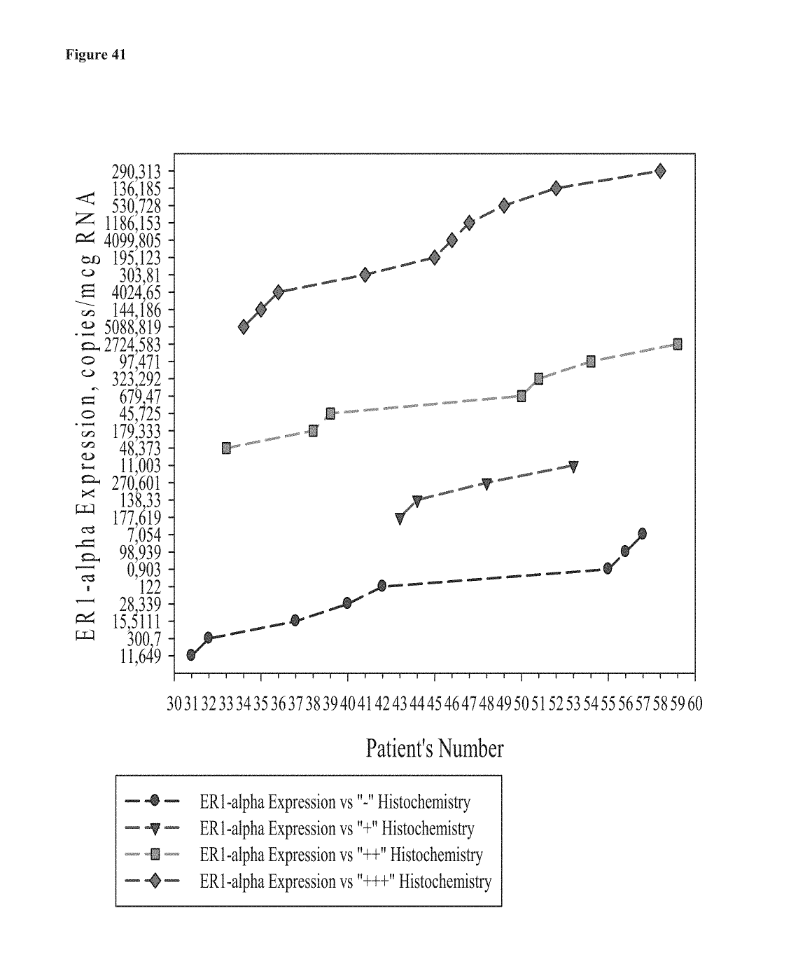

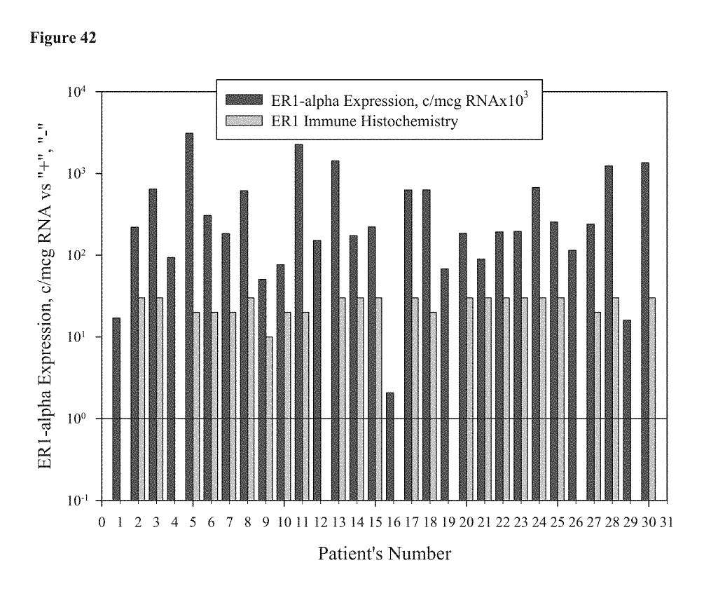

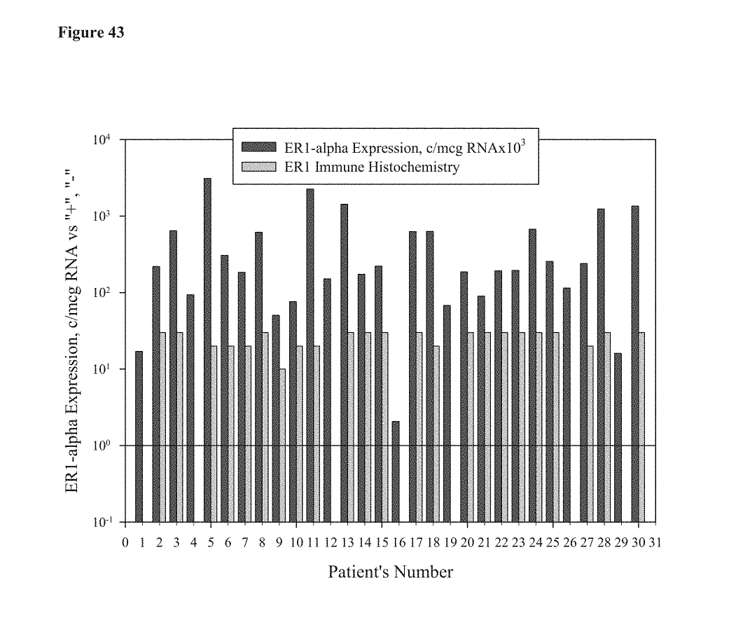

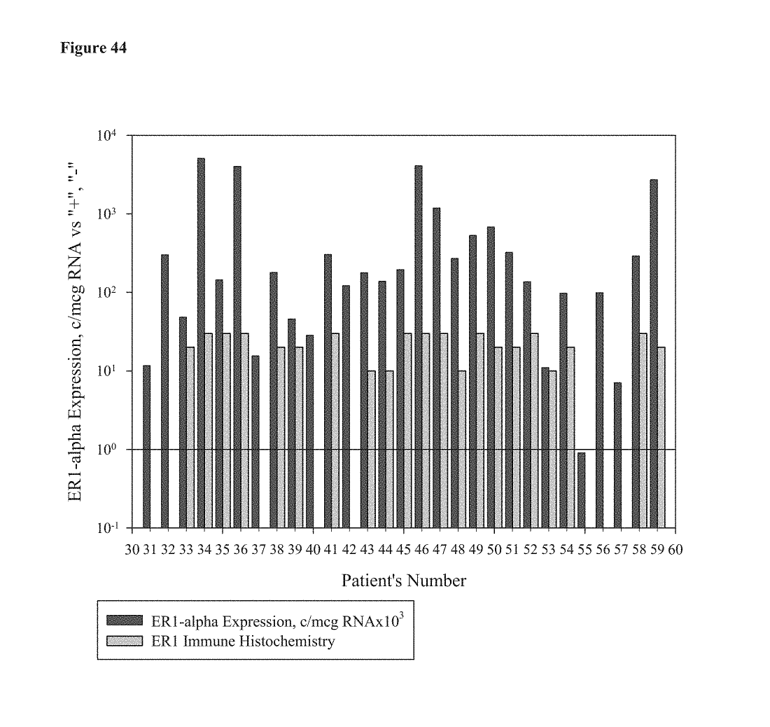

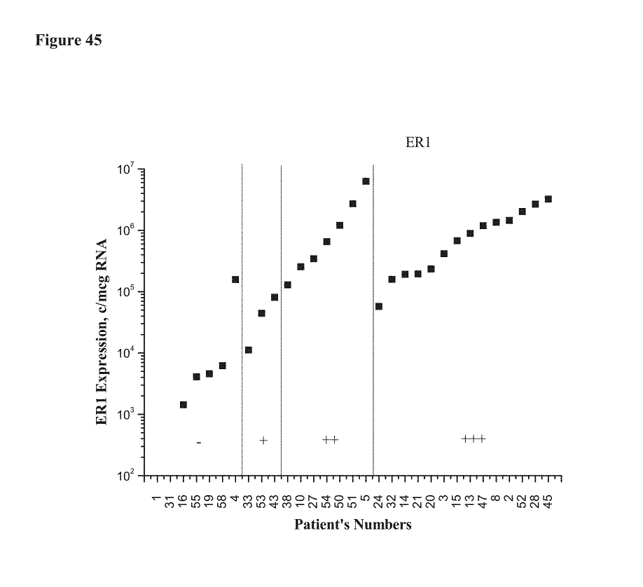

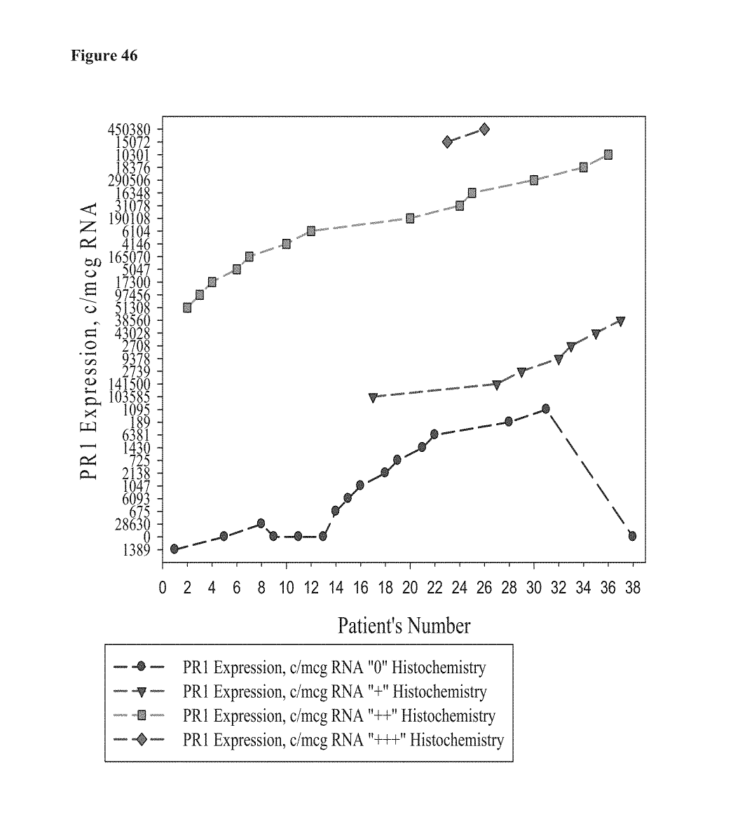

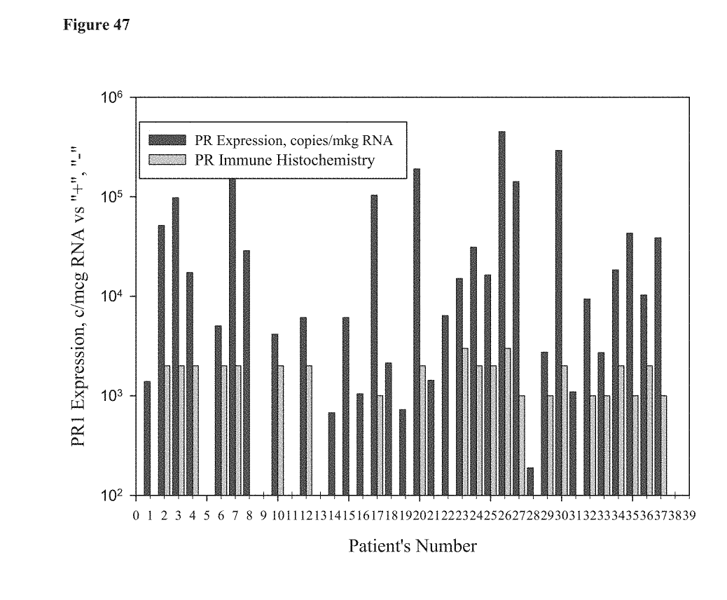

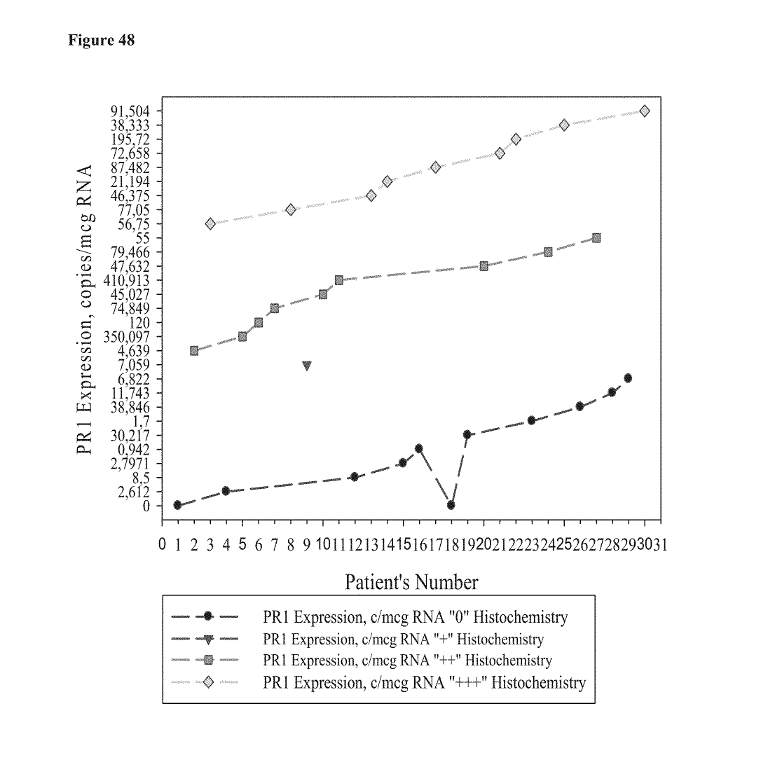

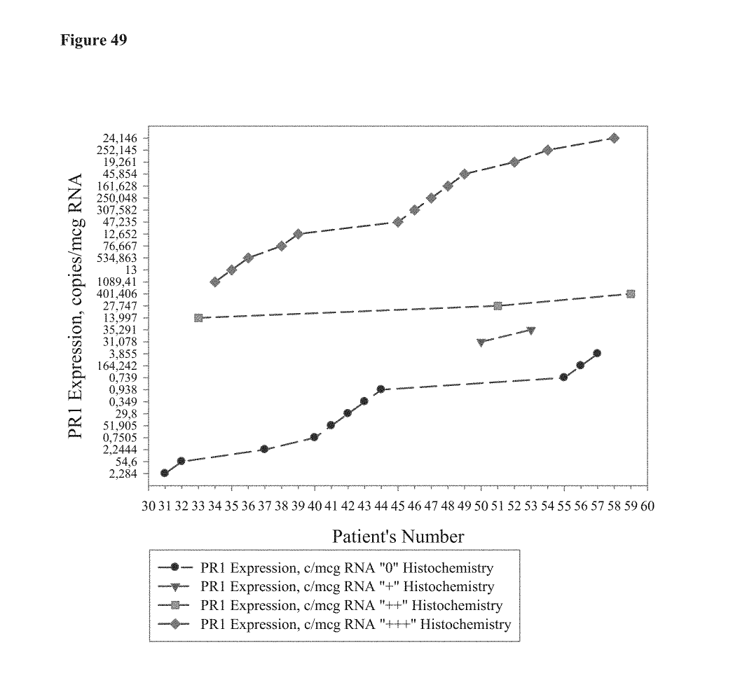

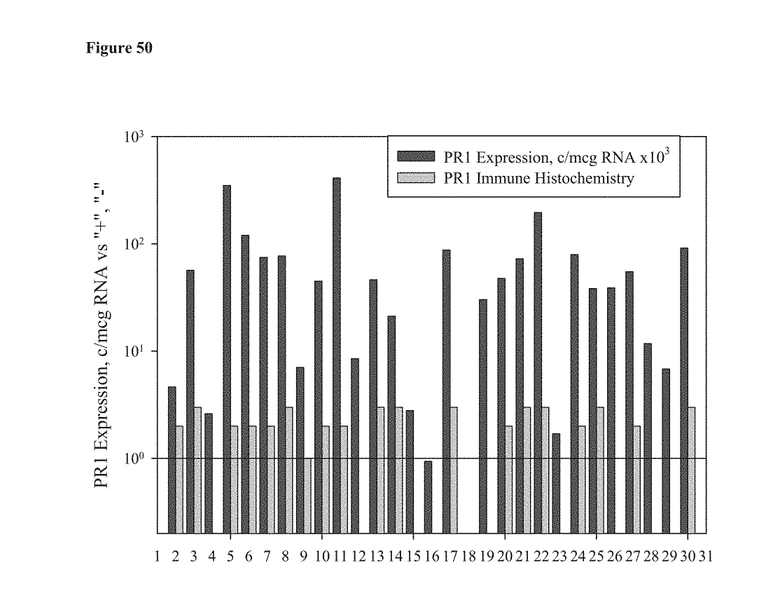

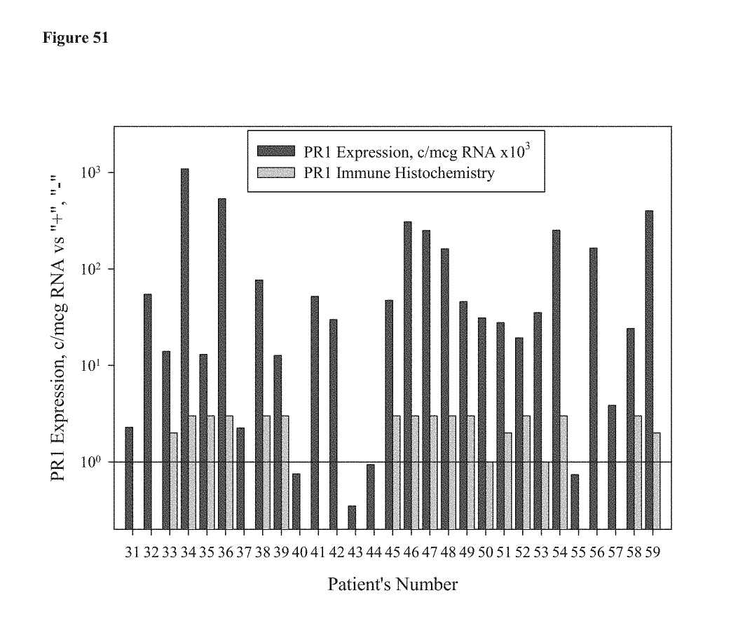

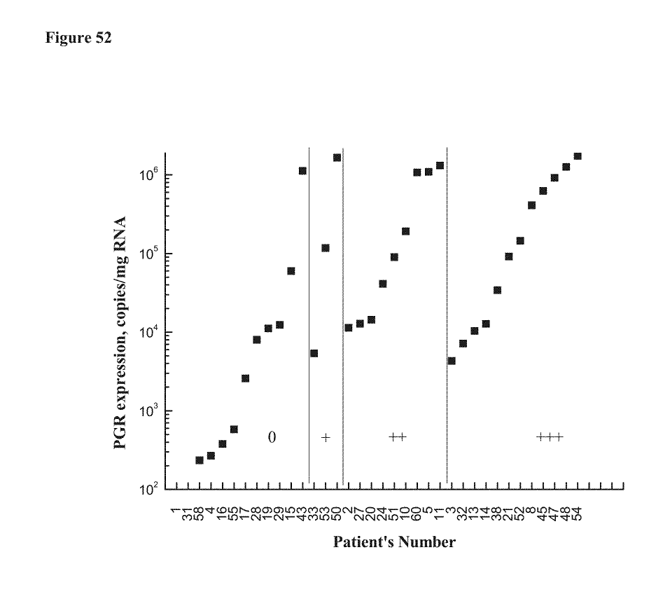

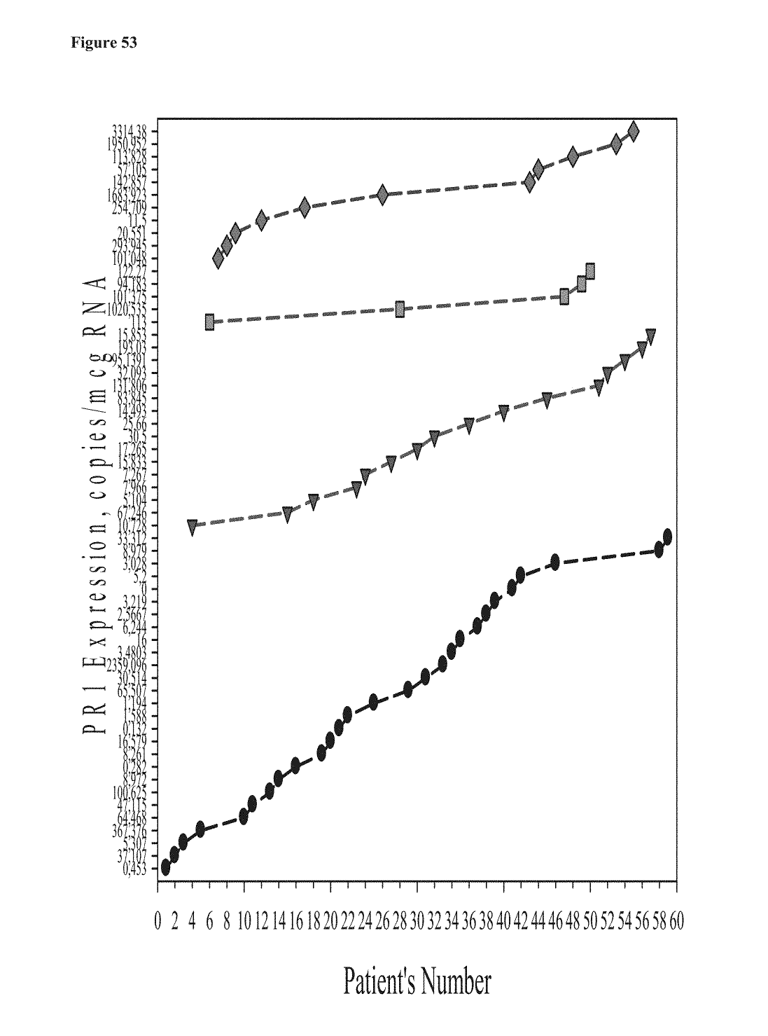

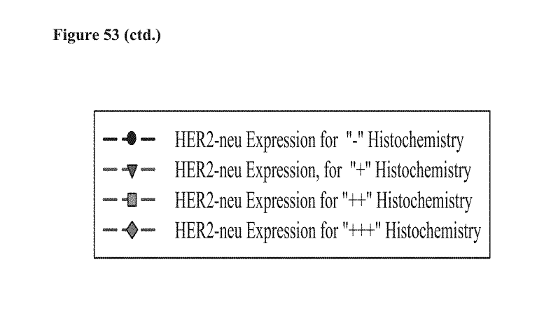

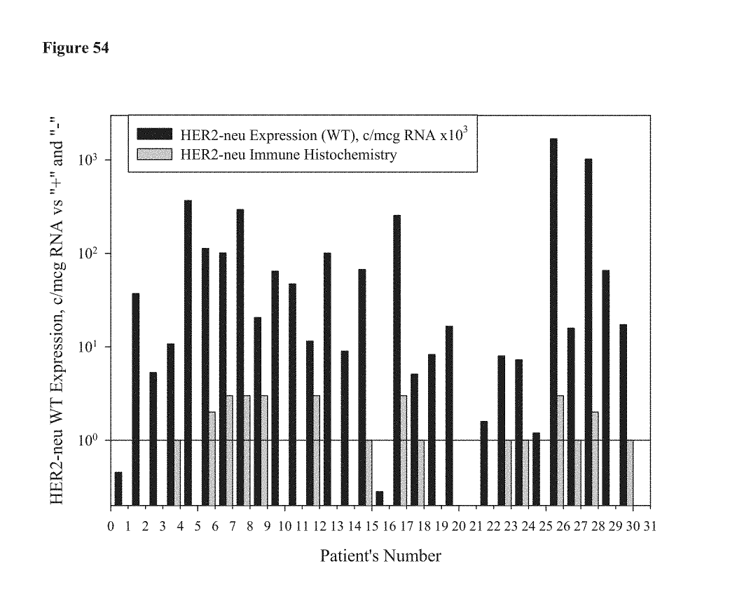

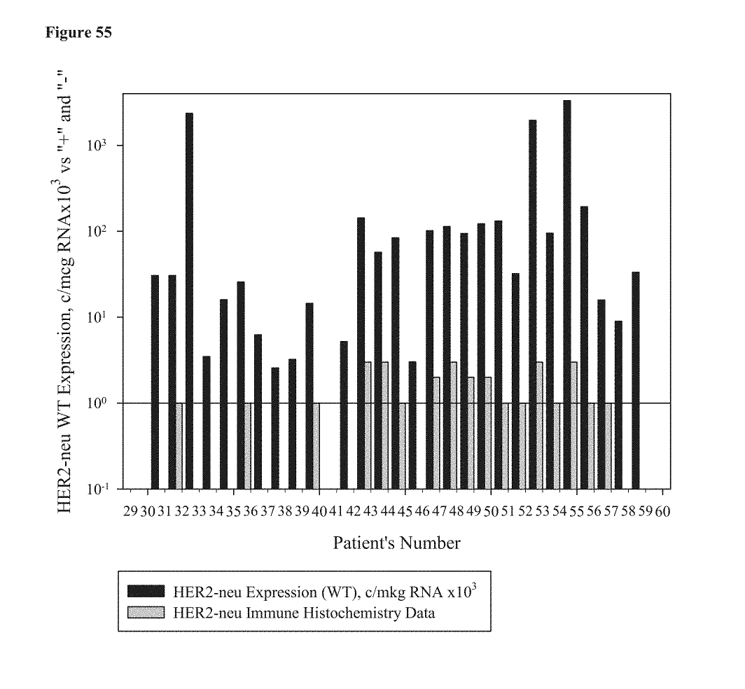

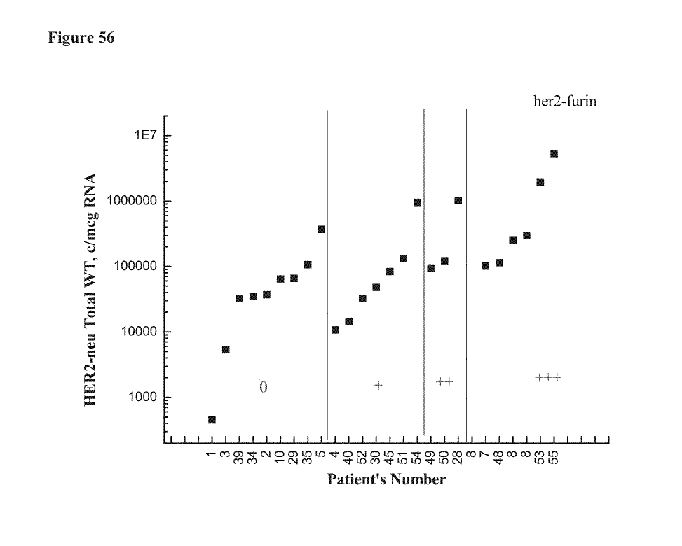

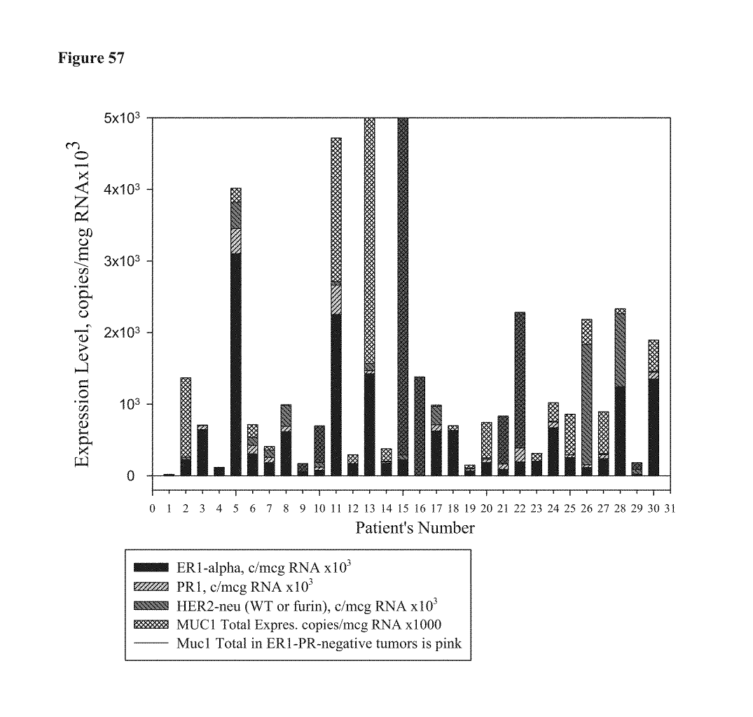

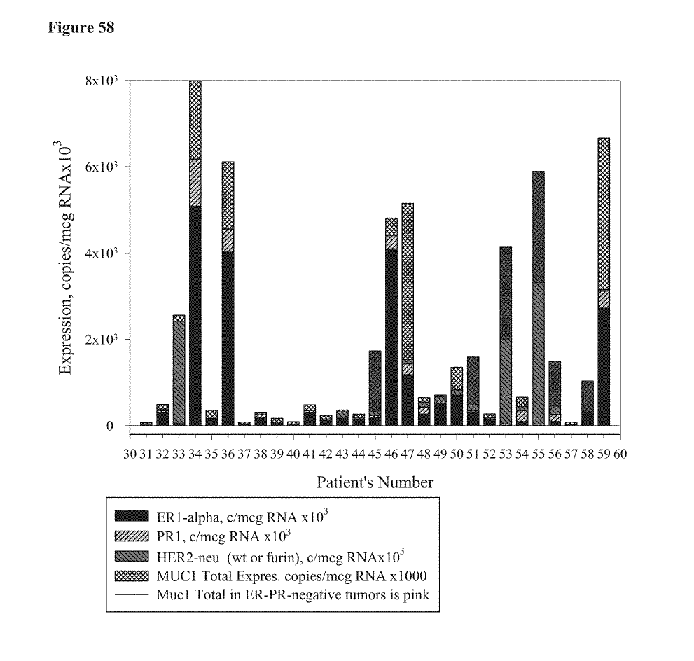

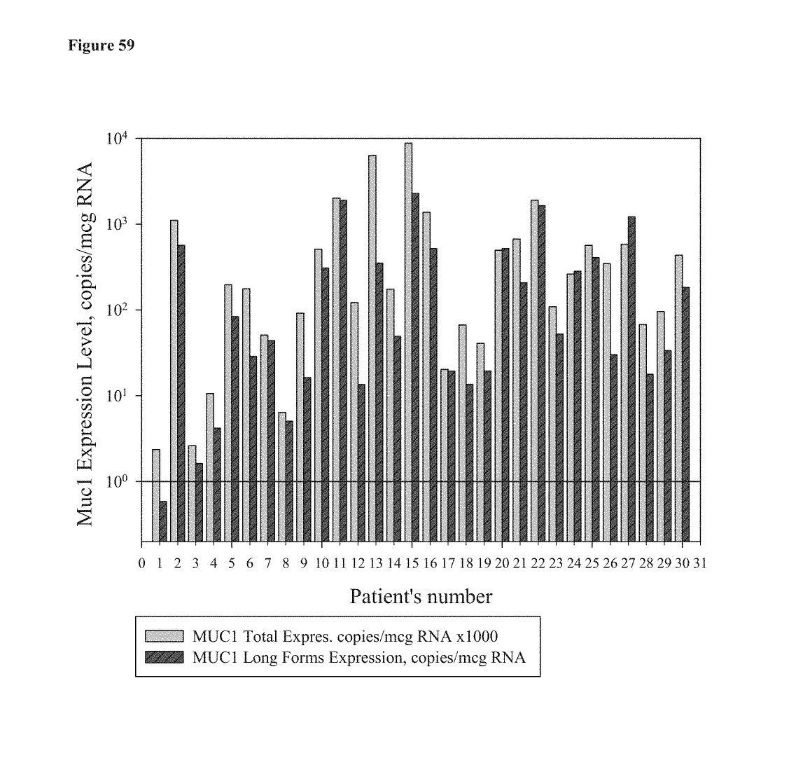

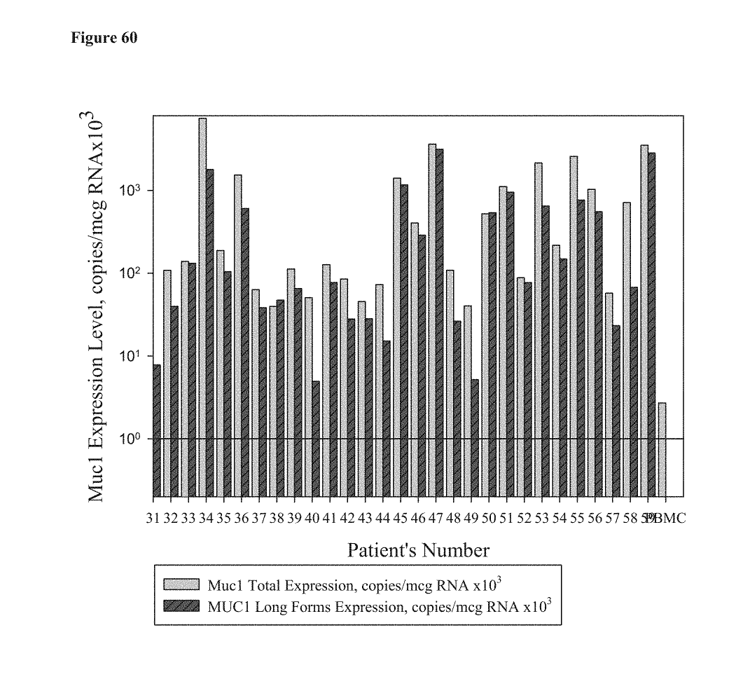

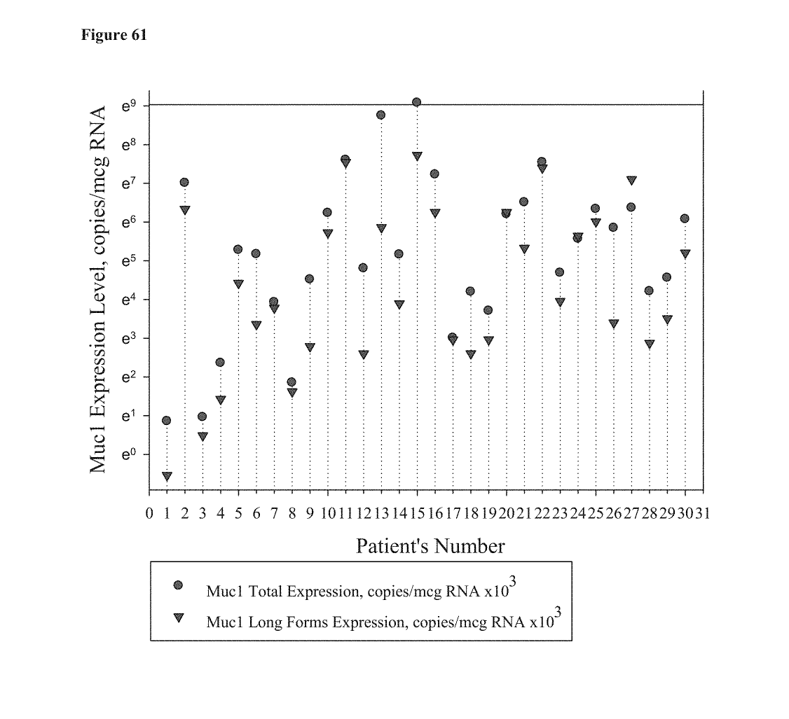

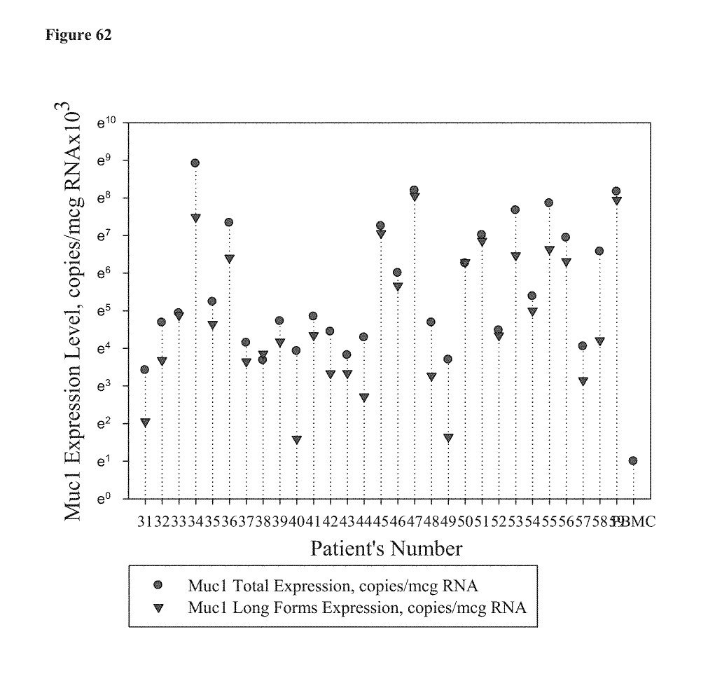

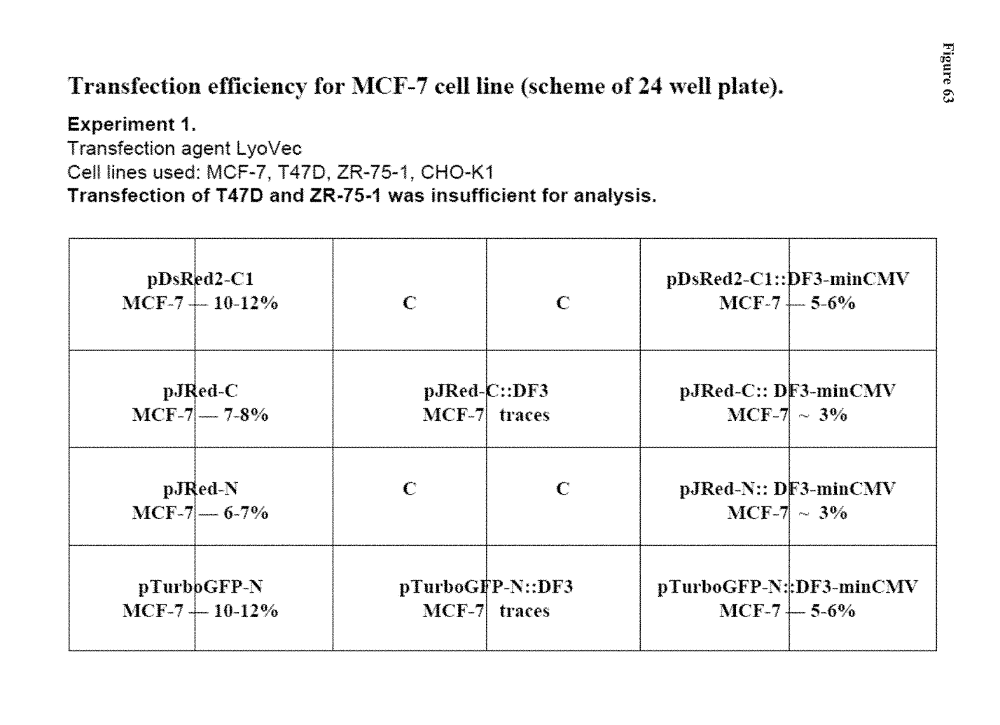

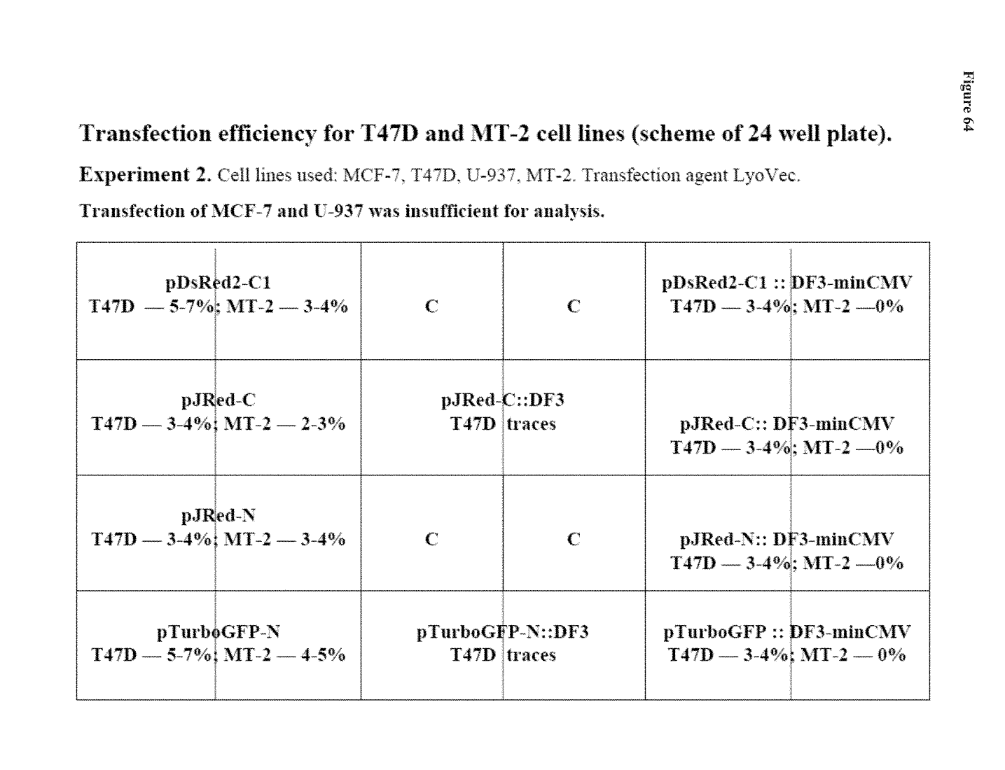

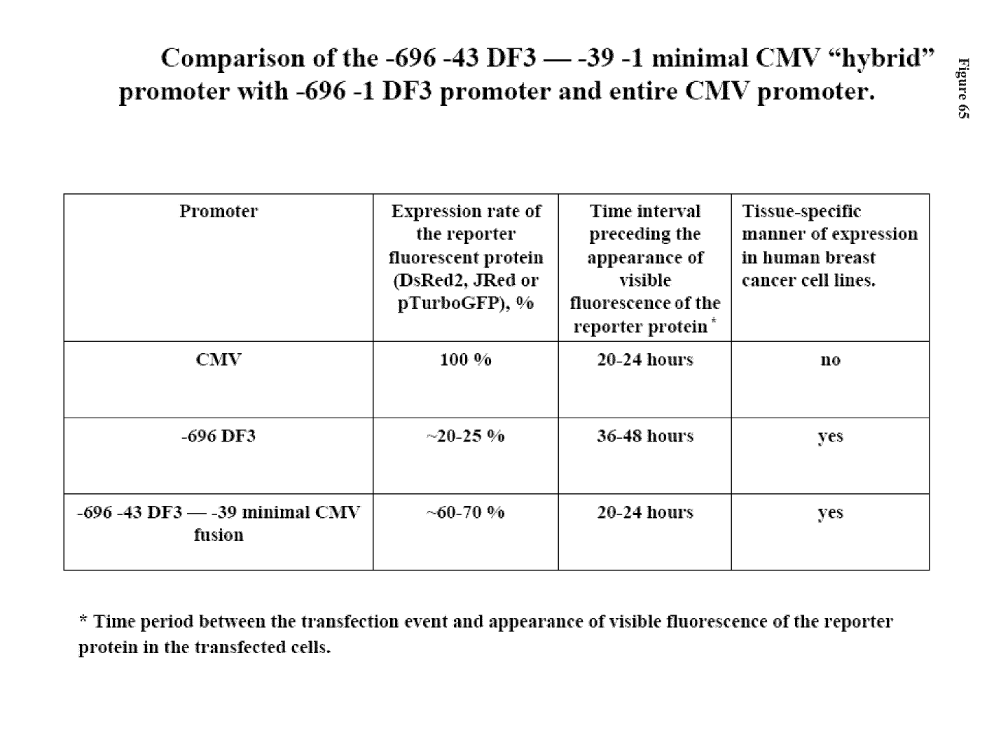

The results are shown in FIGS. 34 to 62.

TABLE 4
Analysis of muc1 expression in breast cancer patients 2008-2009. muc1
expression levels three or more times higher then in MT2 cells is marked.

| | | | Initial RNA | Norm. to initial | |
|---|---|---|---|---|---|
| | | | solution | RNA solution | Norm. |
| Sample | Ct, | Mean, muc1 | conc., | with conc. 1 | to |
| ## | mean | copies/reaction | μg/ml | μg/ml | MT2 |
| 1 | 16.64 | 879449 | 178.1 | 4938 | 1.5 |
| 2 | 15.76 | 1515533 | 49.9 | 30371 | 9.3 |
| 3 | 19.75 | 128969 | 9.3 | 13868 | 4.2 |
| 4 | 13.47 | 6230331 | 49.4 | 126120 | 38.7 |
| 5 | 16.33 | 1062572 | 33.6 | 31624 | 9.7 |
| 6 | 13.72 | 5348309 | 85.7 | 62407 | 19.1 |
| 7 | 17.80 | 428740 | 32.6 | 13151 | 4.0 |
| 8 | 14.98 | 2443972 | 37.5 | 65172 | 20.0 |
| 9 | 14.22 | 3908628 | 152.0 | 25715 | 7.9 |
| 10 | 13.82 | 5013181 | 121.2 | 41363 | 12.7 |
| 11 | 16.38 | 1222057 | 46.8 | 26112 | 8.0 |
| 12 | 28.87 | 413 | 6.8 | 61 | 0.02 |
| 13 | 18.16 | 391160 | 45.6 | 8578 | 2.6 |
| 14 | 12.54 | 14261649 | 49.6 | 287533 | 88.2 |
| 15 | 15.93 | 1631002 | 168.4 | 9685 | 3.0 |
| 16 | 16.87 | 893884 | 19.2 | 46556 | 14.2 |
| 17 | 19.98 | 121809 | 8.4 | 14501 | 4.4 |
| 18 | 13.90 | 5968180 | 72.0 | 82891 | 25.4 |
| 19 | 18.42 | 445995 | 106.4 | 4192 | 1.3 |
| 20 | 18.67 | 383552 | 34.4 | 11150 | 3.4 |
| 21 | 18.48 | 319376 | 136.0 | 2348 | 0.7 |
| 22 | 14.75 | 3467688 | 38.8 | 89373 | 27.4 |
| 23 | 19.68 | 147657 | 12.0 | 12305 | 3.8 |
| 24 | 15.59 | 2028724 | 104.8 | 19358 | 5.9 |
| 25 | 16.03 | 1530540 | 18.8 | 81412 | 25.0 |
| 26 | 13.73 | 6650481 | 27.2 | 244503 | 75.0 |
| 27 | 15.49 | 2525227 | 44.8 | 56367 | 17.3 |
| 28 | 21.37 | 78023 | 48.8 | 1560 | 0.5 |
| 29 | 14.00 | 6066284 | 43.6 | 139135 | 42.3 |
| 30 | 22.26 | 45984 | 14.8 | 3107 | 1.0 |
| 31 | 14.73 | 3951379 | 33.2 | 119017 | 36.5 |
| 32 | 12.22 | 17436621 | 34.0 | 512842 | 157.3 |
| 33 | 16.39 | 1481212 | 122.4 | 12101 | 3.7 |
| 34 | 13.76 | 7009229 | 46.4 | 151061 | 46.3 |
| 35 | 15.51 | 2485450 | 34.0 | 73101 | 22.4 |
| 36 | 17.87 | 616588 | 24.8 | 24862 | 7.6 |
| 37 | 13.13 | 10162979 | 44.0 | 230977 | 70.9 |
| 38 | 13.91 | 6423118 | 73.0 | 87988 | 26.9 |
| MT2 | 16.65 | 873631 | 268.0 | 3260 | 1.0 |
| MT4 | 18.23 | 383859 | 128.8 | 2980 | 0.9 |
| MCF7 | 14.42 | 3468499 | 16.0 | 216781 | 66.0 |
| T47D | 13.54 | 5957377 | 23.0 | 259016 | 79.5 |
| 435 | 17.46 | 502250 | 199.7 | 2515 | 0.8 |
| MOI | 19.75 | 128541 | 47.5 | 2706 | 0.8 |
| 3A/PL | 21.65 | 39641 | 30.4 | 1304 | 0.4 |

2. Development of Antibody Targeted HSV-TK-GCV-based Genetic Vaccines with Selective Activity for Breast Cancer Treatment
2.1 Cloning the HSV-1 and HSV-2 thymidine kinase genes in mammalian expression vectors pDsRed2 and p2FP-RNAi.

The plasmid construction pUT 649 (Cayla) was used as initial HSV-I TK source. The expression product of pDsRed2-TKI construction is HSV-1 TK enzyme, fused with reporter DsRed2 fluorescent protein in its C-terminal region that makes it possible to control the transfection efficiency.

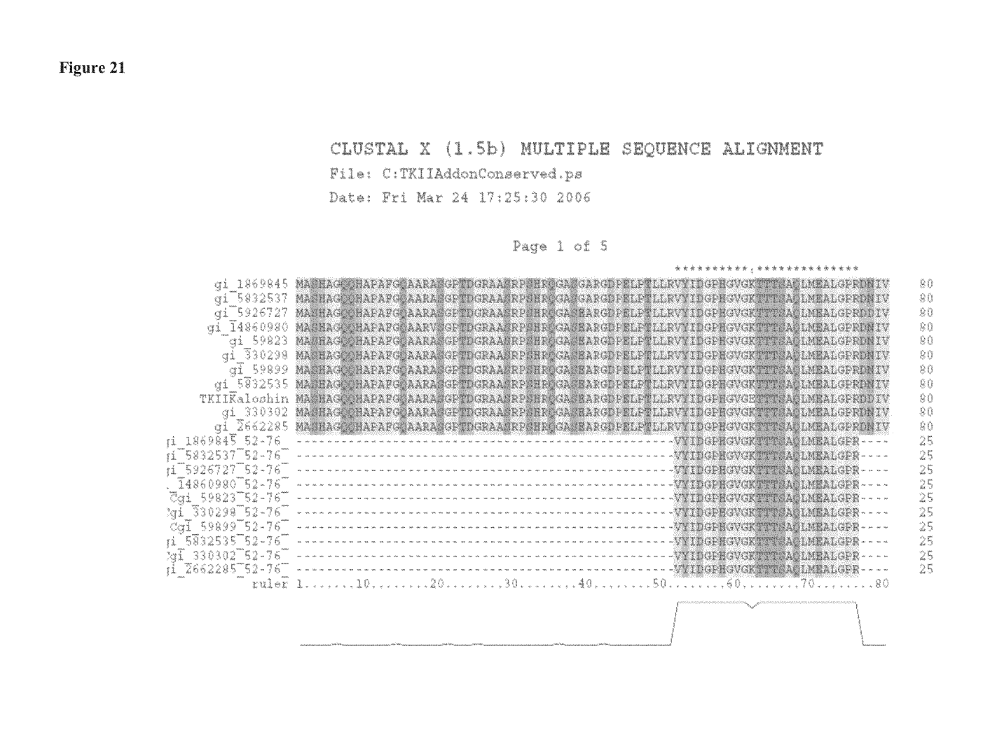

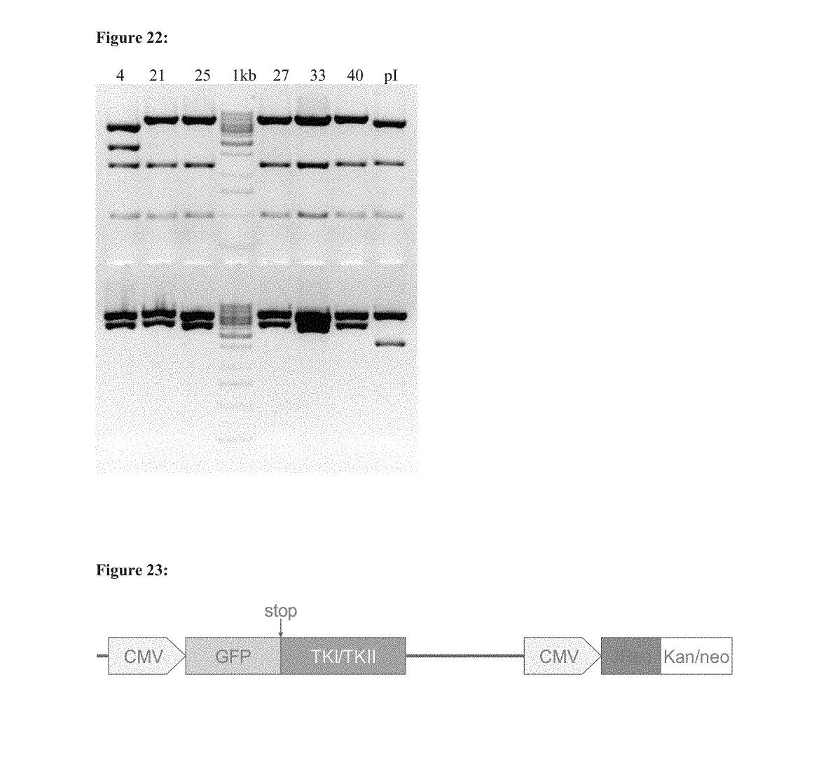

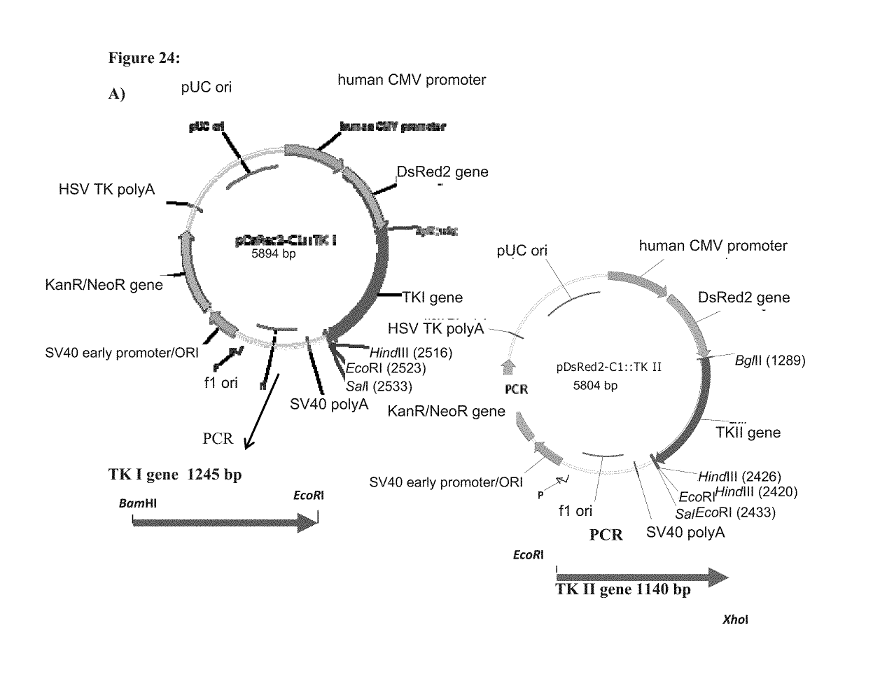

HSV-2 TK gene was amplified from HSV-2 viral isolate as matrix in PCR. The obtained PCR product of 1200 bp in size was cloned in pTZ57R. TKII gene appeared to obtain easily point mutations or deletions cause the luck of ferment's activity in cycles of PCR during re-cloning. E. coli clones, carrying pTZ57R with insert of proper length were selected for plasmid sequencing and analyzed for the presence of significant point mutation. Multiple sequence alignment can be necessary for discovering the disorders (FIG. 21: Multiple amino acid sequence alignment for obtained TKII with reference GenBank data).

HSV-2 TK gene was excised from pTZ57R-TK2 N(224 by Bgl II/Hind III digestion and cloned in pDsRed2-C1. The HSV-2 TK gene in the final plasmid construction pDsRed2-TK2 was sequenced. Plasmids from clones pDsRed2-TK2 No.7 and No.10 (without mutation in HSV-2 TK gene) were prepared in amounts enough for the transfection. At the same time, whereas HSV-1 TK protein is fused with reporter Red2 protein in its C-terminal region in pDsRed2-TK1 vector, this leads to 4-5 fold decrease of the TK-2 activity (as compared with literature data). Therefore, we tried to clone HSV-1 TK and HSV-2 TK genes in vectors, containing IRES sequence between fluorescent reporter gene and TK gene, but the results obtained were unsatisfactory. Finally we tried to clone HSV-1 TK and HSV-2 TK genes in p2FP-RNAi vector. This vector contains two reporter genes, encoding green fluorescent protein TurboGFP and red fluorescent protein JRed, correspondingly. Both reporter genes have CMV promoters. JRed is necessary as positive transfection control. TurboGFP possess cloning site in its' 3′-noncoding region.

Cloning of HSV-1 TK and HSV-2 TK genes in p2FP-RNAi was performed in two steps. On the first step HSV-1 TK and HSV-2 TK genes were excised by Bgl II/Hind III digestion from pDsRed2-TK1 and pTZ57R-TK2 No.24 plasmid constructions, correspondingly, and were cloned in Bgl II and Hind III sites of p2FP-RNAi (see figures).
2.2 Cloning of HSV-1 and HSV-2 thymidine kinase genes in mammalian expression vector pcDNA4/HisMax C.

In order to compare the activities of HSV1 and HSV2 thymidine kinase enzymes expressed in human breast cancer xenografts in SCID mice model it was necessary to obtain the strong expression pattern of the previously cloned HSV1 and HSV2 thymidine kinase genes in mammalian cells. For that purpose these TK genes were cloned in the pcDNA4/HisMax C expression vector, allowing high-level expression in most mammalian cell lines, purification and detection of expressed recombinant proteins.

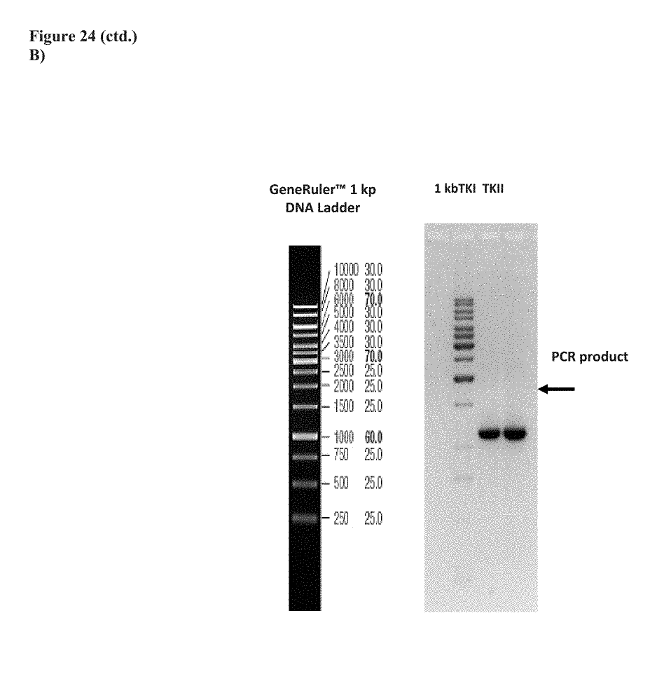

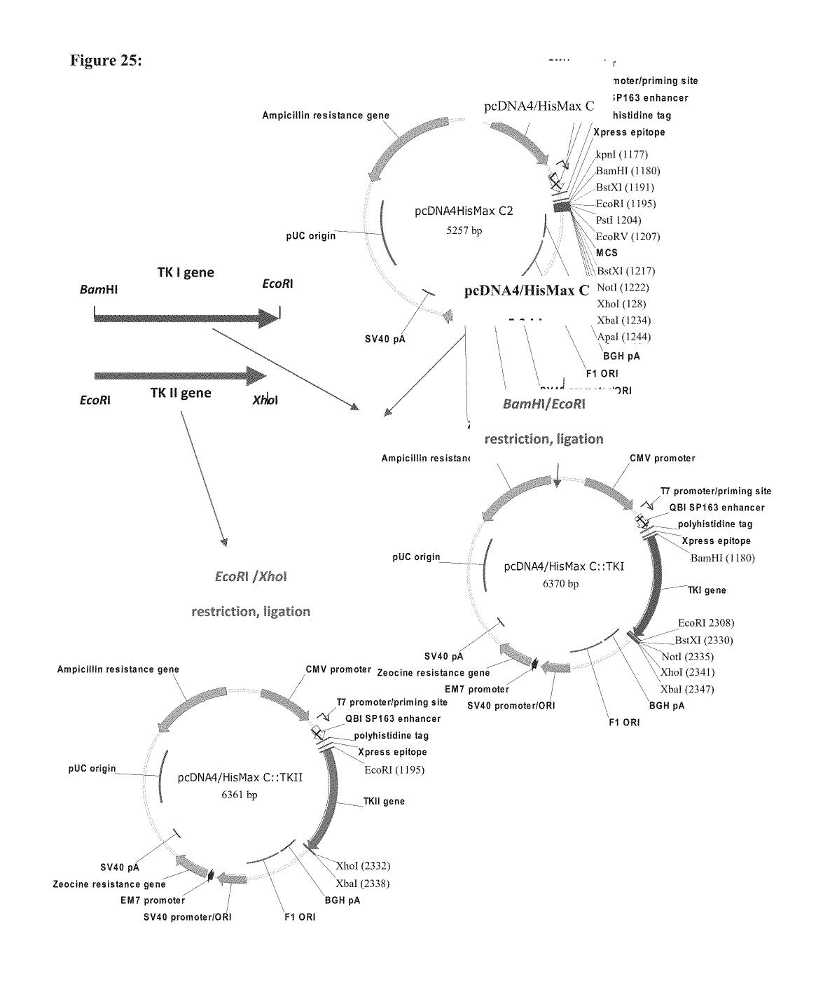

The previously obtained plasmid constructions pDsRed2-C1::TKI and pDsRed2-C1::TKII, containing thymidine kinase genes in fusion with DsRed2 red fluorescent protein, were used as the source of thymidine kinase genes, TKI and TKII, correspondingly. TKI gene was PCR amplified from pDsRed2-C1::TKI matrix using oligonucleotide primers, containing BamHI (forward) and EcoRI (reverse) restriction sites (table 5). The obtained PCR product was digested with BamHI and EcoRI restriction endonucleases, gel purified and cloned in BamHI/EcoRI digested expression vector pcDNA4/HisMax C. TKII gene was PCR amplified from pDsRed2-C1::TKII matrix using oligonucleotide primers, containing EcoRI (forward) and Xhol (reverse) restriction sites (table 5). The obtained PCR product was digested with EcoRI and Xhol restriction endonucleases, gel purified and cloned in EcoRI/Xho/I digested vector pcDNA4/HisMax C. The scheme of cloning TKI and TKII genes in expression vector pcDNA4/HisMax C is presented on FIG. 25.

TABLE 5
Oligonucleotide primers used for amplification
TKI and TKII genes for cloning into eukaryotic
expression vector pcDNA4/HisMax C.

| | | Amplified |
|---|---|---|
| Primer | 5′-3′ nucleotide sequences | fragment |
| TKI for | ATA GGA TCC ATG GCC TCG TAC | TKI gene |
| BamHI | CCC GGC CAT C (SEQ ID No: 120) | |
| TKI rev | ATA GAA TTCTTA TCA CAT CTC ACG | |
| EcoRI | GGC AAA CGT GC (SEQ ID No: 121) | |
| TKII for | ATA GAA TTCATG GCT TCT CAC | TKII gene |
| EcoRI | GCC GGC CAA C (SEQ ID No: 122) | |
| TKII rev | ATA CTC GAGTCA CTA AAC TCC | |
| XhoI | CCC CAC CTC GCG (SEQ ID No: 123) | |

Recognition site for BamHI, EcoRI or Xhol restriction endonuclease respectively is underlined.

Cloning of from HSV-1 and HSV-2 Thymidine Kinase Genes in Mammalian Expression Vector pcDNA4/HisMax C.

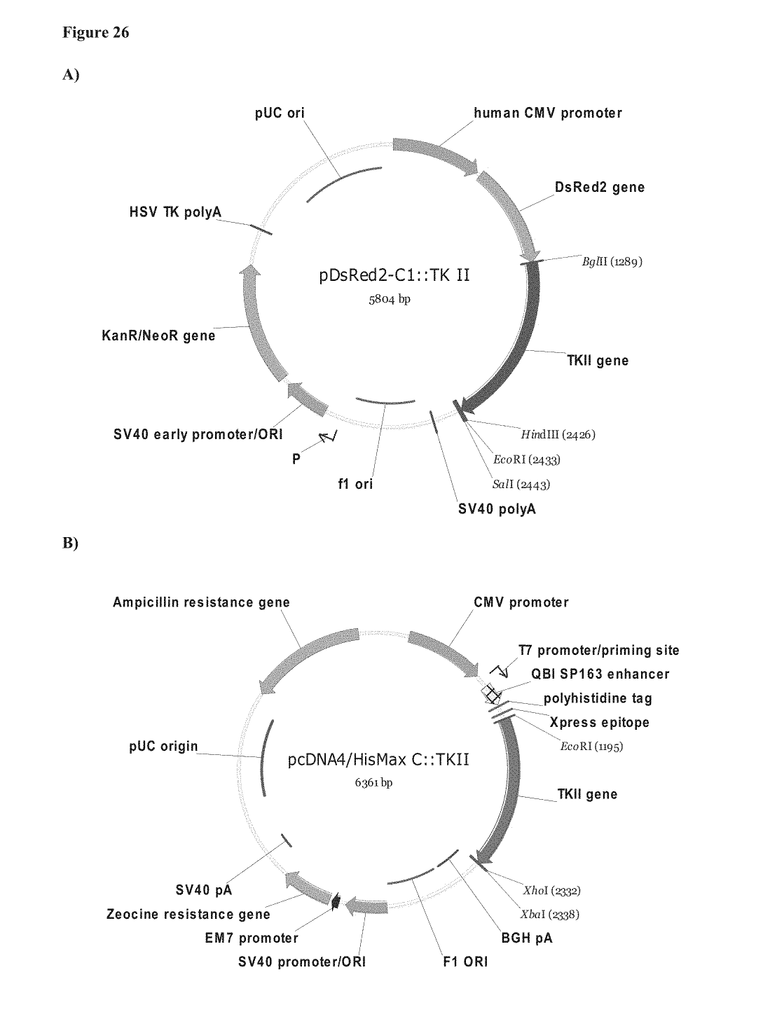

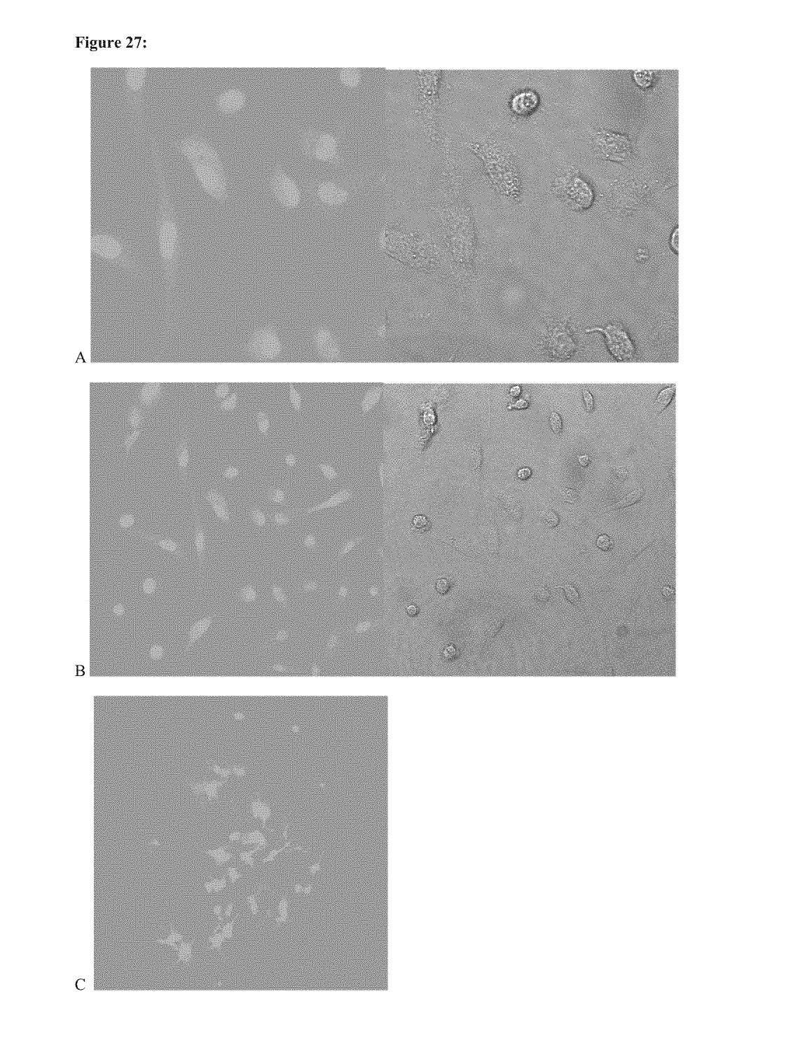

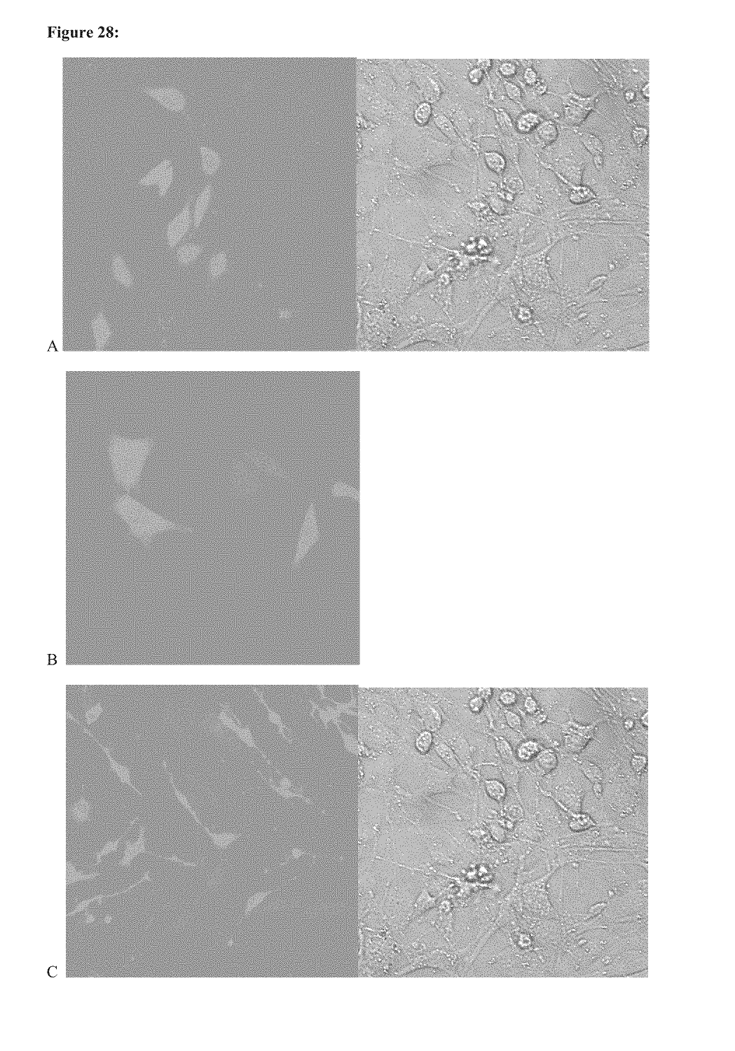

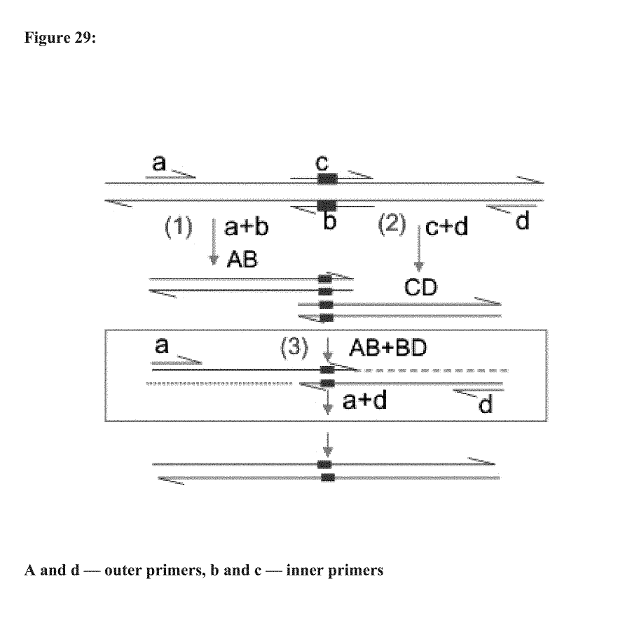

In order to compare the activities of HSV1 and HSV2 thymidine kinase enzymes in mammalian cells and choose the best enzyme for further use in GCV-based vaccines for breast cancer treatment we previously cloned both HSV1 and HSV2 thymidine kinase genes in mammalian expression vector pcDNA4/HisMax C, allowing high-level expression in most mammalian cell lines, purification and detection of expressed recombinant proteins (see report for 2007). But further sequence analysis of the cloned HSV2 thymidine kinase gene (GenBank accession number EF522120) and deduced TK2 protein sequence revealed the presence of several nucleotide substitutions in our TK2 gene (SEQ ID No: 87), leading to changes in amino acid sequence of TK2, as compared to the majority of HSV2 thymidine kinase protein sequences presented in GenBank. In fact, these substitutions may not be critical for TK2 function and impair its function, but may represent (at least some of them) another naturally occurring rare TKII variant (polymorphism). Nevertheless, we decided to secure ourselves against mistakes and to obtain more frequently occurring “classic” TK2 protein sequence, to compare the activities of different TKII variants and choose the best one for further use in vaccine development. In order to correct our TK2 sequence we used the PCR based site-specific mutagenesis approach (FIG. 29). This approach is based on use of two pairs of primers specifically annealing to the sequence of interest: inner and outer pairs. Fully complementary to each other inner primers contain the correct sequence variant and anneal to the region, overlapping the position of single point mutation in original nucleotide sequence, which is necessary to replace. Outer primers anneal to the ends of the correcting sequence. On the first stage two fragments of the sequence of interest are amplified by PCR: the first one is restricted by forward outer primer—reversed inner primer and the second is restricted by forward inner—reversed outer primers. Both PCR products now contain the corrected sequence variant and possess identical region corresponding to inner primer pair. On the second stage the obtained gel-purified primary PCR products are mixed in equal amounts and used in assembly reaction, where after denaturation they anneal to each other by complementary inner primer parts and prime fill in-assembly reaction in the presence of DNA polymerase. Adding of outer primers pair to the reaction leads to PCR amplification of the corrected mutation-free variant of the assembled sequence. In order to correct all nucleotide substitutions in our TK2 sequence we performed several cycles of site-specific mutagenesis. Lists of inner and outer primers used are shown in figures. On the first round of site-specific mutagenesis we corrected nucleotides in positions mut2 (T→G), mut6 (A→G) and mut7 (A→G), that caused corresponding amino acid substitutions F→L in protein sequence. On the second round of site-specific mutagenesis we corrected nucleotide in position mut3 (G→A), that caused corresponding amino acid substitution A→T in protein sequence. On the third round of site-specific mutagenesis we corrected nucleotide in position mut4 (G→A), that caused corresponding amino acid substitution E→K in protein sequence. The corrected sequence was cloned in pcDNA4/HisMax C expression vector. Sequences obtained after each round of site-specific mutagenesis were verified by sequencing. At the same time, it appeared that each round of PCR amplification of the entire TK2 sequence led to segregation of finally obtained sequences differing in length of the mut 5 region. The majority of the obtained amplified TK2 sequences were characterized by arising of short 2-30 bp deletions in this region. This region contains the nucleotide sequence 5′-gaccccgcgccccgaccccga-3′ which is characterized by triple repeat of identical ccccg sequence. We may speculate that in this region DNA polymerase can jump from one identical sequence to neighboring one, it can cause short deletions. In order to obtain more stable TK2 sequence we decided to replace three C nucleotides inside this sequence by G by site-specific mutagenesis, leading to 5′-gaccccgcggccggacccgga-3′ sequence in this region. Such nucleotide substitutions did not lead to amino acid replacements due to the partially degenerative genetic code. Finally, the obtained corrected nucleotide sequence of TK2 gene, encoding protein sequence identical to the several TK2 sequences in presented in GenBank Database was cloned in EcoRl/Xhol sites of pcDNA4/HisMax C expression vector. After that, it was reamplified using primers, containing Bg/II and Hind/III sites, and recloned in Bg/II/HindIII sites of vectors pDsRed2-C1, pTurboGFP-C, pTurboGFP-N, pJRed-C and pJRed-N. Thus, fusions of corrected TK2 gene with genes of reporter fluorescent proteins DsRed2, TurboGFP and JRed, allowing visual control of TK2 protein expression and distribution inside the mammalian cells, were obtained. The obtained corrected TK2 gene was also subjected to one more cycle of site-specific mutagenesis in order to correct nucleotide in position mut1 (G→A), that caused corresponding amino acid substitution D→N in protein sequence. Thus, we finally obtained the corrected nucleotide sequence of TK2 gene, encoding protein sequence identical to the majority of TK2 sequences in GenBank Database. This TK2 sequence was sequentially cloned in pcDNA4/HisMax C expression vector and in colour vectors pDsRed2-C1, pTurboGFP-C, pTurboGFP-N, pJRed-C and pJRed-N.

Initial sequence of TK2 gene. Nucleotides different from the majority of TK2 sequences in GenBank are shown underlined. Correct variants are shown in above therefrom. Problem region is indicated. Nucleotides modified inside the problem region are shown underlined.

| (SEQ ID No: 87) | |
|---|---|
| 1 | ATGGCTTCTC ACGCCGGCCA ACAGCACGCG CCTGCGTTCG |
| | GTCAGGCTGC TCGTGCGAGC GGGCCTACCG ACGGCCGCGC GGCGTCCCGT |
| | CCTAGCCATC |
| | BamHI |
| | A (mut4) ~~~~~~~ |
| 101 | GCCAGGGGGC CTCCGAAGCC CGCGGGGATC CGGAGCTGCC |
| | CACGCTGCTG CGGGTTTATA TAGACGGACC CCACGGGGTG GGGGAGACCA |
| | CCACCTCCGC |
| | PvuII |
| | ~~~~~~ A (mut1) |
| 201 | GCAGCTGATG GAGGCCCTGG GGCCGCGCGA CGATATCGTC |
| | TACGTCCCCG AGCCGATGAC TTACTGGCAG GTGCTGGGGG CCTCCGAGAC |
| | CCTGACGAAC |
| 301 | ATCTACAACA CGCAGCACCG TCTGGACCGC GGCGAGATAT |
| | CGGCCGGGGA GGCGGCGGTG GTAATGACCA GCGCCCAGAT AACAATGAGC |
| | ACGCCTTATG |
| | ApaI |
| | ~~~~~~~ G (mut2) |
| 401 | CGGCGACGGA CGCCGTTTTT GCTCCTCATA TCGGGGGGGA |
| | GGCTGTGGGC CCGCAAGCCC CGCCCCCGGC CCTCACCCTT GTTTTCGACC |
| | GGCACCCTAT |
| 501 | CGCCTCCCTG CTGTGCTACC CGGCCGCGCG GTACCTCATG |
| | GGAAGCATGA CCCCCCAGGC CGTGTTGGCG TTCGTGGCCC TCATGCCCCC |
| | GACCGCGCCC |
| | SmaI |
| | ~~~~~~~ |
| 601 | GGCACGAACC TGGTCCTGGG TGTCCTTCCG GAGGCCGAAC |
| | ACGCCGACCG CCTGGCCAGA CGCCAACGCC CGGGCGAGCG GCTTGACCTG |
| | GCCATGCTGT |
| | PstI |
| | ~~~~~~~ |
| 701 | CCGCCATTCG CCGTGTCTAC GACCTACTCG CCAACACGGT |
| | GCGGTACCTG CAGCGCGGCG GGAGGTGGCG GGAGGACTGG GGCCGGCTGA |
| | CGGGGGTCGC |
| | Problem region (mut 5) |
| | G G G |
| 801 | CGCGGCGACC CCGCGCCCCG ACCCCGAGGA CGGCGCGGGG |
| | TCTCTGCCCC GCATCGAGGA CACGCTGTTT GCCCTGTTCC GCGTTCCCGA |
| | GCTGCTGGCC |
| 901 | CCCAACGGGG ACTTGTACCA CATTTTTGCC TGGGTCTTGG |
| | ACGTCTTGGC CGACCGCCTC CTTCCGATGC ATCTATTTGT CCTGGATTAC |
| | GATCAGTCGC |
| | A (mut3) |
| 1001 | CCGTCGGGTG TCGAGACGCC CTGTTGCGCC TCACCGCCGG |
| | GATGATCCCA GCCCGCGTCA CAACCGCCGG GTCCATCGCC GAGATACGCG |
| | ACCTGGCGCG |
| | G(mut6) G(mut7) |
| 1101 | CACGTTTGCC CGCGAGATGG GGGAAGTTTA G |

List of used inner primers with corrected nucleotides:

TK2 for mut1 D-N

(SEQ ID No: 124)

5′-CCG CGC GAC AAT ATC GTC TAC-3′
TK2 rev mut1 D-N

(SEQ ID No: 125)

5′-GTA GAC GAT ATT GTC GCG CGG-3′
TK2 for mut2 F-L

(SEQ ID No: 126)

5′-GAC GCC GTT TTG GCT CCT C-3′
TK2 rev mut2 F-L

(SEQ ID No: 127)

5′-GAG GAG CCA AAA CGG CGT C-3′
TK2 for mut3 A-T

(SEQ ID No: 128)

5′-GAT GAT CCC AAC CCG CGT CAC-3′
TK2 rev mut3 A-T

(SEQ ID No: 129)

5′-GTG ACG CGG GTT GGG ATC ATC-3′
TK2 for mut4 D-N

(SEQ ID No: 130)

5′-GGT GGG GAA GAC CAC CAC CTC-3′
TK2 rev mut4 D-N

(SEQ ID No: 131)

5′-GAG GTG GTG GTC TTC CCC ACC-3′
TK2 for mut5

(SEQ ID No: 132)

5′-GGC CGG ACC CGG AGG ACG GCG CGG GGT C-3′
TK2 rev mut5

(SEQ ID No: 133)

5′-GTC CTC CGG GTC CGG CCG CGG GGT CGC CGC GGC
GAC-3′

List of used outer primers.
I. For cloning in pcDNA4/HisMax C vector:

TK 2-10 For EcoRI

(SEQ ID No: 134)

5′-ATA GAA TTC ATG GCT TCT CAC GCC GGC CAA C-3′
TK 2-10 Rev Xhol-2Tr

(SEQ ID No: 135)

5′-ATA CTC GAG TCA CTA AAC TCC CCC CAC CTC GCG-3′

II. For cloning in pJRed2-C; pTurboGFP-C; pDsRed2-C1 vectors:

TK2-10 For BgIII

(SEQ ID No: 136)

ATA AGA TCT ATG GCT TCT CAC GCC GGC CAA C-3′
TK 2-10 Rev HindIII-2Tr

(SEQ ID No: 137)

5′-AAT AAA GCT TTC ACT AAA CTC CCC CCA CCT CGC
G-3′

III. For cloning in pJRed2-N; pTurboGFP-N vectors:

TK2-10 For BglII N

(SEQ ID No: 138)

ATA AGA TCT CAT GGC TTC TCA CGC CGG CCA AC-3′
TK 2-10 Rev HindIII-2Tr

(SEQ ID No: 139)

5′-AAT AAA GCT TTC ACT AAA CTC CCC CCA CCT CGC G-
3′

Enhancement of DF3 Promoter.

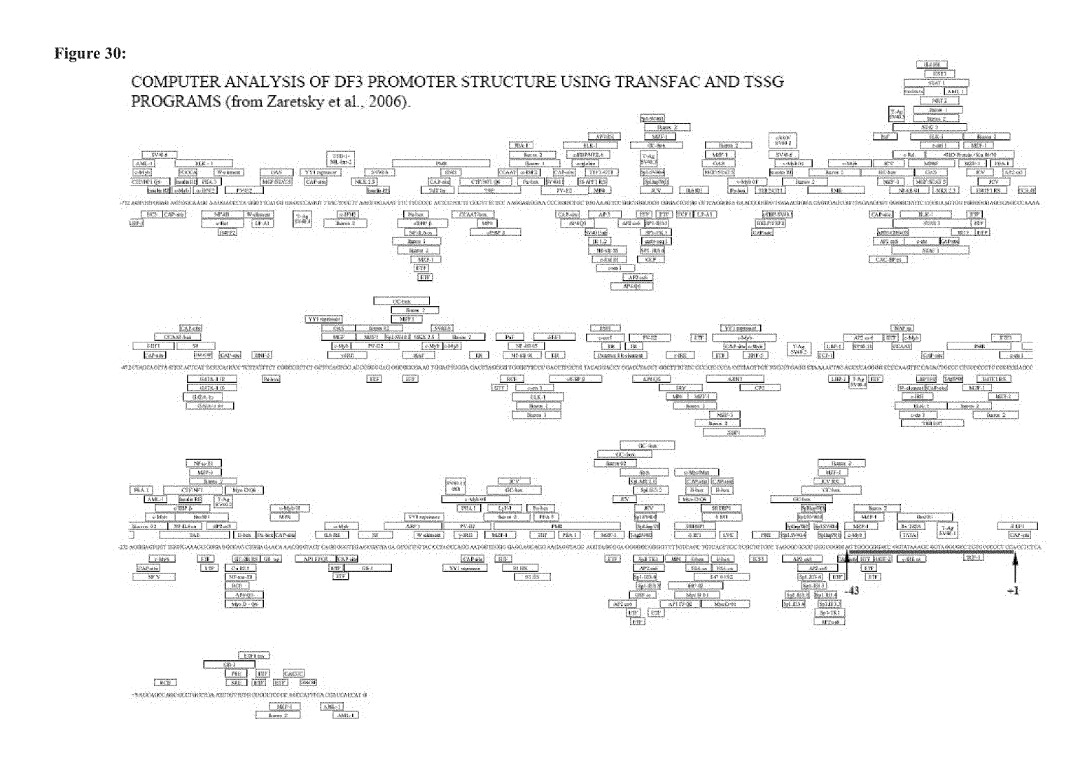

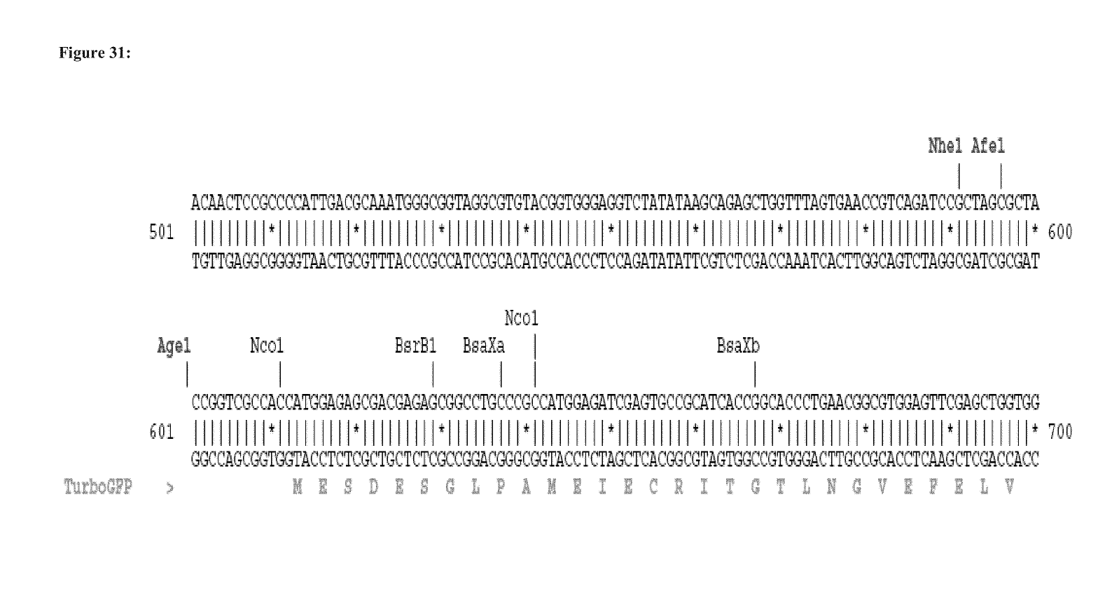

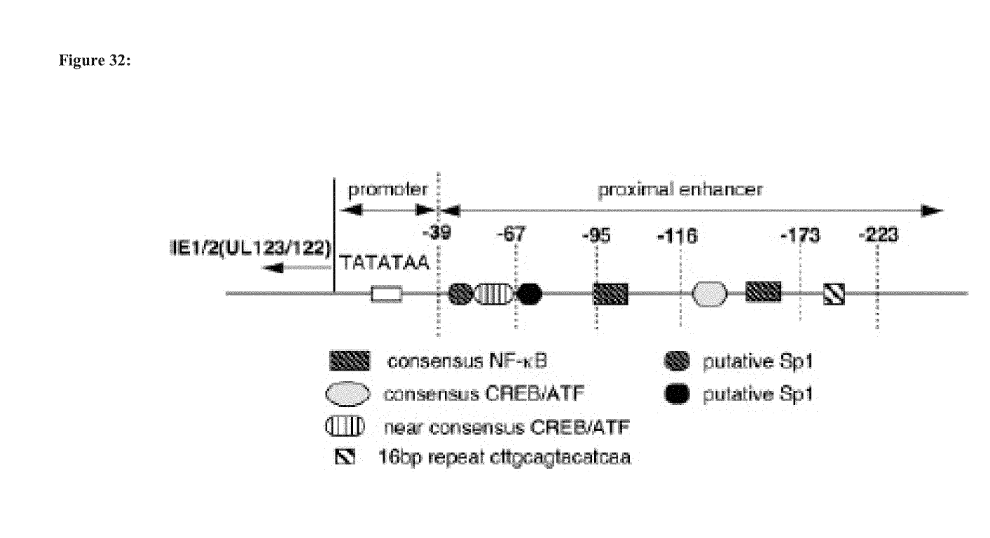

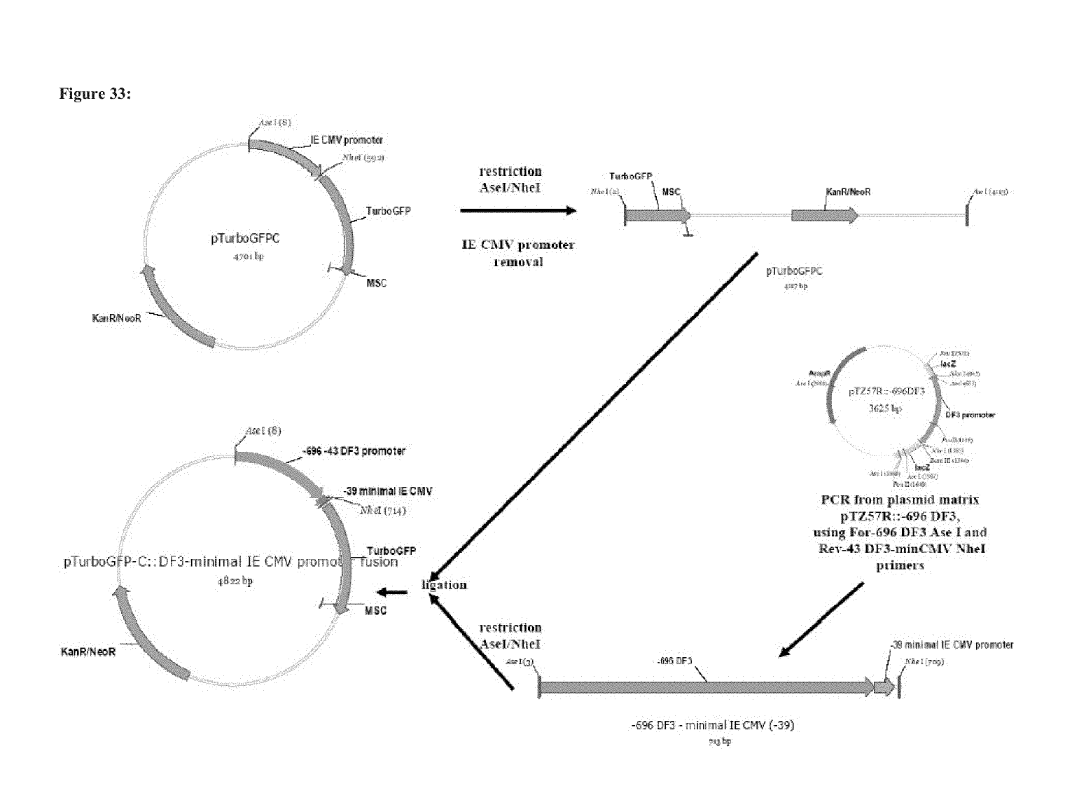

The MUC1 (DF3) gene encodes mucin glycoprotein which is basally expressed in most epithelial cells on their apical surface. At the same time, it is highly overexpressed in human breast cancer cells that make MUC1 protein valuable as a marker in breast cancer diagnostics and prognosis. Moreover, Muc1 expression correlates with the degree of breast tumor differentiation. It is known, that the expression of Muc1 gene is regulated at the transcriptional level by its complex tissue specific promoter (DF3 promoter). This characteristic makes DF3 promoter of great importance for use in development of vaccines for breast cancer treatment. Previously we PCR amplified −696-+31 region of DF3 promoter, using genomic DNA from the cells of human hormone-dependent carcinoma T47D as matrix, and cloned the obtained promoter in pDsRed2-C1 vector (see report for 2006). Cloned DF3 promoter part provided site-specific manner of expression of reporter protein DsRed2 in human breast adenocarcinoma MCF-7 and carcinoma T47D cell lines. At the same time, DF3 promoter appeared to be rather weak, that is characteristic feature of the majority of tissue-specific promoters: expression of reporter protein pDsRed2 under control of DF3 promoter became visible only after 36-48 hours after transfection and tend to decay after rather shot period of time, while in control pDsRed2-C1 vector, where pDsRed2 is under control of strong CMV promoter, the corresponding time of visible DsRed2 expression start was 20-24 hours. Thus, for effective use of the remarkable tissue-specificity of DF3 promoter in breast cancer vaccine development it was necessary to modify DF3 promoter sequence so, that it would acquire features of strong promoters, but, at the same time, retain its tissue-specificity. Computer analysis of DF3 promoter sequence performed by Zaretsky et al., 2006 revealed the extreme complexity of the fine structure of DF3 promoter (FIG. 30), containing numerous overlapping binding sites for transcription regulators and other regulatory proteins. Thus, due to the lack of information of influence of these regulators on the work of DF3 promoter and their interaction with each other, we decided to begin DF3 modification with more easy procedure—modification of its TATA box part, lying close to transcription start point. We decided to replace the original TATA box of DF3 with the corresponding part of strong CMV promoter. From the literature data it is known, that containing TATA box of CMV promoter is defined as region between positions −39 and −1. It is also known, that recombinant CMV virus with the proximal promoter deleted to −39 and retaining only the minimal TATA box promoter element, cannot replicate independently in human fibroblast cells. Thus, we can use this minimal CMV promoter element for enhancement of DF3 promoter, and not be afraid of transcription “leakage” from this minimal CMV TATA box. In order to obtain DF3-minimal CMV (TATA box) promoter fusion we performed PCR using −696 DF3 Asel forward and special −43 DF3-minCMV Nhel reverse primer, containing the “hybrid” DF3-minimal CMV sequence. Previously cloned DF3 promoter sequence was used as matrix for PCR. The obtained PCR product, representing the “hybrid” promoter sequence, was sequenced, double digested with Asel and Nhel restriction endonucleases and gel-purified. CMV promoters were excised from cloning vectors pJRed-C, pJRed-N, pTurboGFP-C, pTurboGFP-N, pDsRed2-C1 by Asel and Nhel restriction. The remaining promoterless vector parts were gel-purified and ligated with the “hybrid” DF3-minimalCMV promoter sequence.

Thus, the following plasmid constructions were finally obtained: pTurboGFP-C:: −696 DF3-minimal IE CMV promoter fusion pTurboGFP-N:: −696 DF3-minimal IE CMV promoter fusion pJRed-C:: −696 DF3-minimal IE CMV promoter fusion pJRed-N:: −696 DF3-minimal IE CMV promoter fusion pDsRed2-C1:: −696 DF3-minimal IE CMV promoter fusion

In all these constructions reporter fluorescent proteins were placed under the control of “hybrid” promoter. We also cloned the original DF3 promoter, which was previously cloned only in pDsRed2-C1 vector, in pJRed-C, pJRed-N, pTurboGFP-C, pTurboGFP-N vectors on the place of CMV in order to have all set of positive controls. In order to have negative controls we also obtained plasmid constructions pTurboGFP-C:: −39 minimal IE CMV promoter and pDsRed2-C1:: −39 minimal IE CMV promoter, containing only minimal −39 CMV promoter part upstream the reporter protein. The obtained plasmid constructions were sent to Cell Biology Group and used for transfection of several cell lines: MCF-7, T47D, ZR-75-1, CHO-K1, U-937, MT-2. The results of two preliminary transfection experiments are shown in tables 1 and 2. The obtained results showed that “hybrid” DF3-minCMV promoter retained the tissue specificity of the original DF3 promoter and, at the same time, it performed at least 2.5 higher expression rate of the reporter fluorescent proteins then DF3 (but it value was about 30% lower then for entire CMV promoter). Time interval preceding the appearance of visible fluorescence of the reporter protein (after the moment of transfection of the cells with plasmid constructions) under control of “hybrid” DF3-minCMV promoter was similar to the corresponding time for entire CMV promoter (20-24 hours) in contrast to original DF3 promoter (36-48 hours). The summary of “hybrid” DF3-minCMV promoter features and its comparison with CMV and DF3 promoters is shown in table 6. Thus, we fulfilled our task and enhanced DF3 promoter without loss of its tissue specificity.

Primers used for construction of −696 DF3-−39−1 minimal CMV promoter fusion by PCR.

For-696 DF3 AseI Asel=VspI

(SEQ ID No: 140)

5′-AAA TTA ATG GAC CCT AGG GTT CAT CGG AGC-3′

Rev-43 DF3 min CMV Nhel

(SEQ ID No: 141)

5′-TTA TGC TAG CGG ATC TGA CGG TTC ACT AAA CCA
GCT CTG CTT ATA TAG ACC TCC CAC TCC CCG CCC GCC
CGC CCT AGG C-3′

Rev-43 DF3 minCMV Nhel reverse complement sequence

(SEQ ID No: 142)

−43 DF3 −39 min IE CMV (TATA box)
5′-GCC TAG GGC GGG CGG GCG GGG AGT GGG AGG TCT ATA
TAA GCA GAG CTG
−1+1 (transcription start)
GTT TAG TGA ACC GTC AGA TCC GCT AGC ATA A-3′
NheI

### REFERENCES

1. “Aromatase Inhibitors” 2^{nd} revised edition, edited by Barrington J. A., Furr, 2008, pub. Birkhäuser: Howell A., Wakeling A. “Clinical studies with anastrozole” pp.110-111;
2. Baker M K, Mikhitarian K, Osta W, Callahan K, Hoda R, Brescia F, Kneuper-Hall R, Mitas M, Cole D J, Gillanders W E “Molecular protection of breast cancer cells in the peripheral blood of advanced-stage breast cancer patient susing multimarker real-time reverse transcription-polymerase chain reaction and a novel porous barrier density gradient centrifugation technology” J. Clinical Cancer Research, 2003 Oct. 15, 9, pp. 4865-4871;
3. Balleine R L, Hunt S M N, Clarke C L “Coexpression of alternatively spliced estrogen and progesterone receptor transcripts in human breast cancer” J Clin Endocrinol Metab. 1999, 84:1370-1377;
4. Bamdat C. C. “Techniques and compositions of diagnostic treatment of cancer” AU 2009213107 A1 Minerva Biotech. Corp. 08.10.2009;
5. Brayman M, Thathiah A, Carson D D (January 2004). “MUC1: a multifunctional cell surface component of reproductive tissue epithelia” Reprod Biol Endocrinol. 2004; 2: 4, doi:10.1186/1477-7827-2-4;
6. Castiglioni F., Tagliabue E., Campiglio M., Pupa S. M., Balsaril A., Menard S. “Role of exon-16-deleted HER2 in breast carcinomas”, Endocrine-Related Cancer, 2006, 13, pp.221-232;
7. Cigna Medical coverage policy: “Genetic Expression Assays for Breast Cancer Prognosis-Oncotype DX™ Breast Cancer Assay” Effective Date Nov. 15, 2011, Coverage Policy Number 0298;
8. Cork D. M. W., T W J Lennard, A J Tyson-Capper “Alternative splicing and the progesterone receptor in breast cancer” 2008, Breast Cancer Research, 10:207
9. Filinova E, Anashkina E. “The method for in vitro study of early pharmacocytokinetis of medicines” RU patent, 2007, IPC A 61 B 1/00, G 01N13/14, RU 2007113034/15;
10. Gendler S J, Lancaster C A, Taylor-Papadimitriou J, Duhig T, Peat N, Burchell J, Pemberton L, Lalani E N, Wilson D (September 1990). “Molecular cloning and expression of human tumor-associated polymorphic epithelial mucin”. J. Biol. Chem. 265 (25): 15286-93;
11. Gendler S J (July 2001). “MUC1, the renaissance molecule”. J. Mammary Gland Biol Neoplasia 6 (3): 339-353;
12. Grupe A. “Molecular Insight into Metastatic Potential and Response to Tamoxifen in Breast Cancer” Celera Multiplex, Celera, ADAPT 2010, Arlington, Sep. 13-16, 2010;
13. Iverson A A, Gillett C, Cane P, Santini C D, Vess T M, Kam-Morgan L, Wang A, Eisenberg M, Rowland C M, Hessling J J, Broder S E, Sninsky J J, Tutt A, Anderson S, Chang S Y. “A single-tube quantitative assay for mRNA levels of hormonal and growth factor receptors in breast cancer specimens” J Mol Diagn. 2009 March; 11(2):117-30;
14. Julian J, Carson D D (May 2002). “Formation of MUC1 metabolic complex is conserved in tumor-derived and normal epithelial cells”. Biochem Biophys Res Commun 293 (4): 1183-1190;
15. Hattrup C L, Gendler S J. “MUC1 alters oncogenic events and transcription in human breast cancer cells” Breast Cancer Res. 2006, 8(4):R37;
16. Hattrup C L, Gendler, S J (2008). “Structure and Function of the Cell Surface (Tethered) Mucins”. Annu. Rev. Physiol. 70: 431-457;
17. Herynk M H and Fuqua S A W “Estrogen Receptor Mutations in Human Disease” Endocr. Rev. 2004, 25:869-898;
18. Hollingsworth M A, Swanson B J “Mucins in cancer: protection and control of the cell surface”. Nat. Rev. Cancer, Jan. 2004, 4 (1): 45-60;
19. Ho S B, Niehans G A, Lyftogt C et al. “Heterogeneity of mucin gene expression in normal and neoplastic tissues” Cancer Res. 1993 Feb. 1; 53(3): 641-51;
20. Huang L, Chen D, Liu D, Yin L, Kharbanda S, Kufe D “MUC1 oncoprotein blocks glycogen synthase kinase 3beta-mediated phosphorylation and degradation of beta-catenin”. Cancer Res Nov. 2005, 65 (22): 10413-10422.
21. Kimura T, Finn O J. “MUC1 immunotherapy is here to stay”, Expert Opin Biol Ther. 2013 January; 13(1):35-49;
22. Kufe D. W. “MUC1, Caspase-8 and DED-containing proteins” Appl. Dana-Farber Cancer Institute, 25.08.2011, US 2011/0207656 A1;
23. Linden, S K; Sheng Y H, Every A L, Miles K M, Skoog E C, Florin T H, Sutton P, McGuckin M A (October 2009). Van Nhieu, Guy Tran. ed. “MUC1 limits Helicobacter pylori infection both by steric hindrance and by acting as a releasable decoy”. PLoS Pathog. 5 (10): el 000617;
24. Linke S. P., Bremer T. M., Diamond C. A. “Diagnostic Markers of Breast Cancer Treatment and Progression and Methods of use thereof” Appl. Prediction Sciences LLC, 07.01.2010, WO 2010/002367 A1;
25. Li, Y; Bharti A, Chen D, Gong J, Kufe D (December 1998). “Interaction of glycogen synthase kinase 3beta with the DF3/MUC1 carcinoma-associated antigen and beta-catenin”. Mol. Cell. Biol. (UNITED STATES) 18 (12): 7216-24;
26. Li, Y.; Chen W., Ren J., Yu W-H., Li Q., Yoshida K., Kufe D. (2003). “DF3/MUC1 signaling in multiple myeloma cells is regulated by interleukin-7”. Cancer Biol. Ther. (United States) 2 (2): 187-93;
27. Li, Y; Kuwahara H, Ren J, Wen G, Kufe D. (March 2001). “The c-Src tyrosine kinase regulates signaling of the human DF3/MUC1 carcinoma-associated antigen with GSK3 beta and beta-catenin”. J. Biol. Chem. (United States) 276 (9): 6061-4;
28. Li, Y; Ren J, Yu W, Li Q, Kuwahara H, Yin L, Carraway K L, Kufe D (September 2001). “The epidermal growth factor receptor regulates interaction of the human DF3/MUC1 carcinoma antigen with c-Src and beta-catenin”. J. Biol. Chem. (United States) 276 (38): 35239-42;
29. Leygue E., Dotzlaw H., Watson P H, Murphy L C. “Altered expression of exon 6 deleted progesterone receptor variant mRNA between normal human breast and breast tumour tissues”, British Journal of Cancer, 1999, 80(3/4), 379-382.
30. Lluch, A., Staroslawska, E., de la Haba-Rodriguez,J., Im, S. A., Pedrini, J. L., Poirier, B., Morandi, P., Semiglazov, V., Srimuninnimit, V., Bianchi, G., Szado, T., Ratnayake, J., Ross, G. Valagussa, P. “Efficacy and safety of neoadjuvant pertuzumab and trastuzumab in women with locally advanced, inflammatory, or early HER2-positive breast cancer (NeoSphere): a randomised multicentre, open-label, phase 2 trial”, JOURNAL Lancet Oncol. 13 (1), 25-32 (2012);
31. Moase E H, Qi W, Ishida T, Gabos Z, Longenecker B M, Zimmermann G L, Ding L, Krantz M, Allen T M. “Anti-MUC1 immunoliposomal doxorubicin in the treatment of murine models of metastatic breast cancer”, Biochim Biophys Acta. 2001 Feb. 9; 1510(1-2):43-55;
32. http://en.wikipedia.org/wiki/MUC1
33. NCBI database maps for primers tailoring information: http://www.ncbi.nlm.nih.gov/gene/4582; http://www.ncbi.nlm.nih.gov/gene/2099; http://www.ncbi.nlm.nih.gov/gene/2064; http://www.ncbi.nlm.nih.gov/gene/5241; http://www.ncbi.nlm.nih.gov/gene/174; http://www.ncbi.nlm.nih.gov/gene/367;
34. Nicolini A., Giardino R., Carpi A, Ferrari P, Anselmi L., Colosimo S., Contee M., Fini M., Giavaresi G., Berti P., Miccoli P. “Metastatic breast cancer: an updating” Biomedicine & Pharmacotherapy 60 (2006) 548-556;
35. Obermair A., Bernd I., Schmid C., Stimpfl M., Ph.D., Fasching B., Preyer O, Leodolter S., Crandon A. J., Zeillinger R. “Novel MUC1 Splice Variants Are Expressed in Cervical Carcinoma” Gynecologic Oncology 2001, 83, 343-347);
36. Pichinuk E, Benhar I, Jacobi O, Chalik M, Weiss L, Ziv R, Sympson C, Karwa A, Smorodinsky N I, Rubinstein D B, Wreschner D H. “Antibody targeting of cell-bound MUC1 SEA domain kills tumor cells. Cancer Res. 2012 Jul 1; 72(13):3324-36;
37. Raina D, Kharbanda S, Kufe D (May 2004). “The MUC1 oncoprotein activates the anti-apoptotic phosphoinositide 3-kinase/Akt and Bcl-xL pathways in rat 3Y1 fibroblasts”. J Biol Chem 279 (20): 20607-20612;
38. Ren J, Bharti A, Raina D, Chen W, Ahmad R, Kufe D (January 2006). “MUC1 oncoprotein is targeted to mitochondria by heregulin-induced activation of c-Src and the molecular chaperone HSP90”. Oncogene 25 (1): 20-31;
39. Ren, Jian; Li Yongqing, Kufe Donald (May. 2002). “Protein kinase C delta regulates function of the DF3/MUC1 carcinoma antigen in beta-catenin signaling”. J. Biol. Chem. (United States) 277 (20): 17616-22;
40. Roy L D, Sahraei M, Subramani D B, Besmer D, Nath S, Tinder T L, Bajaj E, Shanmugam K, Lee Y Y, Hwang S I L, Gendler S J, Mukherjee P (March 2011). “MUC1 enhances invasiveness of pancreatic cancer cells by inducing epithelial to mesenchymal transition”. Oncogene 30 (12): 1449-1459;
41. Schroeder, Joyce A; Adriance C, Thompson M C, Camenisch Todd D, Gendler Sandra J (March 2003). “MUC1 alters beta-catenin-dependent tumor formation and promotes cellular invasion”. Oncogene (England) 22 (9): 1324-32. doi:10.1038/sj.onc.1206291. ISSN 0950-9232. PMID 12618757.
42. Schroeder, J A; Thompson M C, Gardner M M, Gendler S J (April 2001). “Transgenic MUC1 interacts with epidermal growth factor receptor and correlates with mitogen-activated protein kinase activation in the mouse mammary gland”. J. Biol. Chem. (United States) 276 (16): 13057-64;
43. Siegel P M, Ryan E D, Cardiff R D, Muller W J 1999. Elevated expression of activated forms of Neu/ErbB-2 and Erb-3 are involved in the induction of mammarytumors in transgenic mice: implications for human breast cancer. EMBO Journal 18 2149-2164;
44. Singh P K, Hollingsworth M A (August 2006). “Cell surface-associated mucins in signal transduction”. Trends Cell Biol 16 (9): 467-476;
45. Springwald A, Lattrich C, Skrzypczak M, Goerse R, Ortmann O, Treeck O. “Identification of novel transcript variants of estrogen receptor α, β and progesterone receptor gene in human endometrium” Endocrine 2010, 37(3):415-24;
46. Tang C-K., Katsara M., Apostolopoulos V. “Strategies used for MUC1 immunotherapy: human clinical study” Expert Reviews Vaccines, 2008, 7(7):963-975;
47. Tarp M. A, Clausen H. “Mucin-type O-glycosylation and its potential use in drug and vaccine development” Biochimica et Biophysica Acta 1780 (2008) 546-563;
48. Wei X, Xu H, Kufe D “Human MUC1 oncoprotein regulates p53-responsive gene transcription in the genotoxic stress response”. Cancel Cell, Feb. 2005, 7 (2): 167-178;
49. Wei X, Xu H, and Kufe D. “MUC1 Oncoprotein Stabilizes and Activates Estrogen Receptor α” Molecular Cell, Jan. 20, 2006, 21, 295-305;
50. Wolman S R, Pauley R J, Mohamed A N, Dawson P J, Visscher D W, Sarkar F H. “Genetic markers as prognostic indicators in breast cancer” Cancer, 1992 Sep. 15; 70 (6 Suppl): 1765-74;
51. Zaretsky J Z, Sarid R, Aylon Y, Mittelman L A, Wreschner D H, Keydar I. “Analysis of the promoter of the MUC1 gene overexpressed in breast cancer” FEBS Letters 461 (1999) 189-195;
52. Zaretsky J Z, Barnea I, Aylon Y, Gorivodsky M, Wreschner D H and Keydar I. “MUC1 gene overexpressed in breast cancer: structure and transcriptional activity of the MUC1 promoter and role of estrogen receptor alpha (ERα) in regulation of the MUC1 gene expression” Molecular Cancer 2006, 5:57, 05 November 2006, doi:10.1186/1476-4598-5-57;
53. Q X, Hilsenbeck S G, Fuqua S A, Borg A. “Multiple splicing variants of the estrogen receptor are present in individual human breast tumors”. J Steroid Biochem 1996, Mol Biol 59:251-260.

1-40. (canceled)

41. An in vitro method for monitoring therapy and/or for adapting therapy of an epithelial cancer patient, who is subject to a cancer treatment, comprising:
(a) obtaining a tissue sample comprising cancer cells from said patient at a first time point,
(b) determining the expression level of
(i) total membrane-bound Muc1 1 mRNA, or
(ii) total membrane bound Muc1 protein in said tissue sample,
(c) determining the expression level of
(i) the long forms of Muc1 mRNA, or
(ii) the long forms of Muc1 protein in said tissue sample,
(d) determining the ratio between the expression levels of (b) and (c),
(e) repeating steps (a) to (d) at a second time point, which is at least 1 day later than the first time point, preferably at least 1 week later than the first time point, more preferably at least 1 month later than the first time point, even more preferably at least 3, 6, 9 or 12 months later than the first time point,
(f) comparing the ratio of expression levels determined at the first time point and the second time point,
wherein
an increase in ratio between the expression level of (b) and (c) at the second time point compared to the first time point indicates that
(i) the patient is less responsive to said cancer treatment, and
(ii) is responsive to Muc1 based therapy.

42. The method of claim 41, further comprising following steps:
(a1) determining the expression level of
(i) Her-2 mRNA,
(ii) Estrogen Receptor 1 (ER1) isotype 1 mRNA, and
(iii) Progesterone Receptor (PR) mRNA
in said tissue sample of said first time point (al),
(b 1) repeating steps (a1) at said second time point (b 1), which is at least 1 day later than said first time point, preferably at least 1 week later than said first time point, more preferably at least 1 month later than said first time point, even more preferably at least 3, 6, 9 or 12 months later than said first time point,
(c1) comparing the ratio of expression levels determined at said first time point (a1) and said second time point (b 1),
wherein
(i) an increase in ratio between the expression level of (b) and (c) of claim 41 at the second time point compared to the first time point, and
(ii) a decrease in expression level of Estrogen Receptor 1 (ER1) isotype alpha mRNA, and Progesterone receptor (PR) mRNA and optionally Her-2 mRNA at said second time point compared to said first time point,
indicates that the patient is less responsive to said cancer treatment, and is responsive to a Muc1 based therapy,
optionally
i) wherein the epithelial cancer is breast cancer, or
ii) wherein the tissue sample is a blood sample or a breast epithelium sample, or
iii) wherein the cancer treatment is chemotherapy, treatment with aromatase inhibitor(s), an hormone therapy, a treatment with at least one agent directed against HER-2, or a combination thereof,
in particular
wherein the combination is a combination therapy of chemotherapy and treatment with aromatase inhibitor(s), and/or
wherein treatment with aromatase inhibitor(s) is an adjuvant therapy, optionally
wherein hormone therapy is a treatment with at least one agent directed against Estrogen Receptor 1 (ESR1) isotype a and/or progesterone receptor (PR), or
iv) the epithelial cancer is selected from breast cancer, colon cancer, esophageal cancer, gastric cancer, lung cancer, melanoma, bladder cancer, ovarian cancer, prostate cancer and pancreatic cancer.

43. A method for determining malignancy grade or progression of a tumor of a patient suffering from an epithelial tumor, comprising:
(a) obtaining a tissue sample comprising tumor cells from said patient,
(b) determining the expression level of
(i) total membrane-bound Muc1 mRNA, or
(ii) total membrane bound Muc1 protein
in said tissue sample,
(c) determining the expression level of
(i) the long forms of Muc 1 RNA, or
(ii) the long forms of Muc1 protein
in said tissue sample,
wherein
an expression level of (b) higher than the expression level of (c) indicates (α) that said tissue sample is malignant, and/or (β) that the tumor has increased its malignancy grade, and/or (γ) that the patient is progressing and/or is less responsive to the currently applied tumor therapy,
optionally:
wherein
in addition the expression levels of 1, 2, or 3, preferably 3, of the following mRNAs is determined: (i) HER-2, (ii) Estrogen Receptor 1 (ESR1) isotype α, (iii) progesterone receptor (PR) mRNA, or
wherein the tissue is blood, or
wherein
a) said patient hast undergone breast cancer surgery, and/or
b) the cells are obtained after breast surgery, in particular after 2, 3, 6 or more months after breast surgery, and/or
c) the patient was confirmed to be HER-2-positive, Estrogen Receptor 1 (ESR1) isotype α-positive and/or progesterone receptor (PR)-positive and/or responsive to an agent directed against Her-2 or hormone therapy before or at the time of surgery, and/or
d) the patient was confirmed to be HER-2-negative, Estrogen Receptor 1 (ESR1) isotype α-negative and/or progesterone receptor (PR)-negative and/or non-responsive to an agent directed against Her-2 or hormone therapy before or at the time of surgery.

44. The method according to claim 41 or 43, wherein
(a) the long forms of Muc1 RNA are all Muc1 mRNA molecules encoding at least exons III to VII of Muc1 and/or encoding a Muc1 protein comprising up to 39 repeats in the variable number tandem repeat (VNTR) domain, or
(b) the long forms of Muc1 protein comprise at least a part of the variable number tandem repeat (VNTR) domain, or
(c) the expression level of a Muc1 RNA or Muc1 protein is the amount or concentration of the Muc1 RNA or Muc1 protein, which is preferably normalized, or
(d) the expression levels of total membrane-bound Muc1 mRNA and the long forms of Muc1 RNA are determined.

45. The method of claim 41, further comprising:
(g) repeating steps (a) to (f) of claim 41 further 1, 2, 3 or more times at a time point at least 1 day later than the respective previous repetition, preferably at least 1 week later than the respective previous repetition, more preferably at least 1 month later than the respective previous repetition, even more preferably at least 3, 6, 9 or 12 months later than the respective previous repetition,
(h) comparing the ratio of expression levels determined at the different time points, wherein
an increase in ratio between the expression level of (b) and (c) at a later time point compared to an earlier time point indicates that
(i) the patient is less responsive to said treatment, and
(ii) is responsive to Muc1 based therapy,
optionally
wherein the patient determined to be less responsive to said treatment, and to be responsive to a Muc1 based therapy, is determined to suffer from progressive disease, and/or is determined to be responsive to a chemotherapy treatment with high dosage and/or short intervals of chemotherapeutic agent(s) to be administered.

46. The method of claim 42, further comprising:
(i) repeating step (a1) of claim 42 further 1, 2, 3 or more times at a time point at least 1 day later than the respective previous repetition, preferably at least 1 week later than the respective previous repetition, more preferably at least 1 month later than the respective previous repetition, even more preferably at least 3, 6, 9 or 12 months later than the respective previous repetition,
(j) comparing the expression levels determined at the different time points, wherein
(i) an increase in ratio between the expression level of (b) and (c) of claim 41 at a later time point compared to an earlier time point, and
(ii) a decrease in expression level of Estrogen Receptor 1 (ER1) isotype 1 mRNA, and Progesterone receptor (PR) mRNA, and optionally Her-2 mRNA at a later time point compared to an earlier time point
indicates that the patient is less responsive to said treatment, and is responsive to a Muc1 based therapy,
optionally
wherein the patient determined to be less responsive to said treatment, and to be responsive to a Muc1 based therapy, is determined to suffer from progressive disease, and/or is determined to be responsive to a chemotherapy treatment with high dosage and/or short intervals of chemotherapeutic agent(s) to be administered.

47. The method of claim 43, further comprising:
(d) repeating steps (a) to (c) at a time point at least 1 day later, preferably at least 1 week later, more preferably at least 1 month later, even more preferably at least 3, 6, 9 or 12 months later,
wherein an increase in the difference (a)-(b) between the expression level of (a) total membrane-bound Muc1 mRNA, or total membrane bound Muc1 protein and (b) the expression level of the long forms of Muc1 RNA, or the long forms of Muc1 protein at the later time point compared to the earlier time point indicates that (α) that said tissue sample has become more malignant, and/or (β) that the tumor has further increased its malignancy grade, and/or (γ) that the patient is further progressing and/or is less responsive to the currently applied tumor therapy,
optionally wherein said method is further comprising:
(e) further repeating steps (a) to (c) further 1, 2, 3 or more times at a time point at least 1 day later than the respective previous repetition, preferably at least 1 week later than the respective previous repetition, more preferably at least 1 month later than the respective previous repetition, even more preferably at least 3, 6, 9 or 12 months later than the respective previous repetition,
wherein an increase in the difference (a)-(b) between the expression level of (a) total membrane-bound Muc1 1 mRNA, or total membrane bound Muc1 1 protein and (b) the expression level of the long forms of Muc1 1 RNA, or the long forms of Muc1 1 protein at a later time point compared to an earlier time point indicates that (α) that said tissue sample has become more malignant, and/or (β) that the tumor has further increased its malignancy grade, and/or (γ) that the patient is further progressing and/or is less responsive to the currently applied tumor therapy.

48. The method of claim 41 or 43, wherein the expression level(s) of each mRNA is determined by Real-time PCR.

49. The method according to claim 48,
(i) wherein in the context of Real-time PCR normalization is performed, and wherein normalization
(a) is not performed by normalization to the expression of a reference gene, and/or
(b) is performed by determining the total amount of RNA by spectrometry or fluorometry,
or
(ii) wherein Real-time PCR
a) is not performed as multiplex Real-time PCR, and/or
b) is performed by using a single primer pair per Real-time PCR reaction.

50. The method according to claim 48, comprising following steps for determining total membrane-bound Muc1 mRNA:
(a) isolating total RNA from the tissue sample,
(b) reverse transcribing the RNA into cDNA,
(c) performing Real-time PCR using one or more of the following primer pairs (i) to (xii) for determining total membrane-bound Muc1 1 mRNA:
(i)
(SEQ ID No. 1)
CCTCCCCACCCATTTCACC
and
(SEQ ID No. 2)
CTGTAAGCACTGTGAGGAGC
(ii)
(SEQ ID no. 3)
CCTACCATCCTATGAGCGAG
and
(SEQ ID No. 4)
CCCTACAAGTTGGCAGAAGTG
(iii)
(SEQ ID No. 5)
CTACTGAGAAGAATGCTTTGTCTA
and
(SEQ ID No. 6)
GCCTGAACTTAATATTGGAGAGG
(iv)
(SEQ ID No. 7)
CTACTGAGAAGAATGCTTTTAATTCC
and
(SEQ ID No. 8)
GCCTGAACTTAATATTGGAGAGG
(v)
(SEQ ID No. 9)
CTACTGAGAAGAATGCTTTGTCTA
and
(SEQ ID No. 10)
CTCTTGGTAGTAGTCGGTGC
(vi)
(SEQ ID No. 11)
(CCAGCACCGACTACTACCAA
or
(SEQ ID No. 13))
CACCGACTACTACCAAGAGC
and
(SEQ ID No. 12)
CTCTTGGTAGTAGTCGGTGC
(vii)
(SEQ ID No. 14)
CTACTGAGAAGAATGCTTTTAATTCC
and
(SEQ ID No. 15)
GCCTGAACTTAATATTGGAGAGG
(viii)
(SEQ ID No. 16)
CTACTGAGAAGAATGCTTTTAATTCC
and
(SEQ ID No. 17)
(CGGCACTGACAGACAGCCAT
or
(SEQ ID No. 18))
GGCACTGACAGACAGCCATT
(ix)
(SEQ ID No. 19)
CTACTGAGAAGAATGCTTTTAATTCC
and
(SEQ ID No. 20)
CACCCCAGCCCCAGACATT
(x)
(SEQ ID No. 21)
CTACTGAGAAGAATGCTTTTTTGC
and
(SEQ ID No. 22)
AGGCTGCTTCCGTTTTATACTG
(xi)
(SEQ ID No. 23)
CCTCTCCAATATTAAGTTCAGTGA
and
(SEQ ID No. 24)
ACAGACAGCCAAGGCAATGAG
(xii)
(SEQ ID No. 25)
(CCTCTCCAATATTAAGTTCAGTCT
or
(SEQ ID No. 26)
CCTCTCCAATATTAAGTTCAGTC)
and
(SEQ ID No. 27)
ACAGACAGCCAAGGCAATGAG,
and
(d) determining the expression level of total Muc1 mRNA,
optionally:
wherein in step (b), one or more of the primers according to SEQ ID No. 2, 4, 6, 8, 10, 12, 15, 17, 18, 20, 22, 24 and 27 are used for reverse transcribing the RNA into cDNA,
or
wherein following probes are used:
(a)
(SEQ ID No. 34)
TGACACCGGGCACCCAGTCTCC
and/or
(SEQ ID No. 35)
CCACCATGACACCGGGCACCCA
for primer pair (i)
(b)
(SEQ ID No. 36)
TGCAGGTAATGGTGGCAGCAGCC
for primer pair (ii)
(c)
(SEQ ID No. 37)
AGCACCGACTACTACCAAGAGCTGCA
and/or
(SEQID No. 38)
TTTCCTGTCTTTTCACATTTCAAACCTCCAGTT
for primer pair (iii)
(d)
(SEQ ID No. 39)
CAGCACCGACTACTACCAAGAGCTGC
for primer pair (iv)
(e)
(SEQ ID No. 40)
TTTCCTGTCTTTTCACATTTCAAACCTCCAGTT
for primer pair (v)
(f)
(SEQ ID No. 41)
ATGGCTGTCTGTCAGTGCCGCCGAA
for primer pair (vi)
(g)
(SEQ ID No. 42)
AGCACCGACTACTACCAAGAGCTGCA
and/or
(SEQ ID No. 43)
CAGCACCGACTACTACCAAGAGCTGC
for primer pair (vii)
(h)
(SEQ ID No. 44)
AGCACCGACTACTACCAAGAGCTGCA
for primer pair (viii)
(i)
(SEQ ID No. 45)
AGCACCGACTACTACCAAGAGCTGCA
and/or
(SEQ ID No. 46)
CAGCACCGACTACTACCAAGAGCTGC
for primer pair (ix)
(j)
(SEQ ID No. 47)
TTGACTCTGGCCTTCCGAGAAGGTAC
and/or
(SEQ ID No. 48)
CTTCCGAGAAGGTACCATCAATGTCCAC
for primer pair (x)
(k)
(SEQ ID No. 49)
CATCGCGCTGCTGGTGCTGGTCT
and/or
(SEQ ID No. 50)
TGTGCCATTTCCTTTCTCTGCCCAGTC
for primer pair (xi),
and
(l)
(SEQ ID No. 51)
CATCGCGCTGCTGGTGCTGGTCT
for primer pair (xii),
wherein the probes are labeled,
preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe.

51. The method according to claim 48, comprising following steps for determining long forms of Muc1 mRNA:
(a) isolating total RNA from the tissue sample,
(b) reverse transcribing the RNA into cDNA,
(c) performing Real-time PCR using one or more of the following primer pairs (1)-(3) for determining the long forms of Muc1 mRNA:
(1)
(SEQ ID No. 28)
CCACTCTGATACTCCTACCAC
and
(SEQ ID No. 29)
GAAAGAGACCCCAGTAGACAAC,
(2)
(SEQ ID No. 30)
CCTCCCCACCCATTTCACC
and
(SEQ ID No. 31)
CTGTAAGCACTGTGAGGAGC,
(3)
(SEQ ID No. 32)
CACTTCTGCCAACTTGTAGGG,
and
(SEQ ID No. 33)
CCCTACAAGTTGGCAGAAGTG,
and
(d) determining the expression level of long forms of Muc1 mRNA, optionally
wherein following probes are used:
(m)
(SEQ ID No. 52)
AGCCATAGCACCAAGACTGATGCCA
and/or
(SEQ ID No. 53)
ACCTCCTCTCACCTCCTCCAATCACA,
for primer pairs (1) to (3)
wherein the probes are labeled,
preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2.

52. The method of claim 48, wherein the expression level of
(i) total membrane-bound Muc1 mRNA,
(ii) the long forms of Muc 1 RNA,
(iii) Her-2 mRNA,
(iv) Estrogen Receptor 1 (ER1) isotype 1 mRNA, and
(v) Progesterone Receptor (PR) mRNA
is determined,
and wherein
(a) determining the expression level of human HER-2 with Real-time PCR is performed using one or more of the following primer pairs:
(1)
(SEQ ID No. 54
CGTTTGAGITCCATGCCCAATC
and
(SEQ ID No. 55)
TCCTCTGCTGITCACCTCTTG,
(2)
(SEQ ID No. 56)
CACCCACTCCCCTCTGAC
and
(SEQ ID No. 57)
CAGCAGITCTCCGCATCGTG
(3)
(SEQ ID No. 58)
GTGAAACCTGACCTCTCCTAC
and
(SEQ ID No. 59)
CAGCAGTCTCCGCATCGTG,
preferably wherein following probes are used:
(SEQ ID No. 60)
CTGCCTGITCCCTACAACTACCTTTCTAC,
for primer pair (1)
(SEQ ID No. 61)
ATCCTCATCAAGCGACGGCAGCAGAA,
for primer pair (2)
and/or
(SEQ ID No. 62)
AGCAGAGAGCCAGCCCTCTGACGTCCATC
for primer pair (3)
and
wherein the probes are labeled, preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2,
and/or
(b) determining the expression level of human Estrogen Receptor 1 (ESR1) isotype α with Real-time PCR is performed using the following primer pair:
(1)
(SEQ ID No. 63)
CCACTCAACAGCGTGTCTC
and
(SEQ ID No. 64)
GCTCGTTCTCCAGGTAGTAG,
preferably wherein following probe is used:
(SEQ ID No. 65)
TGTCGCCTTTCCTGCAGCCCCAC
and
wherein the probe is labeled, preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2,
and/or
(c) determining the expression level of human progesterone receptor (PR) with Real-time PCR is performed using one or more of the following primer pairs:
(1)
(SEQ ID No. 66)
CTTACAAAACTTCTTGATAACTTGC
and
(SEQ ID No. 68)
GGTTTCACCATCCCTGCCAA
(2)
(SEQ ID No. 67)
CTGTACTGCTTGAATACATTTATCC
and
(SEQ ID No. 68)
GGTTTCACCATCCCTGCCAA,
preferably wherein following probes are used:
(SEQ ID No. 69)
CTTCATCTGTACTGCTTGAATACATTTATCCAG,
for primer pair (1)
and/or
(SEQ ID No. 70)
ATGATGTCTGAAGTTATTGCTGCACAATTACCC
for primer pair (2)
and,
wherein the probes are labeled, preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2.

53. A method of treating an epithelial cancer patient, comprising
(i) administering a therapeutically effective amount of at least one agent for treating cancer,
(ii) performing the method according to claim 41, wherein in case an increase in ratio between the expression level of (b) and (c) at the second time point compared to the first time point is determined,
the administration a therapeutically effective amount of at least one agent for treating cancer is stopped, and/or
a therapeutically effective amount of at least one agent directed against Muc 1 is administered, and/or
a chemotherapeutic regime with high dosage and/or short intervals of chemotherapeutic agent(s) is administered.

54. A method of treating an epithelial cancer patient, comprising
(i) administering a therapeutically effective amount of at least one agent for treating cancer,
(ii) performing the method according to claim 42, wherein in case
(α) an increase in ratio between the expression level of (b) and (c) according to claim 41 at the second time point compared to the first time point is determined, and
(β) a decrease in expression level of Estrogen Receptor 1 (ER1) isotype 1 mRNA, and Progesterone receptor (PR) mRNA and optionally Her-2 mRNA at said second time point compared to said first time point is determined,
the administration a therapeutically effective amount of at least one agent for treating cancer is stopped, and/or
a therapeutically effective amount of at least one agent directed against Muc1 is administered, and/or
a chemotherapeutic regime with high dosage and/or short intervals of chemotherapeutic agent(s) is administered ,optionally,
wherein the at least agent for treating cancer is selected from a chemotherapeutic agent, an aromatase inhibitor, an hormone therapeutic agent, and an agent directed against HER-2, or
wherein the agent directed against Muc1 1 is an antibody or derivative thereof directed against Muc 1.

55. The method according to claim 42, wherein
(i) the at least one agent directed against HER-2 is Herceptin or a functionally active derivative thereof,
(ii) the aromatase inhibitor is an agent for hormone therapy, preferably at least one agent directed against Estrogen Receptor 1 (ESR1) isotype α or progesterone receptor (PR), even more preferably selected from tamoxifen, and a GnRH analogue.

56. A method of treating a tumor patient, comprising performing the method according to claim 43 or 47,
wherein in case
(x) an expression level of (b) higher than the expression level of (c) is determined by performing the method according to claim 43, or
(xx) an increase in the difference (a)-(b) between the expression level of (a) total membrane-bound Muc1 mRNA, or total membrane bound Muc1 protein and (b) the expression level of the long forms of Muc1 RNA, or the long forms of Muc1 protein at the later time point compared to the earlier time point is determined by performing the method according to claim 47,
a tumor therapy is initiated, or the amount or strength of an ongoing therapy is increased.

57. At least one pair of primers selected from (i) to (xv):
(i)
(SEQ ID No. 1)
CCTCCCCACCCATTTCACC
and
(SEQ ID No. 2)
CTGTAAGCACTGTGAGGAGC
(ii)
(SEQ ID No. 3)
CCTACCATCCTATGAGCGAG
and
(SEQ ID No. 4)
CTACAAGTTGGCAGAAGTG
(iii)
(SEQ ID No. 5)
CTACTGAGAAGAATGCTTTGTCTA
and
(SEQ ID No. 6)
GCCTGAACTTAATATTGGAGAGG
(iv)
(SEQ ID No. 7)
CTACTGAGAAGAATGCTTTTAATTCC
and
(SEQ ID No. 8)
GCCTGAACTTAATATTGGAGAGG
(v)
(SEQ ID No. 9)
CTACTGAGAAGAATGCTTTGTCTA
and
(SEQ ID No. 10)
CTCTTGGTAGTAGTCGGTGC
(vi)
(SEQ ID No. 11)
(CCAGCACCGACTACTACCAA
or
(SEQ ID No. 13))
CACCGACTACTACCAAGAGC
and
(SEQ ID No. 12)
CTCTTGGTAGTAGTCGGTGC
(vii)
(SEQ ID No. 14)
CTACTGAGAAGAATGCTTTTAATTCC
and
(SEQ ID No. 15)
GCCTGAACTTAATATTGGAGAGG
(viii)
(SEQ ID No. 16)
CTACTGAGAAGAATGCTTTTAATTCC
and
(SEQ ID No. 17)
(CGGCACTGACAGACAGCCAT
or
(SEQ ID No. 18))
GGCACTGACAGACAGCCATT
(ix)
(SEQ ID No. 19)
CTACTGAGAAGAATGCTTTTAATTCC
and
(SEQ ID No. 20)
CACCCCAGCCCCAGACATT
(x)
(SEQ ID No. 21)
CTACTGAGAAGAATGCTTTTTTGC
and
(SEQ ID No. 22)
AGGCTGCTTCCGTTTTATACTG
(xi)
(SEQ ID No. 23)
CCTCTCCAATATTAAGTTCAGTGA
and
(SEQ ID No. 24)
ACAGACAGCCAAGGCAATGAG
(xii)
(SEQ ID No. 25)
(CCTCTCCAATATTAAGTTCAGTCT
or
(SEQ ID No. 26))
CCTCTCCAATATTAAGTTCAGTC
and
(SEQ ID No. 27)
ACAGACAGCCAAGGCAATGAG
(xiii)
(SEQ ID No. 28)
CCACTCTGATACTCCTACCAC
and
(SEQ ID No. 29)
GAAAGAGACCCCAGTAGACAAC,
(xiv)
(SEQ ID No. 30)
CCTCCCCACCCATTTCACC
and
(SEQ ID No. 31)
CTGTAAGCACTGTGAGGAGC,
(xv)
(SEQ ID No. 32)
CACTTCTGCCAACTTGTAGGG,
and
(SEQ ID No. 33)
CCCTACAAGTTGGCAGAAGTG.

58. A kit comprising at least one pair of primers according to claim 57, and at least one probe, wherein the at least one probe is selected from:
(a)
(SEQ ID No. 34)
TGACACCGGGCACCCAGTCTCC
and/or
(SEQ ID No. 35)
CCACCATGACACCGGGCACCCA
for primer pair (i)
(b)
(SEQ ID No. 36)
TGCAGGTAATGGTGGCAGCAGCC
for primer pair (ii)
(c)
(SEQ ID No. 37)
AGCACCGACTACTACCAAGAGCTGCA
and/or
(SEQ ID No. 38)
TTTCCTGTCTTTTCACATTTCAAACCTCCAGTT
for primer pair (iii)
(d)
(SEQ ID No. 39)
CAGCACCGACTACTACCAAGAGCTGC
for primer pair (iv)
(e)
(SEQ ID No. 40)
TTTCCTGTCTTTTCACATTTCAAACCTCCAGTT
for primer pair (v)
(f)
(SEQ ID No. 41)
ATGGCTGTCTGTCAGTGCCGCCGAA
for primer pair (vi)
(g)
(SEQ ID No. 42)
AGCACCGACTACTACCAAGAGCTGCA
and/or
(SEQ ID No. 43)
CAGCACCGACTACTACCAAGAGCTGC
for primer pair (vii)
(h)
(SEQ ID No. 44)
AGCACCGACTACTACCAAGAGCTGCA
for primer pair (viii)
(i)
(SEQ ID No. 45)
AGCACCGACTACTACCAAGAGCTGCA
and/or
(SEQ ID No. 46)
CAGCACCGACTACTACCAAGAGCTGC
for primer pair (ix)
(j)
(SEQ ID No. 47)
TTGACTCTGGCCTTCCGAGAAGGTAC
and/or
(SEQ ID No. 48)
CTTCCGAGAAGGTACCATCAATGTCCAC
for primer pair (x)
(k)
(SEQ ID No. 49)
CATCGCGCTGCTGGTGCTGGTCT
and/or
(SEQ ID No. 50)
TGTGCCATTTCCTTTCTCTGCCCAGTC
for primer pair (xi)
(l)
(SEQ ID No. 51)
CATCGCGCTGCTGGTGCTGGTCT
for primer pair (xii)
(m)
(SEQ ID No. 52)
AGCCATAGCACCAAGACTGATGCCA
and/or
(SEQ ID No. 53)
ACCTCCTCTCACCTCCTCCAATCACA
for primer pair (xiii), (xiv) and (xv),
and wherein the probes are labeled,
preferably labeled with a fluorescent label and a quencher moiety,
more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2.

59. The kit according to claim 58, further comprising one or more of the following components (a) to (c):
(a) at least one pair of primers selected from (1) to (3):
(1)
(SEQ ID No. 54)
CGTTTGAGITCCATGCCCAATC
and
(SEQ ID No. 55)
TCCTCTGCTGITCACCTCTTG,
(2)
(SEQ ID No. 56)
CACCCACTCCCCTCTGAC
and
(SEQ ID No. 57)
CAGCAGITCTCCGCATCGTG
(3)
(SEQ ID No. 58)
GTGAAACCTGACCTCTCCTAC
and
(SEQ ID No. 59)
CAGCAGTCTCCGCATCGTG,
and optionally at least one probe selected from:
(SEQ ID No. 60)
CTGCCTGITCCCTACAACTACCTTTCTAC,
for primer pair (1)
(SEQ ID No. 61)
ATCCTCATCAAGCGACGGCAGCAGAA,
for primer pair (2)
and
(SEQ ID No. 62)
AGCAGAGAGCCAGCCCTCTGACGTCCATC,
for primer pair (3)
(b) the following primer pair:
(1)
(SEQ ID No. 63)
CCACTCAACAGCGTGTCTC
and
(SEQ ID No. 64)
GCTCGTTCTCCAGGTAGTAG,
and optionally following probe:
(SEQ ID No. 65)
TGTCGCCTTTCCTGCAGCCCCAC,
(c) at least one pair of primers selected from (1) and (2):
(1)
(SEQ ID No. 66)
CTTACAAAACTTCTTGATAACTTGC
and
(SEQ ID No. 68)
GGTTTCACCATCCCTGCCAA
(2)
(SEQ ID No. 67)
CTGTACTGCTTGAATACATTTATCC
and
(SEQ ID No. 68)
GGTTTCACCATCCCTGCCAA,
and optionally at least one probe selected from:
(SEQ ID No. 69)
CTTCATCTGTACTGCTTGAATACATTTATCCAG,
for primer pair (1)
and/or
(SEQ ID No. 70)
ATGATGTCTGAAGTTATTGCTGCACAATTACCC,
for primer pair (2)
wherein the optionally present probes are labeled, preferably labeled with a fluorescent label and a quencher moiety, more preferably wherein the fluorescent label is covalently attached to the nucleotide at the 5′ end of the probe, and the quencher moiety is attached to nucleotide at the 3′ end of the probe or to a nucleotide at least 15 nucleotides downstream of the 5′ end of the probe, even more preferably wherein the fluorescent label is ROX or FAM and the quencher moiety is BHQ2

60. The kit according to claim 58,
further comprising one, two, three or four of the following components (a) to (d):
(a) means for storing a tissue probe, in particular comprising a solution of 95% ethanol in water,
(b) means for isolating RNA from a tissue probe, in particular comprising a buffer for lysing tissue, a buffer for lysing cells, DNAse I and buffers for eluting RNA from a column and/or washing of RNA, preferably wherein the kit does not comprise paraffin,
(c) means for reverse transcribing RNA, in particular comprising a reverse transcriptase, a mixture of dNTPs, primers, and a reaction buffer, in particular wherein the primers are random sequence primers, Oligo(dT) primers or primers specific for the target sequence(s),
(d) means for performing Real-Time PCR, in particular comprising a DNA polymerase, a mixture of dNTPs, primers, and a reaction buffer, in particular wherein the primers are primers specific for the target sequence(s),
preferably wherein the kit comprises components (c), (c) and (d), or (b), (c) and (d).
